(19) United States
(12) Patent Application Publication
(10) Pub. No.: US 2004/0214255 A1
(43) Pub. Date: Oct. 28, 2004

(54) Compositions and methods for treating diabetes

(75) Inventors: Karen Heichman, Salt Lake City, UT (US); Paul Bartel, Salt Lake City, UT (US); Janice Sugiyama, Salt Lake City, UT (US)

Correspondence Address: MYRIAD GENETICS INC., LEGAL DEPARTMENT, 320 WAKARA WAY, SALT LAKE CITY, UT, 84108, US

(73) Assignee: Myriad Genetics, Incorporated, Salt Lake City, UT

(21) Appl. No.: 10/639,067

(22) Filed: Aug. 11, 2003

Related U.S. Application Data
(63) Continuation-in-part of application No. 09/556,941, filed on Apr. 21, 2000, now abandoned.
(60) Provisional application No. 60/130,389, filed on Apr. 22, 1999.
(60) Provisional application No. 60/140,693, filed on Jun. 24, 1999.
(60) Provisional application No. 60/156,947, filed on Sep. 30, 1999.
(60) Provisional application No. 60/163,073, filed on Nov. 2, 1999.
(60) Provisional application No. 60/168,378, filed on Dec. 2, 1999.
(60) Provisional application No. 60/168,376, filed on Dec. 2, 1999.

(51) Int. Cl. C12Q 1/54; C12Q 1/48

(52) U.S. Cl. 435/14; 435/15

(57) ABSTRACT

Protein complexes are provided comprising at least one interacting pair of proteins. The protein complexes are useful in screening assays for identifying compounds effective in modulating the protein complexes, and in treating and/or preventing diseases and disorders associated with the protein complexes and/or their constituent interacting members.

### RELATED U.S. APPLICATION

This application is a continuation-in-part of U.S. patent application Ser. No. 09/556,941 filed Apr. 21, 2000, which is related to U.S. provisional patent applications Serial No. 60/130,389, filed on 22 Apr. 1999, Serial No. 60/140,693, filed on 24 Jun. 1999, Serial No. 60/156,947, filed on 30 Sep. 1999, Serial No. 60/163,073, filed on 2 Nov. 1999, Serial No. 60/168,378, filed on 2 Dec. 1999 and Serial No. 60/168,376, filed on 2 Dec. 1999, each incorporated herein by reference.

### FIELD OF THE INVENTION

The present invention generally relates to methods and compositions for treating diseases, particularly to methods of using and modulating specific proteins and protein-protein interactions for purposes of drug screening and treatment of diseases.

### BACKGROUND OF THE INVENTION

Most drug discovery efforts today employ approaches to empirically identify small molecules that bind particular biological targets in vitro. These approaches generally involve “primary” high throughput screens designed to search vast combinatorial libraries of small molecules for “lead compounds” that often show a relatively weak affinity for the chosen target. However, once such lead compounds are identified in a “primary” high throughput screen, they can be subjected to further iterative rounds of chemical modification and testing by the process known to medicinal chemists as Structure Activity Relationship, or SAR. Generally, after several rounds of SAR-guided modification and in vitro screening, a set of optimized and related drug candidate compounds are subjected to the next phase of testing. This next phase generally involves the in vivo screening of the drug candidates in cell-based assays specifically designed to test the efficacy, toxicity and bioavailablity of the candidates. If the desired effects are obtained with reasonable dosages in these cell-based assays, animal studies are then initiated to determine whether the drug candidates have the desired activity in vivo. Only after careful study in well-defined animal models will a drug candidate be administered to humans in carefully regulated clinical trials.

The success or failure of a drug discovery program is heavily dependent on the identification and selection of druggable targets. In addition, once an appropriate drug target has been identified an efficient, preferably high throughput, screening assay needs to be established for drug screening against that particular drug target, which can be often be difficult to pragmatically achieve. The present invention provides novel drug targets for diseases such as diabetes and Alzheimer's disease and discloses screening assays for identifying potential drugs that may be effective against the diseases through modulating the drug targets.

### SUMMARY OF THE INVENTION

The present invention is based on the discovery of novel interactions between pairs of proteins described in the tables below. The specific interactions lead to the identification of desirable drug targets. In addition, the interactions can lead to the formation of protein complexes both in vitro and in vivo. The protein complexes formed under physiological conditions can mediate the functions and biological activities of the protein complexes and the individual protein members of the complexes. Thus, the protein-protein interactions provided in the tables and the protein complexes formed based on the protein-protein interactions are potential targets for the development of drugs useful in treating or preventing diseases and disorders associated with the protein complexes or interacting members thereof. In accordance with a first aspect of the present invention, isolated protein complexes are provided which are formed by the protein-protein interactions provided in the tables. In addition, homologues, derivatives, and fragments of the interacting proteins may also be used in forming protein complexes. In a specific embodiment, fragments of an interacting pair of proteins described in the tables containing regions responsible for the protein-protein interaction are used in forming a protein complex of the present invention. In another embodiment, at least one interacting protein member in a protein complex of the present invention is a fusion protein containing a protein in the tables or a homologue, derivative, or fragment thereof. In yet another embodiment, a protein complex is provided from a hybrid protein, which comprises, covalently linked together, directly or through a linker, a pair of interacting proteins described in the tables, or homologues, derivatives, or fragments thereof. In addition, nucleic acids encoding the hybrid protein are also provided.

In yet another aspect, the present invention also provides a method for making the protein complexes. The method includes the steps of providing the first protein and the second protein in the protein complexes of the present invention and contacting said first protein with said second protein. In addition, the protein complexes can be prepared by isolation or purification from tissues and cells or produced by recombinant expression of their protein members. The protein complexes can be incorporated into a protein microchip or microarray, which are useful in large-scale high throughput screening assays involving the protein complexes.

In accordance with a second aspect of the invention, antibodies are provided that are immunoreactive with a protein complex of the present invention. In one embodiment, an antibody is selectively immunoreactive with a protein complex of the present invention. In another embodiment, a bifunctional antibody is provided that has two different antigen binding sites, each being specific to a different interacting protein member in a protein complex of the present invention. The antibodies of the present invention can take various forms including polyclonal antibodies, monoclonal antibodies, chimeric antibodies, antibody fragments such as Fv fragments, single-chain Fv fragments (scFv), Fab′ fragments, and F(ab′)₂ fragments. Preferably, the antibodies are partially or fully humanized antibodies. The antibodies of the present invention can be readily prepared using procedures generally known in the art. For example, recombinant libraries such as phage display libraries and ribosome display libraries may be used to screen for antibodies with desirable specificities. In addition, various mutagenesis techniques such as site-directed mutagenesis and PCR diversification may be used in combination with the screening assays.

The present invention also provides detection methods for determining whether there is any aberration in a patient with respect to a protein complex formed by one or more interactions provided in accordance with this invention. In one embodiment, the method comprises detecting an aberrant concentration of the protein complexes of the present invention. Alternatively, the concentrations of one or more interacting protein members (at the protein or cDNA or mRNA level) of a protein complex of the present invention are measured. In addition, the cellular localization, or tissue or organ distribution of a protein complex of the present invention is determined to detect any aberrant localization or distribution of the protein complex. In another embodiment, mutations in one or more interacting protein members of a protein complex of the present invention can be detected. In particular, it is desirable to determine whether the interacting protein members have any mutations that will lead to, or are associated with, changes in the functional activity of the proteins or changes in their binding affinity to other interacting protein members in forming a protein complex of the present invention. In yet another embodiment, the binding constant of the interacting protein members of one or more protein complexes is determined. A kit may be used for conducting the detection methods of the present invention. Typically, the kit contains reagents useful in any of the above-described embodiments of the detection methods, including, e.g., antibodies specific to a protein complex of the present invention or interacting members thereof, and oligonucleotides selectively hybridizable to the cDNAs or mRNAs encoding one or more interacting protein members of a protein complex. The detection methods may be useful in diagnosing a disease or disorder such as diabestes, obesity, Alzheimer's disease and neurodegenerative disorders, staging the disease or disorder, or identifying a predisposition to the disease or disorder.

The present invention also provides screening methods for selecting modulators of a protein complex provided according to the present invention. Screening methods are also provided for selecting modulators of the individual interacting proteins. The compounds identified in the screening methods of the present invention can be useful in modulating the functions or activities of the individual interacting proteins, or the protein complexes of the present invention. They may also be effective in modulating the cellular processes involving the proteins and protein complexes, and in preventing or ameliorating diseases or disorders such as diabestes, obesity, Alzheimer's disease and neurodegenerative disorders.

Thus, test compounds may be screened in in vitro binding assays to identify compounds capable of binding a protein complex of the present invention, or its individual interacting protein members. The assays may include the steps of contacting the protein complex with a test compound and detecting the interaction between the interacting partners. In addition, in vitro dissociation assays may also be employed to select compounds capable of dissociating or destabilizing the protein complexes identified in accordance with the present invention. For example, the assays may entail (1) contacting the interacting members of a protein complex with each other in the presence of a test compound; and (2) detecting the interaction between the interacting members. An in vitro screening assay may also be used to identify compounds that trigger or initiate the formation of, or stabilize, a protein complex of the present invention.

In preferred embodiments, in vivo assays such as yeast two-hybrid assays and various derivatives thereof, preferably reverse two-hybrid assays, are utilized in identifying compounds that interfere with or disrupt the protein-protein interactions discovered according to the present invention. In addition, systems such as yeast two-hybrid assays are also useful in selecting compounds capable of triggering or initiating, enhancing or stabilizing the protein-protein interactions provided in the tables. In a specific embodiment, the screening method includes: (a) providing in a host cell a first fusion protein having a first protein of an interacting protein pair, or a homologue, derivative or fragment thereof, and a second fusion protein having the second protein of the pair, or a homologue, derivative or fragment thereof, wherein a DNA binding domain is fused to one of the first and second proteins while a transcription-activating domain is fused to the other of said first and second proteins; (b) providing in the host cell a reporter gene, wherein the transcription of the reporter gene is determined by the interaction between the first protein and the second protein; (c) allowing the first and second fusion proteins to interact with each other within the host cell in the presence of a test compound; and (d) determining the presence or absence of expression of the reporter gene.

In addition, the present invention also provides a method for selecting a compound capable of modulating a protein-protein interaction in accordance with the present invention, which comprises the steps of (1) contacting a test compound with an interacting protein disclosed in the tables, or a homologue, derivative or fragment thereof; and (2) determining whether said test compound is capable of binding said protein. In a preferred embodiment, the method further includes testing a selected test compound capable of binding said interacting protein for its ability to interfere with a protein-protein interaction according to the present invention involving said interacting protein, and optionally further testing the selected test compound for its ability to modulate cellular activities associated with said interacting protein and/or said protein-protein interaction.

The present invention also relates to a virtual screen method for providing a compound capable of modulating the interaction between the interacting members in a protein complex of the present invention. In one embodiment, the method comprises the steps of providing atomic coordinates defining a three-dimensional structure of a protein complex of the present invention, and designing or selecting, based on said atomic coordinates, compounds capable of interfering with the interaction between the interacting protein members of the protein complex. In another embodiment, the method comprises the steps of providing atomic coordinates defining a three-dimensional structure of an interacting protein described in the tables, and designing or selecting compounds capable of binding the interacting protein based on said atomic coordinates. In preferred embodiments, the method further includes testing a selected test compound for its ability to interfere with a protein-protein interaction provided in accordance with the present invention involving said interacting protein, and optionally further testing the selected test compound for its ability to modulate cellular activities associated with the interacting protein.

The present invention further provides a composition having two expression vectors. One vector contains a nucleic acid encoding a protein of an interacting protein pair according to the present invention, or a homologue, derivative or fragment thereof. Another vector contains the other protein of the interacting pair, or a homologue, derivative or fragment thereof. In addition, an expression vector is also provided containing (1) a first nucleic acid encoding one protein of an interacting protein pair of the present invention, or a homologue, derivative or fragment thereof; and (2) a second nucleic acid encoding the other protein of the interacting pair, or a homologue, derivative or fragment thereof.

Host cells are also provided comprising the expression vector(s). In addition, the present invention also provides a host cell having two expression cassettes. One expression cassette includes a promoter operably linked to a nucleic acid encoding one protein of an interacting pair of the present invention, or a homologue, derivative or fragment thereof. Another expression cassette includes a promoter operably linked to a nucleic acid encoding the other protein of the interacting pair, or a homologue, derivative or fragment thereof. Preferably, the expression cassettes are chimeric expression cassettes with heterologous promoters included.

In specific embodiments of the host cells or expression vectors, one of the two nucleic acids is linked to a nucleic acid encoding a DNA binding domain, and the other is linked to a nucleic acid encoding a transcription-activation domain, whereby two fusion proteins can be encoded.

In accordance with yet another aspect of the present invention, methods are provided for modulating the functions and activities of a protein complex of the present invention, or interacting protein members thereof. The methods may be used in treating or preventing diseases and disorders such as diabestes, obesity, Alzheimer's disease and neurodegenerative disorders. In one embodiment, the method comprises reducing a protein complex concentration and/or inhibiting the functional activities of the protein complex. Alternatively, the concentration and/or activity of one or more interacting members of a protein complex may be reduced or inhibited. Thus, the methods may include administering to a patient an antibody specific to a protein complex or an interacting protein member thereof, or an antisense oligo or ribozyme selectively hybridizable to a gene or mRNA encoding an interacting member of the protein complex. Also useful is a compound identified in a screening assay of the present invention capable of disrupting the interaction between two interacting members of a protein complex, or inhibiting the activities of an interacting member of the protein complex. In addition, gene therapy methods may also be used in reducing the expression of the gene(s) encoding one or more interacting protein members of a protein complex.

In another embodiment, the methods for modulating the functions and activities of a protein complex of the present invention or interacting protein members thereof comprise increasing the protein complex concentration and/or activating the functional activities of the protein complex. Alternatively, the concentration and/or activity of one or more interacting members of a protein complex of the present invention may be increased. Thus, one or more interacting protein members of a protein complex of the present invention may be administered directly to a patient. Or, exogenous genes encoding one or more protein members of a protein complex of the present invention may be introduced into a patient by gene therapy techniques. In addition, a patient needing treatment or prevention may also be administered with compounds identified in a screening assay of the present invention capable of triggering or initiating, enhancing or stabilizing a protein-protein interaction of the present invention.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIGS. 1-75 depict the structures of several embodiments of the siRNA compounds useful in reducing mRNA levels of the proteins in the tables.

### DETAILED DESCRIPTION OF THE INVENTION

### 1. Definitions

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

As used herein, the term “interacting” or “interaction” means that two protein domains, fragments or complete proteins exhibit sufficient physical affinity to each other so as to bring the two “interacting” protein domains, fragments or proteins physically close to each other. An extreme case of interaction is the formation of a chemical bond that results in continual and stable proximity of the two entities. Interactions that are based solely on physical affinities, although usually more dynamic than chemically bonded interactions, can be equally effective in co-localizing two proteins. Examples of physical affinities and chemical bonds include but are not limited to, forces caused by electrical charge differences, hydrophobicity, hydrogen bonds, van der Waals force, ionic force, covalent linkages, and combinations thereof. The state of proximity between the interaction domains, fragments, proteins or entities may be transient or permanent, reversible or irreversible. In any event, it is in contrast to and distinguishable from contact caused by natural random movement of two entities. Typically, although not necessarily, an “interaction” is exhibited by the binding between the interaction domains, fragments, proteins, or entities. Examples of interactions include specific interactions between antigen and antibody, ligand and receptor, enzyme and substrate, and the like.

An “interaction” between two protein domains, fragments or complete proteins can be determined by a number of methods. For example, an interaction is detectable by any commonly accepted approaches, including functional assays such as the two-hybrid systems. Protein-protein interactions can also be determined by various biophysical and biochemical approaches based on the affinity binding between the two interacting partners. Such biochemical methods generally known in the art include, but are not limited to, protein affinity chromatography, affinity blotting, immunoprecipitation, and the like. The binding constant for two interacting proteins, which reflects the strength or quality of the interaction, can also be determined using methods known in the art. See Phizicky and Fields, Microbiol. Rev., 59:94-123 (1995).

As used herein, the term “protein complex” means a composite unit that is a combination of two or more proteins formed by interaction between the proteins. Typically but not necessarily, a “protein complex” is formed by the binding of two or more proteins together through specific non-covalent binding affinities. However, covalent bonds may also be present between the interacting partners. For instance, the two interacting partners can be covalently crosslinked so that the protein complex becomes more stable.

The term “isolated protein complex” means a naturally occurring protein complex present in a composition or environment that is different from that found in its native or original cellular or biological environment in nature. An “isolated protein complex” may also be a protein complex that is not found in nature.

The term “protein fragment” as used herein means a polypeptide that represents a portion of a protein. When a protein fragment exhibits interactions with another protein or protein fragment, the two entities are said to interact through interaction domains that are contained within the entities.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

The term “isolated” when used in reference to nucleic acids (which include gene sequences) of this invention is intended to mean that a nucleic acid molecule is present in a form other than that found in nature.

Thus, an isolated nucleic acid can be a non-naturally occurring nucleic acid. For example, the term “isolated nucleic acid” encompasses “recombinant nucleic acid” which is used herein to mean a hybrid nucleic acid produced by recombinant DNA technology having the specified nucleic acid molecule covalently linked to one or more nucleic acid molecules that are not the nucleic acids naturally flanking the specified nucleic acid in the naturally existing chromosome. One example of recombinant nucleic acid is a hybrid nucleic acid encoding a fusion protein. Another example is an expression vector having the specified nucleic acid inserted in a vector.

The term “isolated nucleic acid” also encompasses nucleic acid molecules that are present in a form other than that found in its original environment in nature with respect to its association with other molecules. In this respect, an “isolated nucleic acid” as used herein means a nucleic acid molecule having only a portion of the nucleic acid sequence in the chromosome but not one or more other portions present on the same chromosome. Thus, an isolated nucleic acid present in a form other than that found in its original environment in nature with respect to its association with other molecules typically includes no more than 10 kb of the naturally occurring nucleic acid sequences that immediately flank the gene in the naturally existing chromosome or genomic DNA. Thus, the term “isolated nucleic acid” encompasses the term “purified nucleic acid,” which means an isolated nucleic acid in a substantially homogeneous preparation substantially free of other cellular components, other nucleic acids, viral materials, or culture medium, or chemical precursors or by-products associated with chemical reactions for chemical synthesis of nucleic acids. Typically, a “purified nucleic acid” can be obtained by standard nucleic acid purification methods, as will be apparent to skilled artisans.

An isolated nucleic acid can be in a vector. However, it is noted that an “isolated nucleic acid” as used herein is distinct from a clone in a conventional library such as a genomic DNA library or a cDNA library in that the clones in a library are still in admixture with almost all the other nucleic acids from a chromosome or a cell.

The term “high stringency hybridization conditions,” when used in connection with nucleic acid hybridization, means hybridization conducted overnight at 42 degrees C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, means hybridization conducted overnight at 37 degrees C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

As used herein, the term “homologue,” when used in connection with a first native protein or fragment thereof that is discovered, according to the present invention, to interact with a second native protein or fragment thereof, means a polypeptide that exhibits an amino acid sequence homology and/or structural resemblance to the first native interacting protein, or to one of the interacting domains of the first native protein such that it is capable of interacting with the second native protein. Typically, a protein homologue of a native protein may have an amino acid sequence that is at least 50%, preferably at least 75%, more preferably at least 80%, 85%, 86%, 87%, 88% or 89%, even more preferably at least 90%, 91%, 92%, 93% or 94%, and most preferably 95%, 96%, 97%, 98% or 99% identical to the native protein. Examples of homologues may be the ortholog proteins of other species including animals, plants, yeast, bacteria, and the like. Homologues may also be selected by, e.g., mutagenesis in a native protein. For example, homologues may be identified by site-specific mutagenesis in combination with assays for detecting protein-protein interactions, e.g., the yeast two-hybrid system described below, as will be apparent to skilled artisans apprised of the present invention. Other techniques for detecting protein-protein interactions include, e.g., protein affinity chromatography, affinity blotting, in vitro binding assays, and the like.

For the purpose of comparing two different nucleic acid or polypeptide sequences, one sequence (test sequence) may be described to be a specific “percent identical to” another sequence (reference sequence) in the present disclosure. In this respect, the percentage identity is determined by the algorithm of Karlin and Altschul, Proc. Natl. Acad. Sci. USA, 90:5873-5877 (1993), which is incorporated into various BLAST programs. Specifically, the percentage identity is determined by the “BLAST 2 Sequences” tool, which is available at http://www.ncbi.nlm.nih.gov/gorf/bl2.html. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter).

The term “derivative,” when used in connection with a first native protein (or fragment thereof) that is discovered, according to the present invention, to interact with a second native protein (or fragment thereof), means a modified form of the first native protein prepared by modifying the side chain groups of the first native protein without changing the amino acid sequence of the first native protein. The modified form, i.e., the derivative should be capable of interacting with the second native protein. Examples of modified forms include glycosylated forms, phosphorylated forms, myristylated forms, ribosylated forms, ubiquitinated forms, and the like. Derivatives also include hybrid or fusion proteins containing a native protein or a fragment thereof. Methods for preparing such derivative forms should be apparent to skilled artisans. The prepared derivatives can be easily tested for their ability to interact with the native interacting partner using techniques known in the art, e.g., protein affinity chromatography, affinity blotting, in vitro binding assays, yeast two-hybrid assays, and the like.

The term “antibody” as used herein encompasses both monoclonal and polyclonal antibodies that fall within any antibody classes, e.g., IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The term “selectively immunoreactive” as used herein means that an antibody is reactive thus binds to a specific protein or protein complex, but not other similar proteins or fragments or components thereof.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes, the ability to maintain the form of a protein complex), antigenicity and immunogenicity, enzymatic activities, etc. Such activities may be detected or assayed by any of a variety of suitable methods as will be apparent to skilled artisans.

The term “compound” as used herein encompasses all types of organic or inorganic molecules, including but not limited proteins, peptides, polysaccharides, lipids, nucleic acids, small organic molecules, inorganic compounds, and derivatives thereof.

As used herein, the term “interaction antagonist” means a compound that interferes with, blocks, disrupts or destabilizes a protein-protein interaction; blocks or interferes with the formation of a protein complex; or destabilizes, disrupts or dissociates an existing protein complex.

The term “interaction agonist” as used herein means a compound that triggers, initiates, propagates, nucleates, or otherwise enhances the formation of a protein-protein interaction; triggers, initiates, propagates, nucleates, or otherwise enhances the formation of a protein complex; or stabilizes an existing protein complex.

Unless otherwise specified, the term “GLUT4” as used herein means the human GLUT4 protein. The usage for naming other proteins should be similar unless otherwise specified in the present disclosure.

### 2. Protein Complexes

Novel protein-protein interactions have been discovered. The protein-protein interactions are provided in the tables below. Specific fragments capable of conferring interacting properties on the interacting proteins have also been identified. The GenBank reference numbers for the cDNA sequences encoding the interacting proteins are also noted in the tables.

TABLE 1
Binding Regions of Glucose Transporter 4 and
Clone C-193 (CARP)

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| Glucose | 463 | 510 | Clone C-193 | 21 | 302 |
| Transporter 4 | | | (GenBank | | |
| (Swiss Protein | | | Accession No. | | |
| Accession No. | | | X83703) | | |
| P14672) | | | | | |

TABLE 2
Binding Regions of Glucose Transporter 1 and
DRAL(FHL2)

| Protein 1 | | | Protein 2 | | |
|---|---|---|---|---|---|
| Name and Swiss | Amino Acid | | Name and | Amino Acid | |
| Protein | Coordinates | | Swiss Protein | Coordinates | |
| Accession No. | Start | Stop | Accession No. | Start | Stop |
| Glucose | 448 | 468 | DRAL(FHL2) | 1 | 280 |
| Transporter 1 | | | (Swiss Protein | | |
| (Swiss Protein | | | Accession No. | | |
| Accession No. | | | Q13229) | | |
| P11166) | | | | | |

TABLE 3
Binding Regions of Glucose Transporter 1 and
Myosin Heavy Chain

| Protein 1 | | | Protein 2 | | |
|---|---|---|---|---|---|
| Name and Swiss | Amino Acid | | Name and | Amino Acid | |
| Protein | Coordinates | | Swiss Protein | Coordinates | |
| Accession No. | Start | Stop | Accession No. | Start | Stop |
| Glucose | 448 | 493 | Myosin Heavy | 1589 | 1909 |
| Transporter 1 | | | Chain (Swiss | | |
| (Swiss Protein | | | Protein | | |
| Accession No. | | | Accession | | |
| P11166) | | | No. P13533) | | |

TABLE 4
Binding Regions of Glucose Transporter 1 and Human Sperm
Surface Protein

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| Glucose | 448 | 493 | Human Sperm | 412 | 526 |
| Transporter 1 | | | Surface Protein | | |
| (Swiss Protein | | | (GenBank | | |
| Accession No. | | | Accession No. | | |
| P11166) | | | X91879) | | |

TABLE 5
Binding Regions of O-linked N-acetylglucosaminyltransferase
(OGTase) and Myosin Heavy Chain

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | Myosin Heavy | 1656 | 1830 |
| (GenBank | | | Chain | | |
| Accession No. | | | (GenBank | | |
| U77413) | | | Accession No. | | |
| | | | AF111785) | | |

TABLE 6
Binding Regions of Insulin-Regulated Membrane-Spanning
Aminopeptidase (IRAP) and 14-3-3 Beta

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| IRAP (GenBank | 129 | 473 | 14-3-3 Beta | 97 | 236 |
| Accession No. | | | (Swiss Protein | | |
| U62768) | | | Accession No. | | |
| | | | P31946) | | |

TABLE 7
Binding Regions of Insulin-Regulated Membrane-Spanning
Aminopeptidase (IRAP) and Human Sperm Surface Protein (HSS)

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| IRAP (GenBank | 1 | 273 | Human Sperm | 408 | 647 |
| Accession No. | | | Surface Protein | | |
| U62768) | | | (GenBank | | |
| | | | Accession No. | | |
| | | | X91879) | | |

TABLE 8
Binding Regions of PI-3 Kinase p110 subunit and Complement
Protein C4

| Protein 1 | | | Protein 2 | | |
|---|---|---|---|---|---|
| Name and Swiss | Amino Acid | | Name and | Amino Acid | |
| Protein | Coordinates | | Swiss Protein | Coordinates | |
| Accession No. | Start | Stop | Accession No. | Start | Stop |
| PI-3 kinase p110 | 1 | 300 | Complement | 1056 | 1277 |
| subunit (Swiss | | | Protein C4 | | |
| Protein | | | (Swiss Protein | | |
| Accession No. | | | Accession No. | | |
| P42338) | | | P01028) | | |

TABLE 9
Binding Regions of PI-3 Kinase p110 subunit and Tenascin XB

| Protein 1 | | | Protein 2 | | |
|---|---|---|---|---|---|
| Name and Swiss | Amino Acid | | Name and | Amino Acid | |
| Protein | Coordinates | | Swiss Protein | Coordinates | |
| Accession No. | Start | Stop | Accession No. | Start | Stop |
| PI-3 kinase p110 | 1 | 300 | Tenascin XB | 3573 | 3787 |
| subunit (Swiss | | | (Swiss Protein | | |
| Protein | | | Accession No. | | |
| Accession No. | | | P78530) | | |
| P42338) | | | | | |

TABLE 10
Binding Regions of PI-3 Kinase p110 subunit and Alpha Acid
Glucosidase (GAA)

| Protein 1 | | | Protein 2 | | |
|---|---|---|---|---|---|
| Name and Swiss | Amino Acid | | Name and | Amino Acid | |
| Protein | Coordinates | | Swiss Protein | Coordinates | |
| Accession No. | Start | Stop | Accession No. | Start | Stop |
| PI-3 kinase p110 | 1 | 300 | GAA (Swiss | 513 | 953 |
| subunit (Swiss | | | Protein | | |
| Protein | | | Accession No. | | |
| Accession No. | | | P10253) | | |
| P42338) | | | | | |

TABLE 11
Binding Regions of C-myc Binding Protein (MM-1) and C-Nap1

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| MM-1 (Swiss | 27 | 175 | C-Nap1 | 279 | 426 |
| Protein | | | (GenBank | | |
| Accession No. | | | Accession No. | | |
| Q99471) | | | AF049105) | | |

TABLE 12
Binding Regions of C-myc Binding Protein (MM-1) and Beta
Spectrin

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| MM-1 (Swiss | 27 | 175 | Beta Spectrin | 1545 | 1789 |
| Protein | | | (Swiss Protein | | |
| Accession No. | | | Accession No. | | |
| Q99471) | | | Q01082) | | |

TABLE 13
Binding Regions of C-myc Binding Protein (MM-1) and
KIAA0477

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| MM-1 (Swiss | 27 | 175 | KIAA0477 | 597 | 850 |
| Protein | | | (GenBank | | |
| Accession No. | | | Accession No. | | |
| Q99471) | | | AB007946) | | |

TABLE 14
Binding Regions of Dynamin and Clathrin Assembly Protein
(CALM)

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| Dynamin (Swiss | 250 | 700 | CALM | 267 | 484 |
| Protein | | | (GenBank | | |
| Accession No. | | | Accession No. | | |
| Q05193) | | | U45976) | | |

TABLE 15
Binding Regions of Dynamin and Proteosome Activator Subunit
Psme3 (Psme3)

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| Dynamin (Swiss | 250 | 700 | Psme3 | 72 | 254 |
| Protein | | | (GenBank | | |
| Accession No. | | | Accession No. | | |
| Q05193) | | | U11292) | | |

TABLE 16
Binding Regions of Nef-Associated Factor 1 beta (Naf1b) and I-
TRAF

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| Naf1b (GenBank | 160 | 500 | I-TRAF | 17 | 424 |
| Accession No. | | | (GenBank | | |
| Q05193) | | | Accession No. | | |
| | | | U59863) | | |

TABLE 17
Binding Regions of Akt kinase 1 (Akt1) and NuMA1

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| Akt1 (Swiss | 1 | 118 | NuMA1 | 185 | 361 |
| Protein | | | (GenBank | | |
| Accession No. | | | Accession No. | | |
| P31749) | | | Z11584) | | |

TABLE 18
Binding Regions of Akt kinase 2 (Akt2) and NuMA1

| Protein 1 | | | Protein 2 | | |
|---|---|---|---|---|---|
| Name and Swiss | Amino Acid | | Name and | Amino Acid | |
| Protein | Coordinates | | GenBank | Coordinates | |
| Accession No. | Start | Stop | Accession No. | Start | Stop |
| Akt2 (Swiss | 1 | 152 | NuMA1 | 98 | 365 |
| Protein | | | (GenBank | | |
| Accession No. | | | Accession No. | | |
| P31751) | | | Z11584) | | |

TABLE 19
Binding Regions of Akt kinase 2 (Akt2) and BAP31

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| Akt2 (GenBank | 276 | 408 | BAP31 | 111 | 247 |
| Accession No. | | | (GenBank | | |
| M95936) | | | Accession No. | | |
| | | | NM00574) | | |

TABLE 20
Binding Regions of Akt kinase 2 (Akt2) and Beta Adaptin

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| Akt2 (GenBank | 276 | 408 | Beta Adaptin | 214 | 594 |
| Accession No. | | | (GenBank | | |
| M95936) | | | Accession No. | | |
| | | | M95936) | | |

TABLE 21
Binding Regions of OGTase and Desmin

| Protein 1 | | | Protein 2 | | |
|---|---|---|---|---|---|
| Name and | Amino Acid | | Name and Swiss | Amino Acid | |
| GenBank | Coordinates | | Protein | Coordinates | |
| Accession No. | Start | Stop | Accession No. | Start | Stop |
| OGTase | 250 | 361 | Desmin (Swiss | 219 | 449 |
| (GenBank | | | Protein Accession | | |
| Accession No. | | | No. P17661) | | |
| U77413) | | | | | |

TABLE 22
Binding Regions of OGTase and Alpha-karyopherin

| Protein 1 | | | Protein 2 | | |
|---|---|---|---|---|---|
| Name and | Amino Acid | | Name and Swiss | Amino Acid | |
| GenBank | Coordinates | | Protein | Coordinates | |
| Accession No. | Start | Stop | Accession No. | Start | Stop |
| OGTase | 250 | 361 | Alpha- | 152 | 444 |
| (GenBank | | | karyopherin | | |
| Accession No. | | | (Swiss Protein | | |
| U77413) | | | Accession No. | | |
| | | | P52294) | | |

TABLE 23
Binding Regions of OGTase and Glutaminyl tRNA Synthetase

| Protein 1 | | | Protein 2 | | |
|---|---|---|---|---|---|
| Name and | Amino Acid | | Name and Swiss | Amino Acid | |
| GenBank | Coordinates | | Protein | Coordinates | |
| Accession No. | Start | Stop | Accession No. | Start | Stop |
| OGTase | 250 | 361 | Glutaminyl tRNA | 36 | 161 |
| (GenBank | | | Synthetase (Swiss | | |
| Accession No. | | | Protein Accession | | |
| U77413) | | | No. P47897) | | |

TABLE 24
Binding Regions of OGTase and BAB55262

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | BAB55262 | 640 | 958 |
| (GenBank | | | (GenBank | | |
| Accession No. | | | Accession No. | | |
| U77413) | | | AF131780) | | |

TABLE 25
Binding Regions of PTP1b VAP-A and

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| PTP1b (Swiss | 280 | 436 | VAP-A | 3 | 242 |
| Protein | | | (GenBank | | |
| Accession No. | | | Accession No. | | |
| P18031) | | | AF086627) | | |

TABLE 26
Binding Regions of Rab4 and Alpha-catenin-like Protein

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| Rab4 (Swiss | 1 | 214 | Alpha-catenin- | 510 | 648 |
| Protein | | | like Protein | | |
| Accession No. | | | (GenBank | | |
| P20338) | | | Accession No. | | |
| | | | U97067) | | |

TABLE 27
Binding Regions of Rab4 and Rab2

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| Rab4 (Swiss | 1 | 214 | Rab2 (Swiss | 27 | 177 |
| Protein | | | Protein Accession | | |
| Accession No. | | | No. P08886) | | |
| P20338) | | | | | |

TABLE 28
Binding Regions of Glucose Transporter 4 and PN7065

| Protein 1 | | Protein 2 | |
|---|---|---|---|
| Name and | Amino Acid | | Amino Acid |
| Swiss Protein | Coordinates | | Coordinates |

| Accession No. | Start | Stop | Name | Start | Stop |
|---|---|---|---|---|---|
| Glucose | 463 | 510 | PN7065 | 276 | 487 |
| Transporter 4 | | | | | |
| (Swiss Protein | | | | | |
| Accession No. | | | | | |
| P14672) | | | | | |

TABLE 29
Binding Regions of Glucose Transporter 4 and PN7386

| Protein 1 | | Protein 2 | |
|---|---|---|---|
| Name and | Amino Acid | | Amino Acid |
| Swiss Protein | Coordinates | | Coordinates |

| Accession No. | Start | Stop | Name | Start | Stop |
|---|---|---|---|---|---|
| Glucose | 478 | 510 | PN7386 | 1 | 319 |
| Transporter 4 | | | | | |
| (Swiss Protein | | | | | |
| Accession No. | | | | | |
| P14672) | | | | | |

TABLE 30
Binding Regions of OGTase and PN6931

| Protein 1 | | Protein 2 | |
|---|---|---|---|
| Name and | Amino Acid | | Amino Acid |
| GenBank | Coordinates | | Coordinates |

| Accession No. | Start | Stop | Name | Start | Stop |
|---|---|---|---|---|---|
| OGTase | 250 | 361 | PN6931 | 11 | 292 |
| (GenBank | | | | | |
| Accession No. | | | | | |
| U77413) | | | | | |

TABLE 31
Binding Regions of Naf1b and PN7582

| Protein 1 | | Protein 2 | |
|---|---|---|---|
| Name and | Amino Acid | | Amino Acid |
| GenBank | Coordinates | | Coordinates |

| Accession No. | Start | Stop | Name | Start | Stop |
|---|---|---|---|---|---|
| (GenBank | 41 | 486 | PN7582 | 1 | 88 |
| Accession No. | | | | | |
| AJ011896) | | | | | |

TABLE 32
Binding Regions of OGTase and Talin

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | Talin (GenBank | 812 | 1148 |
| (GenBank | | | Accession No. | | |
| Accession No. | | | AF078828) | | |
| U77413) | | | | | |

TABLE 33
Binding Regions of OGTase and MOP2

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| OGTase | 250 | 361 | MOP2 (Swiss | 397 | 546 |
| (GenBank | | | Protein Accession | | |
| Accession No. | | | No. Q99814) | | |
| U77413) | | | | | |

TABLE 34
Binding Regions of OGTase and KIAA0443

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | KIAA0443 | 1156 | 1369 |
| (GenBank | | | (GenBank | | |
| Accession No. | | | Accession No. | | |
| U77413) | | | AB007903) | | |

TABLE 35
Binding Regions of OGTase and EGR1

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| OGTase | 250 | 361 | EGR1(Swiss | 415 | 544 |
| (GenBank | | | Protein Accession | | |
| Accession No. | | | No. P18146) | | |
| U77413) | | | | | |

TABLE 36
Binding Regions of OGTase and Dynamin II

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| OGTase | 250 | 361 | Dynamin II | 592 | 744 |
| (GenBank | | | (Swiss Protein | | |
| Accession No. | | | Accession No. | | |
| U77413) | | | P50570) | | |

TABLE 37
Binding Regions of OGTase and INT-6

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | INT-6 (GenBank | 452 | 942 |
| (GenBank | | | Accession No. | | |
| Accession No. | | | U62962) | | |
| U77413) | | | | | |

TABLE 38
Binding Regions of OGTase and HSPC028

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | HSPC028 | 20 | 148 |
| (GenBank | | | (GenBank | | |
| Accession No. | | | Accession No. | | |
| U77413) | | | AF083246) | | |

TABLE 39
Binding Regions of OGTase and BAP31

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| OGTase | 250 | 361 | BAP31 (Swiss | 116 | 215 |
| (GenBank | | | Protein Accession | | |
| Accession No. | | | No. P51572) | | |
| U77413) | | | | | |

TABLE 40
Binding Regions of OGTase and Interferon-Ind Protein

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| OGTase | 250 | 361 | Interferon-Ind | −37 | 91 |
| (GenBank | | | Protein (Swiss | | |
| Accession No. | | | Protein Accession | | |
| U77413) | | | No. P09913) | | |

TABLE 41
Binding Regions of Glut4 and Beta-catenin

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| Glut4 (Swiss | 463 | 510 | Beta-catenin | 579 | 782 |
| Protein | | | (Swiss Protein | | |
| Accession No. | | | Accession No. | | |
| P14672) | | | P35222) | | |

TABLE 42
Binding Regions of Glut4 and Alpha-SNAP

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| Glut4 (Swiss | 463 | 510 | Alpha-SNAP | 36 | 241 |
| Protein | | | (Swiss Protein | | |
| Accession No. | | | Accession No. | | |
| P14672) | | | P54920) | | |

TABLE 43
Binding Regions of Glut4 and MAPKKK6

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| Glut4 (Swiss | 463 | 510 | MAPKKK6 | 824 | 1012 |
| Protein | | | (GenBank | | |
| Accession No. | | | Accession No. | | |
| P14672) | | | AF100318) | | |

TABLE 44
Binding Regions of Glut4 and Tropomyosin 3

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| Glut4 (Swiss | 434 | 510 | Tropomyosin 3 | 171 | 286 |
| Protein | | | (Swiss Protein | | |
| Accession No. | | | Accession No. | | |
| P14672) | | | P06753) | | |

TABLE 45
Binding Regions of Insulin-Regulated Membrane-Spanning
Aminopeptidase (IRAP) and SG2NA

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| IRAP | 750 | 1026 | SG2NA(Swiss | 623 | 713 |
| (GenBank | | | Protein Accession | | |
| Accession No. | | | No. P70483) | | |
| U62768) | | | | | |

TABLE 46
Binding Regions of Insulin-Regulated Membrane-Spanning
Aminopeptidase (IRAP) and Slap-2

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| IRAP | 1 | 273 | (GenBank | 236 | 453 |
| (GenBank | | | Accession No. | | |
| Accession No. | | | AF100750) | | |
| U62768) | | | | | |

TABLE 47
Binding Regions of OGTase and 14-3-3 Epsilon

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| OGTase | 250 | 450 | 14-3-3 Epsilon | 21 | 232 |
| (GenBank | | | (Swiss Protein | | |
| Accession No. | | | Accession No. | | |
| U77413) | | | P29360) | | |

TABLE 48
Binding Regions of PI-3K85 and Chromagranin

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| PI-3K85 (Swiss | 1 | 250 | Chromagranin | 2 | 322 |
| Protein | | | (Swiss Protein | | |
| Accession No. | | | Accession No. | | |
| P27986) | | | P13521) | | |

TABLE 49
Binding Regions of PI-3K85 and SLP-76

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| PI-3K85 (Swiss | 320 | 440 | SLP-76 | 291 | 486 |
| Protein | | | (GenBank | | |
| Accession No. | 600 | 724 | Accession No. | 291 | 477 |
| P27986) | | | U20158) | | |

TABLE 50
Binding Regions of PI-3K85 and 14-3-3-Zeta

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| PI-3K85 (Swiss | 1 | 50 | 14-3-3-Zeta | 79 | 246 |
| Protein | | | (Swiss Protein | | |
| Accession No. | | | Accession No. | | |
| P27986) | | | P29213) | | |

TABLE 51
Binding Regions of PI-3K85 and 14-3-3-Eta

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| PI-3K85 (Swiss | 1 | 50 | 14-3-3-Eta (Swiss | 91 | 246 |
| Protein | | | Protein Accession | | |
| Accession No. | | | No. Q04917) | | |
| P27986) | | | | | |

TABLE 52
Binding Regions of PI-3K85 and TACC2

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| PI-3K85 (Swiss | 600 | 725 | TACC2 | 350 | 600 |
| Protein | | | (GenBank | | |
| Accession No. | | | Accession No. | | |
| P27986) | | | AF095791) | | |

TABLE 53
Binding Regions of GLUT4 and MM-1

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| GLUT4 (Swiss | 463 | 510 | MM-1 (GenBank | 27 | 168 |
| Protein | | | Accession No. | | |
| Accession No. | | | D89667) | | |
| P14672) | | | | | |

TABLE 54
Binding Regions of GLUT1 and KIAA0144

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| GLUT1 (Swiss | 203 | 275 | KIAA0144 | 691 | 965 |
| Protein | | | (GenBank | | |
| Accession No. | | | Accession No. | | |
| P11166) | | | D63478) | | |

TABLE 55
Binding Regions of GLUT1 and Dynamin

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| GLUT1 (Swiss | 463 | 492 | Dynamin | 104 | 365 |
| Protein | | | (Genbank | | |
| Accession No. | | | Accession No. | | |
| P11166) | | | L07807) | | |

TABLE 56
Binding Regions of GLUT1 and Type I Transmembrane Receptor
Seizure-Related Protein (PSK-1)

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| GLUT1 (Swiss | 463 | 492 | PSK-1 (GenBank | 28 | 402 |
| Protein | | | Accession No. | | |
| Accession No. | | | AF131749) | | |
| P11166) | | | | | |

TABLE 57
Binding Regions of IRAP and VAP-A

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| IRAP | 1 | 273 | VAP-A | 3 | 243 |
| (GenBank | | | (GenBank | | |
| Accession No. | | | Accession No. | | |
| U62768) | | | AF086627) | | |

TABLE 58
Binding Regions of OGTase and NAF1a

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | NAF1a | 41 | 486 |
| (GenBank | | | (GenBank | | |
| Accession No. | | | Accession No. | | |
| U77413) | | | AJ011895) | | |

TABLE 59
Binding Regions of OGTase and Alpha-2-Catenin

| Protein 1 | | | Protein 2 | | |
|---|---|---|---|---|---|
| Name and | Amino Acid | | Name and | Amino Acid | |
| GenBank | Coordinates | | GenBank | Coordinates | |
| Accession No. | Start | Stop | Accession No. | Start | Stop |
| OGTase | 250 | 361 | Alpha-2-Catenin | 366 | 649 |
| (GenBank | | | (GenBank | | |
| Accession No. | | | Accession No. | | |
| U77413) | | | M94151) | | |

TABLE 60
Binding Regions of PI-3K110 and TRIP15

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| PI-3K110 | 1 | 300 | TRIP15 | 24 | 233 |
| (Swiss Protein | | | (GenBank | | |
| Accession No. | | | Accession No. | | |
| P42338) | | | L40388) | | |

TABLE 61
Binding Regions of GLUT4 and 14-3-3-Zeta

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| GLUT4 (Swiss | 463 | 483 | 14-3-3-Zeta | 1 | 210 |
| Protein | 463 | 510 | (Swiss Protein | 1 | 200 |
| Accession No. | | | Accession No. | | |
| P14672) | | | P29213) | | |

TABLE 62
Binding Regions of GLUT4 and KIAA0282

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| GLUT4 (Swiss | 463 | 510 | KIAA0282 | 227 | 380 |
| Protein | | | (GenBank | | |
| Accession No. | | | Accession No. | | |
| P14672) | | | D87458) | | |

TABLE 63
Binding Regions of IRAP and PTPZ

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| IRAP | 1 | 273 | PTPZ (Swiss | 1470 | 1829 |
| (GenBank | | | Protein Accession | | |
| Accession No. | | | No. P23471) | | |
| U62768) | | | | | |

TABLE 64
Binding Regions of IRAP and β-Spectrin

| Protein 1 | | | Protein 2 | | |
|---|---|---|---|---|---|
| Name and | Amino Acid | | Name and | Amino Acid | |
| GenBank | Coordinates | | Swiss Protein | Coordinates | |
| Accession No. | Start | Stop | Accession No. | Start | Stop |
| IRAP | 750 | 1026 | β-Spectrin (Swiss | 1592 | 2096 |
| (GenBank | | | Protein Accession | | |
| Accession No. | | | No. Q01082) | | |
| U62768) | | | | | |

TABLE 65
Binding Regions of PP5 and HSP89

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| PP5 (Swiss | 1 | 150 | HSP89 (Swiss | 537 | 700 |
| Protein | | | Protein Accession | | |
| Accession No. | | | No. P07900) | | |
| P53041) | | | | | |

TABLE 66
Binding Regions of PP5 and Tankyrase

| Protein 1 | | | Protein 2 | | |
|---|---|---|---|---|---|
| Name and | Amino Acid | | Name and | Amino Acid | |
| Swiss Protein | Coordinates | | GenBank | Coordinates | |
| Accession No. | Start | Stop | Accession No. | Start | Stop |
| PP5 (Swiss | 1 | 150 | Tankyrase | 603 | 725 |
| Protein | | | (GenBank | | |
| Accession No. | | | Accession No. | | |
| P53041) | | | AF082557) | | |

TABLE 67
Binding Regions of PI-3K110 and APP

| Protein 1 | | | Protein 2 | | |
|---|---|---|---|---|---|
| Name and | Amino Acid | | Name and | Amino Acid | |
| Swiss Protein | Coordinates | | Swiss Protein | Coordinates | |
| Accession No. | Start | Stop | Accession No. | Start | Stop |
| PI-3K110 | 1 | 300 | APP (Swiss | 432 | 594 |
| (Swiss Protein | | | Protein Accession | | |
| Accession No. | | | No. P05067) | | |
| P42338) | | | | | |

### 2.1. Biological Significance

One of the key questions which must be answered in order to understand and treat non-insulin dependent diabetes mellitus (NIDDM) is how glucose uptake is regulated in the cell. It is known that in adipose and muscle tissues there exists an insulin-regulated membrane-spanning glucose transporter called Glut4 that shuttles between the interior of the cell to the plasma membrane by means of vesicle-mediated endocytosis and exocytosis (Garvey et al., 1998; Haruta et al., 1995; Pessin et al., 1999). Fat and muscle cells from diabetic patients appear to be defective in this type of regulated glucose transport (Zierath et al., 1998). The mechanisms by which this transport to the plasma membrane and return back to the interior of the cell are regulated are not well understood. Toward this end, there has been much interest in analyzing the Glut4 protein and the additional factors that participate in glucose uptake in fat and muscle cells. By understanding the regulation of glucose transport in normal cells, medical interventions can be discovered that would serve to increase sugar uptake in the cells of diabetic patients.

One approach to understanding how glucose transport is achieved has been to use the Glut4 molecule as a molecular bait in the search for other molecules which can interact with it. In this way, it might be possible to find ways to influence the function of Glut4 and perhaps find ways to cause it to go to the plasma membrane and subsequently remove glucose from the outside of the cell and bring it into the interior. We have been able to demonstrate that clone C-193 (known as CARP in other systems) can bind to Glut4. CARP is a cytokine-inducible gene that reportedly acts as a negative regulator of cardiac-specific genes (Jeyaseelan et al., 1997). This interaction may serve as a tie between heart disease and diabetes.

We have also been able to detect the interaction of Glut4 with two novel proteins that have been named PN7065 and PN7386. PN7065 is 99% identical to the human homolog of mouse SNF1-like kinase. PN7065 is also 80% identical to rat salt-inducible protein kinase (SIK), and 52% identical to mouse SIK2, but is most identical over the N-terminal half of the protein. All of these proteins appear to belong to the AMP-activated protein kinase family. AMPK is an energy sensor that responds to such environmental cues as hypoxia (Hardie et al., 1997, Eur. J. Biochem. 246: 259-273), fuel deprivation (Salt et al., 1998, Biochem. J. 335: 533-539), and exercise (Vavvas et al., 1997 J. Biol. Chem. 272: 13255-13261), and causes increased glucose uptake in an insulin-independent manner. PN7065 is also 40% identical to AMPK-alpha1 over the kinase region.

An interesting aspect of the interaction between Glut4 and PN7065 is the roles that PN7065 may play in diabetes based on its homologies to SIK2 and AMPK. SIK2 is specifically expressed in adipose, phosphorylates IRS 1 on a serine residue, and is more highly expressed in db/db mice (J. Biol. Chem. May 2003, 278: 18440). SIK2 and/or PN7065 may therefore modulate insulin signaling and be involved in insulin resistance. In a recent paper it was shown that glucose autoregulates its uptake in an AMPK-dependent manner (Itani et al., 2003, Diabetes, 52: 1635-1640). Using EDL rat skeletal muscle preparations, Itani et al, showed that there is an inverse correlation between glucose concentration and AMPK phosphorylation and activity. There is also an inverse correlation between high glucose concentrations and glucose uptake. Hayashi et al. (Diabetes 2000, 49: 527-531) had previously suggested that regulation of glucose uptake by AMPK is related to changes in Glut4 distribution. Based on the reported roles of AMPK family members in insulin signaling and glucose uptake, it is highly likely that PN7065 may have a related function(s). The role of PN7065 as a ligand of Glut4 further supports a role for PN7065 in diabetes or insulin resistance.

PN7386 is identical to a human chromosome 20 clone called 850H21 (GenBank accession AL031680) that is uncharacterized in the literature. It is possible that the protein product of this clone may participate in protein trafficking or in the signal transduction mechanism that regulates this process.

We have been able to detect four more proteins, beta-catenin, alpha-SNAP, tropomyosin 3 and MAPKKK6, which can bind to Glut4. Beta-catenin is a protein containing so-called Armadillo repeats that is involved in two important cellular processes: signal transduction via the Wingless pathway and cell adhesion (Ben-Ze'ev et al., 1998). This interaction between Glut4 and beta-catenin may shed light on the regulation of insulin-responsive glucose transport since it links the transporter to an important signaling pathway. The alpha-SNAP is an important mediator in the cellular process of intracellular transport (St-Denis, et al., 1998). The finding that alpha-SNAP and Glut4 interact provides a link between glucose transportation and the machinery required to perform movement of the glucose transporter between the outside and the interior of the cell. Glut4 has been demonstrated to interact with tropomyosin 3, a protein involved in muscle contraction (Squire et al., 1998). This interaction may represent a link between glucose uptake and muscle function. Finally, the Glut4 glucose transporter has been shown to interact with a putative protein kinase, MAPKKK6. This enzyme was identified by virtue of its ability to bind to another protein kinase (Wang et al., 1998). Once again, this may provide a clue to the mechanism of regulation of glucose transport since Glut4 can interact with another potential signal tranduction mediator.

We have been able to detect two proteins which can bind to Glut4, 14-3-3 zeta and the efp-like protein (KIAA0282). The 14-3-3 zeta is a signal transduction protein which has been shown to interact specifically with phospherine residues (Thorson et al., 1998). 14-3-3 zeta is part of a pathway that links the insulin receptor molecule at the cell surface to the Glut4 protein located either in the interior of the cell or also at the cell surface. Interestingly, the same small region of Glut4 that interacts with 14-3-3 zeta has been shown to be phosphorylated on a critical serine residue by the kinase Akt-2 that has also been implicated in glucose uptake (Kupriyanova et al., 1999). The efp-like protein is a putative transcription factor (Orimo et al., 1995) that may be regulated by the Glut4 protein's influence on the transcriptional activation of various genes involved in cellular metabolism.

We have been able to detect an additional protein, which can bind to Glut4. This protein is called MM-1 and was identified by virtue of its ability to interact with the proto-oncogene c-myc (Mori et al., 1998). We have identified the large centrosomal protein C-Nap1 as an interactor of MM-1. C-Nap1 was originally identified as a protein that could interact with the Nek2 cell cycle-regulated protein kinase (Fry et al., 1998). The finding that MM-1 can interact with C-Nap1 serves to tie Glut4 and glucose transport in general to the control of the cell cycle. The second protein shown to interact with MM-1 is beta spectrin. Spectrins give flexibility to the cell and also act as a scaffold for other cellular proteins (Grum et al., 1999). Interestingly, we have linked beta spectrin to glucose transport and Diabetes in a previous finding where it was shown that beta spectrin could interact with the vesicle-associated protein IRAP. The finding that MM-1 can bind to beta spectrin further strengthens the argument that beta spectrin plays a role in glucose transport. MM-1 was shown to bind to a third protein, KIAA0477, which was originally isolated from brain, although its tissue distribution is not known. The finding that KIAA0477 interacts with MM-1 suggests that KIAA0477 plays a role in glucose transport or in some cellular function associated with vesicular transport.

Another approach to understanding the mechanism of glucose transport has been to use the Glut1 molecule (Hresko et al., 1994), a gene highly related to Glut4 and possessing similar biological function, as a molecular bait in the search for other molecules that can interact with it. We have identified three more proteins which can bind to Glut1: DRAL, HSS and myosin heavy chain. DRAL is a LIM domain-containing protein that is also known as FHL-2 or SLIM3. DRAL was identified as a protein that was expressed in normal muscle tissue culture cells but was down-regulated in cancerous rhabdomyosarcoma cells (Genini et al., 1997). It is possible that DRAL plays a critical role in the terminal differentiation of muscle cells such as cardiac muscle, and that its misregulation could result in an undifferetiated cancerous phenotype. Glut1 was also shown to interact with HSS or human sperm surface protein. HSS is a testis-specific protein that has no known function (Shankar et al., 1998). It does contain a putative transmembrane domain and a leucine-zipper dimerization domain. Since it interacts with Glut1 and it is presumably membrane-bound, HSS could potentially act with Glut1 or Glut4 to affect glucose transport in the testis. Glut1 has also been demonstrated to interact with a form of the myosin heavy chain. Myosin heavy chain plays a key role in muscle structure and contraction (Eddinger et al., 1998). The interaction between Glut1 and myosin suggests that glucose uptake and muscle function may be interrelated via the association of these two proteins.

We identified dynamin as an interactor of Glut1. Dynamin has been implicated in the movement of glucose transporters via vesicular trafficking and likely plays a critical role in endocytosis (or movement from the cell surface to the interior of the cell) (Kao et al., 1998). We found two proteins that could interact with Dynamin. The first protein is called CALM, and it is a clathrin assembly protein similar to the AP-3 family of adaptor proteins. CALM was originally found in a lymphoid myeloid leukemia cell line containing a chromosome translocation resulting in the fusion of the AF10 gene with CALM (Dreyling et al., 1996). Clathrin and its associated proteins have a long history of involvement in the transport of vesicles from the cell surface to the interior of the cell. The association of dynamin and CALM further supports this role and ties CALM to glucose transport. Dynamin also binds to a proteosome activator subunit termed Psme3. The human Psme3 gene maps to the region of the BRCA1 gene, and its function was deduced by its similarity to the mouse gene that is also referred to as the Ki antigen (Kohda et al., 1998). Since the proteosome is required for the post-translational processing and the specific degradation of certain proteins, the finding that Psme3 can bind to dynamin implies that this type of protease activity may play a key role in glucose transport.

We have identified two more proteins which can bind to Glut1, DRAL/FHL2 and cardiac muscle myosin heavy chain. The first protein, DRAL/FHL2, is a protein shown to be down-regulated in rhabdomyosarcoma (Genini et al., 1997). It is entirely composed of LIM domains, polypeptide motifs that form double zinc fingers and may function by facilitating binding to nucleic acid or other proteins. The same region of Glut1 has been shown to bind to a cardiac muscle myosin heavy chain (MYSA)(Metzger et al., 1999), which is known to function in muscle contraction and cell structure.

We have identified three proteins which can bind to Glut1, dynamin, KIAA0144, and type 1 transmembrane receptor (seizure-related protein) (PSK-1). Dynamin has been implicated in the movement of glucose transporters via vesicular trafficking and likely plays a critical role in endocytosis (or movement from the cell surface to the interior of the cell) (Kao et al., 1998). The region of the KIAA0144 gene that contacts Glut1 is highly enriched for serine, threonine and proline residues, possibly providing a clue to its function. Other proteins with similar “STP” domains include the extracellular portions of cell surface receptors. Finally, a potential translation product of PSK-1 bears a striking resemblance to a previously identified mouse gene called SEZ-6. This gene was found by virtue of its increased transcript levels in brain tissue following exposure to a seizure producing drug (Shimizu-Nishikawa et al., 1995).

The insulin-regulated membranse-spanning aminopeptidase or IRAP (also known as vp165, gp160 and oxytocinase) shows similar tissue distribution to Glut4, is expressed in the CNS, and co-localizes with the Glut4 transporter in specified endocytic vesicles (Keller et al., 1995; Malide et al., 1997). Since expression of the N-terminal fragment of IRAP has been shown to result in the translocation of Glut4 to the plasma membrane, IRAP is thought to play a key role in glucose transport (Waters et al., 1997).

We have detected the interaction of IRAP with two proteins, 14-3-3 beta and HSS. The 14-3-3 family of proteins are critical signal transduction proteins that bind to phosphoserine residues (Jin et al., 1996). The interaction of IRAP with 14-3-3 beta strongly suggests that the function of IRAP could be regulated by phosphorylation and by the subsequent binding by 14-3-3 family members. Since IRAP and the Glut4 glucose transporter co-localize in the same intracellular vesicles, it is possible that Glut4 may participate in signal transduction mechanisms mediated by the 14-3-3 proteins such as 14-3-3 beta. IRAP has also been shown to bind to the HSS protein described above. Like Glut1 and Glut4, IRAP is membrane-bound and could potentially bind to HSS in the membrane. This finding points to HSS as playing a role in glucose transport or in other important functions performed in intracellular vesicles.

We have detected the interaction of IRAP with two more proteins. The N-terminal portion of IRAP has been shown to interact with SLAP-2. The rabbit homolog of SLAP-2 has been demonstrated to localize to the sarcolemma or the membrane of muscle cells although its function has not been elucidated (Wigle et al., 1997). SLAP-2 may play a role in vesicular transport or may at least participate in it since it has been shown to be membrane-associated and localizes to both the cell membrane as well as to intracellular stores in the endoplasmic reticulum. The C-terminal extracellular portion of IRAP has been demonstrated to interact with SG2NA. SG2NA is a cell cycle nuclear autoantigen that contains so-called WD-40 repeats that are present in a variety of signal transduction proteins (Muro et al., 1995). It is possible that SG2NA binding to IRAP is part of complex regulatory mechanism of glucose transport.

We have detected the interaction of IRAP with one more protein. The N-terminal portion of IRAP has been shown to interact VAMP-associated protein A (VAP-A or VAP-33). This protein has been implicated in intracellular transport (specifically exocytosis or movement to the cell surface) in A. californica and likely plays a similar role in humans (Skehel et al., 1995; Weir et al., 1998).

We have detected the interaction of IRAP with three proteins. The N-terminal portion of IRAP has been shown to interact with another signal transduction protein, the zeta polypeptide of protein tyrosine phosphatase (PTPZ). This protein could play a role in regulating glucose transport by dephosphorylating critical proteins that cause or prevent glucose transport (Nishiwaki et al., 1998). Numerous studies have indicated that there is a link between insulin resistant diabetes (Type II) and Alzheimer's disease. Gasparini et al., Trends Pharmacol Sci, 23:288-293 (2002); Hoyer, J Neural Transm, 109:341-360 (2002); Hoyer, J Neural Transm, 109:991-1002 (2002). Another insulin-regulated protein-secreted APP (“sAPP”)-binds the signal transduction phosphatase PTPZ, thereby stimulating neuroprotective effects, which are notably absent in Alzheimer's disease patients. Therefore, the discovery that IRAP and PTPZ interact with each other, points to the likelihood that the co-occurrence of non-insulin dependent diabetes and Alzheimer's disease may be treated by modulating the interaction between IRAP and PTPZ.

The C-terminal portion of IRAP has also been shown to interact with non-erythrocytic beta-spectrin. This protein is thought to be involved in secretion and could play a role in the movement of Glut 4 vesicles through IRAP (Hu et al., 1992).

Protein phosphatase 5 (PP5) is a TPR domain containing protein that seems to be part of larger multiprotein complexes that possess several cellular functions (Silverstein et al., 1997). PP5 has been demonstrated to interact with another related heat shock protein, Hsp89, and also with tankyrase.

Phosphatidyl inositol-3 kinase is a very important signal transduction protein and likely plays a critical role in Glut4-mediated glucose uptake (Shankar et al., 1998). This protein participates in transmitting signals from the outside of the cell into the interior. It is composed of two subunits, the p85 regulatory subunit and the p110 catalytic subunit, and functions by facilitating the transmission of signals from the outside of the cell into the interior. PI-3 kinase has been implicated in insulin-regulation and glucose uptake since one of its functions involves the insulin receptor (Martin et al., 1996). Further, the movement of Glut4 between the plasma membrane and the interior of the cell depends on the action of the PI-3 kinase signal transduction pathway since inhibitors of this kinase prevent the cycling of Glut4.

The p85 regulatory subunit of PI-3 kinase has been shown to interact with several proteins. Here we report the identification of five more proteins that can interact with p85. SLP-76 is a tyrosine phosphoprotein that participates in T cell signaling (Clements et al., 1998; Jackman et al., 1995). It is thought that SLP-76 acts as a so-called adaptor protein since it plays a role in intermediate steps of signal transduction. This is achieved by bridging factors that act at the plasma membrane with other molecules that perform functions within the interior of the cell. Our results indicate that SLP-76 may play a critical role in the PI-3 kinase signal transduction pathway by virtue of its ability to bind the p85 regulatory subunit. The p85 subunit of PI-3 kinase has also been demonstrated to bind to two more important signal transduction proteins: 14-3-3 zeta and 14-3-3 eta. These proteins bind specifically to phosphoserine residues in a number of proteins (Oghira et al., 1997; Thorson et al., 1998; Yaffe et al., 1997). Interestingly, our studies have shown that the Glut4 glucose transporter can also interact with 14-3-3 zeta. Thus, PI-3 kinase can also be connected to glucose uptake mechanisms by its interaction with the 14-3-3 signal transduction proteins. PI-3 kinase p85 was shown to interact with chromogranin C, a neuroendocrine secretory granule protein in the granin family (Ozawa et al., 1995). Members of the granin family localize to specialized secretory vesicles and are thought to serve an important function in protein sorting and secretion (Leitner et al., 1999). Finally, TACC2 has been shown to interact with PI-3 kinase p85. TACC2 is a member of a family of “transforming coiled coil” proteins that have been implicated in cellular growth control and cancer (Still et al., 1999). TACC2's interaction with p85 demonstrates that it may be a part of an important signal transduction pathway.

The p110 catalytic subunit of PI-3 kinase has been found to interact with the p85 and p55 subunits of the same enzyme complex as well as non-subunit interactions. The p10 subunit of this protein has also been shown to interact with complement protein C4, tenascin XB and alpha acid glucosidase (GAA). The complement C4 protein plays a key role in activating the classical complement pathway, and it is involved in evoking histamine release from basophils and mast cells. Naturally occurring deficiencies of C4 have been correlated with a number of immune-related human diseases such as Systemic Lupus Erythmatosus, kidney disease, hepatitis, dementia and the propensity for recurrent infections (Mascart-Lemone et al., 1983; Vergani et al., 1985; Waters et al., 1997; Nerl et al., 1984; Lhotta et al., 1990). The finding that C4 interacts with the p 110 subunit of PI-3 kinase suggests that the function of C4 is somehow regulated by this signal transduction, perhaps in response to some immunologic stimulus. Consequently, inhibitors of C4 and other complement proteins (such as C1) may be used as therapeutics in treating or preventing diabetes.

The p110 catalytic subunit of PI-3 kinase has been demonstrated to interact with tenascin XB. Tenascin XB is an extracellular structural protein. Deficiency of tenascin XB is linked to the connective tissue disorder Ehler-Danlos syndrome (Burch et al., 1997). This finding that p110 can bind to tenascin XB suggest that the function of tenascin XB could be modified or regulated by the PI-3 kinase signal trasduction pathway. PI-3 kinase p110 has also been shown to bind to GAA or alph acid glucosidase. GAA is a lysosomal enzyme that catalyzes the release of glucose from glycosylated substrates, and defects in GAA result in a glycogen storage disease (Raben et al., 1995). The finding that PI-3 kinase can bind to GAA suggests that GAA activity may be influenced by the PI-3 kinase signaling mechanism. Our previous results have linked the PI-3 kinase to human diseases such as Diabetes and Alzheimer's disease. These new findings therefore link complement protein C4, tenascin XB and alpha acid glucosidase to these diseases as well as to the other diseases already described.

The p110 subunit of this protein has been shown to interact with the thyroid hormone interacting protein TRIP15. TRIP15 is part of a larger multiprotein complex termed the signalsome that is likely to be involved in cell signaling (Seeger et al., 1998). Our results have linked the PI-3 kinase to human diseases such as Diabetes and Alzheimer's disease.

We have detected the interaction of PI-3 kinase p110 subunit with the β-amyloid precursor protein (APP). In brief, there is now growing evidence that APP metabolism and AP generation are central events to AD pathogenesis. For a more extensive review of APP and AD pathogenesis, see U.S. patent application Ser. No. 09/466,139, filed 21 Dec. 1999 and international patent application No. PCT/US99/30396, filed 21 Dec. 1999, each incorporated herein by reference. Although several candidates (cathepsin G, cathepsin D, chymotrypsin, and others) have been suggested, the β-secretase enzyme has not yet been identified. Even less is known about α- and γ-secretases. The biochemical link between the presenilins and APP processing has not been established. The proteins that mediate the neurotrophic and neuroprotective effects of sAPP are unknown. This last point is of utmost importance because an alteration of APP metabolism could result in both the generation of a toxic product (Aβ) and the impairment of sAPP trophic activity (Saitoh et al. 1994; Roch et al. 1993; Saitoh and Roch, 1995). In this respect, it is interesting that one APP mutation associated with Alzheimer's results in a defective neurite extension activity of sAPP (Li et al., 1997). Moreover, the balance of phosphorylation cascades is deeply altered in Alzheimer brains (Saitoh and Roch, 1995; Jin and Saitoh, 1995; Mook-Jung and Saitoh, 1997; Saitoh et al., 1991; Shapiro et al., 1991). Since APP is thought to play a major role in the pathology of Alzheimer's disease, the elucidation of a tie between PI-3 kinase and APP provides a new target for discovering the treatment for this neurological disorder (Russo et al., 1998). O-linked N-acetylglucosaminyltransferase or OGTase is an enzyme implicated in intracellular signal transduction (Kreppel et al., 1997). It has been speculated that OGTase may play a key role in glucose uptake and may therefore participate in the Diabetes related pathways (Cooksey et al., 1999). OGTase has been shown to also interact with myosin heavy chain. The binding of myosin heavy chain to OGTase suggests that the function of myosin in muscle structure or function could be influenced by OGTase in its signal transductive capacity. This could potentially affect many cellular processes in the muscle cell, including glucose transport. OGTase has been shown to bind to a novel protein termed PN6931. PN6931 is very similar to the mouse kinesin light chain gene (GenBank accession AF055666). Kinesin is a molecular motor involved in cellular transport and chromosome movement (Kirchner et al., 1999). Perhaps by post-translationally modifying this novel kinesin-like protein, OGTase can influence its function.

Amino acids 250 to 450 of OGTase has been shown to interact with a member of the 14-3-3 protein family, 14-3-3 epsilon. The 14-3-3 proteins function in intracellular signal transduction pathways by specifically binding to phosphoserine residues on other critical signaling molecules (Ogihara et al., 1997; Yaffe et al., 1997). The interaction between OGTase and 14-3-3 epsilon may serve to link two different signal transduction pathways, one which involves phosphorylation and a second which involves another type of protein modification.

Amino acids 250 to 450 of OGTase has been shown to interact with two proteins, alpha-2 catenin and Naf1a. Alpha-catenin is a protein related to vinculins that functions in cell-cell contact by binding to cadherins (Rudiger et al., 1998). The same region of OGTase can interact with Naf1b, a protein identified by virtue of its ability to bind the Nef gene product of HIV (Fukushi et al., 1999). Over-expression of Naf1a was observed to cause an increase in cell surface expression of the CD4 antigen, therefore this protein may also function in intracellular trafficking and could potentially participate in a number of diseases related to this general process such as Diabetes and Alzheimer's disease.

We identified Naf1b as an interactor of OGTase. Naf1b is a protein which was identified by virtue of its ability to bind the Nef gene product of HIV (Fukushi et al., 1999). Over-expression of Naf1b was observed to cause an increase in cell surface expression of the CD4 antigen, therefore this protein may also function in intracellular trafficking and could potentially participate in a number of diseases related to this general process such as Diabetes and Alzheimer's disease. The TRAF-interacting protein I-TRAF was found to interact with Naf1b. I-TRAF appears to act as a regulator of the TRAF signal transduction pathway that transmits signals from TNF (tumor necrosis factor) receptor family members (Rothe et al., 1996). The observation that Naf1b interacts with I-TRAF serves to link an extracellular stimulated signal transduction mechanism with the OGTase pathway involved in glucose transport. Naf1b was also found to interact with a novel protein fragment called PN7582. It is very possible that PN7582 may participate in protein trafficking or signal transduction by its association with Naf1b.

OGTase was found to interact with four new interactors. The first interactor, desmin, is a cytoplasmic intermediate filament protein found in muscle (Capetanaki et al., 1998). It functions in striated muscle by connecting myofibrils with themselves and with the plasma membrane. Desmin is broken down into three functional domains, the head, rod and tail, and OGTase can bind to the rod structure.

The second protein shown to interact with OGTase is called alpha-karyopherin. Alpha-karyopherin (also known as importin and SRP1) is a ubiquitously expressed protein that plays a role in trafficking nuclear localization signal-containing proteins into the nucleus through the nuclear pore (Moroianu, 1997). Alpha-karyopherin also interacts with the HIV preintegration complex and plays a role in nuclear importation of the H IV viral genome. Consequently, inhibitiors of alpha-karyopherin or the HIV preintegration complex may be useful therapeutics in HIV or diabetes.

The third protein that OGTase has been shown to bind is glutanimyl-tRNA synthetase. The aminoacyl-tRNA synthetases not only play a key role in protein synthesis, but recent studies have shown that they also impact a number of other cellular processes such as tRNA processing, RNA splicing, RNA trafficking, apoptosis, and transcriptional and translational regulation (Martinis et al., 1999).

The fourth protein shown to bind to OGTase is clone 25100 (BAB55262) (similar to protein disulfide isomerase ER-60 precursor). The gene for BAB55262 was isolated from human infant brain and appears to encode a small protein with no structural characteristics that shed light on its function. All four of these OGTase-interacting proteins may act as substrates for OGTase or may affect its function in some way. The finding that they interact with OGTase suggests that they may play a role in glucose transport or in the pathogenesis of Diabetes.

Our studies have demonstrated that OGTase can interact with a variety of proteins that can fall into a number of functional categories. Two proteins that have been implicated in vesicular transport bind to OGTase. The first is called BAP31, and it likely plays a critical role in sorting and transporting membrane proteins between intracellular compartments (Annaert et al., 1997). The second protein is called dynamin II, and it is implicated in the movement of transport vesicles from the plasma membrane to sites within the interior of the cell (Sontag et al., 1994). OGTase has been demonstrated to interact with two proteins that function in transcription, as well. The first is called EGR1 for early growth response protein 1, and it has been shown to be highly induced in cells following mitogenic stimulation (Sukhatme et al., 1988). It is a zinc-finger containing protein, and following its own stimulation, goes on to activate the transcription of genes involved in mitogenesis and differentiation. Additionally, OGTase can interact with MOP2, a basic helix-loop-helix containing transcription factor that is involved in the induction of oxygen regulated genes (Hogenesch et al., 1997). OGTase binds to a structural protein called talin. Talin is a cytoplasmic protein that serves to link integrins with the actin cytoskeleton (Calderwood et al., 1999). Finally, OGTase has been shown to bind to five proteins of unknown or poorly characterized function. OGTase binds to Int-6, a protein that has also been demonstrated to bind to the HTLV-I Tax transactivator and it is a component of promyelocytic leukemia nuclear bodies (Desbois et al., 1996). OGTase binds to interferon-induced protein 54, an uncharacterized protein that shows a large increase in its transcript following treatment with alpha- and beta-interferons (Levy et al., 1986). Lastly, HSPC028, KIAA0443, and hypothetical protein BAB55262 interact with OGTase.

Akt kinase is a serine/threonine protein kinase that has been implicated in insulin-regulated glucose transport and the development of non-insulin dependent diabetes mellitus (Krook et al., 1998). Akt1 and Akt2 were both shown to interact with the nuclear mitotic apparatus protein NuMA1. NuMA1 displays a distinct pattern of immunofluorescent staining that varies throughout the cell cycle. It is nuclear throughout interphase but re-localizes to the spindle apparatus during mitosis (Lydersen et al., 1980). Phosphorylation is thought to play a critical role in NuMA function. It appears to become phosphorylated just prior to mitosis and becomes dephosphorylated after mitosis has occurred in the G1 phase of the cell cycle mitosis (Sparks et al., 1995). Akt1 and Akt2 may be capable of phosphorylating NuMA, especially since the region of NuMA that interacts with Akt2 (amino acids 98 to 365) contains 23 serines and 9 threonines. Akt2 was also shown to interact with two proteins involved in vesicular transport. The first protein, BAP31, likely plays a role in the movement of membrane-bound proteins from the Golgi appartus to the plasma membrane (Annaert et al., 1997). Since BAP31 was also shown to interact with OGTase in our previous experiments, two signal transduction proteins implicated in glucose transport have been tied to BAP31. Akt2 was also shown to bind to another vesicular transport protein termed beta-adaptin or AP2-beta. Beta-adaptin is a part of the AP2 coat assembly complex that links clathrin and to transmembrane receptors resident in coated vesicles (Pearse, 1989). This finding suggests a role for Akt2 in the regulation of endocytosis, while the finding that Akt2 binds to BAP31 provides for a tie between Akt2 and exocytosis. All of the proteins that can interact with the Akt kinase may play a role in glucose transport by virtue of their association with Akt.

PTP1b is a protein tyrosine phosphatase that plays a critical role in signal transduction. It has been implicated as a negative regulator of insulin-responsive glucose transport (Chen et al., 1997), and therefore it has been used in an attempt to find more proteins involved in this function, perhaps by acting as substrates. PTP1b was shown to interact with VAMP-associated protein A (VAP-A or VAP-33). This protein has been implicated in intracellular transport (specifically exocytosis or movement to the cell surface) in A. californica and likely plays a similar role in humans (Skehel et al., 1995; weir et al., 1998). The interaction between PTP1b and VAP-A serves as a potential tie between PTP1b and IRAP since RAP was also shown to bind to VAP-A. RAP, insulin-regulated membrane-spanning aminopeptidase (also known as vp165, gp160 and oxytocinase) co-localizes with the Glut4 transporter in specified endocytic vesicles (Keller et al., 1995; Malide et al., 1997). Since expression of the N-terminal fragment of IRAP has been shown to result in the translocation of Glut4 to the plasma membrane, IRAP is thought to play a key role in glucose transport (Waters et al., 1997). Thus, the result that PTP1b interacts with VAP-A serves to strengthen the tie between PTP1b and VAP-A to glucose transport and Diabetes.

The small GTP-binding protein Rab4 is another signal transduction factor that has been implicated in the regulation of insulin-stimulated glucose uptake (Vollenweider et al., 1997; Cormont et al., 1996). Rab4, therefore, has also been used to find additional proteins involved in glucose transport and in Diabetes. Two proteins have been shown to bind to Rab4, an alpha-catenin-like protein (sometimes called alpha-catulin) and another small GTP-binding protein called Rab2. The alpha-catenin-like protein resembles alpha-catenin and vinculin however its function has not yet been well-characterized (Janssens et al., 1999). Alpha-catenin itself is a protein related to vinculins that functions in cell-cell contact by binding to cadherins (Rudinger et al., 1998). Interestingly, alpha-catenin was found in our previous studies to bind to OGTase and, as a consequence, has been implicated in glucose transport. The second protein shown to bind to Rab4 is Rab2. These two small GTP-binding proteins are highly related with a 50% amino acid identity. Rab2 plays an important role in vesicular transport in the cell (Tisdale et al., 1998). The finding that Rab4 and Rab2 interact suggests that they may be capable of influencing each others cellular functions, thus Rab2 could potentially affect glucose transport.

The present invention provides isolated PN31302 nucleic acid molecules. The nucleic acid molecules can be in the form of DNA, RNA, or a chimera or hybrid thereof, and can be in any physical structures including single-stranded or double-stranded molecules, or in the form of a triple helix. In one embodiment, the isolated PN31302 nucleic acid molecule has a sequence of SEQ ID NO:255, SEQ ID NO:257, SEQ ID NO:259, SEQ ID NO:261, SEQ ID NO:263, SEQ ID NO:265, or SEQ ID NO:267 or the complement thereof.

In addition, nucleic acid molecules are also contemplated, which are capable of specifically hybridizing, under stringent hybridization conditions, to a nucleic acid molecule having the sequence of SEQ ID NO:255, SEQ ID NO:257, SEQ ID NO:259, SEQ ID NO:261, SEQ ID NO:263, SEQ ID NO:265, or SEQ ID NO:267 or the complement thereof. Preferably, such nucleic acid molecules encode a polypeptide having the sequence of SEQ ID NO:256, SEQ ID NO:258, SEQ ID NO:260, SEQ ID NO:262, SEQ ID NO:264, SEQ ID NO:266, or SEQ ID NO:268 or fragment thereof.

In another embodiment, an isolated nucleic acid molecule is provided, which has a sequence that is at least 50%, preferably at least 60%, more preferably at least 75%, 80%, 82%, 85%, even more preferably at least 90%, 91%, 92%, 93%, 94%, 95%, 96%, 97%, 98%, or 99% identical to the sequence of SEQ ID NO:255, SEQ ID NO:257, SEQ ID NO:259, SEQ ID NO:261, SEQ ID NO:263, SEQ ID NO:265, or SEQ ID NO:267 or the complement thereof. Preferably, such nucleic acid molecules encode a polypeptide having the sequence of SEQ ID NO:256, SEQ ID NO:258, SEQ ID NO:260, SEQ ID NO:262, SEQ ID NO:264, SEQ ID NO:266, or SEQ ID NO:268.

As is apparent to skilled artisans, homologous nucleic acids or nucleic acids capable of hybridizing with a nucleic acid of the sequence of SEQ ID NO:255, SEQ ID NO:257, SEQ ID NO:259, SEQ ID NO:261, SEQ ID NO:263, SEQ ID NO:265, or SEQ ID NO:267 can be prepared by manipulating a nucleic acid molecule having a sequence of SEQ ID NO:255, SEQ ID NO:257, SEQ ID NO:259, SEQ ID NO:261, SEQ ID NO:263, SEQ ID NO:265, or SEQ ID NO:267. For example, various nucleotide substitutions, deletions or insertions can be incorporated into the nucleic acid molecule by standard molecular biology techniques. As will be apparent to skilled artisans, such nucleic acids are useful irrespective of whether they encode a functional protein. For example, they can be used as probes for isolating and/or detecting nucleic acids. Nevertheless, preferably the homologous nucleic acids or the nucleic acids capable of hybridizing with a nucleic acid of the sequence of SEQ ID NO:255, SEQ ID NO:257, SEQ ID NO:259, SEQ ID NO:261, SEQ ID NO:263, SEQ ID NO:265, or SEQ ID NO:267 encode a polypeptide having one or more PN31302 activities.

In addition, nucleic acid molecules that encode the proteins having an amino acid sequence of SEQ ID NO:256, SEQ ID NO:258, SEQ ID NO:260, SEQ ID NO:262, SEQ ID NO:264, SEQ ID NO:266, or SEQ ID NO:268 are also intended to fall within the scope of the present invention. As will be immediately apparent to a skilled artisan, due to genetic code degeneracy, such nucleic acid molecules can be designed conveniently by nucleotide substitutions in the wild-type nucleotide sequence of SEQ ID NO:255, SEQ ID NO:257, SEQ ID NO:259, SEQ ID NO:261, SEQ ID NO:263, SEQ ID NO:265, or SEQ ID NO:267.

In addition, the present invention further encompasses nucleic acid molecules encoding a protein that has a sequence that is at least 75%, preferably at least 85%, 90%, 91%, 92%, 93%, or 94%, and more preferably at least 95%, 96%, 97%, 98%, or 99% identical to the sequence of SEQ ID NO:256, SEQ ID NO:258, SEQ ID NO:260, SEQ ID NO:262, SEQ ID NO:264, SEQ ID NO:266, or SEQ ID NO:268. The various nucleic acid molecules may be produced by chemical synthesis and/or recombinant techniques based on an isolated PN31302 nucleic acid molecule having a sequence of SEQ ID NO:255, SEQ ID NO:257, SEQ ID NO:259, SEQ ID NO:261, SEQ ID NO:263, SEQ ID NO:265, or SEQ ID NO:267.

In another embodiment of the present invention, oligonucleotides are provided having a contiguous span of at least 10, 12, 15, 16, 17, 18, 19, 20, 21, 22, 23, 24, 25, 26, 27, 28, 29, 30, 35, 50, 75, 100, 200, 300, or 431 nucleotides of the sequence of SEQ ID NO:255, SEQ ID NO:257, SEQ ID NO:259, SEQ ID NO:261, SEQ ID NO:263, SEQ ID NO:265, or SEQ ID NO:267, or the complement thereof. Preferably, the oligonucleotides are less than the full length of the sequence of SEQ ID NO:255, SEQ ID NO:257, SEQ ID NO:259, SEQ ID NO:261, SEQ ID NO:263, SEQ ID NO:265, or SEQ ID NO:267, more preferably no greater than 400, 200, 100, or 50 nucleotides in length. In a preferred embodiment, the oligonucleotides have a length of about 12-18, 19-25, 26-34, 35-50, or 51-100 nucleotides. In a specific embodiment, the oligonucleotide is a sequence encoding a contiguous span of at least 4, 5, 6, 7, 8, 9, 10, 11, 12, 13, 14, 15, 17, 20, 22, 25, 30, 35, 50, 100, 150, 200, 300, 400, or 500 amino acids of SEQ ID NO:256, SEQ ID NO:258, SEQ ID NO:260, SEQ ID NO:262, SEQ ID NO:264, SEQ ID NO:266, or SEQ ID NO:268. In another specific embodiment, the oligonucleotide is an antisense oligo as described in Section 11.2.2. In another specific embodiment, the oligonucleotide is a ribozyme molecule as described in Section 11.2.3. In yet another specific embodiment, the oligonucleotide can serve as a primer for nucleic acid amplification reactions, such as the Polymerase Chain Reaction (PCR).

The present invention further encompasses oligonucleotides that have a length of at least 10, 12, 15, 17, 18, 19, 20, 21, 22, 23, 24, 25, 30, 50, 75, 100, 200, 300, 400, 500, or 600 nucleotides and preferably no greater than 800, 600, 400, 200 or 100 nucleotides, and are at least 85%, 90%, 92% or 94%, and more preferably at least 95%, 96%, 97%, 98%, or 99% identical to a contiguous span of nucleotides of the sequence of SEQ ID NO:255, SEQ ID NO:257, SEQ ID NO:259, SEQ ID NO:261, SEQ ID NO:263, SEQ ID NO:265, or SEQ ID NO:267 or the complement thereof. The oligonucleotides can have a length of about 12-18, 19-25, 26-34, 35-50, 51-100, 101-250, 251-500, or 501-800 nucleotides. In a preferred embodiment, the oligonucleotides have a length of about 12-100, 15-75, 17-50, 21-50, or preferably 25-50 nucleotides. Preferably, the oligonucleotide is a sequence encoding a contiguous span of at least 4, 5, 6, 7, 8, 9, 10, 11, 12, 13, 14, 15, 17, 20, 22, 25, 30, 35, 50, 100, or 143 amino acids of SEQ-ID NO:256, SEQ ID NO:258, SEQ ID NO:260, SEQ ID NO:262, SEQ ID NO:264, SEQ ID NO:266, or SEQ ID NO:268.

In addition, oligonucleotides are also contemplated having a length of at least 10, 12, 15, 17, 18, 19, 20, 21, 22, 23, 24, 25, 30, 50, 75, 100, 200, 300, 400, 500, or 600 nucleotides and preferably no greater than 800, 600, 400, 200 or 100 nucleotides, and capable of hybridizing to the nucleotide sequence of SEQ ID NO:255, SEQ ID NO:257, SEQ ID NO:259, SEQ ID NO:261, SEQ ID NO:263, SEQ ID NO:265, or SEQ ID NO:267, or the complement thereof, under stringent hybridization conditions. In a preferred embodiment, the oligonucleotides have a length of about 12-100, 15-75, 17-50, 21-50, or preferably 25-50 nucleotides. In another preferred embodiment, the oligonucleotides capable of hybridizing to the nucleotide sequence of SEQ ID NO:255, SEQ ID NO:257, SEQ ID NO:259, SEQ ID NO:261, SEQ ID NO:263, SEQ ID NO:265, or SEQ ID NO:267 or the complement thereof encode a contiguous span of at least 7, 8, 9, 10, 11, 12, 13, 14, 15, 16, 17, 18, 19, 20, 25, 30, 50, 100, 150, 200, 300, 400, or 500 amino acids of SEQ ID NO:256, SEQ ID NO:258, SEQ ID NO:260, SEQ ID NO:262, SEQ ID NO:264, SEQ ID NO:266, or SEQ ID NO:268.

As will be apparent to skilled artisans, the various oligonucleotides of the present invention are useful as probes for detecting nucleic acids in cells and tissues. They can also be used as primers for procedures including the amplification of nucleic acids or homologues thereof, sequencing nucleic acids, and the detection of mutations in nucleic acids or homologues thereof. In addition, the oligonucleotides may be used to encode a fragment, epitope or domain of proteins or a homologue thereof, which is useful in a variety of applications including use as antigenic epitopes for preparing antibodies against proteins.

It should be understood that the nucleic acid molecules of the present invention may be in standard forms with conventional nucleotide bases and backbones, but can also be in various modified forms, e.g., having therein modified nucleotide bases or backbones. Examples of modified nucleotide bases or backbones described in Section 11.2.2 in the context of modified antisense compounds should be equally applicable in this respect.

The present invention also provides isolated polypeptides. Tthe present invention also encompasses a polypeptide having an amino acid sequence that is at least 50%, preferably at least 60%, more preferably at least 75%, 80%, 85%, 90%, 91%, 92%, 93%, 94%, and even more preferably at least 95%, 96%, 97%, 98%, or 99% identical to the amino acid sequence of SEQ ID NO:256, SEQ ID NO:258, SEQ ID NO:260, SEQ H) NO:262, SEQ ID NO:264, SEQ ID NO:266, or SEQ ID NO:268. In a specific embodiment, the homologous polypeptide is a naturally occurring protein variant of a protein identified in a human population. Such a variant may be identified by assaying the nucleic acids or protein in a population, as is generally known in the art. In another embodiment, the present invention also provides an isolated polypeptide that is encoded by an isolated nucleic acid molecule that specifically hybridizes fully to the isolated PN31302 nucleic acid molecule of SEQ ID NO:255, SEQ ID NO:257, SEQ ID NO:259, SEQ ID NO:261, SEQ ID NO:263, SEQ ID NO:265, or SEQ ID NO:267, or the complement thereof under moderately or highly stringent conditions.

The present invention further encompasses fragments proteins having a contiguous span of at least 6, 7, 8, 9, 10, 11, 12, 13, 14, 15, 16, 17, 18, 19, 20, 25, or at least 50 amino acids of the sequence of SEQ ID NO:256, SEQ ID NO:258, SEQ ID NO:260, SEQ if) NO:262, SEQ ID NO:264, SEQ ID NO:266, or SEQ ID NO:268, but less than the full length of the sequence of SEQ ID NO:256, SEQ ID NO:258, SEQ ID NO:260, SEQ ID NO:262, SEQ ID NO:264, SEQ ID NO:266, or SEQ ID NO:268. For example, such fragments can be generated as a result of the deletion of a contiguous span of a certain number of amino acids from either or both of the amino and carboxyl termini of the PN31302 protein having the sequence of SEQ ID NO:256, SEQ ID NO:258, SEQ ID NO:260, SEQ ID NO:262, SEQ ID NO:264, SEQ ID NO:266, or SEQ ID NO:268 In specific embodiments, a polypeptide is provided including a contiguous span of at least 4, 5, 6, 7, 8, 9, 10, 11, 12, 13, 14, 15, 18, 20, 25, or at least 50 amino acids of the sequence of SEQ ID NO:256, SEQ ID NO:258, SEQ ID NO:260, SEQ ID NO:262, SEQ ID NO:264, SEQ ID NO:266, or SEQ ID NO:268. In other specific embodiments, the polypeptide fragments contain immunogenic or antigenic epitopes. Such epitopes can be readily determined by computer programs such as MacVector from International Biotechnologies, Inc. and Protean from DNAStar. In addition, epitopes can also be selected experimentally by any methods known in the art, e.g., in U.S. Pat. Nos. 4,833,092 and 5,194,392, both of which are incorporated herein by reference.

In addition, the present invention is also directed to polypeptides that are homologous to the foregoing polypeptide fragments. Such a homologous polypeptide may have the same length as one of the foregoing polypeptide fragments of the present invention (e.g., from 5 to 50, from 5 to 30, or from 7 to 25, or preferably 8 to 20 amino acids) but has an amino acid sequence that is at least 75%, 80%, 85%, 90%, preferably at least 95%, 96%, 97%, 98%, or, more preferably, at least 99% identical to the amino acid sequence of the corresponding polypeptide fragment.

Additionally, the present invention further relates to a hybrid polypeptide having any one of the foregoing polypeptides of the present invention covalently linked to another polypeptide. Such other polypeptide can also be one of the foregoing polypeptides of the present invention. Alternatively, such other polypeptide is not one of the foregoing polypeptides of the present invention. The covalent linkage in the hybrid polypeptide of the present invention can be merely a covalent bond between the two components of the hybrid polypeptide. Alternatively, any linker molecules may be used. For example, a peptide or a non-peptidic organic molecule may be used as a linker molecule.

Because of the involvement of the proteins in the physiological pathways disclosed herein, the present invention provides a list of uses of these proteins and DNA encoding these proteins for the development of diagnostic and therapeutic tools useful in the physiological pathways.

### 2.2. Protein Complexes

Accordingly, the present invention provides protein complexes formed by interacting pairs of proteins described in the tables. The present invention also provides protein complexes in which one or more of the interacting protein members are native proteins or homologues, derivatives or fragments of native proteins.

Thus, for example, one interacting partner in a protein complex can be a complete native GLUT4, a GLUT4 homologue capable of interacting with, e.g., PN7065, a GLUT4 derivative, a derivative of the GLUT4 homologue, a GLUT4 fragment capable of interacting with PN7065 (GLUT4 fragment(s) containing the coordinates shown in Table 1), a derivative of the GLUT4 fragment, or a fusion protein containing (1) complete native GLUT4, (2) a GLUT4 homologue capable of interacting with PN7065 or (3) a GLUT4 fragment capable of interacting with PN7065. Besides native PN7065, useful interacting partners for GLUT4 or a homologue or derivative or fragment thereof also include homologues of PN7065 capable of interacting with GLUT4, derivatives of the native or homologue PN7065 capable of interacting with GLUT4, fragments of the PN7065 capable of interacting with GLUT4 (e.g., a fragment containing the identified interacting regions shown in Table 1), derivatives of the PN7065 fragments, or fusion proteins containing (1) a complete PN7065, (2) a PN7065 homologue capable of interacting with GLUT4 or (3) a PN7065 fragment capable of interacting with GLUT4.

PN7065 fragments capable of interacting with GLUT4 can be identified by the combination of molecular engineering of a PN7065-encoding nucleic acid and a method for testing protein-protein interaction. For example, the coordinates in Table 1 can be used as starting points and various PN7065 fragments falling within the coordinates can be generated by deletions from either or both ends of the coordinates. The resulting fragments can be tested for their ability to interact with GLUT4 using any methods known in the art for detecting protein-protein interactions (e.g., yeast two-hybrid method). Alternatively, various PN7065 fragments can be made by chemical synthesis. The PN7065 fragments can then be tested for their ability to interact with GLUT4 using any method known in the art for detecting protein-protein interactions. Examples of such methods include protein affinity chromatography, affinity blotting, in vitro binding assays, yeast two-hybrid assays, and the like. Likewise, GLUT4 fragments capable of interacting with PN7065 can also be identified in a similar manner.

Other protein complexes can be formed in a similar manner based on other interactions provided in the tables.

In a specific embodiment of the protein complex of the present invention, two or more interacting partners are directly fused together, or covalently linked together through a peptide linker, forming a hybrid protein having a single unbranched polypeptide chain. Thus, the protein complex may be formed by “intramolecular” interactions between two portions of the hybrid protein. Again, one or both of the fused or linked interacting partners in this protein complex may be a native protein or a homologue, derivative or fragment of a native protein.

The protein complexes of the present invention can also be in a modified form. For example, an antibody selectively immunoreactive with the protein complex can be bound to the protein complex. In another example, a non-antibody modulator capable of enhancing the interaction between the interacting partners in the protein complex may be included. Alternatively, the protein members in the protein complex may be cross-linked for purposes of stabilization. Various crosslinking methods may be used. For example, a bifunctional reagent in the form of R—S—S—R′ may be used in which the R and R′ groups can react with certain amino acid side chains in the protein complex forming covalent linkages. See e.g., Traut et al., in Creighton ed., Protein Function: A Practical Approach, IRL Press, Oxford, 1989; Baird et al., J. Biol. Chem., 251:6953-6962 (1976). Other useful crosslinking agents include, e.g., Denny-Jaffee reagent, a heterbiofunctional photoactivable moiety cleavable through an azo linkage (See Denny et al., Proc. Natl. Acad. Sci. USA, 81:5286-5290 (1984)), and ¹²⁵I-1{S-[N-(3-iodo-4-azidosalicyl)cysteaminyl]-2-thiopyridine}, a cysteine-specific photocrosslinking reagent (see Chen et al., Science, 265:90-92 (1994)).

The above-described protein complexes may further include any additional components, e.g., other proteins, nucleic acids, lipid molecules, monosaccharides or polysaccharides, ions, etc.

### 2.3. Methods of Preparing Protein Complexes

The protein complex of the present invention can be prepared by a variety of methods. Specifically, a protein complex can be isolated directly from an animal tissue sample, preferably a human tissue sample containing the protein complex. Alternatively, a protein complex can be purified from host cells that recombinantly express the members of the protein complex. As will be apparent to a skilled artisan, a protein complex can be prepared from a tissue sample or recombinant host cells by coimmunoprecipitation using an antibody immunoreactive with an interacting protein partner, or preferably an antibody selectively immunoreactive with the protein complex as will be discussed in detail below.

The antibodies can be monoclonal or polyclonal. Coimmunoprecipitation is a commonly used method in the art for isolating or detecting bound proteins. In this procedure, generally a serum sample or tissue or cell lysate is admixed with a suitable antibody. The protein complex bound to the antibody is precipitated and washed. The bound protein complexes are then eluted.

Alternatively, immunoaffinity chromatography and immunoblotting techniques may also be used in isolating the protein complexes from native tissue samples or recombinant host cells using an antibody immunoreactive with an interacting protein partner, or preferably an antibody selectively immunoreactive with the protein complex. For example, in protein immunoaffinity chromatography, the antibody is covalently or non-covalently coupled to a matrix (e.g., Sepharose), which is then packed into a column. Extract from a tissue sample, or lysate from recombinant cells is passed through the column where it contacts the antibodies attached to the matrix. The column is then washed with a low-salt solution to wash away the unbound or loosely (non-specifically) bound components. The protein complexes that are retained in the column can be then eluted from the column using a high-salt solution, a competitive antigen of the antibody, a chaotropic solvent, or sodium dodecyl sulfate (SDS), or the like. In immunoblotting, crude proteins samples from a tissue sample extract or recombinant host cell lysate are fractionated by polyacrylamide gel electrophoresis (PAGE) and then transferred to a membrane, e.g., nitrocellulose. Components of the protein complex can then be located on the membrane and identified by a variety of techniques, e.g., probing with specific antibodies.

In another embodiment, individual interacting protein partners may be isolated or purified independently from tissue samples or recombinant host cells using similar methods as described above. The individual interacting protein partners are then combined under conditions conducive to their interaction thereby forming a protein complex of the present invention. It is noted that different protein-protein interactions may require different conditions. As a starting point, for example, a buffer having 20 mM Tris-HCl, pH 7.0 and 500 mM NaCl may be used. Several different parameters may be varied, including temperature, pH, salt concentration, reducing agent, and the like. Some minor degree of experimentation may be required to determine the optimum incubation condition, this being well within the capability of one skilled in the art once apprised of the present disclosure.

In yet another embodiment, the protein complex of the present invention may be prepared from tissue samples or recombinant host cells or other suitable sources by protein affinity chromatography or affinity blotting. That is, one of the interacting protein partners is used to isolate the other interacting protein partner(s) by binding affinity thus forming protein complexes. Thus, an interacting protein partner prepared by purification from tissue samples or by recombinant expression or chemical synthesis may be bound covalently or non-covalently to a matrix, e.g., Sepharose, which is then packed into a chromatography column. The tissue sample extract or cell lysate from the recombinant cells can then be contacted with the bound protein on the matrix. A low-salt solution is used to wash off the unbound or loosely bound components, and a high-salt solution is then employed to elute the bound protein complexes in the column. In affinity blotting, crude protein samples from a tissue sample or recombinant host cell lysate can be fractionated by polyacrylamide gel electrophoresis (PAGE) and then transferred to a membrane, e.g., nitrocellulose. The purified interacting protein member is then bound to its interacting protein partner(s) on the membrane forming protein complexes, which are then isolated from the membrane.

It will be apparent to skilled artisans that any recombinant expression methods may be used in the present invention for purposes of expressing the protein complexes or individual interacting proteins. Generally, a nucleic acid encoding an interacting protein member can be introduced into a suitable host cell. For purposes of forming a recombinant protein complex within a host cell, nucleic acids encoding two or more interacting protein members should be introduced into the host cell.

Typically, the nucleic acids, preferably in the form of DNA, are incorporated into a vector to form expression vectors capable of directing the production of the interacting protein member(s) once introduced into a host cell. Many types of vectors can be used for the present invention. Methods for the construction of an expression vector for purposes of this invention should be apparent to skilled artisans apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.

Generally, the expression vectors include an expression cassette having a promoter operably linked to a DNA encoding an interacting protein member. The promoter can be a native promoter, i.e., the promoter found in naturally occurring cells to be responsible for the expression of the interacting protein member in the cells. Alternatively, the expression cassette can be a chimeric one, i.e., having a heterologous promoter that is not the native promoter responsible for the expression of the interacting protein member in naturally occurring cells. The expression vector may further include an origin of DNA replication for the replication of the vectors in host cells. Preferably, the expression vectors also include a replication origin for the amplification of the vectors in, e.g., E. coli, and selection marker(s) for selecting and maintaining only those host cells harboring the expression vectors. Additionally, the expression cassettes preferably also contain inducible elements, which function to control the transcription from the DNA encoding an interacting protein member. Other regulatory sequences such as transcriptional enhancer sequences and translation regulation sequences (e.g., Shine-Dalgarno sequence) can also be operably included in the expression cassettes. Termination sequences such as the polyadenylation signals from bovine growth hormone, SV40, lacZ and AcMNPV polyhedral protein genes may also be operably linked to the DNA encoding an interacting protein member in the expression cassettes. An epitope tag coding sequence for detection and/or purification of the expressed protein can also be operably linked to the DNA encoding an interacting protein member such that a fusion protein is expressed. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies immunoreactive with many epitope tags are generally commercially available. The expression vectors may also contain components that direct the expressed protein extracellularly or to a particular intracellular compartment. Signal peptides, nuclear localization sequences, endoplasmic reticulum retention signals, mitochondrial localization sequences, myristoylation signals, palmitoylation signals, and transmembrane sequences are examples of optional vector components that can determine the destination of expressed proteins. When it is desirable to express two or more interacting protein members in a single host cell, the DNA fragments encoding the interacting protein members may be incorporated into a single vector or different vectors.

The thus constructed expression vectors can be introduced into the host cells by any techniques known in the art, e.g., by direct DNA transformation, microinjection, electroporation, viral infection, lipofection, gene gun, and the like. The expression of the interacting protein members may be transient or stable. The expression vectors can be maintained in host cells in an extrachromosomal state, i.e., as self-replicating plasmids or viruses. Alternatively, the expression vectors can be integrated into chromosomes of the host cells by conventional techniques such as selection of stable cell lines or site-specific recombination. In stable cell lines, at least the expression cassette portion of the expression vector is integrated into a chromosome of the host cells.

The vector construct can be designed to be suitable for expression in various host cells, including but not limited to bacteria, yeast cells, plant cells, insect cells, and mammalian and human cells. Methods for preparing expression vectors for expression in different host cells should be apparent to a skilled artisan.

Homologues and fragments of the native interacting protein members can also be easily expressed using the recombinant methods described above. For example, to express a protein fragment, the DNA fragment incorporated into the expression vector can be selected such that it only encodes the protein fragment. Likewise, a specific hybrid protein can be expressed using a recombinant DNA encoding the hybrid protein. Similarly, a homologue protein may be expressed from a DNA sequence encoding the homologue protein. A homologue-encoding DNA sequence may be obtained by manipulating the native protein-encoding sequence using recombinant DNA techniques. For this purpose, random or site-directed mutagenesis can be conducted using techniques generally known in the art. To make protein derivatives, for example, the amino acid sequence of a native interacting protein member may be changed in predetermined manners by site-directed DNA mutagenesis to create or remove consensus sequences for, e.g., phosphorylation by protein kinases, glycosylation, ribosylation, myristolation, palmytoylation, ubiquitination, and the like. Alternatively, non-natural amino acids can be incorporated into an interacting protein member during the synthesis of the protein in recombinant host cells. For example, photoreactive lysine derivatives can be incorporated into an interacting protein member during translation by using a modified lysy1-tRNA. See, e.g., Wiedmann et al., Nature, 328:830-833 (1989); Musch et al., Cell, 69:343-352 (1992). Other photoreactive amino acid derivatives can also be incorporated in a similar manner. See, e.g., High et al., J. Biol. Chem., 368:28745-28751 (1993). Indeed, the photoreactive amino acid derivatives thus incorporated into an interacting protein member can function to cross-link the protein to its interacting protein partner in a protein complex under predetermined conditions.

In addition, derivatives of the native interacting protein members of the present invention can also be prepared by chemically linking certain moieties to amino acid side chains of the native proteins.

If desired, the homologues and derivatives thus generated can be tested to determine whether they are capable of interacting with their intended partners to form protein complexes. Testing can be conducted by e.g., the yeast two-hybrid system or other methods known in the art for detecting protein-protein interaction.

A hybrid protein as described above having any interacting pair of the proteins described in the tables, or a homologue, derivative, or fragment thereof covalently linked together by a peptide bond or a peptide linker can be expressed recombinantly from a chimeric nucleic acid, e.g., a DNA or mRNA fragment encoding the fusion protein. Accordingly, the present invention also provides a nucleic acid encoding the hybrid protein of the present invention. In addition, an expression vector having incorporated therein a nucleic acid encoding the hybrid protein of the present invention is also provided. The methods for making such chimeric nucleic acids and expression vectors containing them will be apparent to skilled artisans apprised of the present disclosure.

### 2.4. Protein Microchip

In accordance with another embodiment of the present invention, a protein microchip or microarray is provided having one or more of the protein complexes and/or antibodies selectively immunoreactive with the protein complexes of the present invention. Protein microarrays are becoming increasingly important in both proteomics research and protein-based detection and diagnosis of diseases. The protein microarrays in accordance with this embodiment of the present invention will be useful in a variety of applications including, e.g., large-scale or high-throughput screening for compounds capable of binding to the protein complexes or modulating the interactions between the interacting protein members in the protein complexes.

The protein microarray of the present invention can be prepared in a number of methods known in the art. An example of a suitable method is that disclosed in MacBeath and Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarray of the present invention can also be made by the method disclosed in PCT Publication No. WO 99/36576 assigned to Packard Bioscience Company, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Alternatively, the proteins and protein complexes of the present invention can be incorporated into a commercially available protein microchip, e.g., the ProteinChip System from Ciphergen Biosystems Inc., Palo Alto, Calif. The ProteinChip System comprises metal chips having a treated surface, which interact with proteins. Basically, a metal chip surface is coated with a silicon dioxide film. The molecules of interest such as proteins and protein complexes can then be attached covalently to the chip surface via a silane coupling agent.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

### 3. Antibodies

In accordance with another aspect of the present invention, an antibody immunoreactive against a protein complex of the present invention is provided. In one embodiment, the antibody is selectively immunoreactive with a protein complex of the present invention. Specifically, the phrase “selectively immunoreactive with a protein complex” as used herein means that the immunoreactivity of the antibody of the present invention with the protein complex is substantially higher than that with the individual interacting members of the protein complex so that the binding of the antibody to the protein complex is readily distinguishable from the binding of the antibody to the individual interacting member proteins based on the strength of the binding affinities. Preferably, the binding constants differ by a magnitude of at least 2 fold, more preferably at least 5 fold, even more preferably at least 10 fold, and most preferably at least 100 fold. In a specific embodiment, the antibody is not substantially immunoreactive with the interacting protein members of the protein complex.

The antibodies of the present invention can be readily prepared using procedures generally known in the art. See, e.g., Harlow and Lane, Antibodies: A Laboratory Manual, Cold Spring Harbor Press, 1988. Typically, the protein complex against which an immunoreactive antibody is desired is used as the antigen for producing an immune response in a host animal. In one embodiment, the protein complex used consists of the native proteins. Preferably, the protein complex includes only protein fragments containing interacting regions provided in the tables. As a result, a greater portion of the total antibodies may be selectively immunoreactive with the protein complexes. The interaction domains can be selected from, e.g., those regions summarized in Table 1. In addition, various techniques known in the art for predicting epitopes may also be employed to design antigenic peptides based on the interacting protein members in a protein complex of the present invention to increase the possibility of producing an antibody selectively immunoreactive with the protein complex. Suitable epitope-prediction computer programs include, e.g., MacVector from International Biotechnologies, Inc. and Protean from DNAStar.

In a specific embodiment, a hybrid protein as described above in Section 2.1 is used as an antigen which has a first protein that is any one of the proteins described in the tables, or a homologue, derivative, or fragment thereof covalently linked by a peptide bond or a peptide linker to a second protein which is the interacting partner of the first protein, or a homologue, derivative, or fragment of the second protein. In a preferred embodiment, the hybrid protein consists of two interacting domains selected from the regions identified in a table above, or homologues or derivatives thereof, covalently linked together by a peptide bond or a linker molecule.

The antibody of the present invention can be a polyclonal antibody to a protein complex of the present invention. To produce the polyclonal antibody, various animal hosts can be employed, including, e.g., mice, rats, rabbits, goats, guinea pigs, hamsters, etc. A suitable antigen which is a protein complex of the present invention or a derivative thereof as described above can be administered directly to a host animal to illicit immune reactions. Alternatively, it can be administered together with a carrier such as keyhole limpet hemocyanin (KLH), bovine serum albumin (BSA), ovalbumin, and Tetanus toxoid. Optionally, the antigen is conjugated to a carrier by a coupling agent such as carbodiimide, glutaraldehyde, and MBS. Any conventional adjuvants may be used to boost the immune response of the host animal to the protein complex antigen. Suitable adjuvants known in the art include but are not limited to Complete Freund's Adjuvant (which contains killed mycobacterial cells and mineral oil), incomplete Freund's Adjuvant (which lacks the cellular components), aluminum salts, MF59 from Chiron (Emeryville, Calif.), monophospholipid, synthetic trehalose dicorynomycolate (TDM) and cell wall skeleton (CWS) both from Corixa Corp. (Seattle, Wash.), non-ionic surfactant vesicles (NISV) from Proteus International PLC (Cheshire, U.K.), and saponins. The antigen preparation can be administered to a host animal by subcutaneous, intramuscular, intravenous, intradermal, or intraperitoneal injection, or by injection into a lymphoid organ.

The antibodies of the present invention may also be monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No. 4,376,110, which is incorporated herein by reference. Essentially, B-lymphocytes producing a polyclonal antibody against a protein complex of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein complex of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific desired protein may then be isolated from the library. The isolated cDNA can be introduced into a suitable host cell for the expression of the protein. Thus, recombinant techniques can be used to produce specific native antibodies, hybrid antibodies capable of simultaneous reaction with more than one antigen, chimeric antibodies (e.g., the constant and variable regions are derived from different sources), univalent antibodies that comprise one heavy and light chain pair coupled with the Fc region of a third (heavy) chain, Fab proteins, and the like. See U.S. Pat. No. 4,816,567; European Patent Publication No. 0088994; Munro, Nature, 312:597 (1984); Morrison, Science, 229:1202 (1985); Oi et al., BioTechniques, 4:214 (1986); and Wood et al., Nature, 314:446-449 (1985), all of which are incorporated herein by reference. Antibody fragments such as Fv fragments, single-chain Fv fragments (scFv), Fab′ fragments, and F(ab′)₂ fragments can also be recombinantly produced by methods disclosed in, e.g., U.S. Pat. No. 4,946,778; Skerra & Plückthun, Science, 240:1038-1041(1988); Better et al., Science, 240:1041-1043 (1988); and Bird, et al., Science, 242:423-426 (1988), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv. Immunol., 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. See e.g., U.S. Pat. Nos. 5,530,101; 5,545,806; 6,075,181; PCT Publication No. WO 94/02602; Green et. al., Nat. Genetics, 7: 13-21 (1994); and Lonberg et al., Nature 368: 856-859 (1994), all of which are incorporated herein by reference. The transgenic non-human host animal may be immunized with suitable antigens such as a protein complex of the present invention or one or more of the interacting protein members thereof to illicit specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library that is subsequently screened for clones encoding humanized antibodies with desired binding specificities.

In yet another embodiment, a bifunctional antibody is provided that has two different antigen binding sites, each being specific to a different interacting protein member in a protein complex of the present invention. The bifunctional antibody may be produced using a variety of methods known in the art. For example, two different monoclonal antibody-producing hybridomas can be fused together. One of the two hybridomas may produce a monoclonal antibody specific against an interacting protein member of a protein complex of the present invention, while the other hybridoma generates a monoclonal antibody immunoreactive with another interacting protein member of the protein complex. The thus formed new hybridoma produces different antibodies including a desired bifunctional antibody, i.e., an antibody immunoreactive with both of the interacting protein members. The bifunctional antibody can be readily purified. See Milstein and Cuello, Nature, 305:537-540 (1983).

Alternatively, a bifunctional antibody may also be produced using heterobifunctional crosslinkers to chemically link two different monoclonal antibodies, each being immunoreactive with a different interacting protein member of a protein complex. Therefore, the aggregate will bind to two interacting protein members of the protein complex. See Staerz et al, Nature, 314:628-631(1985); Perez et al, Nature, 316:354-356 (1985).

In addition, bifunctional antibodies can also be produced by recombinantly expressing light and heavy chain genes in a hybridoma that itself produces a monoclonal antibody. As a result, a mixture of antibodies including a bifunctional antibody is produced. See DeMonte et al, Proc. Natl. Acad. Sci., USA, 87:2941-2945 (1990); Lenz and Weidle, Gene, 87:213-218 (1990).

Preferably, a bifunctional antibody in accordance with the present invention is produced by the method disclosed in U.S. Pat. No. 5,582,996, which is incorporated herein by reference. For example, two different Fabs can be provided and mixed together. The first Fab can bind to an interacting protein member of a protein complex, and has a heavy chain constant region having a first complementary domain not naturally present in the Fab but capable of binding a second complementary domain. The second Fab is capable of binding another interacting protein member of the protein complex, and has a heavy chain constant region comprising a second complementary domain not naturally present in the Fab but capable of binding to the first complementary domain. Each of the two complementary domains is capable of stably binding to the other but not to itself. For example, the leucine zipper regions of c-fos and c-jun oncogenes may be used as the first and second complementary domains. As a result, the first and second complementary domains interact with each other to form a leucine zipper thus associating the two different Fabs into a single antibody construct capable of binding to two antigenic sites.

Other suitable methods known in the art for producing bifunctional antibodies may also be used, which include those disclosed in Holliger et al., Proc. Nat'l Acad. Sci. USA, 90:6444-6448 (1993); de Kruif et al., J. Biol. Chem., 271:7630-7634 (1996); Coloma and Morrison, Nat. Biotechnol., 15:159-163 (1997); Muller et al., FEBS Lett., 422:259-264 (1998); and Muller et al., FEBS Lett., 432:45-49 (1998), all of which are incorporated herein by reference.

### 4. Methods of Detecting Protein Complexes

Another aspect of the present invention relates to methods for detecting the protein complexes of the present invention, particularly for determining the concentration of a specific protein complex in a patient sample.

In one embodiment, the concentration of a protein complex of the present invention is determined in cells, tissue, or an organ of a patient. For example, the protein complex can be isolated or purified from a patient sample obtained from cells, tissue, or an organ of the patient and the amount thereof is determined. As described above, the protein complex can be prepared from cells, tissue or organ samples by coimmunoprecipitation using an antibody immunoreactive with an interacting protein member, a bifunctional antibody that is immunoreactive with two or more interacting protein members of the protein complex, or preferably an antibody selectively immunoreactive with the protein complex. When bifunctional antibodies or antibodies immunoreactive with only free interacting protein members are used, individual interacting protein members not complexed with other proteins may also be isolated along with the protein complex containing such individual proteins. However, they can be readily separated from the protein complex using methods known in the art, e.g., size-based separation methods such as gel filtration, or by subtracting the protein complex from the mixture using an antibody specific against another individual interacting protein member. Additionally, proteins in a sample can be separated in a gel such as polyacrylamide gel and subsequently immunoblotted using an antibody immunoreactive with the protein complex.

Alternatively, the concentration of the protein complex can be determined in a sample without separation, isolation or purification. For this purpose, it is preferred that an antibody selectively immunoreactive with the specific protein complex is used in an immunoassay. For example, immunocytochemical methods can be used. Other well known antibody-based techniques can also be used including, e.g., enzyme-linked immunosorbent assay (ELISA), radioimmunoassay (RIA), immunoradiometric assays (IRMA), fluorescent immunoassays, protein A immunoassays, and immunoenzymatic assays (IEMA). See e.g., U.S. Pat. Nos. 4,376,110 and 4,486,530, both of which are incorporated herein by reference.

In addition, since a specific protein complex is formed from its interacting protein members, if one of the interacting protein members is at a relatively low concentration in a patient, it may be reasonably expected that the concentration of the protein complex in the patient may also be low. Therefore, the concentration of an individual interacting protein member of a specific protein complex can be determined in a patient sample which can then be used as a reasonably accurate indicator of the concentration of the protein complex in the sample. For this purpose, antibodies against an individual interacting protein member of a specific complex can be used in any one of the methods described above. In a preferred embodiment, the concentration of each of the interacting protein members of a protein complex is determined in a patient sample and the relative concentration of the protein complex is then deduced.

In addition, the relative protein complex concentration in a patient can also be determined by determining the concentration of the mRNA encoding an interacting protein member of the protein complex. Preferably, each interacting protein member's mRNA concentration in a patient sample is determined. For this purpose, methods for determining mRNA concentration generally known in the art may all be used. Examples of such methods include, e.g., Northern blot assay, dot blot assay, PCR assay (preferably quantitative PCR assay), in situ hybridization assay, and the like.

As discussed above, each interaction between members of an interacting protein pair of the present invention suggests that the proteins and/or the protein complexes formed by such proteins may be involved in common biological processes and disease pathways. In addition, the interactions under physiological conditions may lead to the formation of protein complexes in vivo. The protein complexes are expected to mediate the functions and biological activities of the interacting members of the protein complexes. Thus, aberrations in the protein complexes or the individual proteins and the degree of the aberration may be indicators for the diseases or disorders. These aberrations may be used as parameters for classifying and/or staging one of the above-described diseases. In addition, they may also be indicators for patients' response to a drug therapy.

Association between a physiological state (e.g., physiological disorder, predisposition to the disorder, a disease state, response to a drug therapy, or other physiological phenomena or phenotypes) and a specific aberration in a protein complex of the present invention or an individual interacting member thereof can be readily determined by comparative analysis of the protein complex and/or the interacting members thereof in a normal population and an abnormal or affected population. Thus, for example, one can study the concentration, localization and distribution of a particular protein complex, mutations in the interacting protein members of the protein complex, and/or the binding affinity between the interacting protein members in both a normal population and a population affected with a particular physiological disorder described above. The study results can be compared and analyzed by statistical means. Any detected statistically significant difference in the two populations would indicate an association. For example, if the concentration of the protein complex is statistically significantly higher in the affected population than in the normal population, then it can be reasonably concluded that higher concentration of the protein complex is associated with the physiological disorder.

Thus, once an association is established between a particular type of aberration in a particular protein complex of the present invention or in an interacting protein member thereof and a physiological disorder or disease or predisposition to the physiological disorder or disease, then the particular physiological disorder or disease or predisposition to the physiological disorder or disease can be diagnosed or detected by determining whether a patient has the particular aberration.

Accordingly, the present invention also provides a method for diagnosing in a patient a disease or physiological disorder, or a predisposition to the disease or disorder, such as diabestes, obesity, Alzheimer's disease and neurodegenerative disorders by determining whether there is any aberration in the patient with respect to a protein complex identified according to the present invention. The same protein complex is analyzed in a normal individual and is compared with the results obtained in the patient. In this manner, any protein complex aberration in the patient can be detected. As used herein, the term “aberration” when used in the context of protein complexes of the present invention means any alterations of a protein complex including increased or decreased concentration of the protein complex in a particular cell or tissue or organ or the total body, altered localization of the protein complex in cellular compartments or in locations of a tissue or organ, changes in binding affinity of an interacting protein member of the protein complex, mutations in an interacting protein member or the gene encoding the protein, and the like. As will be apparent to a skilled artisan, the term “aberration” is used in a relative sense. That is, an aberration is relative to a normal condition.

As used herein, the term “diagnosis” means detecting a disease or disorder or determining the stage or degree of a disease or disorder. The term “diagnosis” also encompasses detecting a predisposition to a disease or disorder, determining the therapeutic effect of a drug therapy, or predicting the pattern of response to a drug therapy or xenobiotics. The diagnosis methods of the present invention may be used independently, or in combination with other diagnosing and/or staging methods known in the medical art for a particular disease or disorder.

Thus, in one embodiment, the method of diagnosis is conducted by detecting, in a patient, the concentrations of one or more protein complexes of the present invention using any one of the methods described above, and determining whether the patient has an aberrant concentration of the protein complexes.

The diagnosis may also be based on the determination of the concentrations of one or more interacting protein members (at the protein, cDNA or mRNA level) of a protein complex of the present invention. An aberrant concentration of an interacting protein member may indicate a physiological disorder or a predisposition to a physiological disorder.

In another embodiment, the method of diagnosis comprises determining, in a patient, the cellular localization, or tissue or organ distribution of a protein complex of the present invention and determining whether the patient has an aberrant localization or distribution of the protein complex. For example, immunocytochemical or immunohistochemical assays can be performed on a cell, tissue or organ sample from a patient using an antibody selectively immunoreactive with a protein complex of the present invention. Antibodies immunoreactive with both an individual interacting protein member and a protein complex containing the protein member may also be used, in which case it is preferred that antibodies immunoreactive with other interacting protein members are also used in the assay. In addition, nucleic acid probes may also be used in in situ hybridization assays to detect the localization or distribution of the mRNAs encoding the interacting protein members of a protein complex. Preferably, the mRNA encoding each interacting protein member of a protein complex is detected concurrently.

In yet another embodiment, the method of diagnosis of the present invention comprises detecting any mutations in one or more interacting protein members of a protein complex of the present invention. In particular, it is desirable to determine whether the interacting protein members have any mutations that will lead to, or are associated with, changes in the functional activity of the proteins or changes in their binding affinity to other interacting protein members in forming a protein complex of the present invention. Examples of such mutations include but are not limited to, e.g., deletions, insertions and rearrangements in the genes encoding the protein members, and nucleotide or amino acid substitutions and the like. In a preferred embodiment, the domains of the interacting protein members that are responsible for the protein-protein interactions, and lead to protein complex formation, are screened to detect any mutations therein. For example, genomic DNA or cDNA encoding an interacting protein member can be prepared from a patient sample, and sequenced. The thus obtained sequence may be compared with known wild-type sequences to identify any mutations. Alternatively, an interacting protein member may be purified from a patient sample and analyzed by protein sequencing or mass spectrometry to detect any amino acid sequence changes. Any methods known in the art for detecting mutations may be used, as will be apparent to skilled artisans apprised of the present disclosure.

In another embodiment, the method of diagnosis includes determining the binding constant of the interacting protein members of one or more protein complexes. For example, the interacting protein members can be obtained from a patient by direct purification or by recombinant expression from genomic DNAs or cDNAs prepared from a patient sample encoding the interacting protein members. Binding constants represent the strength of the protein-protein interaction between the interacting protein members in a protein complex. Thus, by measuring binding constants, subtle aberrations in binding affinity may be detected.

A number of methods known in the art for estimating and determining binding constants in protein-protein interactions are reviewed in Phizicky and Fields, et al., Microbiol. Rev., 59:94-123 (1995), which is incorporated herein by reference. For example, protein affinity chromatography may be used. First, columns are prepared with different concentrations of an interacting protein member, which is covalently bound to the columns. Then a preparation of an interacting protein partner is run through the column and washed with buffer. The interacting protein partner bound to the interacting protein member linked to the column is then eluted. A binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, proteins can sediment as a protein complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J. Mol. Biol., 220:307-324 (1991)), the Hummel-Dreyer method of equilibrium gel filtration (See e.g., Hummel and Dreyer, Biochim. Biophys. Acta, 63:530-532 (1962)) and large-zone equilibrium gel filtration (See e.g., Gilbert and Kellett, J. Biol. Chem., 246:6079-6086 (1971)), sedimentation equilibrium (See e.g., Rivas and Minton, Trends Biochem., 18:284-287 (1993)), fluorescence methods such as fluorescence spectrum (See e.g., Otto-Bruc et al, Biochemistry, 32:8632-8645 (1993)) and fluorescence polarization or anisotropy with tagged molecules (See e.g., Weiel and Hershey, Biochemistry, 20:5859-5865 (1981)), solution equilibrium measured with immobilized binding protein (See e.g., Nelson and Long, Biochemistry, 30:2384-2390 (1991)), and surface plasmon resonance (See e.g., Panayotou et al., Mol. Cell. Biol., 13:3567-3576 (1993)).

In another embodiment, the diagnosis method of the present invention comprises detecting protein-protein interactions in functional assay systems such as the yeast two-hybrid system. Accordingly, to determine the protein-protein interaction between two interacting protein members that normally form a protein complex in normal individuals, cDNAs encoding the interacting protein members can be isolated from a patient to be diagnosed. The thus cloned cDNAs or fragments thereof can be subcloned into vectors for use in yeast two-hybrid systems. Preferably a reverse yeast two-hybrid system is used such that failure of interaction between the proteins may be positively detected. The use of yeast two-hybrid systems or other systems for detecting protein-protein interactions is known in the art and is described below in Section 5.3.1.

A kit may be used for conducting the diagnosis methods of the present invention. Typically, the kit should contain, in a carrier or compartmentalized container, reagents useful in any of the above-described embodiments of the diagnosis method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a protein complex of the present invention. In addition, antibodies against individual interacting protein members of the protein complexes may also be included. The antibodies may be labeled with a detectable marker such as radioactive isotopes, or enzymatic or fluorescence markers. Alternatively secondary antibodies such as labeled anti-IgG and the like may be included for detection purposes. Optionally, the kit can include one or more of the protein complexes of the present invention prepared or purified from a normal individual or an individual afflicted with a physiological disorder associated with an aberration in the protein complexes or an interacting protein member thereof. In addition, the kit may further include one or more of the interacting protein members of the protein complexes of the present invention prepared or purified from a normal individual or an individual afflicted with a physiological disorder associated with an aberration in the protein complexes or an interacting protein member thereof. Suitable oligonucleotide primers useful in the amplification of the genes or cDNAs for the interacting protein members may also be provided in the kit. In particular, in a preferred embodiment, the kit includes a first oligonucleotide selectively hybridizable to the mRNA or cDNA encoding one member of an interacting pair of proteins and a second oligonucleotide selectively hybridizable to the mRNA or cDNA encoding the other of the interacting pair. Additional oligonucleotides hybridizing to a region of the genes encoding an interacting pair of proteins may also be included. Such oligonucleotides may be used as PCR primers for, e.g., quantitative PCR amplification of mRNAs encoding the interacting proteins, or as hybridizing probes for detecting the mRNAs. The oligonucleotides may have a length of from about 8 nucleotides to about 100 nucleotides, preferably from about 12 to about 50 nucleotides, and more preferably from about 15 to about 30 nucleotides. In addition, the kit may also contain oligonucleotides that can be used as hybridization probes for detecting the cDNAs or mRNAs encoding the interacting protein members. Preferably, instructions for using the kit or reagents contained therein are also included in the kit.

### 5. Use of Protein Complexes or Interacting Protein Members Thereof in Screening Assays for Modulators

The protein complexes of the present invention and interacting members thereof can also be used in screening assays to identify modulators of the protein complexes, and/or the interacting proteins. In addition, homologues, derivatives or fragments of the interacting proteins provided in this invention may also be used in such screening assays. As used herein, the term “modulator” encompasses any compounds that can cause any form of alteration of the biological activities or functions of the proteins or protein complexes, including, e.g., enhancing or reducing their biological activities, increasing or decreasing their stability, altering their affinity or specificity to certain other biological molecules, etc. In addition, the term “modulator” as used herein also includes any compounds that simply bind any of the proteins described in the tables, and/or the proteins complexes of the present invention. For example, a modulator can be an “interaction antagonist” capable of interfering with or disrupting or dissociating protein-protein interaction between an interacting pair of proteins identified in the tables, or homologues, fragments or derivatives thereof. A modulator can also be an “interaction agonist” that initiates or strengthens the interaction between the protein members of a protein complex of the present invention, or homologues, fragments or derivatives thereof.

In addition, the discovery of protein ligands of the present invention allows the use of screening assays to identify modulators of individual proteins of the protein complexes. Typical high-throughput screening assays involve measuring the modulation of the enzymatic activity of a protein. However, typical high-throughput screening assays are not applicable to proteins that exhibit little or no measurable enzymatic activity. The present discovery of novel ligands of proteins allows a screen to be setup that does not utilize enzymatic activity measurements. Consequently, the present invention enables a non-enzymatic high-throughput assay to be performed for modulators of individual proteins and/or protein complexes described in the tables.

Accordingly, the present invention provides screening methods for selecting modulators of any of the proteins described in the tables, or a mutant form thereof, or a protein-protein interaction between an interacting pair of proteins provided in the present invention, or homologues, fragments or derivatives thereof.

The selected compounds can be tested for their ability to modulate (interfere with or strengthen) the interaction between the interacting partners within the protein complexes of the present invention. In addition, the compounds can also be further tested for their ability to modulate (inhibit or enhance) cellular functions such as cellular glucose transport in cells as well as their effectiveness in treating diseases such as diabestes, obesity, Alzheimer's disease and neurodegenerative disorders.

The modulators selected in accordance with the screening methods of the present invention can be effective in modulating the functions or activities of individual interacting proteins, or the protein complexes of the present invention. For example, compounds capable of binding to the protein complexes may be capable of modulating the functions of the protein complexes. Additionally, compounds that interfere with, weaken, dissociate or disrupt, or alternatively, initiate, facilitate or stabilize the protein-protein interaction between the interacting protein members of the protein complexes can also be effective in modulating the functions or activities of the protein complexes. Thus, the compounds identified in the screening methods of the present invention can be made into therapeutically or prophylactically effective drugs for preventing or ameliorating diseases, disorders or symptoms caused by or associated with a protein complex or an interacting member thereof. Alternatively, they may be used as leads to aid the design and identification of therapeutically or prophylactically effective compounds for diseases, disorders or symptoms caused by or associated with the protein complex or interacting protein members thereof. The protein complexes and/or interacting protein members thereof in accordance with the present invention can be used in any of a variety of drug screening techniques. Drug screening can be performed as described herein or using well-known techniques, such as those described in U.S. Pat. Nos. 5,800,998 and 5,891,628, both of which are incorporated herein by reference.

### 5.1. Test Compounds

Any test compounds may be screened in the screening assays of the present invention to select modulators of the protein complexes or interacting members thereof. By the term “selecting” or “select” compounds it is intended to encompass both (a) choosing compounds from a group previously unknown to be modulators of a protein complex or interacting protein members thereof; and (b) testing compounds that are known to be capable of binding, or modulating the functions and activities of, a protein complex or interacting protein members thereof. Both types of compounds are generally referred to herein as “test compounds.” The test compounds may include, by way of example, proteins (e.g., antibodies, small peptides, artificial or natural proteins), nucleic acids, and derivatives, mimetics and analogs thereof, and small organic molecules having a molecular weight of no greater than 10,000 daltons, more preferably less than 5,000 daltons. Preferably, the test compounds are provided in library formats known in the art, e.g., in chemically synthesized libraries, recombinantly expressed libraries (e.g., phage display libraries), and in vitro translation-based libraries (e.g., ribosome display libraries).

For example, the screening assays of the present invention can be used in the antibody production processes described in Section 3 to select antibodies with desirable specificities. Various forms of antibodies or derivatives thereof may be screened, including but not limited to, polyclonal antibodies, monoclonal antibodies, bifunctional antibodies, chimeric antibodies, single chain antibodies, antibody fragments such as Fv fragments, single-chain Fv fragments (scFv), Fab′ fragments, and F(ab′)₂ fragments, and various modified forms of antibodies such as catalytic antibodies, and antibodies conjugated to toxins or drugs, and the like. The antibodies can be of any types such as IgG, IgE, IgA, or IgM. Humanized antibodies are particularly preferred. Preferably, the various antibodies and antibody fragments may be provided in libraries to allow large-scale high throughput screening. For example, expression libraries expressing antibodies or antibody fragments may be constructed by a method disclosed, e.g., in Huse et al., Science, 246:1275-1281 (1989), which is incorporated herein by reference. Single-chain Fv (scFv) antibodies are of particular interest in diagnostic and therapeutic applications. Methods for providing antibody libraries are also provided in U.S. Pat. Nos. 6,096,551; 5,844,093; 5,837,460; 5,789,208; and 5,667,988, all of which are incorporated herein by reference.

Peptidic test compounds may be peptides having L-amino acids and/or D-amino acids, phosphopeptides, and other types of peptides. The screened peptides can be of any size, but preferably have less than about 50 amino acids. Smaller peptides are easier to deliver into a patient's body. Various forms of modified peptides may also be screened. Like antibodies, peptides can also be provided in, e.g., combinatorial libraries. See generally, Gallop et al., J. Med. Chem., 37:1233-1251 (1994). Methods for making random peptide libraries are disclosed in, e.g., Devlin et al., Science, 249:404-406 (1990). Other suitable methods for constructing peptide libraries and screening peptides therefrom are disclosed in, e.g., Scott and Smith, Science, 249:386-390 (1990); Moran et al., J. Am. Chem. Soc., 117:10787-10788 (1995) (a library of electronically tagged synthetic peptides); Stachelhaus et al., Science, 269:69-72 (1995); U.S. Pat. Nos. 6,156,511; 6,107,059; 6,015,561; 5,750,344; 5,834,318; 5,750,344, all of which are incorporated herein by reference. For example, random-sequence peptide phage display libraries may be generated by cloning synthetic oligonucleotides into the gene III or gene VIII of an E. coli filamentous phage. The thus generated phage can propagate in E. coli. and express peptides encoded by the oligonucleotides as fusion proteins on the surface of the phage. Scott and Smith, Science, 249:368-390 (1990). Alternatively, the “peptides on plasmids” method may also be used to form peptide libraries. In this method, random peptides may be fused to the C-terminus of the E. coli. Lac repressor by recombinant technologies and expressed from a plasmid that also contains Lac repressor-binding sites. As a result, the peptide fusions bind to the same plasmid that encodes them.

Small organic or inorganic non-peptide non-nucleotide compounds are preferred test compounds for the screening assays of the present invention. They too can be provided in a library format. See generally, Gordan et al. J. Med. Chem., 37:1385-1401 (1994). For example, benzodiazepine libraries are provided in Bunin and Ellman, J. Am. Chem. Soc., 114:10997-10998 (1992), which is incorporated herein by reference. Methods for constructing and screening peptoid libraries are disclosed in Simon et al., Proc. Natl. Acad. Sci. USA, 89:9367-9371 (1992). Methods for the biosynthesis of novel polyketides in a library format are described in McDaniel et al, Science, 262:1546-1550 (1993) and Kao et al., Science, 265:509-512 (1994). Various libraries of small organic molecules and methods of construction thereof are disclosed in U.S. Pat. Nos. 6,162,926 (multiply-substituted fullerene derivatives); 6,093,798 (hydroxamic acid derivatives); 5,962,337 (combinatorial 1,4-benzodiazepin-2,5-dione library); 5,877,278 (Synthesis of N-substituted oligomers); 5,866,341 (compositions and methods for screening drug libraries); 5,792,821 (polymerizable cyclodextrin derivatives); 5,766,963 (hydroxypropylamine library); and 5,698,685 (morpholino-subunit combinatorial library), all of which are incorporated herein by reference.

Other compounds such as oligonucleotides and peptide nucleic acids (PNA), and analogs and derivatives thereof may also be screened to identify clinically useful compounds. Combinatorial libraries of oligonucleotides are also known in the art. See Gold et al., J. Biol. Chem., 270:13581-13584 (1995).

### 5.2. In vitro Screening Assays

The test compounds may be screened in an in vitro assay to identify compounds capable of binding the protein complexes or interacting protein members thereof in accordance with the present invention. For this purpose, a test compound is contacted with a protein complex or an interacting protein member thereof under conditions and for a time sufficient to allow specific interaction between the test compound and the target components to occur, thereby resulting in the binding of the compound to the target, and the formation of a complex. Subsequently, the binding event is detected.

Various screening techniques known in the art may be used in the present invention. The protein complexes and the interacting protein members thereof may be prepared by any suitable methods, e.g., by recombinant expression and purification. The protein complexes and/or interacting protein members thereof (both are referred to as “target” hereinafter in this section) may be free in solution. A test compound may be mixed with a target forming a liquid mixture. The compound may be labeled with a detectable marker. Upon mixing under suitable conditions, the binding complex having the compound and the target may be co-immunoprecipitated and washed. The compound in the precipitated complex may be detected based on the marker on the compound.

In a preferred embodiment, the target is immobilized on a solid support or on a cell surface. Preferably, the target can be arrayed into a protein microchip in a method described in Section 2.3. For example, a target may be immobilized directly onto a microchip substrate such as glass slides or onto multi-well plates using non-neutralizing antibodies, i.e., antibodies capable of binding to the target but do not substantially affect its biological activities. To affect the screening, test compounds can be contacted with the immobilized target to allow binding to occur to form complexes under standard binding assay conditions. Either the targets or test compounds are labeled with a detectable marker using well-known labeling techniques. For example, U.S. Pat. No. 5,741,713 discloses combinatorial libraries of biochemical compounds labeled with NMR active isotopes. To identify binding compounds, one may measure the formation of the target-test compound complexes or kinetics for the formation thereof. When combinatorial libraries of organic non-peptide non-nucleic acid compounds are screened, it is preferred that labeled or encoded (or “tagged”) combinatorial libraries are used to allow rapid decoding of lead structures. This is especially important because, unlike biological libraries, individual compounds found in chemical libraries cannot be amplified by self-amplification. Tagged combinatorial libraries are provided in, e.g., Borchardt and Still, J. Am. Chem. Soc., 116:373-374 (1994) and Moran et al., J. Am. Chem. Soc., 117:10787-10788 (1995), both of which are incorporated herein by reference.

Alternatively, the test compounds can be immobilized on a solid support, e.g., forming a microarray of test compounds. The target protein or protein complex is then contacted with the test compounds. The target may be labeled with any suitable detection marker. For example, the target may be labeled with radioactive isotopes or fluorescence marker before binding reaction occurs. Alternatively, after the binding reactions, antibodies that are immunoreactive with the target and are labeled with radioactive materials, fluorescence markers, enzymes, or labeled secondary anti-Ig antibodies may be used to detect any bound target thus identifying the binding compound. One example of this embodiment is the protein probing method. That is, the target provided in accordance with the present invention is used as a probe to screen expression libraries of proteins or random peptides. The expression libraries can be phage display libraries, in vitro translation-based libraries, or ordinary expression cDNA libraries. The libraries may be immobilized on a solid support such as nitrocellulose filters. See e.g., Sikela and Hahn, Proc. Natl. Acad. Sci. USA, 84:3038-3042 (1987). The probe may be labeled with a radioactive isotope or a fluorescence marker. Alternatively, the probe can be biotinylated and detected with a streptavidin-alkaline phosphatase conjugate. More conveniently, the bound probe may be detected with an antibody.

In a specific embodiment, a protein complex used in the screening assay includes a hybrid protein as described in Section 2.1, which is formed by fusion of two interacting protein members or fragments or interaction domains thereof. The hybrid protein may also be designed such that it contains a detectable epitope tag fused thereto. Suitable examples of such epitope tags include sequences derived from, e.g., influenza virus hemagglutinin (HA), Simian Virus 5 (V5), polyhistidine (6×His), c-myc, lacZ, GST, and the like.

In addition, a known ligand capable of binding to the target can be used in competitive binding assays. Complexes between the known ligand and the target can be formed and then contacted with test compounds. The ability of a test compound to interfere with the interaction between the target and the known ligand is measured. One exemplary ligand is an antibody capable of specifically binding the target. Particularly, such an antibody is especially useful for identifying peptides that share one or more antigenic determinants of the target protein complex or interacting protein members thereof.

In a specific preferred embodiment, the target is one member of an interacting pair of proteins disclosed according the present invention, or a homologue, derivative or fragment thereof, and the competitive ligand is the other member of the interacting pair of proteins, or a homologue, derivative or fragment thereof. Preferably, either the target or the ligand or both are labeled with or detectable marker. Alternatively, either the target or the ligand or both are fusion proteins that contain a detectable epitope tag having one or more sequences derived from, e.g., influenza virus hemagglutinin (HA), Simian Virus 5 (V5), polyhistidine (6×His), c-myc, lacZ, GST, and the like. For example, a target protein such as GLUT4 can be contacted with test compounds in the presence of one of its binding interactors identified according to the present invention, e.g., PN7065, and the interaction of GLUT4 and its interactor can be determined. Compounds that modulate or bind GLUT4 may be identified by their activity of modulating GLUT4's binding with its interactor, e.g., PN7065. Preferably, the selected modulators can be further tested in an assay useful in determining their efficacy in modulating cellular glucose transport or insulin response.

Thus, for example, the target can be immobilized to a solid support. The ligand can be a fusion protein having a fragment of an interactor of the target protein fused to an epitope tag, e.g., c-myc. The ligand can be contacted with the target in the presence or absence of one or more test compounds. Both ligand molecules associated with the immobilized target and ligand molecules not associated with the target can be detected with, e.g., an antibody against the c-myc tag. As a result, test compounds capable of binding the target or ligand, or disrupting the protein-protein interaction between the target and ligand can be identified or selected.

Test compounds may also be screened in an in vitro assay to identify compounds capable of dissociating the protein complexes identified in the tables above. Thus, for example, any one of the interacting pairs of proteins described in the tables above can be contacted with a test compound and the integrity of the protein complex can be assessed. Conversely, test compounds may also be screened to identify compounds capable of enhancing the interactions between the constituent members of the protein complexes formed by the interactions described in the tables. The assays can be conducted in a manner similar to the binding assays described above. For example, the presence or absence of a particular pair of interacting proteins can be detected by an antibody selectively immunoreactive with the protein complex formed by those two proteins. Thus, after incubation of the protein complex with a test compound, an immunoprecipitation assay can be conducted with the antibody. If the test compound disrupts the protein complex, then the amount of immunoprecipitated protein complex in this assay will be significantly less than that in a control assay in which the same protein complex is not contacted with the test compound. Similarly, two proteins—the interaction between which is to be enhanced—may be incubated together with a test compound. Thereafter, a protein complex formed by the two interacting proteins may be detected by the selectively immunoreactive antibody. The amount of protein complex may be compared to that formed in the absence of the test compound. Various other detection methods may be suitable in the dissociation assay, as will be apparent to a skilled artisan apprised of the present disclosure.

In another emobodiment of the present invention, a modulator of the PN7065 can be selected by contacting a test compound with PN7065 and measuring the activity of PN7065 in the presence of the test compound. For example, the kinase activity of PN7065 can be measured when PN7065 is contacted with a test compound. Various kinase activity assays that are known in the art can be employed to measure the kinase activity of PN7065 in the presence of a test compound.

### 5.3. In vivo Screening Assays

Test compounds can also be screened in any in vivo assays to select modulators of the protein complexes or interacting protein members thereof in accordance with the present invention. For example, any in vivo assays known in the art to be useful in identifying compounds capable of strengthening or interfering with the stability of the protein complexes of the present invention may be used.

In a specific example, a screening assay for modulators of a GLUT4 is performed by using PN7065 as a ligand for GLUT4. In this screen, GLUT4 is contacted with test compounds in the presence of PN7065 and the levels of GLUT4-PN7065 protein complex formed when GLUT4 is contacted with the test compound in the presence of PN7065 is detected. The ability of the test compounds to modulate GLUT4 is determined by comparing the level of GLUT4-PN7065 complex formed when GLUT4 is contacted with test compounds to the level formed in the absence of test compounds. If the level of GLUT4-PN7065 protein complex formed when GLUT4 is contacted with the test compound then the test compound is a modulator of GLUT4.

To screen peptidic compounds for modulators of GLUT4, the two-hybrid systems described in Section 4 may be used in the screening assays in which the GLUT4 protein is expressed in, e.g., a bait fusion protein and the peptidic test compounds are expressed in, e.g., prey fusion proteins.

To screen for modulators of the protein-protein interaction between GLUT4 and a GLUT4-interacting protein, the methods of the present invention typically comprise contacting the GLUT4 protein with the GLUT4-interacting protein in the presence of a test compound, and determining the interaction between the GLUT4 protein and the GLUT4-interacting protein. In a preferred embodiment, a two-hybrid system, e.g., a yeast two-hybrid system as described in detail in Section 4 is employed.

### 5.3.1. Two-Hybrid Assays

In a preferred embodiment, one of the yeast two-hybrid systems or their analogous or derivative forms is used. Examples of suitable two-hybrid systems known in the art include, but are not limited to, those disclosed in U.S. Pat. Nos. 5,283,173; 5,525,490; 5,585,245; 5,637,463; 5,695,941; 5,733,726; 5,776,689; 5,885,779; 5,905,025; 6,037,136; 6,057,101; 6,114,111; and Bartel and Fields, eds., The Yeast Two-Hybrid System, Oxford University Press, New York, N.Y., 1997, all of which are incorporated herein by reference.

Typically, in a classic transcription-based two-hybrid assay, two chimeric genes are prepared encoding two fusion proteins: one contains a transcription activation domain fused to an interacting protein member of a protein complex of the present invention or an interaction domain or fragment of the interacting protein member, while the other fusion protein includes a DNA binding domain fused to another interacting protein member of the protein complex or a fragment or interaction domain thereof. For the purpose of convenience, the two interacting protein members, fragments or interaction domains thereof are referred to as “bait fusion protein” and “prey fusion protein,” respectively. The chimeric genes encoding the fusion proteins are termed “bait chimeric gene” and “prey chimeric gene,” respectively. Typically, a “bait vector” and a “prey vector” are provided for the expression of a bait chimeric gene and a prey chimeric gene, respectively.

### 5.3.1.1. Vectors

Many types of vectors can be used in a transcription-based two-hybrid assay. Methods for the construction of bait vectors and prey vectors should be apparent to skilled artisans in the art apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. 11, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Rothstein in DNA Cloning: A Practical Approach, Vol. 11, Ed. DM Glover, IRL Press, Wash., D.C., 1986.

Generally, the bait and prey vectors include an expression cassette having a promoter operably linked to a chimeric gene for the transcription of the chimeric gene. The vectors may also include an origin of DNA replication for the replication of the vectors in host cells and a replication origin for the amplification of the vectors in, e.g., E. coli, and selection marker(s) for selecting and maintaining only those host cells harboring the vectors. Additionally, the expression cassette preferably also contains inducible elements, which function to control the expression of a chimeric gene. Making the expression of the chimeric genes inducible and controllable is especially important in the event that the fusion proteins or components thereof are toxic to the host cells. Other regulatory sequences such as transcriptional enhancer sequences and translation regulation sequences (e.g., Shine-Dalgarno sequence) can also be included in the expression cassette. Termination sequences such as the bovine growth hormone, SV40, lacZ and AcMNPV polyhedral polyadenylation signals may also be operably linked to a chimeric gene in the expression cassette. An epitope tag coding sequence for detection and/or purification of the fusion proteins can also be operably linked to the chimeric gene in the expression cassette. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies to many epitope tags are generally commercially available. The vectors can be introduced into the host cells by any techniques known in the art, e.g., by direct DNA transformation, microinjection, electroporation, viral infection, lipofection, gene gun, and the like. The bait and prey vectors can be maintained in host cells in an extrachromosomal state, i.e., as self-replicating plasmids or viruses. Alternatively, one or both vectors can be integrated into chromosomes of the host cells by conventional techniques such as selection of stable cell lines or site-specific recombination.

The in vivo assays of the present invention can be conducted in many different host cells, including but not limited to bacteria, yeast cells, plant cells, insect cells, and mammalian cells. A skilled artisan will recognize that the designs of the vectors can vary with the host cells used. In one embodiment, the assay is conducted in prokaryotic cells such as Escherichia coli, Salmonella, Klebsiella, Pseudomonas, Caulobacter, and Rhizobium. Suitable origins of replication for the expression vectors useful in this embodiment of the present invention include, e.g., the ColE1, pSC101, and M13 origins of replication. Examples of suitable promoters include, for example, the T7 promoter, the lacZ promoter, and the like. In addition, inducible promoters are also useful in modulating the expression of the chimeric genes. For example, the lac operon from bacteriophage lambda plac5 is well known in the art and is inducible by the addition of IPTG to the growth medium. Other known inducible promoters useful in a bacteria expression system include pL of bacteriophage λ, the trp promoter, and hybrid promoters such as the tac promoter, and the like.

In addition, selection marker sequences for selecting and maintaining only those prokaryotic cells expressing the desirable fusion proteins should also be incorporated into the expression vectors. Numerous selection markers including auxotrophic markers and antibiotic resistance markers are known in the art and can all be useful for purposes of this invention. For example, the bla gene, which confers ampicillin resistance, is the most commonly used selection marker in prokaryotic expression vectors. Other suitable markers include genes that confer neomycin, kanamycin, or hygromycin resistance to the host cells. In fact, many vectors are commercially available from vendors such as Invitrogen Corp. of Carlsbad, Calif., Clontech Corp. of Palo Alto, Calif., and Stratagene Corp. of La Jolla, Calif., and Promega Corp. of Madison, Wis. These commercially available vectors, e.g., pBR322, pSPORT, pBluescriptIISK, pcDNAI, and pcDNAII all have a multiple cloning site into which the chimeric genes of the present invention can be conveniently inserted using conventional recombinant techniques. The constructed expression vectors can be introduced into host cells by various transformation or transfection techniques generally known in the art.

In another embodiment, mammalian cells are used as host cells for the expression of the fusion proteins and detection of protein-protein interactions. For this purpose, virtually any mammalian cells can be used including normal tissue cells, stable cell lines, and transformed tumor cells. Conveniently, mammalian cell lines such as CHO cells, Jurkat T cells, NIH 3T3 cells, HEK-293 cells, CV-1 cells, COS-1 cells, HeLa cells, VERO cells, MDCK cells, W138 cells, and the like are used. Mammalian expression vectors are well known in the art and many are commercially available. Examples of suitable promoters for the transcription of the chimeric genes in mammalian cells include viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Inducible promoters can also be used. Suitable inducible promoters include, for example, the tetracycline responsive element (TRE) (See Gossen et al., Proc. Natl. Acad. Sci. USA, 89:5547-5551 (1992)), metallothionein IIA promoter, ecdysone-responsive promoter, and heat shock promoters. Suitable origins of replication for the replication and maintenance of the expression vectors in mammalian cells include, e.g., the Epstein Barr origin of replication in the presence of the Epstein Barr nuclear antigen (see Sugden et al., Mole. Cell. Biol., 5:410-413 (1985)) and the SV40 origin of replication in the presence of the SV40 T antigen (which is present in COS-1 and COS-7 cells) (see Margolskee et al., Mole. Cell. Biol., 8:2837 (1988)). Suitable selection markers include, but are not limited to, genes conferring resistance to neomycin, hygromycin, zeocin, and the like. Many commercially available mammalian expression vectors may be useful for the present invention, including, e.g., pCEP4, pcDNAI, pIND, pSecTag2, pVAX1, pcDNA3.1, and pBI-EGFP, and pDisplay. The vectors can be introduced into mammalian cells using any known techniques such as calcium phosphate precipitation, lipofection, electroporation, and the like. The bait vector and prey vector can be co-transformed into the same cell or, alternatively, introduced into two different cells which are subsequently fused together by cell fusion or other suitable techniques.

Viral expression vectors, which permit introduction of recombinant genes into cells by viral infection, can also be used for the expression of the fusion proteins. Viral expression vectors generally known in the art include viral vectors based on adenovirus, bovine papilloma virus, murine stem cell virus (MSCV), MFG virus, and retrovirus. See Sarver, et al., Mol. Cell. Biol., 1: 486 (1981); Logan & Shenk, Proc. Natl. Acad. Sci. USA, 81:3655-3659 (1984); Mackett, et al., Proc. Natl. Acad. Sci. USA, 79:7415-7419 (1982); Mackett, et al., J. Virol., 49:857-864 (1984); Panicali, et al., Proc. Natl. Acad. Sci. USA, 79:4927-4931 (1982); Cone & Mulligan, Proc. Natl. Acad. Sci. USA, 81:6349-6353 (1984); Mann et al., Cell, 33:153-159 (1993); Pear et al., Proc. Natl. Acad. Sci. USA, 90:8392-8396 (1993); Kitamura et al., Proc. Natl. Acad. Sci. USA, 92:9146-9150 (1995); Kinsella et al., Human Gene Therapy, 7:1405-1413 (1996); Hofmann et al., Proc. Natl. Acad. Sci. USA, 93:5185-5190 (1996); Choate et al., Human Gene Therapy, 7:2247 (1996); WO 94/19478; Hawley et al., Gene Therapy, 1:136 (1994) and Rivere et al., Genetics, 92:6733 (1995), all of which are incorporated by reference.

Generally, to construct a viral vector, a chimeric gene according to the present invention can be operably linked to a suitable promoter. The promoter-chimeric gene construct is then inserted into a non-essential region of the viral vector, typically a modified viral genome. This results in a viable recombinant virus capable of expressing the fusion protein encoded by the chimeric gene in infected host cells. Once in the host cell, the recombinant virus typically is integrated into the genome of the host cell. However, recombinant bovine papilloma viruses typically replicate and remain as extrachromosomal elements.

In another embodiment, the detection assays of the present invention are conducted in plant cell systems. Methods for expressing exogenous proteins in plant cells are well known in the art. See generally, Weissbach & Weissbach, Methods for Plant Molecular Biology, Academic Press, NY, 1988; Grierson & Corey, Plant Molecular Biology, 2d Ed., Blackie, London, 1988. Recombinant virus expression vectors based on, e.g., cauliflower mosaic virus (CaMV) or tobacco mosaic virus (TMV) can all be used. Alternatively, recombinant plasmid expression vectors such as Ti plasmid vectors and Ri plasmid vectors are also useful. The chimeric genes encoding the fusion proteins of the present invention can be conveniently cloned into the expression vectors and placed under control of a viral promoter such as the 35S RNA and 19S RNA promoters of CaMV or the coat protein promoter of TMV, or of a plant promoter, e.g., the promoter of the small subunit of RUBISCO and heat shock promoters (e.g., soybean hsp17.5-E or hsp17.3-B promoters).

In addition, the in vivo assay of the present invention can also be conducted in insect cells, e.g., Spodoptera frugiperda cells, using a baculovirus expression system. Expression vectors and host cells useful in this system are well known in the art and are generally available from various commercial vendors. For example, the chimeric genes of the present invention can be conveniently cloned into a non-essential region (e.g., the polyhedrin gene) of an Autographa californica nuclear polyhedrosis virus (AcNPV) vector and placed under control of an AcNPV promoter (e.g., the polyhedrin promoter). The non-occluded recombinant viruses thus generated can be used to infect host cells such as Spodoptera frugiperda cells in which the chimeric genes are expressed. See U.S. Pat. No. 4,215,051.

In a preferred embodiment of the present invention, the fusion proteins are expressed in a yeast expression system using yeasts such as Saccharomyces cerevisiae, Hansenula polymorpha, Pichia pastoris, and Schizosaccharomyces pombe as host cells. The expression of recombinant proteins in yeasts is a well-developed field, and the techniques useful in this respect are disclosed in detail in The Molecular Biology of the Yeast Saccharomyces, Eds. Strathern et al., Vols. I and II, Cold Spring Harbor Press, 1982; Ausubel et al., Current Protocols in Molecular Biology, New York, Wiley, 1994; and Guthrie and Fink, Guide to Yeast Genetics and Molecular Biology, in Methods in Enzymology, Vol. 194, 1991, all of which are incorporated herein by reference. Sudbery, Curr. Opin. Biotech., 7:517-524 (1996) reviews the successes in the art of expressing recombinant proteins in various yeast species; the entire content and references cited therein are incorporated herein by reference. In addition, Bartel and Fields, eds., The Yeast Two-Hybrid System, Oxford University Press, New York, N.Y., 1997 contains extensive discussions of recombinant expression of fusion proteins in yeasts in the context of various yeast two-hybrid systems, and cites numerous relevant references. These and other methods known in the art can all be used for purposes of the present invention. The application of such methods to the present invention should be apparent to a skilled artisan apprised of the present disclosure.

Generally, each of the two chimeric genes is included in a separate expression vector (bait vector and prey vector). Both vectors can be co-transformed into a single yeast host cell. As will be apparent to a skilled artisan, it is also possible to express both chimeric genes from a single vector. In a preferred embodiment, the bait vector and prey vector are introduced into two haploid yeast cells of opposite mating types, e.g., a-type and α-type, respectively. The two haploid cells can be mated at a desired time to form a diploid cell expressing both chimeric genes.

Generally, the bait and prey vectors for recombinant expression in yeast include a yeast replication origin such as the 2μ origin or the ARSH4 sequence for the replication and maintenance of the vectors in yeast cells. Preferably, the vectors also have a bacteria origin of replication (e.g., ColE1) and a bacteria selection marker (e.g., amp^{R} marker, i.e., bla gene). Optionally, the CEN6 centromeric sequence is included to control the replication of the vectors in yeast cells. Any constitutive or inducible promoters capable of driving gene transcription in yeast cells may be employed to control the expression of the chimeric genes. Such promoters are operably linked to the chimeric genes. Examples of suitable constitutive promoters include but are not limited to the yeast ADH1, PGK1, TEF2, GPD1, HIS3, and CYC1 promoters. Examples of suitable inducible promoters include but are not limited to the yeast GAL1 (inducible by galactose), CUP 1 (inducible by Cu⁺⁺), and FUS 1 (inducible by pheromone) promoters; the AOX/MOX promoter from H. polymorpha and P. pastoris (repressed by glucose or ethanol and induced by methanol); chimeric promoters such as those that contain LexA operators (inducible by LexA-containing transcription factors); and the like. Inducible promoters are preferred when the fusion proteins encoded by the chimeric genes are toxic to the host cells. If it is desirable, certain transcription repressing sequences such as the upstream repressing sequence (URS) from SPO13 promoter can be operably linked to the promoter sequence, e.g., to the 5′ end of the promoter region. Such upstream repressing sequences function to fine-tune the expression level of the chimeric genes.

Preferably, a transcriptional termination signal is operably linked to the chimeric genes in the vectors. Generally, transcriptional termination signal sequences derived from, e.g., the CYC1 and ADH1 genes can be used.

Additionally, it is preferred that the bait vector and prey vector contain one or more selectable markers for the selection and maintenance of only those yeast cells that harbor one or both chimeric genes. Any selectable markers known in the art can be used for purposes of this invention so long as yeast cells expressing the chimeric gene(s) can be positively identified or negatively selected. Examples of markers that can be positively identified are those based on color assays, including the lacZ gene (which encodes β-galactosidase), the firefly luciferase gene, secreted alkaline phosphatase, horseradish peroxidase, the blue fluorescent protein (BFP), and the green fluorescent protein (GFP) gene (see Cubitt et al., Trends Biochem. Sci., 20:448-455 (1995)). Other markers allowing detection by fluorescence, chemiluminescence, UV absorption, infrared radiation, and the like can also be used. Among the markers that can be selected are auxotrophic markers including, but not limited to, URA3, HIS3, TRP1, LEU2, LYS2, ADE2, and the like. Typically, for purposes of auxotrophic selection, the yeast host cells transformed with bait vector and/or prey vector are cultured in a medium lacking a particular nutrient. Other selectable markers are not based on auxotrophies, but rather on resistance or sensitivity to an antibiotic or other xenobiotic. Examples of such markers include but are not limited to chloramphenicol acetyl transferase (CAT) gene, which confers resistance to chloramphenicol; CAN1 gene, which encodes an arginine permease and thereby renders cells sensitive to canavanine (see Sikorski et al., Meth. Enzymol., 194:302-318 (1991)); the bacterial kanamycin resistance gene (kan^{R}), which renders eukaryotic cells resistant to the aminoglycoside G418 (see Wach et al., Yeast, 10:1793-1808 (1994)); and CYH2 gene, which confers sensitivity to cycloheximide (see Sikorski et al., Meth. Enzymol., 194:302-318 (1991)). In addition, the CUP1 gene, which encodes metallothionein and thereby confers resistance to copper, is also a suitable selection marker. Each of the above selection markers may be used alone or in combination. One or more selection markers can be included in a particular bait or prey vector. The bait vector and prey vector may have the same or different selection markers. In addition, the selection pressure can be placed on the transformed host cells either before or after mating the haploid yeast cells.

As will be apparent, the selection markers used should complement the host strains in which the bait and/or prey vectors are expressed. In other words, when a gene is used as a selection marker gene, a yeast strain lacking the selection marker gene (or having mutation in the corresponding gene) should be used as host cells. Numerous yeast strains or derivative strains corresponding to various selection markers are known in the art. Many of them have been developed specifically for certain yeast two-hybrid systems. The application and optional modification of such strains with respect to the present invention will be apparent to a skilled artisan apprised of the present disclosure. Methods for genetically manipulating yeast strains using genetic crossing or recombinant mutagenesis are well known in the art. See e.g., Rothstein, Meth. Enzymol., 101:202-211 (1983). By way of example, the following yeast strains are well known in the art, and can be used in the present invention upon necessary modifications and adjustment:

L40 strain which has the genotype AMTa his3Δ200 trp1-901 leu2-3,112 ade2 LYS2::(lexAop)4-HIS3 URA3::(lexAop)8)-lacZ;

EGY48 strain which has the genotype MATα trp1 his3 ura3 6ops-LEU2; and

MaV103 strain which has the genotype MATα ura3-52 leu2-3,112 trp1-901 his3Δ200 ade2-101 gal4Δ gal80Δ SPAL10::URA3 GAL1::HIS3::lys2 (see Kumar et al., J. Biol. Chem. 272:13548-13554 (1997); Vidal et al., Proc. Natl. Acad. Sci. USA, 93:10315-10320 (1996)). Such strains are generally available in the research community, and can also be obtained by simple yeast genetic manipulation. See, e.g., The Yeast Two-Hybrid System, Bartel and Fields, eds., pages 173-182, Oxford University Press, New York, N.Y., 1997.

In addition, the following yeast strains are commercially available:

Y190 strain which is available from Clontech, Palo Alto, Calif. and has the genotype MATa gal4 gal80 his3Δ200 trp1-901 ade2-101 ura3-52 leu2-3, 112 URA3::GAL1-lacZ LYS2::GAL1-HIS3 cyh^{r}; and

YRG-2 Strain which is available from Stratagene, La Jolla, Calif. and has the genotype MATα ura3-52 his3-200 ade2-101 lys2-801 trp1-901 leu2-3, 112 gal4-542 gal80-538 LYS2::GAL1-HIS3 URA3::GAL1/CYC1-lacZ.

In fact, different versions of vectors and host strains specially designed for yeast two-hybrid system analysis are available in kits from commercial vendors such as Clontech, Palo Alto, Calif. and Stratagene, La Jolla, Calif., all of which can be modified for use in the present invention.

### 5.3.1.2. Reporters

Generally, in a transcription-based two-hybrid assay, the interaction between a bait fusion protein and a prey fusion protein brings the DNA-binding domain and the transcription-activation domain into proximity forming a functional transcriptional factor that acts on a specific promoter to drive the expression of a reporter protein. The transcription activation domain and the DNA-binding domain may be selected from various known transcriptional activators, e.g., GAL4, GCN4, ARD1, the human estrogen receptor, E. coli LexA protein, herpes simplex virus VP16 (Triezenberg et al., Genes Dev. 2:718-729 (1988)), the E. coli B42 protein (acid blob, see Gyuris et al., Cell, 75:791-803 (1993)), NF-KB p65, and the like. The reporter gene and the promoter driving its transcription typically are incorporated into a separate reporter vector. Alternatively, the host cells are engineered to contain such a promoter-reporter gene sequence in their chromosomes. Thus, the interaction or lack of interaction between two interacting protein members of a protein complex can be determined by detecting or measuring changes in the assay system's reporter. Although the reporters and selection markers can be of similar types and used in a similar manner in the present invention, the reporters and selection markers should be carefully selected in a particular detection assay such that they are distinguishable from each other and do not interfere with each other's function.

Many different types of reporters are useful in the screening assays. For example, a reporter protein may be a fusion protein having an epitope tag fused to a protein. Commonly used and commercially available epitope tags include sequences derived from, e.g., influenza virus hemagglutinin (HA), Simian Virus 5 (V5), polyhistidine (6×His), c-myc, lacZ, GST, and the like. Antibodies specific to these epitope tags are generally commercially available. Thus, the expressed reporter can be detected using an epitope-specific antibody in an immunoassay.

In another embodiment, the reporter is selected such that it can be detected by a color-based assay. Examples of such reporters include, e.g., the lacZ protein (β-galactosidase), the green fluorescent protein (GFP), which can be detected by fluorescence assay and sorted by flow-activated cell sorting (FACS) (See Cubitt et al., Trends Biochem. Sci., 20:448-455 (1995)), secreted alkaline phosphatase, horseradish peroxidase, the blue fluorescent protein (BFP), and luciferase photoproteins such as aequorin, obelin, mnemiopsin, and berovin (See U.S. Pat. No. 6,087,476, which is incorporated herein by reference).

Alternatively, an auxotrophic factor is used as a reporter in a host strain deficient in the auxotrophic factor. Thus, suitable auxotrophic reporter genes include, but are not limited to, URA3, HIS3, TRP1, LEU2, LYS2, ADE2, and the like. For example, yeast cells containing a mutant URA3 gene can be used as host cells (Ura⁻phenotype). Such cells lack URA3-encoded functional orotidine-5′-phosphate decarboxylase, an enzyme required by yeast cells for the biosynthesis of uracil. As a result, the cells are unable to grow on a medium lacking uracil. However, wild-type orotidine-5′-phosphate decarboxylase catalyzes the conversion of a non-toxic compound 5-fluoroorotic acid (5-FOA) to a toxic product, 5-fluorouracil. Thus, yeast cells containing a wild-type URA3 gene are sensitive to 5-FOA and cannot grow on a medium containing 5-FOA. Therefore, when the interaction between the interacting protein members in the fusion proteins results in the expression of active orotidine-5′-phosphate decarboxylase, the Ura⁻ (Foa^{R}) yeast cells will be able to grow on a uracil deficient medium (SC-Ura plates). However, such cells will not survive on a medium containing 5-FOA. Thus, protein-protein interactions can be detected based on cell growth.

Additionally, antibiotic resistance reporters can also be employed in a similar manner. In this respect, host cells sensitive to a particular antibiotic are used. Antibiotic resistance reporters include, for example, the chloramphenicol acetyl transferase (CAT) gene and the kan^{R} gene, which confer resistance to G418 in eukaryotes, and kanamycin in prokaryotes, respectively.

### 5.3.1.3. Screening Assays for Interaction Antagonists

The screening assays of the present invention are useful for identifying compounds capable of interfering with, disrupting, or dissociating the protein-protein interactions formed between members of the interacting protein pairs disclosed in the tables above, or between mutant and wild type, or mutant and mutant forms of these proteins. Since the protein complexes of the present invention are associated with diabestes, obesity, Alzheimer's disease and neurodegenerative disorders (either directly through their known cellular roles or functions or through the association of mutant forms of these proteins with the disease, or indirectly—through their interactions with other proteins known to be linked to diabestes, obesity, Alzheimer's disease and neurodegenerative disorders), disruption or dissociation of particular protein-protein interactions may be desirable to ameliorate the disease condition, or to alleviate disease symptoms. Alternatively, if the disease or disorder is associated with increased expression of any of the proteins presented in the tables, or with expression of a mutant form, or forms, of these proteins, then the disease or disorder may be ameliorated, or symptoms reduced, by weakening or dissociating the interaction between the interacting proteins in patients. Also, if a disease or disorder is associated with a mutant form of an interacting protein that form stronger protein-protein interactions with its protein partner than its wild type counterpart, then the disease or disorder may be treated with a compound that weakens, disrupts or interferes with the interaction between the mutant protein and its interacting partner.

In a screening assay for an interaction antagonist, a first protein, which is a protein selected from any of the protein pairs described in the tables (or a homologue, fragment or derivative thereof), or a mutant form of the first protein (or a homologue, fragment or derivative thereof), and a second protein, which is the interacting partner of the first protein identified in the tables above (or a homologue, fragment or derivative thereof), or a mutant form of the second protein (or a homologue, fragment or derivative thereof), are used as test proteins expressed in the form of fusion proteins as described above for purposes of a two-hybrid assay. The fusion proteins are expressed in a host cell and allowed to interact with each other in the presence of one or more test compounds.

In a preferred embodiment, a counterselectable marker is used as a reporter such that a detectable signal (e.g., appearance of color or fluorescence, or cell survival) is present only when the test compound is capable of interfering with the interaction between the two test proteins. In this respect, the reporters used in various “reverse two-hybrid systems” known in the art may be employed. Reverse two-hybrid systems are disclosed in, e.g., U.S. Pat. Nos. 5,525,490; 5,733,726; 5,885,779; Vidal et al., Proc. Natl. Acad. Sci. USA, 93:10315-10320 (1996); and Vidal et al., Proc. Natl. Acad. Sci. USA, 93:10321-10326 (1996), all of which are incorporated herein by reference.

Examples of suitable counterselectable reporters useful in a yeast system include the URA3 gene (encoding orotidine-5′-decarboxylase, which converts 5-fluroorotic acid (5-FOA) to the toxic metabolite 5-fluorouracil), the CAN1 gene (encoding arginine permease, which transports the toxic arginine analog canavanine into yeast cells), the GAL1 gene (encoding galactokinase, which catalyzes the conversion of 2-deoxygalactose to toxic 2-deoxygalactose-1-phosphate), the LYS2 gene (encoding α-aminoadipate reductase, which renders yeast cells unable to grow on a medium containing α-aminoadipate as the sole nitrogen source), the MET15 gene (encoding O-acetylhomoserine sulfhydrylase, which confers on yeast cells sensitivity to methyl mercury), and the CYH2 gene (encoding L29 ribosomal protein, which confers sensitivity to cycloheximide). In addition, any known cytotoxic agents including cytotoxic proteins such as the diphtheria toxin (DTA) catalytic domain can also be used as counterselectable reporters. See U.S. Pat. No. 5,733,726. DTA causes the ADP-ribosylation of elongation factor-2 and thus inhibits protein synthesis and causes cell death. Other examples of cytotoxic agents include ricin, Shiga toxin, and exotoxin A of Pseudomonas aeruginosa.

For example, when the URA3 gene is used as a counterselectable reporter gene, yeast cells containing a mutant URA3 gene can be used as host cells (Ura⁻ Foa^{R} phenotype) for the in vivo assay. Such cells lack URA3-encoded functional orotidine-5′-phosphate decarboxylase, an enzyme required for the biosynthesis of uracil. As a result, the cells are unable to grow on media lacking uracil. However, because of the absence of a wild-type orotidine-5′-phosphate decarboxylase, the yeast cells cannot convert non-toxic 5-fluoroorotic acid (5-FOA) to a toxic product, 5-fluorouracil. Thus, such yeast cells are resistant to 5-FOA and can grow on a medium containing 5-FOA. Therefore, for example, to screen for a compound capable of disrupting interactions between GLUT4 (or a homologue, fragment or derivative thereof), or a mutant form of GLUT4 (or a homologue, fragment or derivative thereof), and PN7065 (or a homologue, fragment or derivative thereof), or a mutant form of PN7065 (or a homologue, fragment or derivative thereof), GLUT4 (or a homologue, fragment or derivative thereof) is expressed as a fusion protein with a DNA-binding domain of a suitable transcription activator while PN7065 (or a homologue, fragment or derivative thereof) is expressed as a fusion protein with a transcription activation domain of a suitable transcription activator. In the host strain, the reporter URA3 gene may be operably linked to a promoter specifically responsive to the association of the transcription activation domain and the DNA-binding domain. After the fusion proteins are expressed in the Ura⁻Foa^{R} yeast cells, an in vivo screening assay can be conducted in the presence of a test compound with the yeast cells being cultured on a medium containing uracil and 5-FOA. If the test compound does not disrupt the interaction between GLUT4 and PN7065, active URA3 gene product, i.e., orotidine-5′-decarboxylase, which converts 5-FOA to toxic 5-fluorouracil, is expressed. As a result, the yeast cells cannot grow. On the other hand, when the test compound disrupts the interaction between GLUT4 and PN7065, no active orotidine-5′-decarboxylase is produced in the host yeast cells. Consequently, the yeast cells will survive and grow on the 5-FOA-containing medium. Therefore, compounds capable of interfering with or dissociating the interaction between GLUT4 and PN7065 can thus be identified based on colony formation.

As will be apparent, the screening assay of the present invention can be applied in a format appropriate for large-scale screening. For example, combinatorial technologies can be employed to construct combinatorial libraries of small organic molecules or small peptides. See generally, e.g., Kenan et al., Trends Biochem. Sc., 19:57-64 (1994); Gallop et al., J. Med. Chem., 37:1233-1251 (1994); Gordon et al., J. Med. Chem., 37:1385-1401 (1994); Ecker et al., Biotechnology, 13:351-360 (1995). Such combinatorial libraries of compounds can be applied to the screening assay of the present invention to isolate specific modulators of particular protein-protein interactions. In the case of random peptide libraries, the random peptides can be co-expressed with the fusion proteins of the present invention in host cells and assayed in vivo. See e.g., Yang et al., Nucl. Acids Res., 23:1152-1156 (1995). Alternatively, they can be added to the culture medium for uptake by the host cells.

Conveniently, yeast mating is used in an in vivo screening assay. For example, haploid cells of a-mating type expressing one fusion protein as described above are mated with haploid cells of α-mating type expressing the other fusion protein. Upon mating, the diploid cells are spread on a suitable medium to form a lawn. Drops of test compounds can be deposited onto different areas of the lawn. After culturing the lawn for an appropriate period of time, drops containing a compound capable of modulating the interaction between the particular test proteins in the fusion proteins can be identified by stimulation or inhibition of growth in the vicinity of the drops.

The screening assays of the present invention for identifying compounds capable of modulating protein-protein interactions can also be fine-tuned by various techniques to adjust the thresholds or sensitivity of the positive and negative selections. Mutations can be introduced into the reporter proteins to adjust their activities. The uptake of test compounds by the host cells can also be adjusted. For example, yeast high uptake mutants such as the erg6 mutant strains can facilitate yeast uptake of the test compounds. See Gaber et al., Mol. Cell. Biol., 9:3447-3456 (1989). Likewise, the uptake of the selection compounds such as 5-FOA, 2-deoxygalactose, cycloheximide, α-aminoadipate, and the like can also be fine-tuned.

Generally, a control assay is performed in which the above screening assay is conducted in the absence of the test compound. The result of this assay is then compared with that obtained in the presence of the test compound.

### 5.3.1.4. Screening Assays for Interaction Agonists

The screening assays of the present invention can also be used to identify compounds that trigger or initiate, enhance or stabilize the protein-protein interactions formed between members of the interacting protein pairs disclosed in the tables above, or between combinations of mutant and wild type forms of such proteins, or pairs of mutant proteins. For example, if a disease or disorder is associated with the decreased expression of any one of the individual proteins, or one of the protein pairs selected from the tables, then the disease or disorder may be treated by strengthening or stabilizing the interactions between the interacting partner proteins in patients. Alternatively, if a disease or disorder is associated with a mutant form, or forms, of the interacting proteins that exhibit weakened or abolished interactions with their binding partner(s), then the disease or disorder may be treated with a compound that initiates or stabilizes the interaction between the mutant form, or forms, of the interacting proteins.

Thus, a screening assay can be performed in the same manner as described above, except that a positively selectable marker is used. For example, a first protein, which is any protein selected from the proteins described in the tables (or a homologue, fragment, or derivative thereof), or a mutant form of the first protein (or a homologue, fragment, or derivative thereof), and a second protein, which is an interacting partner of the first protein (or a homologue, fragment, or derivative thereof), or a mutant form of the second protein (or a homologue, fragment, or derivative thereof), are used as test proteins expressed in the form of fusion proteins as described above for purposes of a two-hybrid assay. The fusion proteins are expressed in host cells and are allowed to interact with each other in the presence of one or more test compounds.

A gene encoding a positively selectable marker such as β-galatosidase may be used as a reporter gene such that when a test compound enables, enhances or strengthens the interaction between a first protein, (or a homologue, fragment, or derivative thereof), or a mutant form of the first protein (or a homologue, fragment, or derivative thereof), and a second protein (or a homologue, fragment, or derivative thereof), or a mutant form of the second (or a homologue, fragment, or derivative thereof), β-galatosidase is expressed. As a result, the compound may be identified based on the appearance of a blue color when the host cells are cultured in a medium containing X-Gal.

Generally, a control assay is performed in which the above screening assay is conducted in the absence of the test compound. The result of this assay is then compared with that obtained in the presence of the test compound.

### 5.4. Optimization of the Identified Compounds

Once test compounds are selected that are capable of modulating the interaction between the interacting protein pairs of proteins described in the tables, or modulating the activity or intracellular levels of their constituent proteins, a secondary assay can be performed to confirm the specificity and effect of the compounds selected in the primary screens. Exemplary secondary assays are cell-based assays or animal based assays.

For example, the effect of test compounds on glucose transport can be tested in a secondary assay, which can be an assay that measures cellular glucose transport, Glut4 translocation, insulin mediated cellular transport. The test compounds can be alternatively or further tested in animal models as exemplified in the examples provided below. In one example of a secondary assay, twenty four well transwell units and 3T3-L1 adipocytes, are used for monitoring deoxyglucose uptake. Specifically, 3T3-L1 adipocytes, which exhibit high rates of insulin-sensitive glucose transport, are washed twice with PBS +Ca/Mg and then incubated overnight in a 24 well plate with DMEM-low glucose+0.5% BSA at 37° C. After incubation, aspirate the wells and wash the adipocytes with warm glucose assay buffer. Add 250 μl of buffer to wells 1-4 and add 250 μl of insulin solutions or 250 μl of insulin/test compounds solutions to wells 5-24. Pre-incubate at 37° C. for 10 minutes and then add 25 μl of 10 μM insulin to wells 13-24. Incubate at 37° C. for 20 minutes and then add 10 μl of 0.5 μCi ³H-deoxyglucose to the wells. Incubate at 37° C. for another 10 minutes and then remove the plate and immediately place onto a slushy ice bath. Aspirate the wells without tipping the plate and then wash three times with ice cold assay buffer. Aspirate the wells completely for the final wash and add 250 μl 0.2% SDS/0.2N NaOH (fresh) to all the wells of the plate. Pipet 1 ml scintillation fluid to 24 well TopCount plate. Scrape the cells and pipet a few times to detach the cells from the plate and then add all of the cells to the appropriate Scint fluid filled wells. Mix the wells, cover with a clear top, and wipe with anti-static. Determine the deoxyglucose uptake using the MRector ³H program to count radioactivity.

In addition, once test compounds are selected that are capable of modulating the proteins in the tables or the interaction between the interacting protein pairs of proteins described in the tables, or modulating the activity or intracellular levels of their constituent proteins, a data set including data defining the identity or characteristics of the test compounds can be generated. The data set may include information relating to the properties of a selected test compound, e.g., chemical structure, chirality, molecular weight, melting point, etc. Alternatively, the data set may simply include assigned identification numbers understood by the researchers conducting the screening assay and/or researchers receiving the data set as representing specific test compounds. The data or information can be cast in a transmittable form that can be communicated or transmitted to other researchers, particularly researchers in a different country. Such a transmittable form can vary and can be tangible or intangible. For example, the data set defining one or more selected test compounds can be embodied in texts, tables, diagrams, molecular structures, photographs, charts, images or any other visual forms. The data or information can be recorded on a tangible media such as paper or embodied in computer-readable forms (e.g., electronic, electromagnetic, optical or other signals). The data in a computer-readable form can be stored in a computer usable storage medium (e.g., floppy disks, magnetic tapes, optical disks, and the like) or transmitted directly through a communication infrastructure. In particular, the data embodied in electronic signals can be transmitted in the form of email or posted on a website on the Internet or Intranet. In addition, the information or data on a selected test compound can also be recorded in an audio form and transmitted through any suitable media, e.g., analog or digital cable lines, fiber optic cables, etc., via telephone, facsimile, wireless mobile phone, Internet phone and the like.

Thus, the information and data on a test compound selected in a screening assay described above or by virtual screening as discussed below can be produced anywhere in the world and transmitted to a different location. For example, when a screening assay is conducted offshore, the information and data on a selected test compound can be generated and cast in a transmittable form as described above. The data and information in a transmittable form thus can be imported into the U.S. or transmitted to any other countries, where the data and information may be used in further testing the selected test compound and/or in modifying and optimizing the selected test compound to develop lead compounds for testing in clinical trials.

Compounds can also be selected based on structural models of the target protein or protein complex and/or test compounds. In addition, once an effective compound is identified, structural analogs or mimetics thereof can be produced based on rational drug design with the aim of improving drug efficacy and stability, and reducing side effects. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990).

In this respect, structural information on the target protein or protein complex is obtained. Preferably, atomic coordinates defining a three-dimensional structure of the target protein or protein complex can be obtained. For example, each of the interacting pairs can be expressed and purified. The purified interacting protein pairs are then allowed to interact with each other in vitro under appropriate conditions. Optionally, the interacting protein complex can be stabilized by crosslinking or other techniques. The interacting complex can be studied using various biophysical techniques including, e.g., X-ray crystallography, NMR, computer modeling, mass spectrometry, and the like. Likewise, structural information can also be obtained from protein complexes formed by interacting proteins and a compound that initiates or stabilizes the interaction of the proteins. Methods for obtaining such atomic coordinates by X-ray crystallography, NMR, and the like are known in the art and the application thereof to the target protein or protein complex of the present invention should be apparent to skilled persons in the art of structural biology. See Smyth and Martin, Mol. Pathol., 53:8-14 (2000); Oakley and Wilce, Clin. Exp. Pharmacol. Physiol., 27(3):145-151 (2000); Ferentz and Wagner, Q. Rev. Biophys., 33:29-65 (2000); Hicks, Curr. Med. Chem., 8(6):627-650 (2001); and Roberts, Curr. Opin. Biotechnol., 10:42-47 (1999).

In addition, understanding of the interaction between the proteins of interest in the presence or absence of a modulator can also be derived by mutagenic analysis using a yeast two-hybrid system or other methods for detecting protein-protein interactions. In this respect, various mutations can be introduced into the interacting proteins and the effect of the mutations on protein-protein interaction examined by a suitable method such as the yeast two-hybrid system.

Various mutations including amino acid substitutions, deletions and insertions can be introduced into a protein sequence using conventional recombinant DNA technologies. Generally, it is particularly desirable to decipher the protein binding sites. Thus, it is important that the mutations introduced only affect protein-protein interactions and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Methods Enzymol., 202:301-306 (1991); Bass et al., Proc. Natl. Acad. Sci. USA, 88:4498-4502 (1991); Bennet et al., J. Biol. Chem., 266:5191-5201 (1991); Diamond et al., J. Virol., 68:863-876 (1994). Using this technique, charged or hydrophobic amino acid residues of the interacting proteins are replaced by alanine, and the effect on the interaction between the proteins is analyzed using e.g., the yeast two-hybrid system. For example, the entire protein sequence can be scanned in a window of five amino acids. When two or more charged or hydrophobic amino acids appear in a window, the charged or hydrophobic amino acids are changed to alanine using standard recombinant DNA techniques. The thus-mutated proteins are used as “test proteins” in the above-described two-hybrid assays to examine the effect of the mutations on protein-protein interaction. Preferably, the mutational analyses are conducted both in the presence and in the absence of an identified modulator compound. In this manner, the domains or residues of the proteins important to protein-protein interaction and/or the interaction between the modulator compound and the interacting proteins can be identified.

Based on the information obtained, structural relationships between the interacting proteins, as well as between the identified modulators and the interacting proteins are elucidated. For example, for the identified modulators (i.e., lead compounds), the three-dimensional structure and chemical moieties critical to their modulating effect on the interacting proteins are revealed. Using this information and various techniques known in the art of molecular modeling (i.e., simulated annealing), medicinal chemists can then design analog compounds that might be more effective modulators of the protein-protein interactions of the present invention. For example, the analog compounds might show more specific or tighter binding to their targets, and thereby might exhibit fewer side effects, or might have more desirable pharmacological characteristics (e.g., greater solubility).

In addition, if the lead compound is a peptide, it can also be analyzed by the alanine scanning technique and/or the two-hybrid assay to determine the domains or residues of the peptide important to its modulating effect on particular protein-protein interactions. The peptide compound can be used as a lead molecule for rational design of small organic molecules or peptide mimetics. See Huber et al., Curr. Med. Chem., 1: 13-34 (1994).

The domains, residues or moieties critical to the modulating effect of the identified compound constitute the active region of the compound known as its “pharmacophore.” Once the pharmacophore has been elucidated, a structural model can be established by a modeling process that may incorporate data from NMR analysis, X-ray diffraction data, alanine scanning, spectroscopic techniques and the like. Various techniques including computational analysis (e.g., molecular modeling and simulated annealing), similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs energy minimization and molecular dynamics functions, and QUANTA program, which performs construction, graphic modeling and analysis of molecular structure. Such programs allow interactive construction, modification, and visualization of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. In addition, in the case of a peptide lead compound, the peptide or mimetics thereof can be cyclized, e.g., by linking the N-terminus and C-terminus together, to increase its stability. These rationally designed compounds are further tested. In this manner, pharmacologically acceptable and stable compounds with improved efficacy and reduced side effects can be developed. The compounds identified in accordance with the present invention can be incorporated into a pharmaceutical formulation suitable for administration to an individual.

In addition, the structural models or atomic coordinates defining a three-dimensional structure of the target protein or protein complex can also be used in virtual screen to select compounds capable of modulating the target protein or protein complex. Various methods of computer-based virtual screen using atomic coordinates are generally known in the art. For example, U.S. Pat. No. 5,798,247 (which is incorporated herein by reference) discloses a method of identifying a compound (specifically, an interleukin converting enzyme inhibitor) by determining binding interactions between an organic compound and binding sites of a binding cavity within the target protein. The binding sites are defined by atomic coordinates.

The compounds designed or selected based on rational drug design or virtual screen can be tested for their ability to modulate (interfere with or strengthen) the interaction between the interacting partners within the protein complexes of the present invention. In addition, the compounds can also be further tested for their ability to modulate (inhibit or enhance) cellular functions such as cellular glucose transport in cells as well as their effectiveness in treating diseases such as diabestes, obesity, Alzheimer's disease and neurodegenerative disorders.

Following the selection of desirable compounds according to the methods disclosed above, the methods of the present invention further provide for the manufacture of the selected compounds. Compounds found to desirably modulate the interaction between the interacting protein pairs of proteins of the present invention, or to desirably modulate the activity or intracellular levels of their constituent proteins, can be manufactured for further experimental studies, or for therapeutic use.

### 6. Therapeutic Applications

As described above, the interactions between the interacting pairs of proteins of the present invention suggest that these proteins and/or the protein complexes formed by them may be involved in common biological processes and disease pathways. The protein complexes may mediate the functions of the individual proteins of each interacting protein pair, or of the interacting pairs themselves, in the biological processes or disease pathways. Thus, one may modulate such biological processes or treat diseases by modulating the functions and activities of any of the individual proteins described in the tables, and/or a protein complex comprising some combination of these proteins. As used herein, modulating a protein selected from the tables, or a protein complex comprising some combination of these proteins means altering (enhancing or reducing) the intracellular concentrations or activities of the proteins or protein complexes, e.g., increasing the concentrations of a particular protein described in the tables, or a protein complex comprising some combination of these proteins, enhancing or reducing their biological activities, increasing or decreasing their stability, altering their affinity or specificity to certain other biological molecules, etc. For example, a pair of interacting proteins listed in the tables may be involved in cellular glucose transport. Thus, assays such as those described in Section 4 may be used in determining the effect of an aberration in a particular protein complex or an interacting member thereof on cellular glucose transport. In addition, it is also possible to determine, using the same assay methods, the presence or absence of an association between a protein complex of the present invention or an interacting member thereof and a physiological disorder or disease such as diabestes, obesity, Alzheimer's disease and neurodegenerative disorders or predisposition to a physiological disorder or disease.

Once such associations are established, the diagnostic methods as described in Section 4 can be used in diagnosing the disease or disorder, or a patient's predisposition to it. In addition, various in vitro and in vivo assays may be employed to test the therapeutic or prophylactic efficacies of the various therapeutic approaches described in Sections 6.2 and 6.3 that are aimed at modulating the functions and activities of a particular protein complex of the present invention, or an interacting member thereof. Similar assays can also be used to test whether the therapeutic approaches described in Sections 6.2 and 6.3 result in the modulation of cellular glucose transport. The cell model or transgenic animal model described in Section 7 may be employed in the in vitro and in vivo assays.

In accordance with this aspect of the present invention, methods are provided for modulating (promoting or inhibiting) a protein complex of the present invention formed by the interactions described in the tables. The human cells can be in in vitro cell or tissue cultures. The methods are also applicable to human cells in a patient.

In one embodiment, the concentration of a protein complex formed by the interactions described in the tables is reduced in the cells. Various methods can be employed to reduce the concentration of the protein complex. For example, the protein complex concentration can be reduced by interfering with the interactions between the interacting protein partners. Hence, compounds capable of interfering with interactions between interacting pairs of proteins identified in the tables can be administered to the cells in vitro or in vivo in a patient. Such compounds can be compounds capable of binding specific proteins listed in the tables. They can also be antibodies immunoreactive with specific proteins identified in the tables. Also, the compounds can be small peptides derived from a first interacting protein of the present invention, or a mimetic thereof, that are capable of binding a second protein of the present invention, the second protein being a binding partner of the first protein as shown in the tables above.

In another embodiment, the method of modulating the protein complex includes inhibiting the expression of any of the individual proteins described in the tables. The inhibition can be at the transcriptional, translational, or post-translational level. For example, antisense compounds and ribozyme compounds can be administered to human cells in cultures or in human bodies. In addition, RNA interference technologies may also be employed to administer to cells double-stranded RNA or RNA hairpins capable of “knocking down” the expression of any of the interacting proteins of the present invention.

In the various embodiments described above, preferably the concentrations or activities of both partners in an interacting pair of proteins of the present invention are reduced or inhibited, or the concentration or activitie of a single constituent protein of a protein complex formed by the interactions described in the tables is reduced or inhibited.

In yet another embodiment, an antibody selectively immunoreactive with a pair of interacting proteins identified in the tables is administered to cells in vitro or in human bodies to inhibit the protein complex activities and/or reduce the concentration of the protein complex in the cells or patient.

Further provided by the present invention is a method of treatment of a disease or disorder comprising identifying a patient that has a particular disease or disorder, shows symptoms of having a particular disease or disorder, is predisposed to, or at risk of developing a particular disease or disorder, and treating the disease or disorder by modulating a protein or protein-protein interaction according to the present invention.

### 6.1. Applicable Diseases

Fat and muscle cells from diabetic patients appear to be defective in the regulation of glucose transport (Zierath et al., 1998). Therefore, it is thought that abnormal glucose uptake may be an important factor developing non-insulin dependent diabetes mellitus (NIDDM). Because the proteins and protein interactions in the tables are believed to play a role in regulating glucose uptake, they are also thought to play a role in developing diabetes. For example, Glut4 is known to shuttle between the interior of the cell to the plasma membrane by means of vesicle-mediated endocytosis and exocytosis (Garvey et al., 1998; Haruta et al., 1995; Pessin et al., 1999). In addition, IRAP co-localizes with the Glut4 transporter in specified endocytic vesicles (Keller et al., 1995; Malide et al., 1997) and expression of the N-terminal fragment of IRAP has been shown to result in the translocation of Glut4 to the plasma membrane. Consequently, modulating the proteins and protein interactions of the tables is thought to be effective in preventing and/or treating diabetes.

In addition, the roles of the proteins in the tables in cellular glucose transport indicate that modulating the proteins and protein interactions in the tables will be effective in combating diseases such as diabestes, obesity, Alzheimer's disease and neurodegenerative disorders. For example, CARP's role as a negative regulator of cardiac-specific genes (Jeyaseelan et al., 1997) suggests that modulating CARP and/or its ligands may be an effective therapeutic for heart disease. Exaples of applicable cardiovascular diseases include, but are not limited to, atheroscherosis and other forms of arteriosclerosis; vasculitides including giant cell arteritis, Takayasu's arteritis, polyarteritis nodosa, Kawasaki syndrome, microscopic polyangiitis, Wegner's granulomatosis, Buerger's disease, infectious arteritis; Raynaud's disease; aneurysms; tumors of blood vessels such as hemangioma, glomus tumor, vascular ectasias, bacillary angiomatosis, hemangioendothelioma, angiosarcoma, hemangiopericytoma, and Kaposi's sarcoma; congestive heart failure; ischemic heart diseases (e.g., angina pectoris, myocardial infarction, chronic ischemic heart disease); hypertensive heart disease; valvular heart diseases (e.g., degerative calcific aortic valve stenosis, myxomatous degeneration of the mitral valve, rheumatic heart disease, and endocarditis); myocardial diseases including cardiomyopathy and myocarditis; pericarditis; cardiac tumors; and various congenital heart diseases.

In addition, APP's involvement in Alzheimer's disease suggests that modulating APP and/or its ligands may be effective in treating or preventing Alzheimer's disease, dementia, and/or other neurodegenerative diseases. Furthermore, alpha-karyopherin's role in the nuclear transport of the HIV preintegration complex and NAF1b's ability to bind the Nef gene product of HIV (Fukushi et al., 1999) suggests that modulating the proteins or protein interactions of the table can treat or prevent viral infections such as aids. Therefore, the methods of the present invention may also be useful in treating or preventing diseases or disorders associated with viral infection in animals, including, but not limited to infections of, hepatitis A, hepatitis B, hepatitis C, encephalitis, influenza virus, respiratory syncytial virus, herpes simplex virus, herpes zoster virus, varicella virus, cytomegalovirus, retroviruses, etc. In one specific embodiment, the methods relate to treating or preventing diseases and disorders caused by lentiviruses or retroviruses, particularly AIDS and/or AIDS-related conditions. The methods comprise modulating the functions and activities of the protein complexes of the tables or the interacting protein members thereof identified in accordance with the present invention. As used herein, the term “HIV infection” generally encompasses infection of a host animal, particularly a human host, by the human immunodeficiency virus (HIV) family of retroviruses including, but not limited to, HIV I, HIV II, HIV III, and the like. Thus, treating HIV infection will encompass the treatment of a person who is a carrier of any of the HIV family of retroviruses or a person who is diagnosed of active AIDS, as well as the treatment or prophylaxis of the AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD⁴⁺ T cells), and AIDS (which is defined by more serious AIDS-defining illnesses and/or a decline in the circulating CD4 cell count to below a level that is compatible with effective immune function). In addition, “treating or preventing HIV infection” will also encompass treating suspected infection by HIV after suspected past exposure to HIV by e.g., blood transfusion, exchange of body fluids, bites, accidental needle stick, or exposure to patient blood during surgery.

The term “treating AIDS” means treating a patient who exhibits more serious AIDS-defining illnesses and/or a decline in the circulating CD4 cell count to below a level that is compatible with effective immune function. The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Thus, the term “preventing AIDS” as used herein means preventing in a patient who has HIV infection or is suspected to have HIV infection or is at risk of HIV infection from developing AIDS (which is characterized by more serious AIDS-defining illnesses and/or a decline in the circulating CD4 cell count to below a level that is compatible with effective immune function) and/or AIDS-related conditions.

### 6.2. Inhibiting Protein Complex or Interacting Protein Members Thereof

In one aspect of the present invention, methods are provided for reducing in cells or tissue the concentration and/or activity of a protein complex identified in accordance with the present invention that comprises one or more of the interacting pairs of proteins described in the tables. In addition, methods are also provided for reducing in cells or tissue the concentration and/or activity of any of the individual proteins identified in the tables. By reducing the concentration of a protein complex and/or one or more of the protein constituents of the protein complex and/or inhibiting the functional activities of the protein complex and/or one or more of the protein constituents of the protein complex, the diseases involving such a protein complex or protein constituents of the protein complex may be treated or prevented.

### 6.2.1. Antibody Therapy

In one embodiment, an antibody may be administered to cells or tissue in vitro or to patients. The antibody administered may be immunoreactive with any of the individual proteins described in the tables, or with one of the protein complexes of the present invention. Suitable antibodies may be monoclonal or polyclonal that fall within any antibody class, e.g., IgG, IgM, IgA, IgE, etc. The antibody suitable for this invention may also take a form of various antibody fragments including, but not limited to, Fab and F(ab′)₂, single-chain fragments (scFv), and the like. In another embodiment, an antibody selectively immunoreactive with the protein complex formed from at least one of the interacting pairs of proteins described in the tables, is administered to cells or tissue in vitro or in to patient. In yet another embodiment, an antibody specific to an individual protein selected from any of the tables is administered to cells or tissue in vitro or in a patient. Methods for making the antibodies of the present invention should be apparent to a person of skill in the art, especially in view of the discussions in Section 3 above. The antibodies can be administered in any suitable form via any suitable route as described in Section 8 below. Preferably, the antibodies are administered in a pharmaceutical composition together with a pharmaceutically acceptable carrier.

Alternatively, the antibodies may be delivered by a gene-therapy approach. That is, nucleic acids encoding the antibodies, particularly single-chain fragments (scFv), may be introduced into cells or tissue in vitro or in a patient such that desirable antibodies may be produced recombinantly in vivo from the nucleic acids. For this purpose, the nucleic acids with appropriate transcriptional and translation regulatory sequences can be directly administered into the patient. Alternatively, the nucleic acids can be incorporated into a suitable vector as described in Sections 2.2 and 5.3.1.1 and delivered into cells or tissue in vitro or in a patient along with the vector. The expression vector containing the nucleic acids can be administered directly to cells or tissue in vitro or in a patient. It can also be introduced into cells, preferably cells derived from a patient to be treated, and subsequently delivered into the patient by cell transplantation. See Section 6.3.2 below.

### 6.2.2. siRNA Therapy

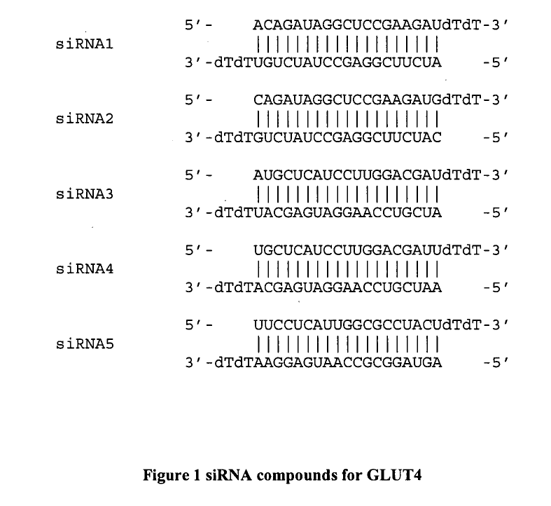

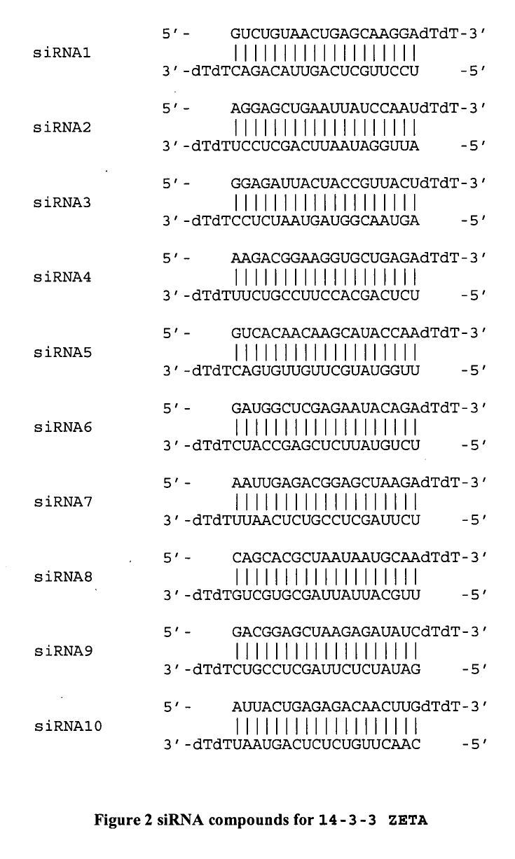

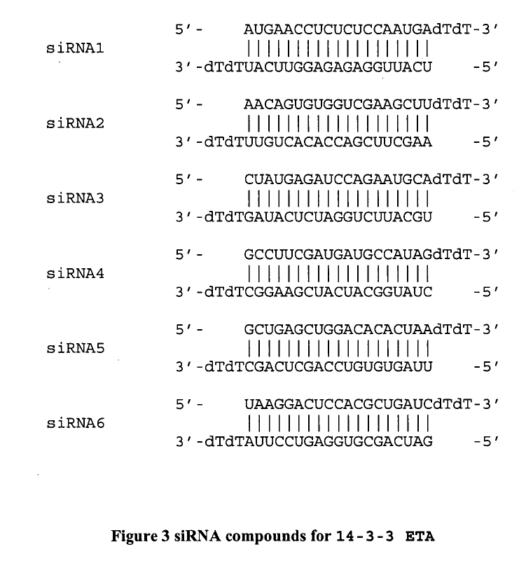

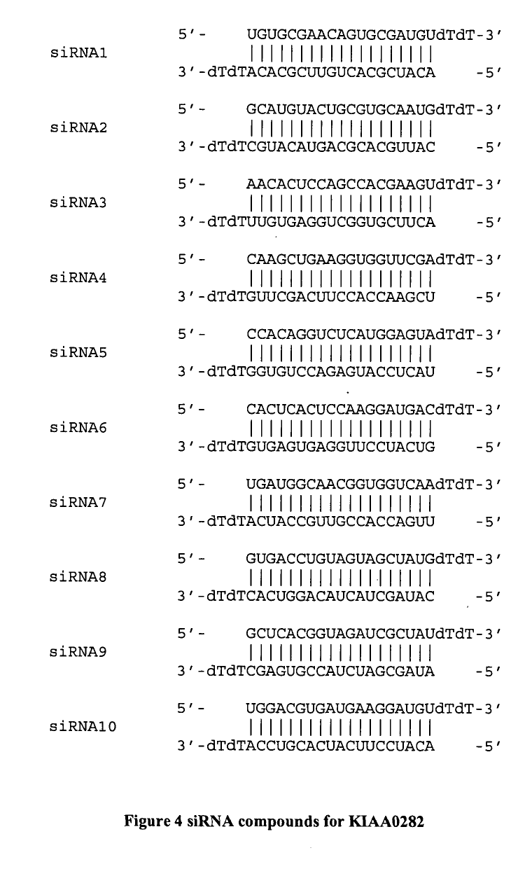

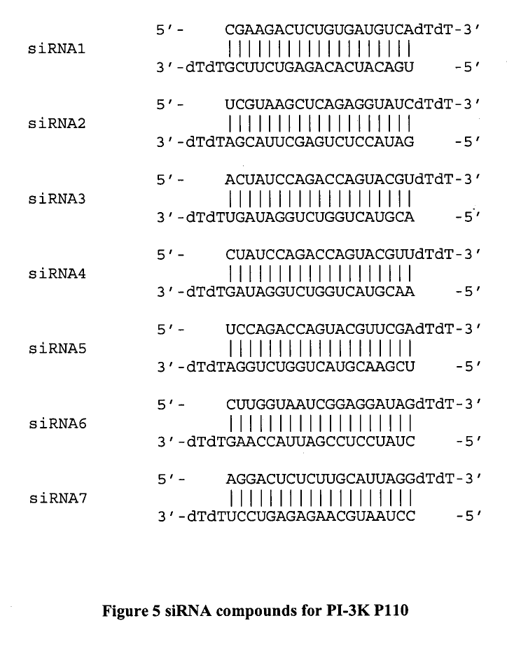

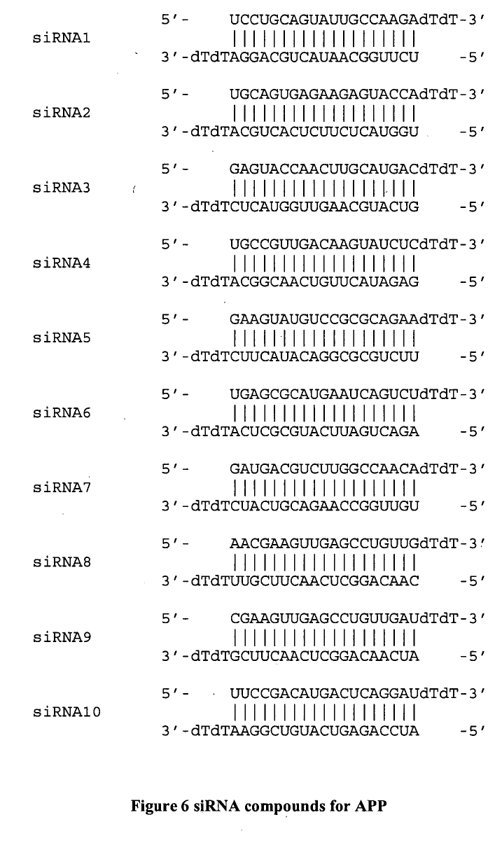

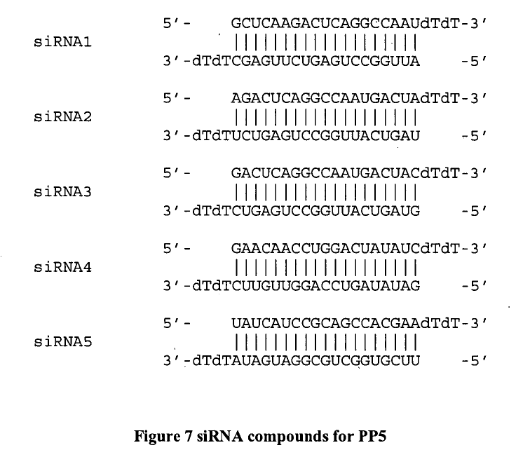

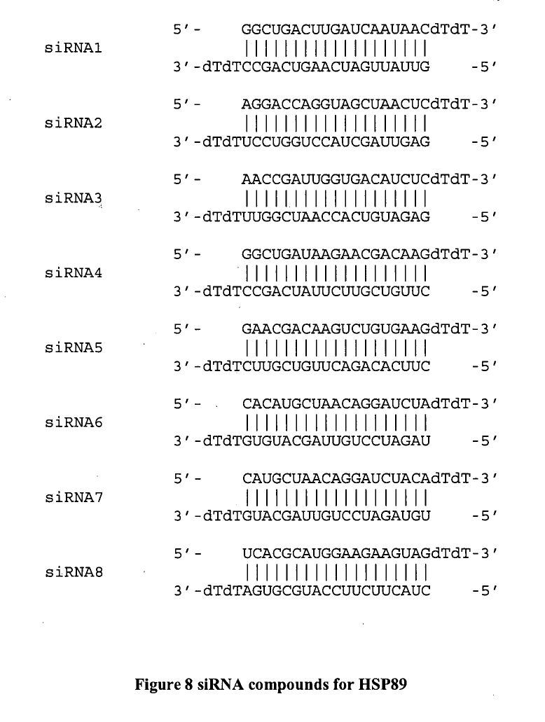

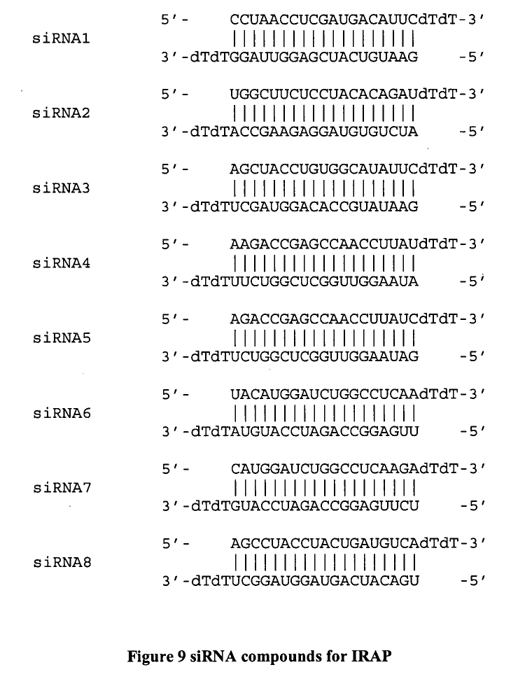

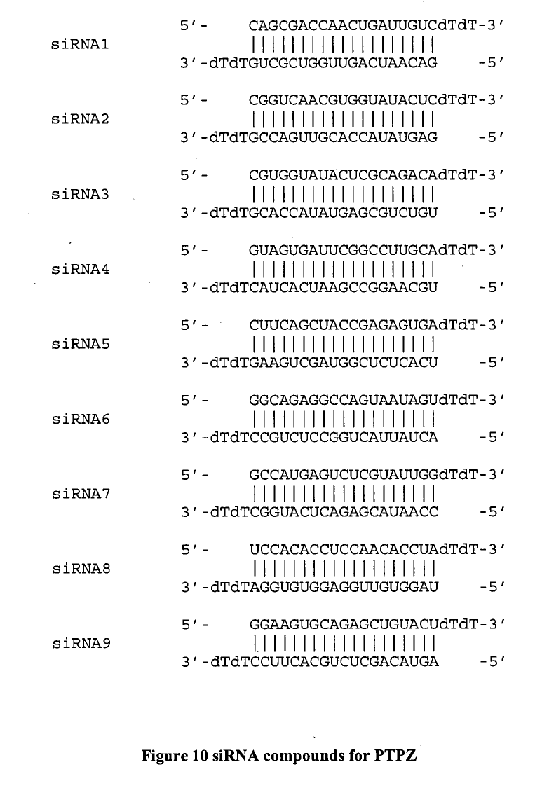

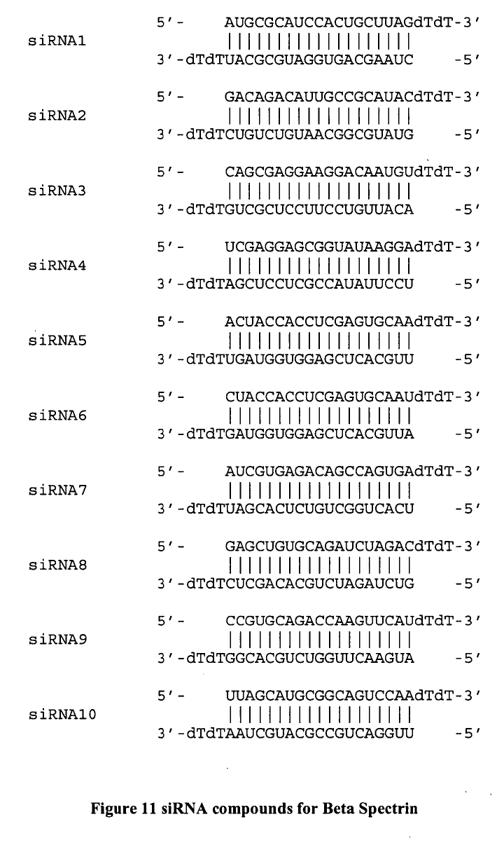

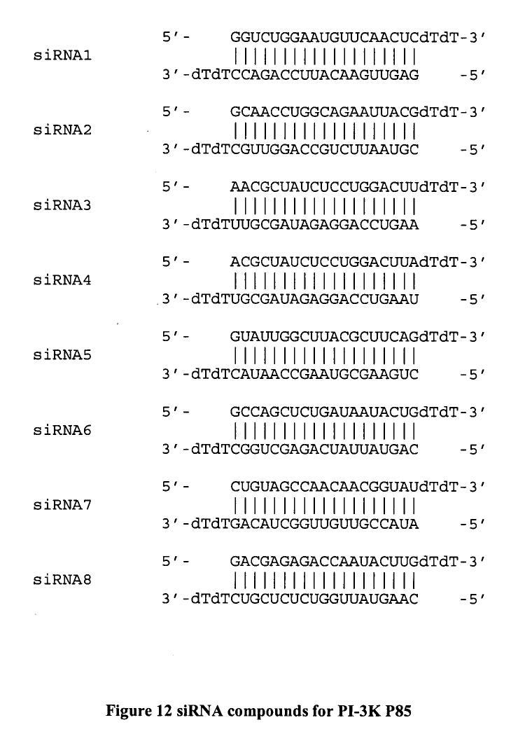

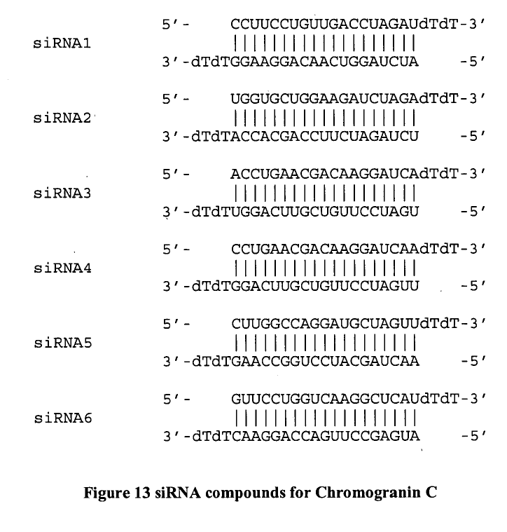

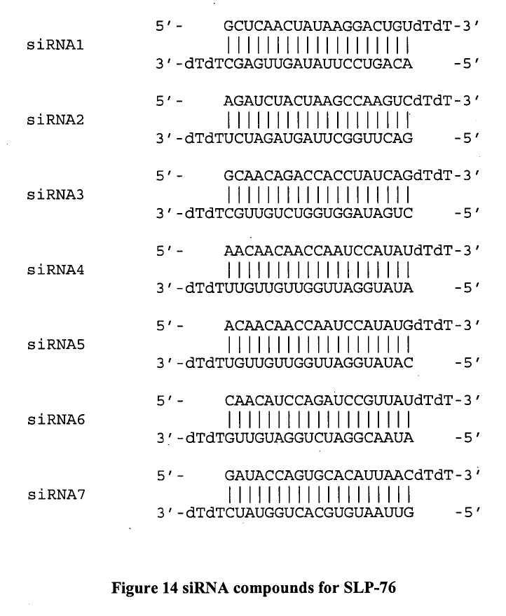

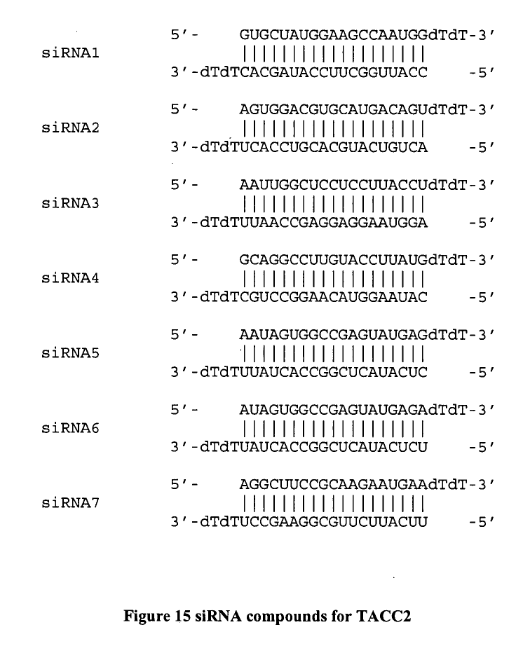

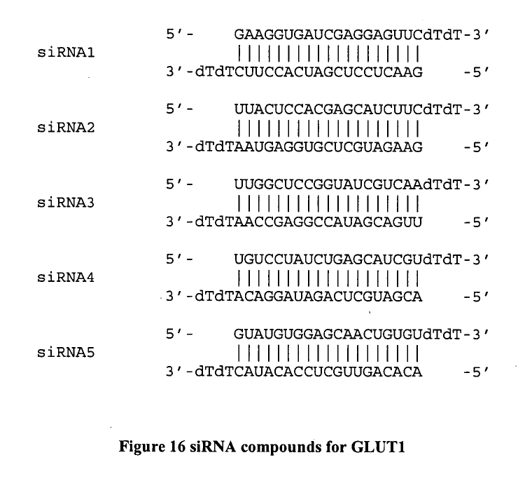

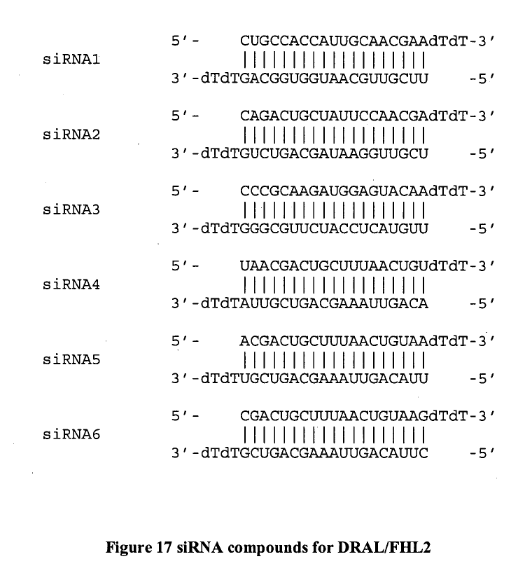

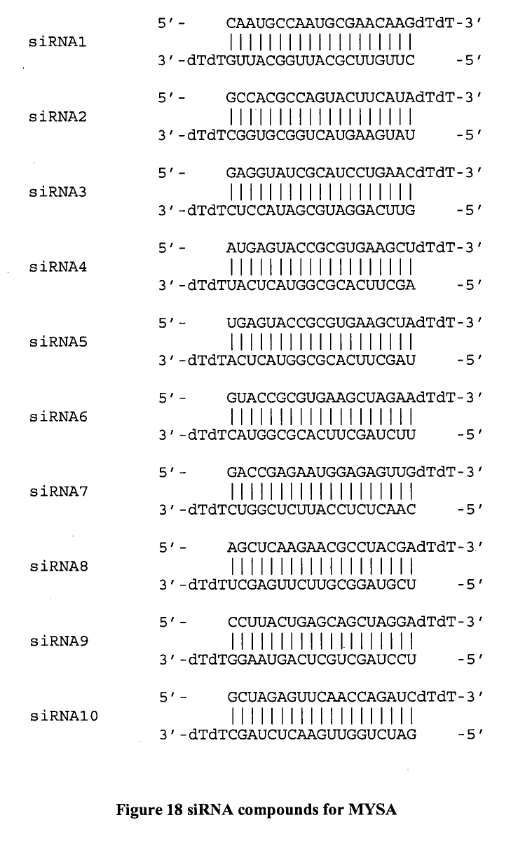

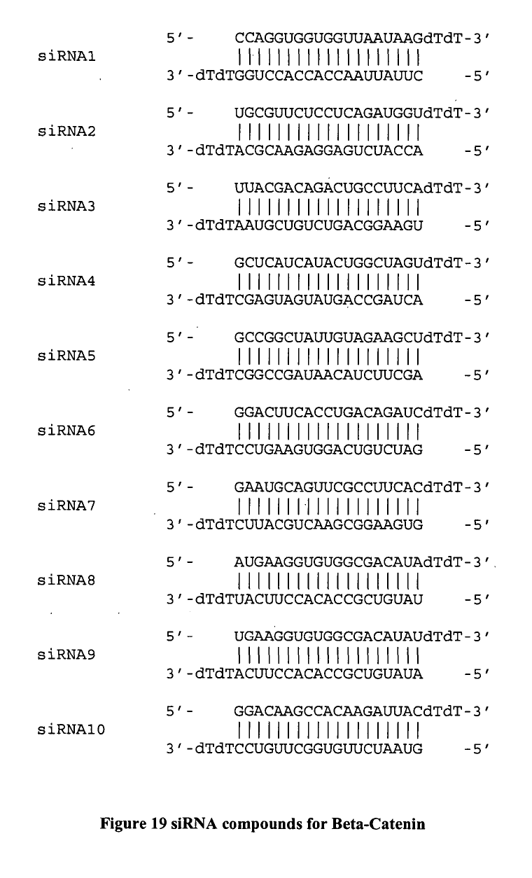

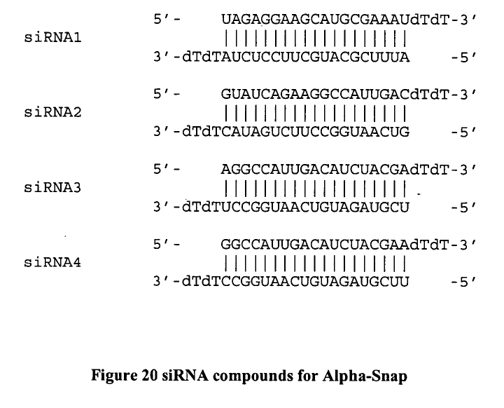

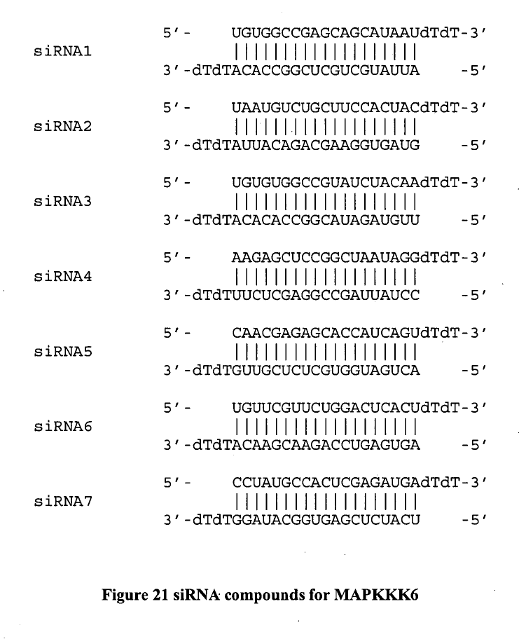

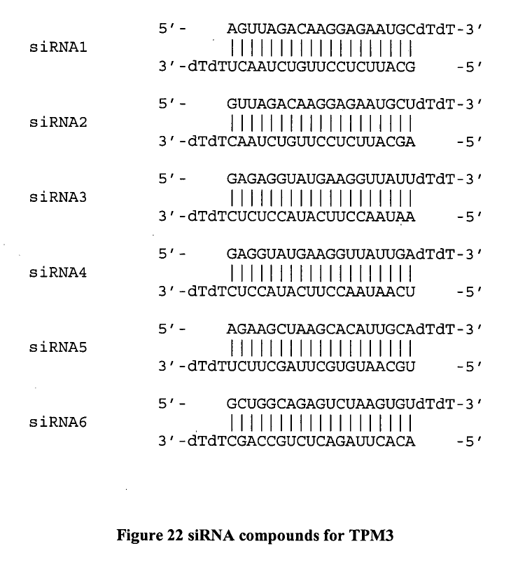

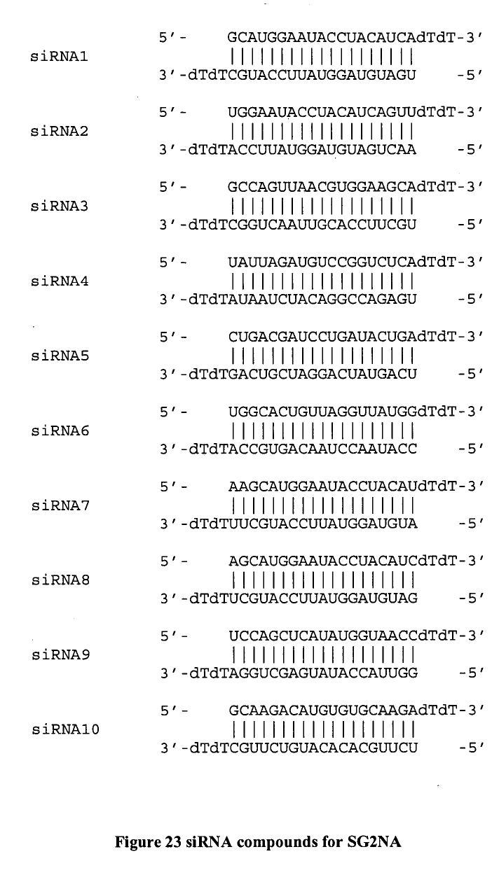

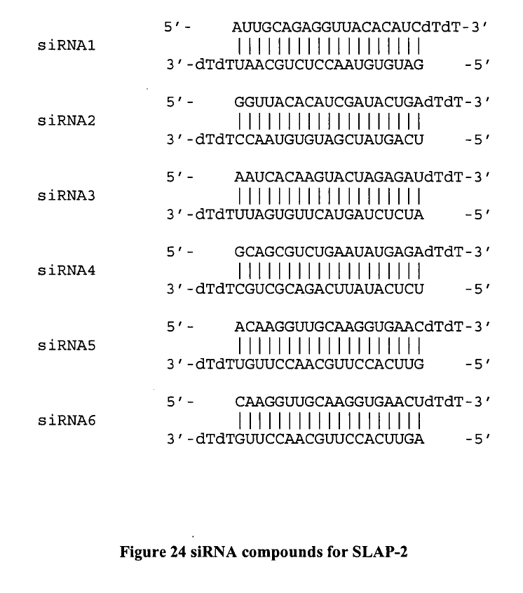

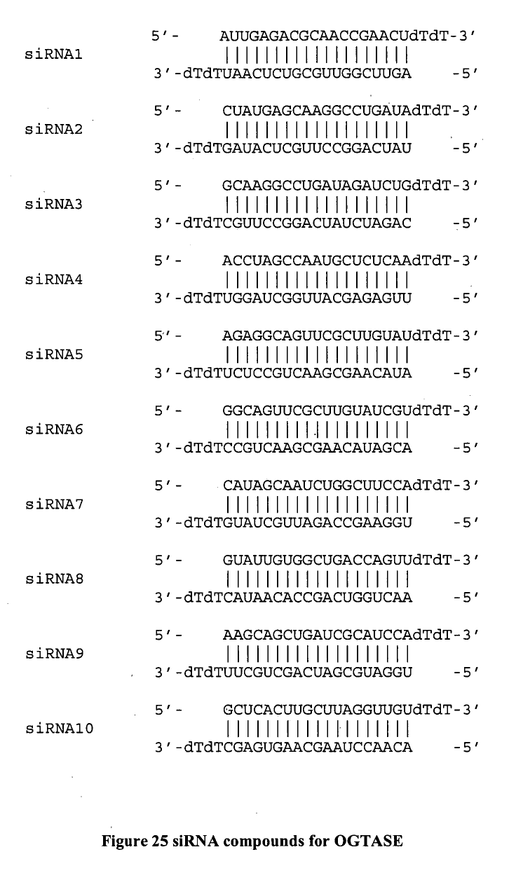

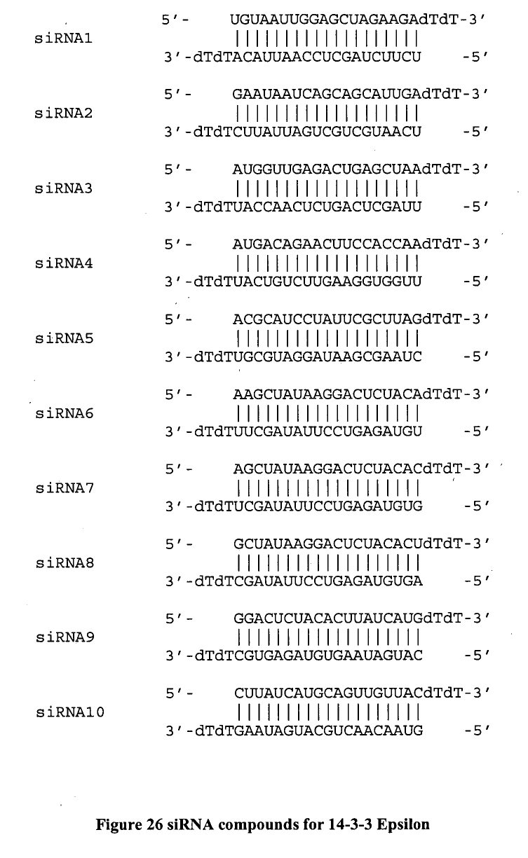

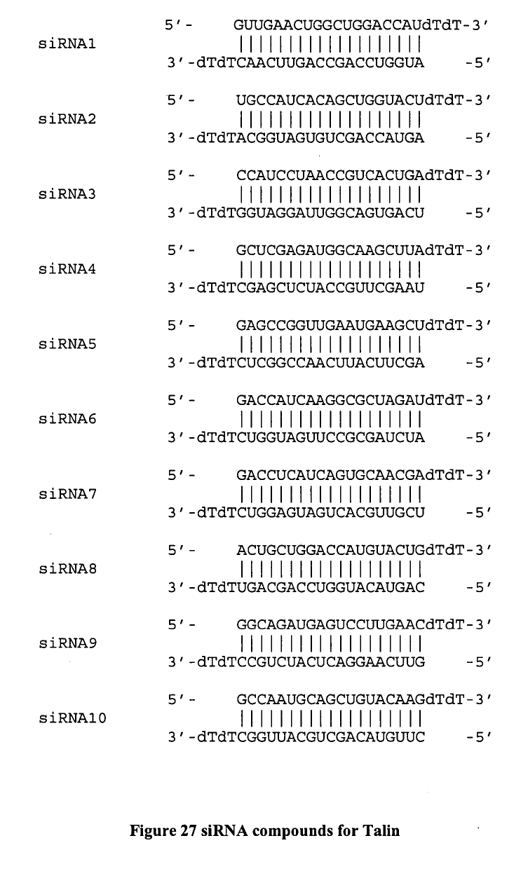

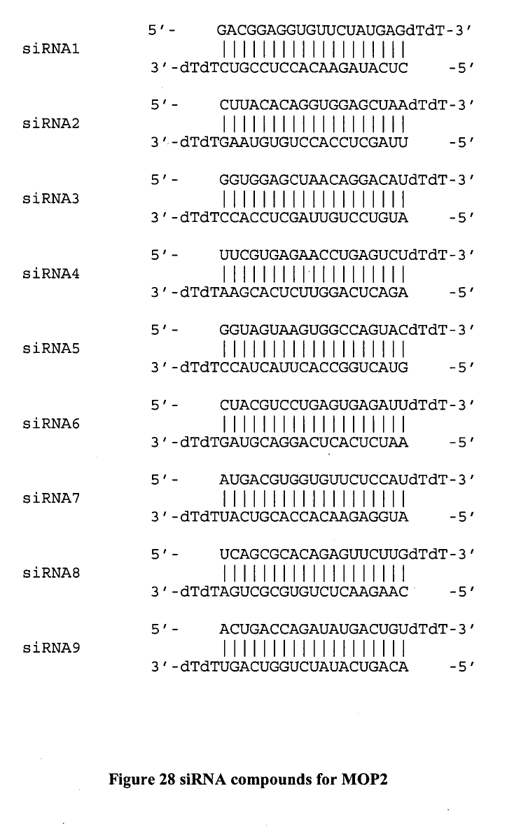

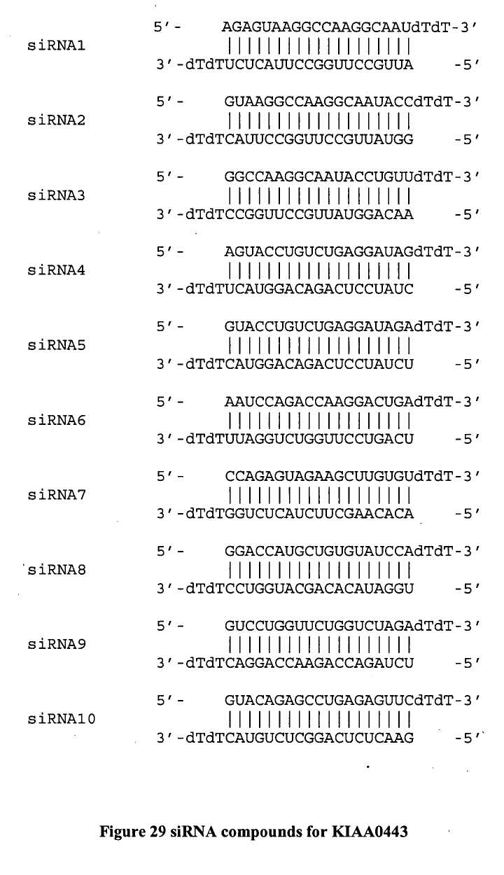

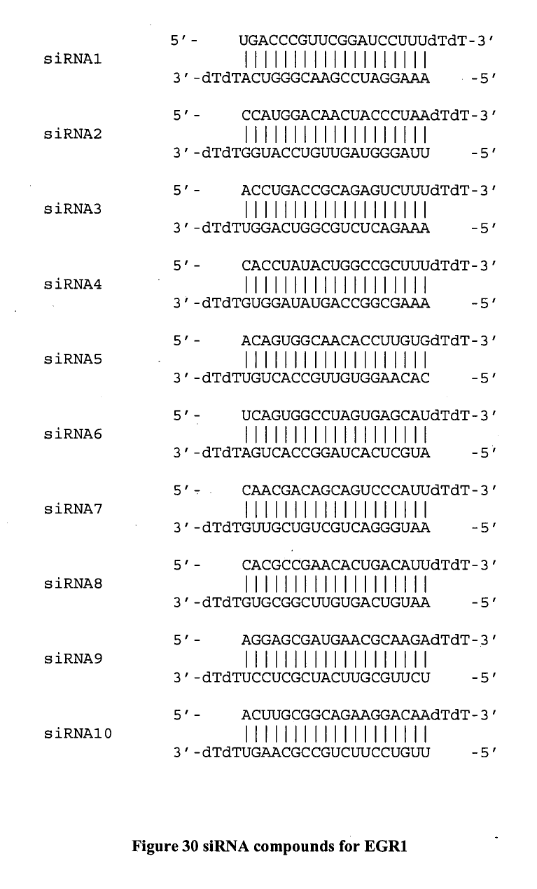

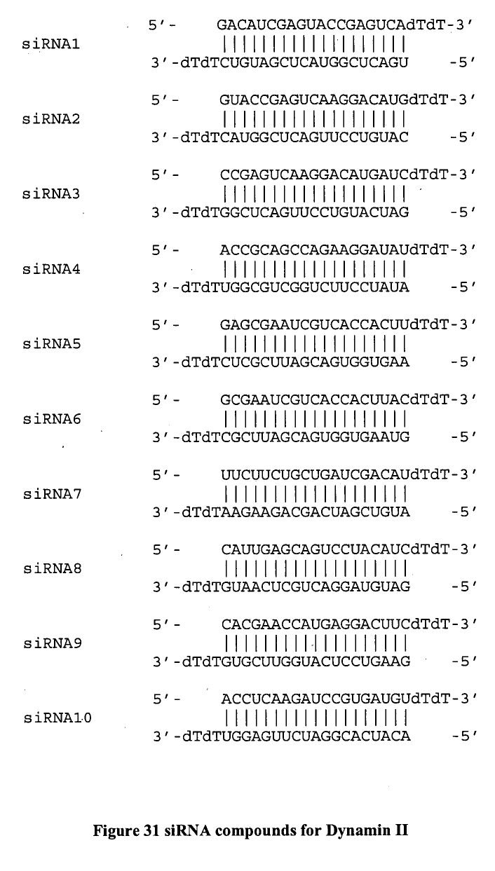

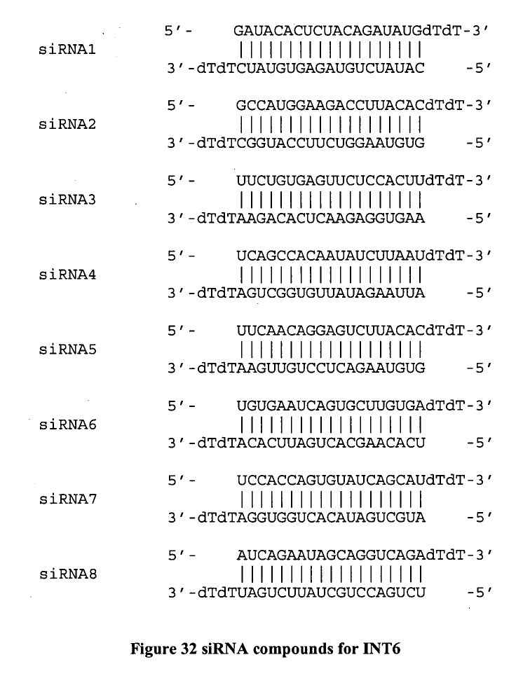

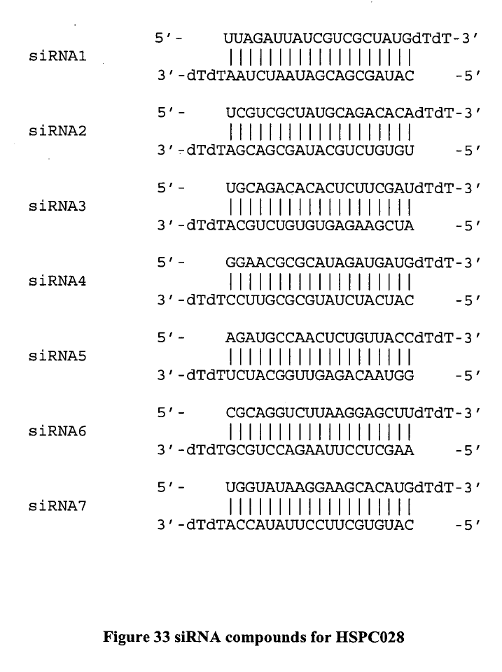

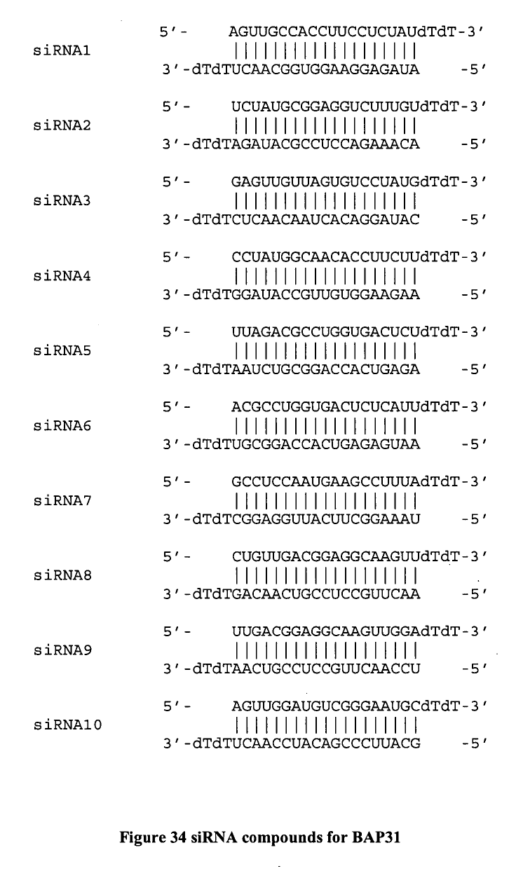

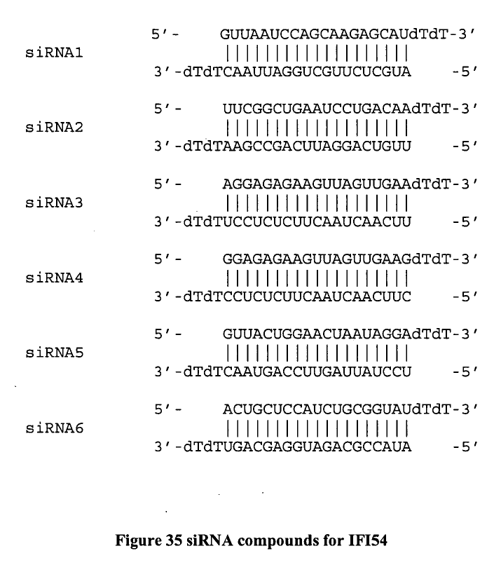

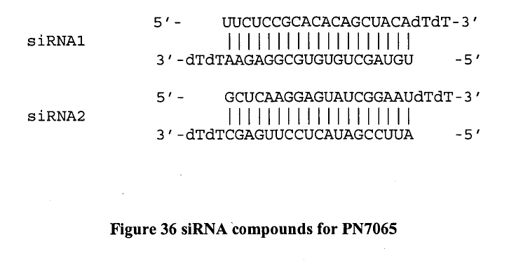

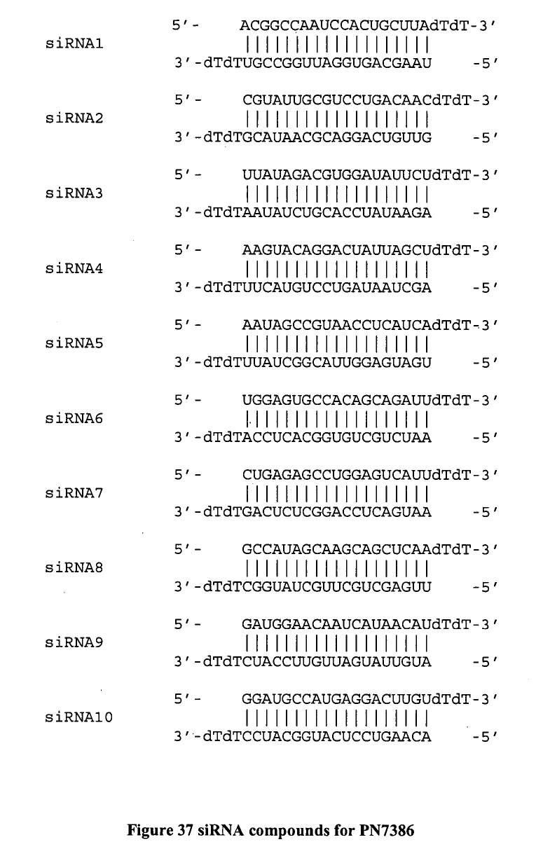

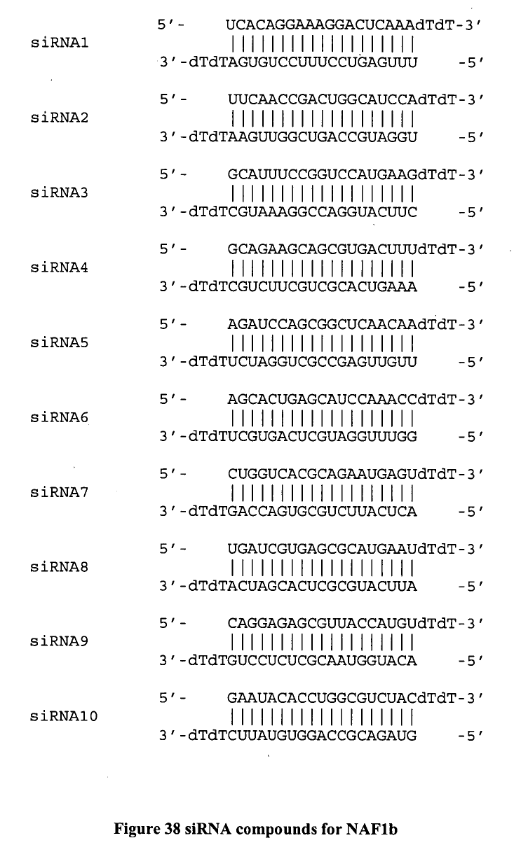

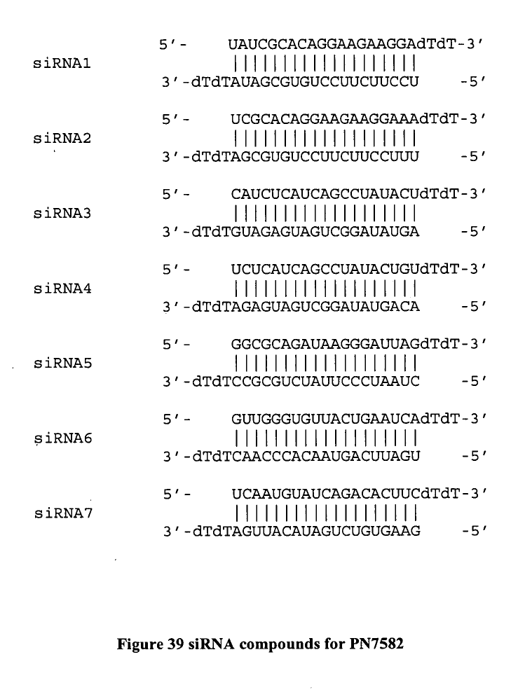

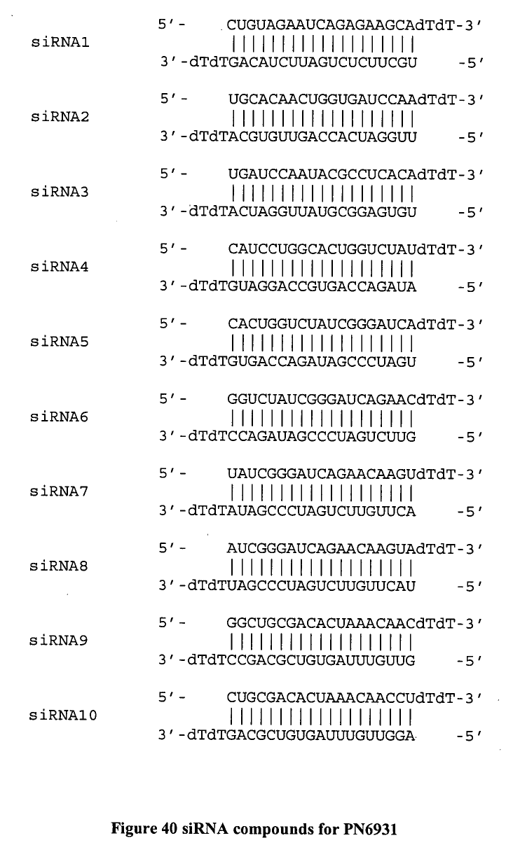

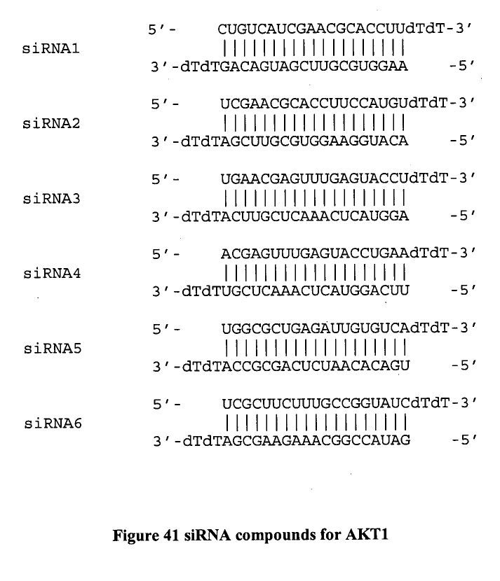

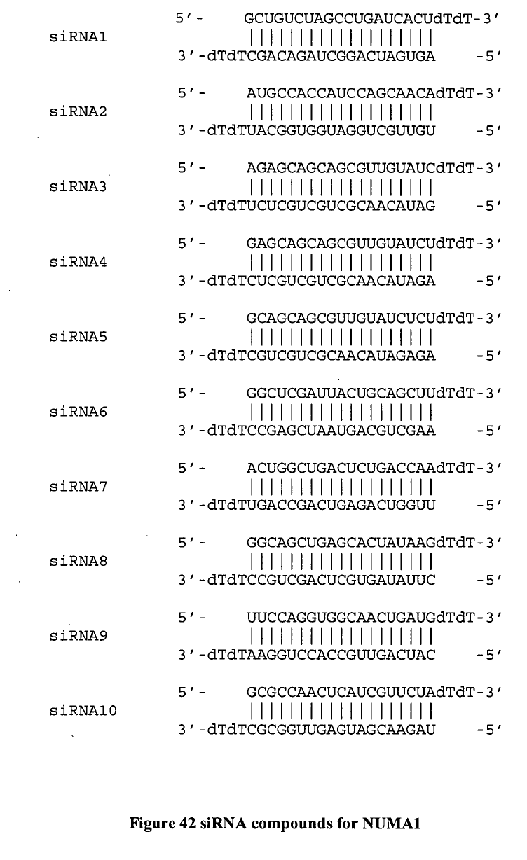

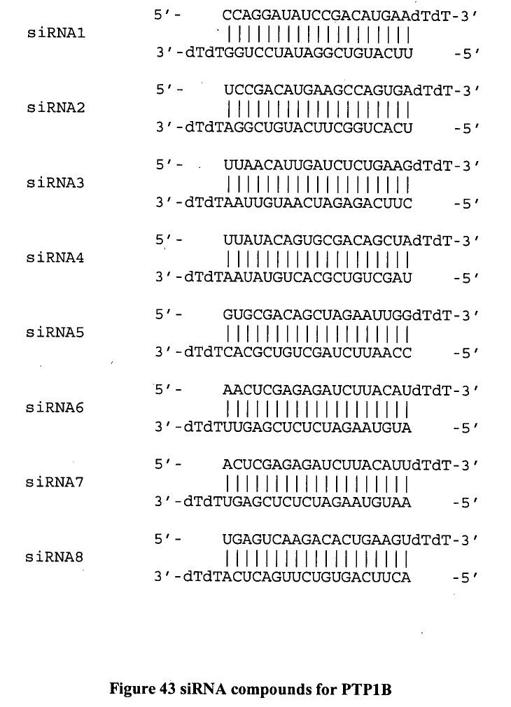

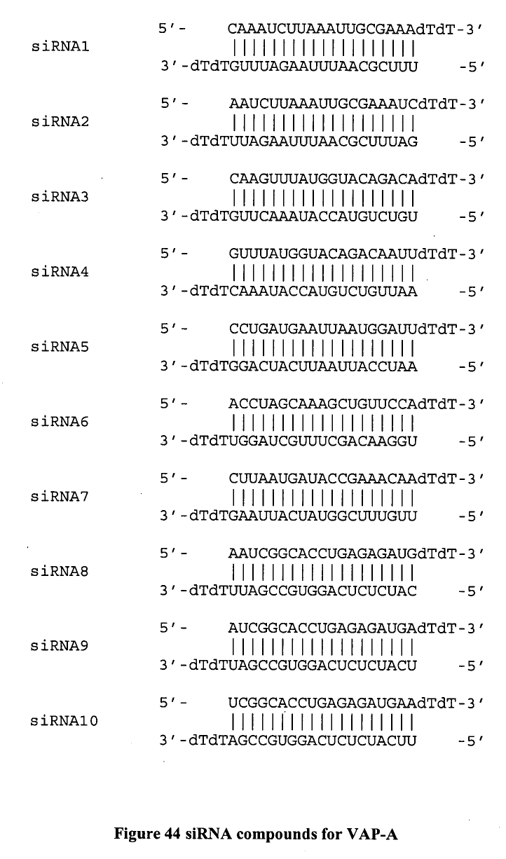

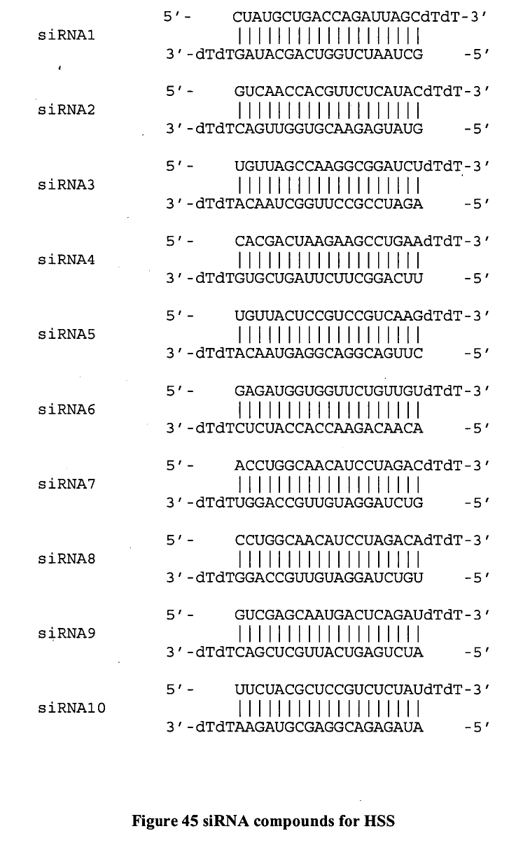

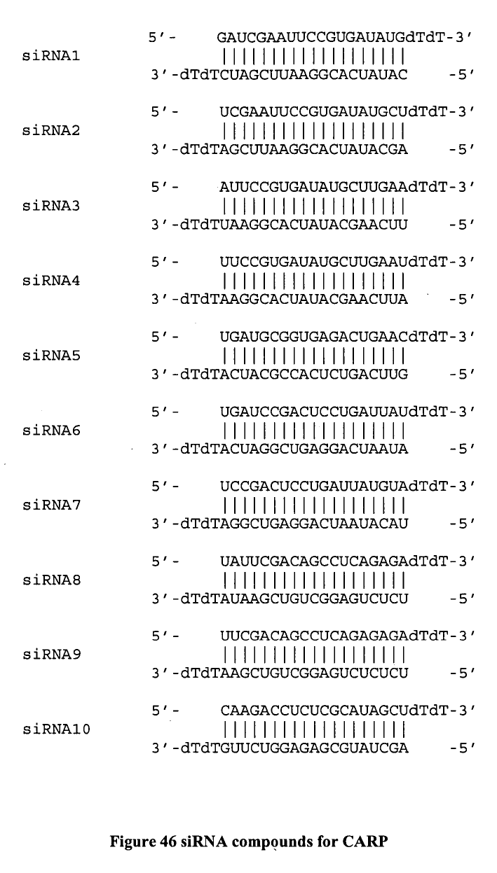

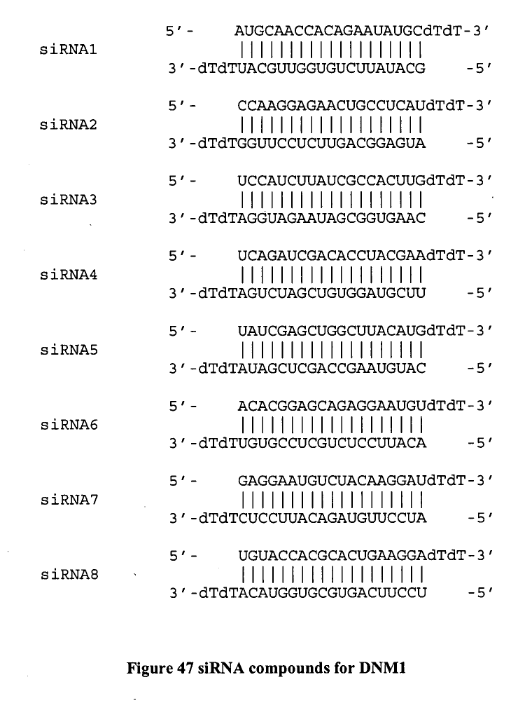

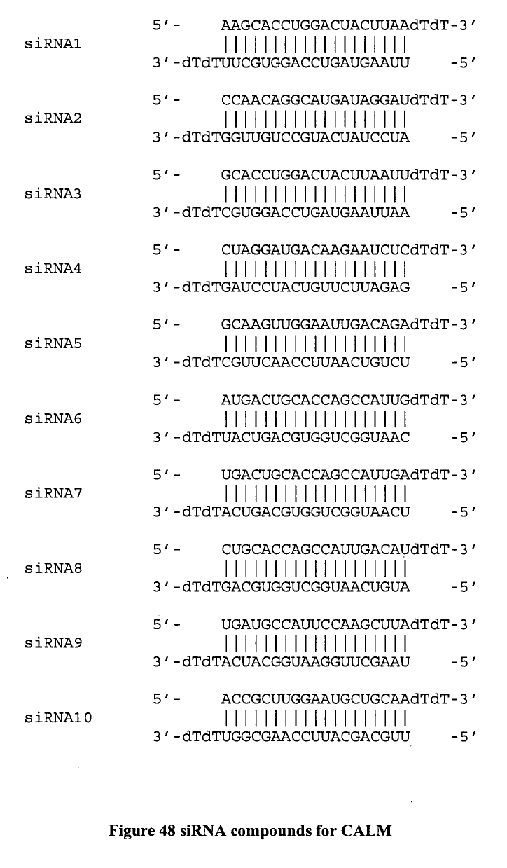

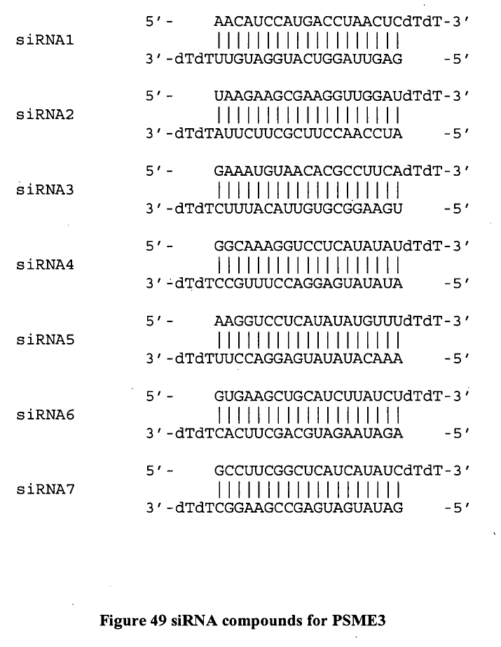

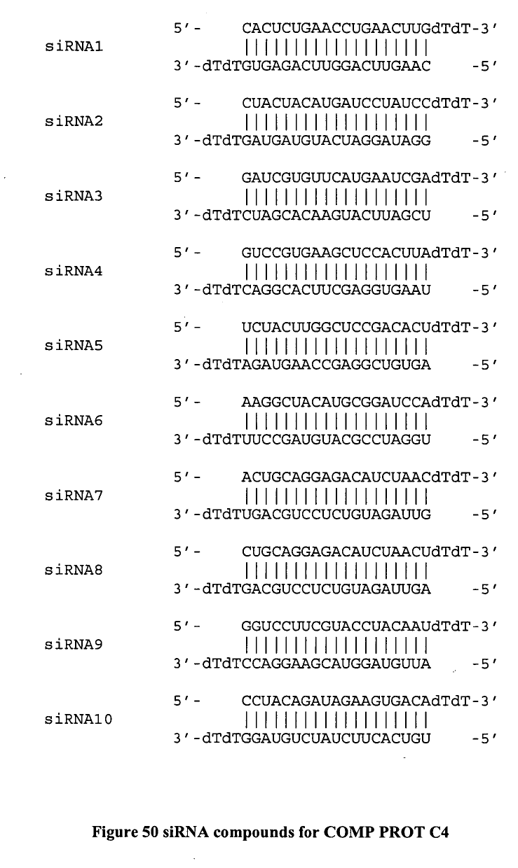

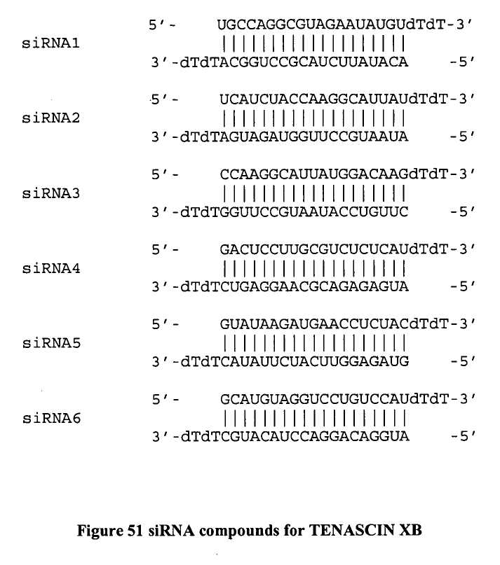

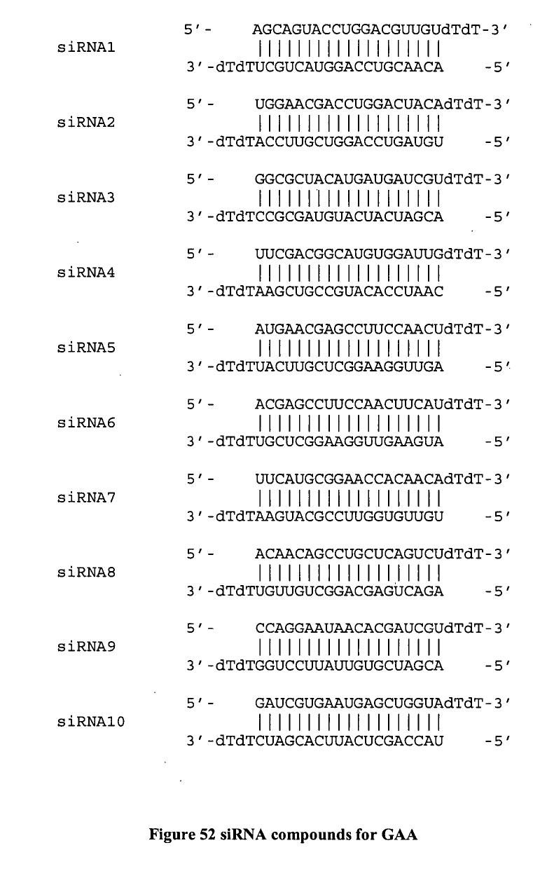

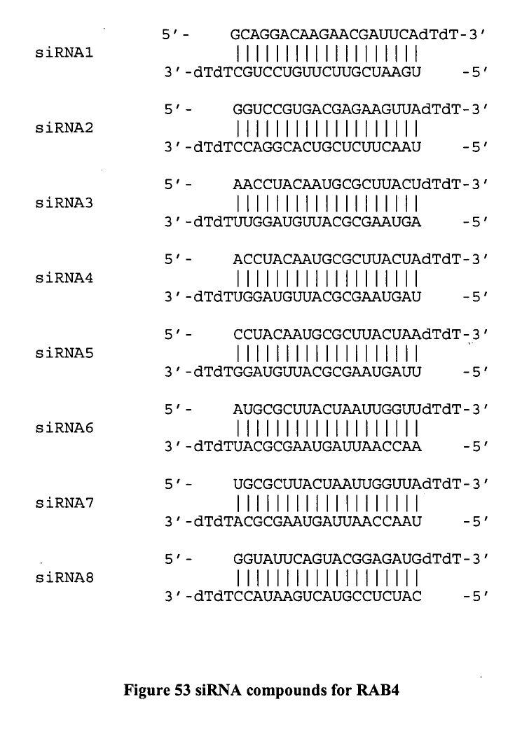

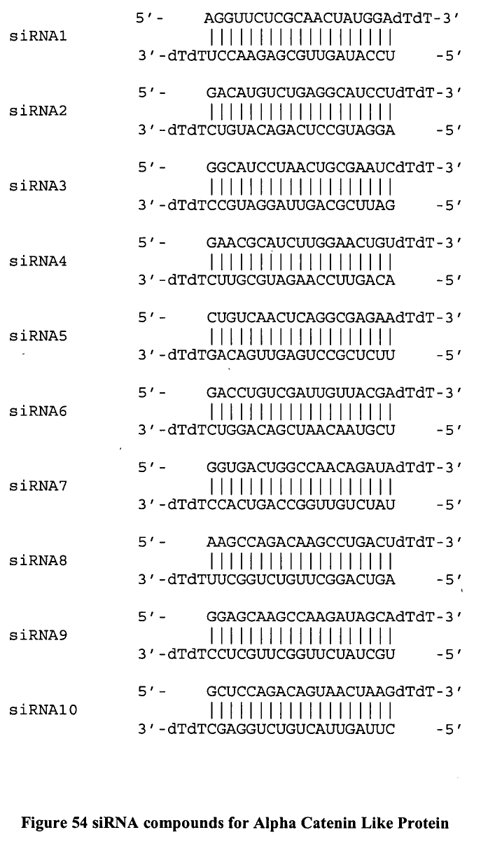

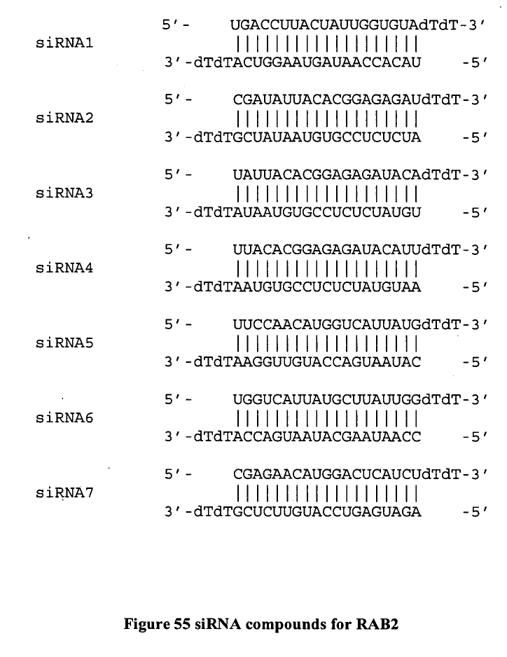

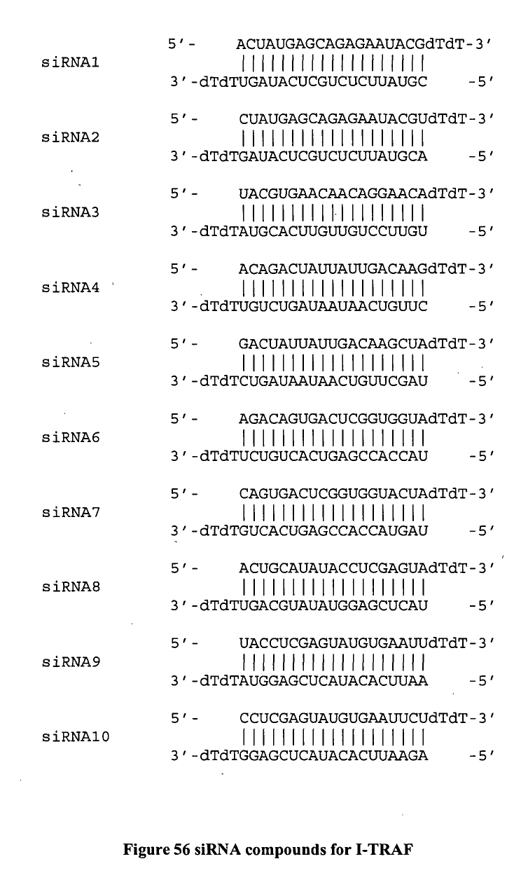

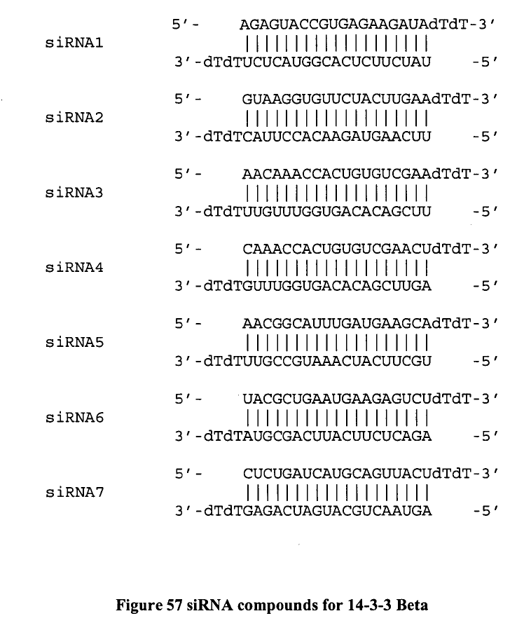

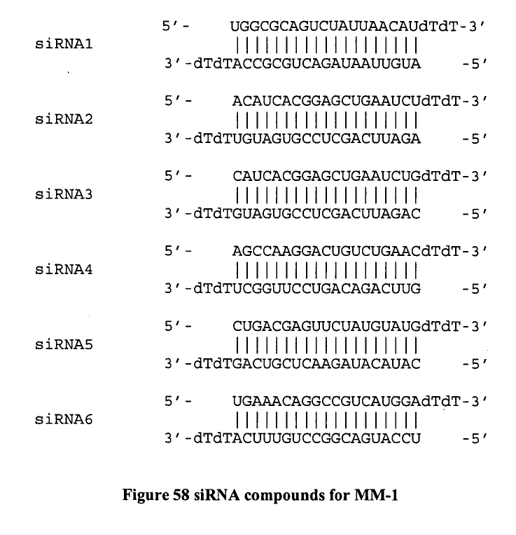

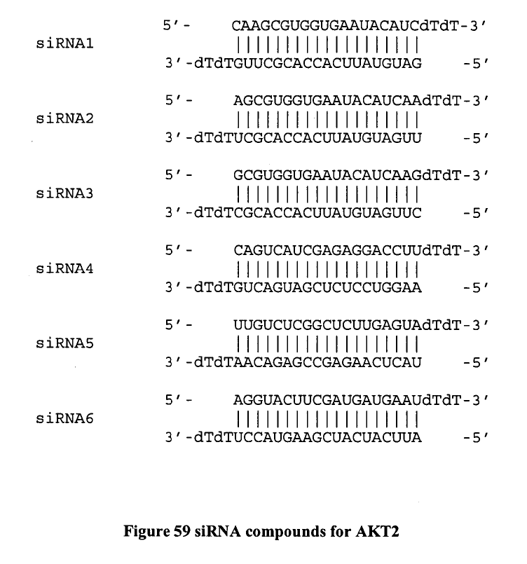

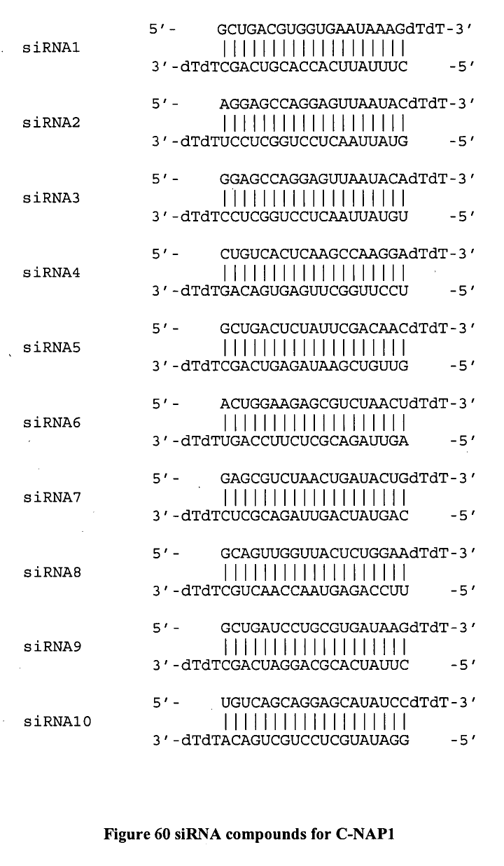

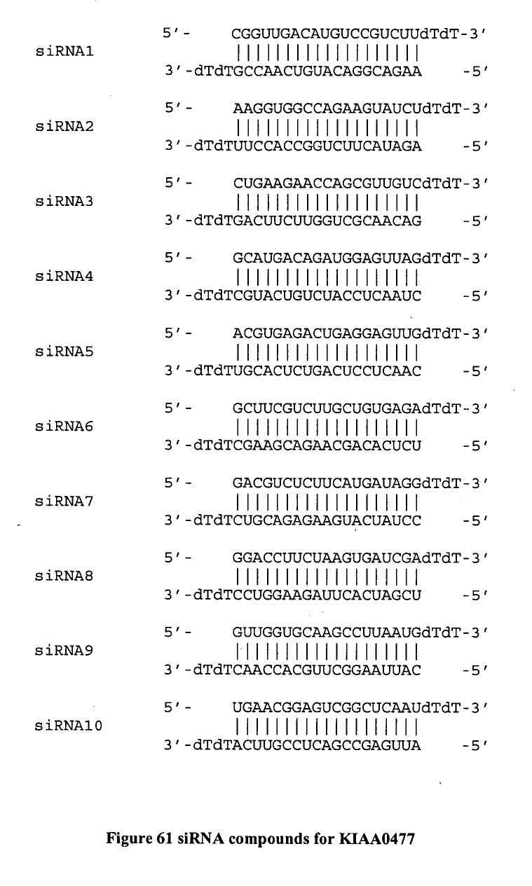

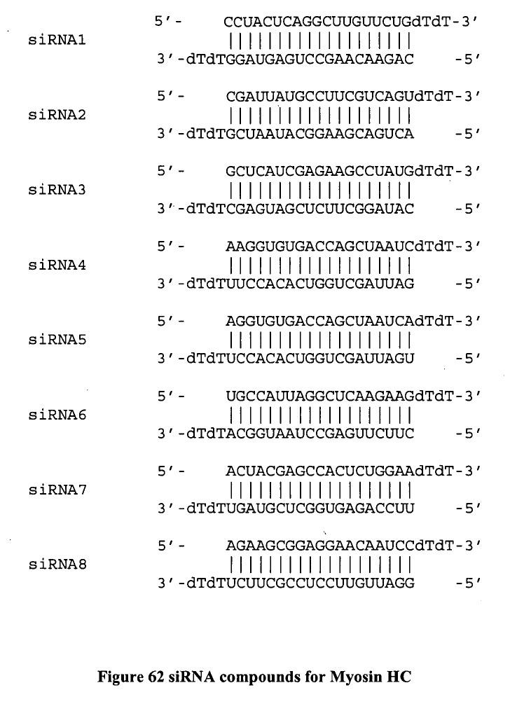

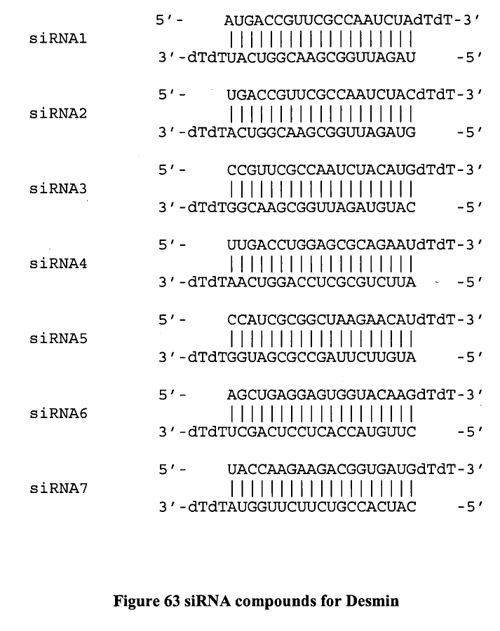

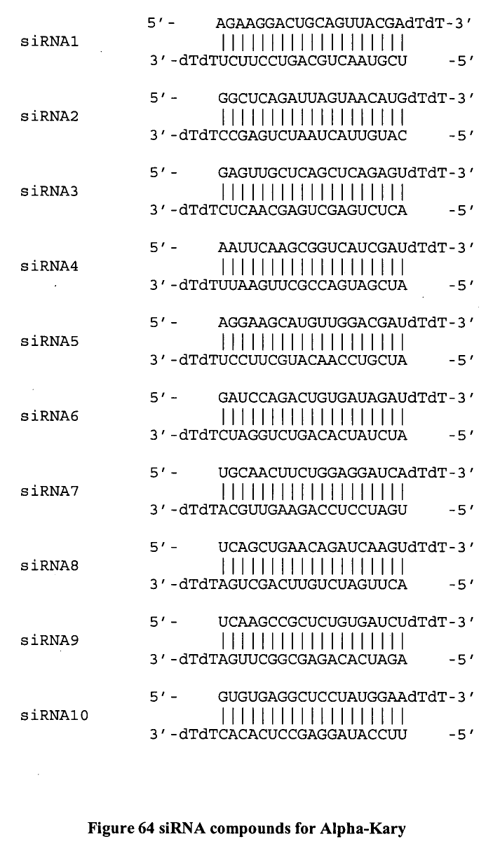

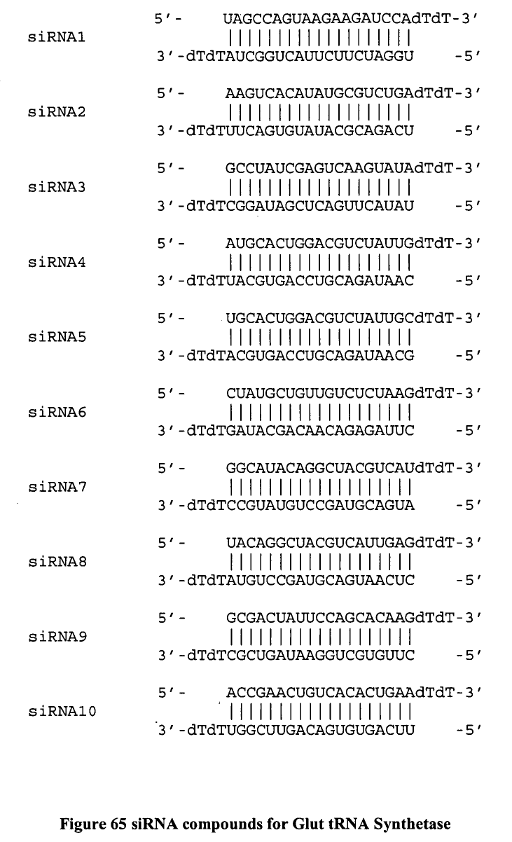

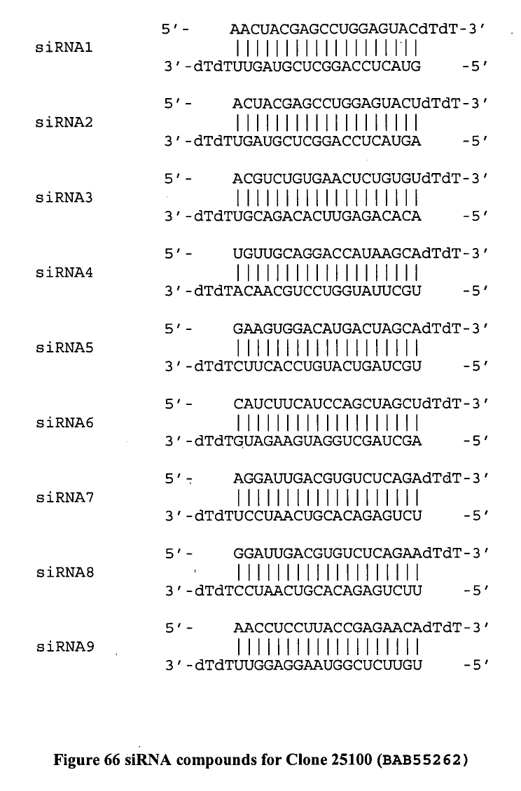

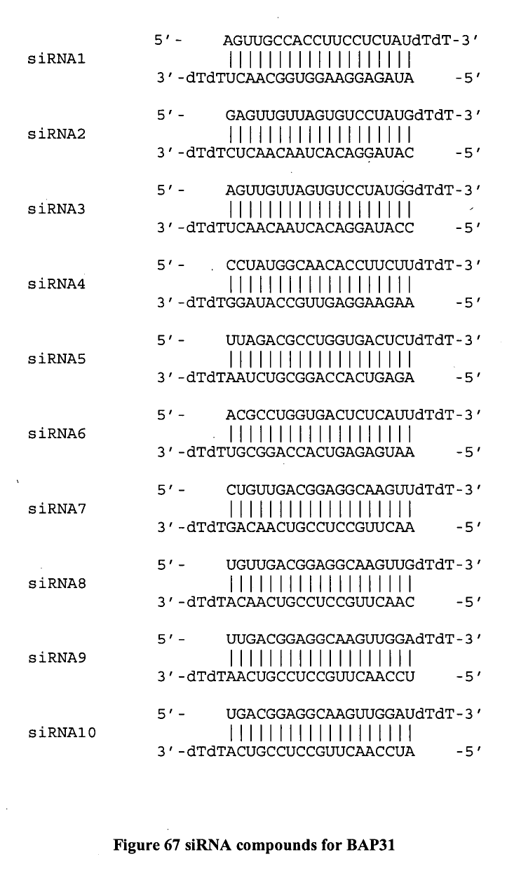

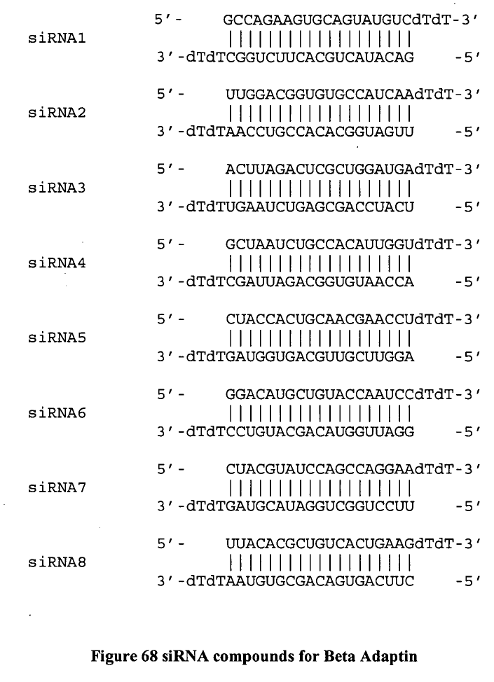

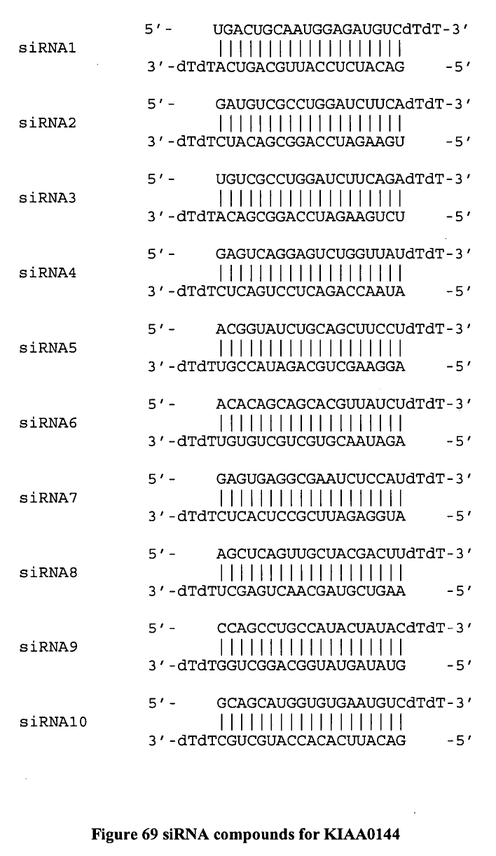

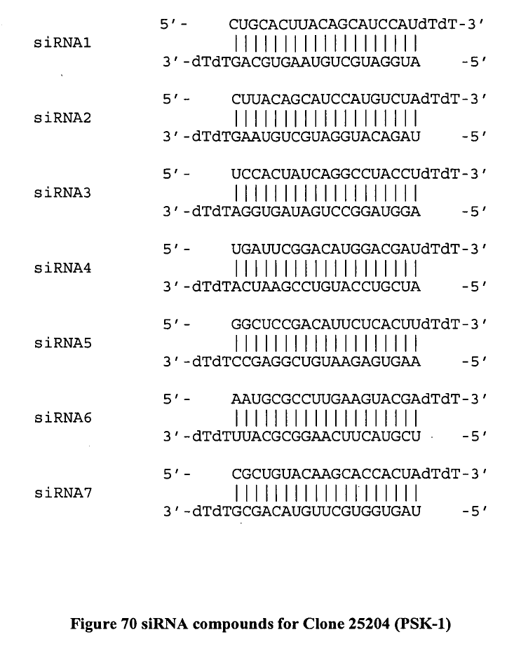

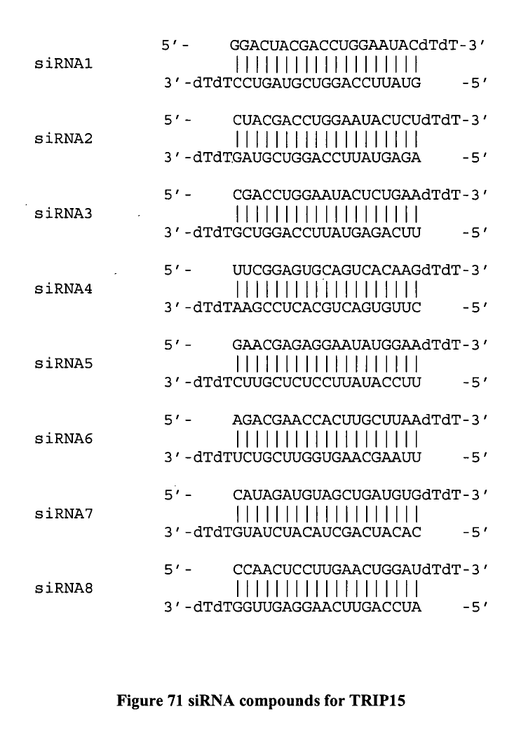

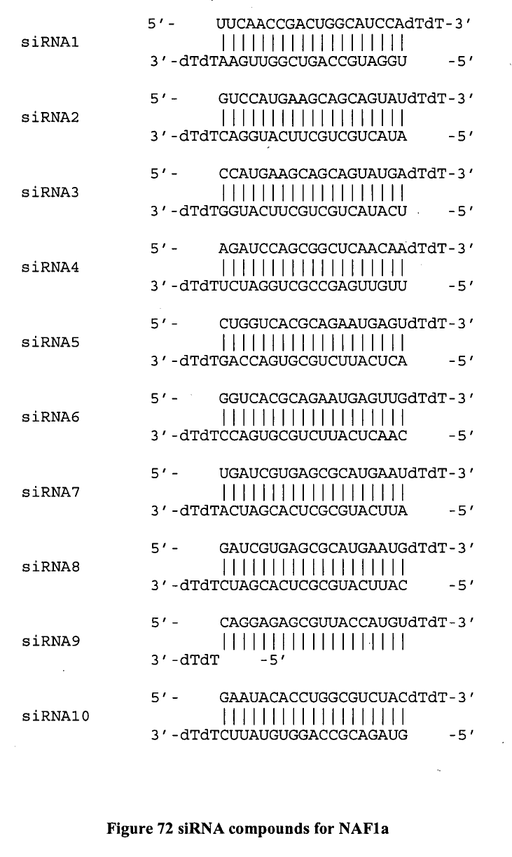

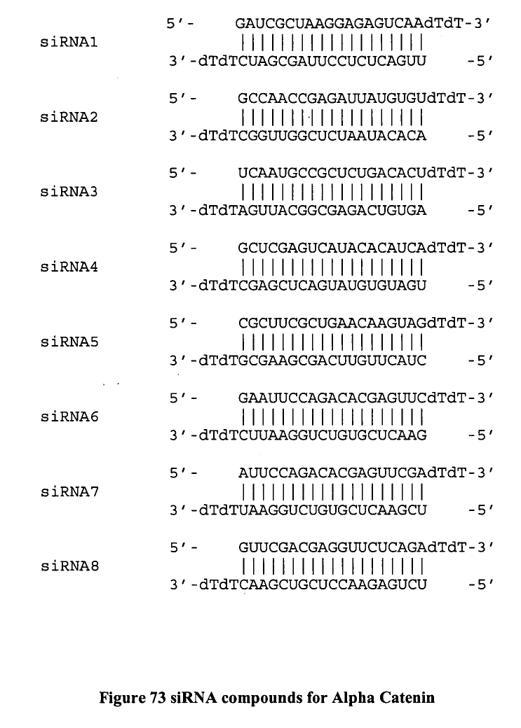

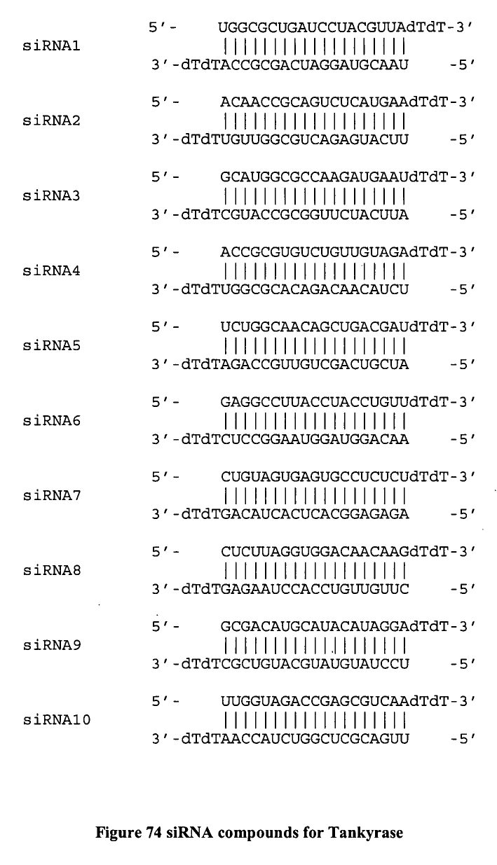

In another embodiment, double-stranded small interfering RNA (siRNA) compounds specific to nucleic acids encoding one or more interacting protein members of a protein complex identified in the present invention are administered to cells or tissue in vitro or in a patient to be therapeutically or prophylactically treated. FIGS. 1-75 depict the structures of several embodiments of the siRNA compounds useful in reducing mRNA levels of the proteins in the tables.

As is generally known in the art now, siRNA compounds are RNA duplexes comprising two complementary single-stranded RNAs of 21 nucleotides that form 19 base pairs and possess 3′ overhangs of two nucleotides. See Elbashir et al., Nature 411:494-498 (2001); and PCT Publication Nos. WO 00/44895; WO 01/36646; WO 99/32619; WO 00/01846; WO 01/29058; WO 99/07409; and WO 00/44914. When appropriately targeted via its nucleotide sequence to a specific mRNA in cells, an siRNA can specifically suppress gene expression through a process known as RNA interference (RNAi). See e.g., Zamore & Aronin, Nature Medicine, 9:266-267 (2003). siRNAs can reduce the cellular level of specific mRNAs, and decrease the level of proteins coded by such mRNAs. siRNAs utilize sequence complementarity to target an mRNA for destruction, and are sequence-specific. Thus, they can be highly target-specific, and in mammals have been shown to target mRNAs encoded by different alleles of the same gene. Because of this precision, side effects typically associated with traditional drugs can be reduced or eliminated. In addition, they are relatively stable, and like antisense and ribozyme molecules, they can also be modified to achieve improved pharmaceutical characteristics, such as increased stability, deliverability, and ease of manufacture. Moreover, because siRNA molecules take advantage of a natural cellular pathway, i.e., RNA interference, they are highly efficient in destroying targeted mRNA molecules. As a result, it is relatively easy to achieve a therapeutically effective concentration of an siRNA compound in patients. Thus, siRNAs are a promising new class of drugs being actively developed by pharmaceutical companies.

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. The gene silencing effect persisted without diminution for 10 days after the intravenous injection. The injected siRNA was effective in protecting the mice from liver failure and fibrosis. Song et al., Nature Medicine, 9:347-351 (2003). Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002).

The siRNA compounds provided according to the present invention can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845-7854 (1987) and Scaringe et al., Nucleic Acids Res., 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

The siRNA compounds can also be various modified equivalents of the siRNA structures. As used herein, “modified equivalent” means a modified form of a particular siRNA compound having the same target-specificity (i.e., recognizing the same mRNA molecules that complement the unmodified particular siRNA compound). Thus, a modified equivalent of an unmodified siRNA compound can have modified ribonucleotides, that is, ribonucleotides that contain a modification in the chemical structure of an unmodified nucleotide base, sugar and/or phosphate (or phospodiester linkage). As is known in the art, an “unmodified ribonucleotide” has one of the bases adenine, cytosine, guanine, and uracil joined to the 1′ carbon of beta-D-ribo-furanose.

Preferably, modified siRNA compounds contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Modified forms of siRNA compounds can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxymethyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyluridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyladenosine, beta-D-mannosylqueosine, uridine-5-oxyacetic acid, 2-thiocytidine, threonine derivatives, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, PCT Publication No. WO 92/07065; PCT Publication No. WO 93/15187; and Limbach et al., Nucleic Acids Res., 22:2183 (1994), each of which is incorporated herein by reference in its entirety.

In addition, modified siRNA compounds can also have substituted or modified sugar moieties, e.g., 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Modified siRNA compounds may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

Preferably, the 3′ overhangs of the siRNAs of the present invention are modified to provide resistance to cellular nucleases. In one embodiment the 3′ overhangs comprise 2′-deoxyribonucleotides. In a preferred embodiment (depicted in FIG. 1) these 3′ overhangs comprise a dinucleotide made of two 2′-deoxythymine residues (i.e., dTdT) linked by a 5′-3′ phosphodiester linkage.

siRNA compounds may be administered to mammals by various methods through different routes. For example, they can be administered by intravenous injection. See Song et al., Nature Medicine, 9:347-351 (2003). They can also be delivered directly to a particular organ or tissue by any suitable localized administration methods. Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002). Alternatively, they may be delivered encapsulated in liposomes, by iontophoresis, or by incorporation into other vehicles such as hydrogels, cyclodextrins, biodegradable nanocapsules, and bioadhesive microspheres.

In addition, they may also be delivered by a gene therapy approach, using a DNA vector from which siRNA compounds in, e.g., small hairpin form (shRNA), can be transcribed directly. Recent studies have demonstrated that while double-stranded siRNAs are very effective at mediating RNAi, short, single-stranded, hairpin-shaped RNAs can also mediate RNAi, presumably because they fold into intramolecular duplexes that are processed into double-stranded siRNAs by cellular enzymes. Sui et al., Proc. Natl. Acad. Sci. U.S.A., 99:5515-5520 (2002); Yu et al., Proc. Natl. Acad. Sci. U.S.A., 99:6047-6052 (2002); and Paul et al., Nature Biotech., 20:505-508 (2002)). This discovery has significant and far-reaching implications, since the production of such shRNAs can be readily achieved in vivo by transfecting cells or tissues with DNA vectors bearing short inverted repeats separated by a small number of (e.g., 3 to 9) nucleotides that direct the transcription of such small hairpin RNAs. Additionally, if mechanisms are included to direct the integration of the transcription cassette into the host cell genome, or to ensure the stability of the transcription vector, the RNAi caused by the encoded shRNAs, can be made stable and heritable. Not only have such techniques been used to “knock down” the expression of specific genes in mammalian cells, but they have now been successfully employed to knock down the expression of exogenously expressed transgenes, as well as endogenous genes in the brain and liver of living mice. See generally Hannon, Nature. 418:244-251 (2002) and Shi, Trends Genet., 19:9-12 (2003); see also Xia et al., Nature Biotech., 20:1006-1010 (2002).

Besides the siRNA compounds provided in FIGS. 1-74, additional siRNA compounds targeted at different sites of the nucleic acids encoding one or more interacting protein members of a protein complex identified in the present invention may also be designed and synthesized according to general guidelines provided herein and generally known to skilled artisans. See e.g., Elbashir, et al. (Nature 411: 494-498 (2001). For example, guidelines have been compiled into “The siRNA User Guide” which is available at the following web address: www.mpibpc.gwdg.de/abteilungen/100/105/sirna.html.

Additionally, to assist in the design of siRNAs for the efficient RNAi-mediated silencing of any target gene, several siRNA supply companies maintain web-based design tools that utilize these general guidelines for “picking” siRNAs when presented with the mRNA or coding DNA sequence of the target gene. Examples of such tools can be found at the web sites of Dharmacon, Inc. (Lafayette, Colo.), Ambion, Inc. (Austin, Tex.), and Qiagen, Inc. (Valencia, Calif.), among others. Generally speaking, when provided with an mRNA or coding DNA sequence, these design tools scan the sequence for potential siRNA targets, using several distinct criteria. For example, the design tools may scan for an open reading frame and limit further scanning to that region of sequence. They may then scan for a particular dinucleotide, the most desirable of which being AA, or alternatively CA, GA or TA. Upon finding one of these dinucleotides, they will then examine the dinucleotide and the 19 nucleotides immediately 3′ of it for G/C content, nucleotide triplets (esp. GGG & CCC), and, using a BLAST algorithm search, for whether or not the 19 nucleotide sequence is unique to a specific target gene in the human genome. The features that make for an “ideal” target sequence are: (1) a 5′-most dinucleotide sequence of AA, or, less preferably, CA, GA or TA; (2) a G/C content of approximately 30-50%; (3) lack of trinucleotide repeats, especially GGG and CCC, and (4) being unique to the target gene (i.e., sequences that share no significant homology with genes other than the one being targeted), so that other genes are not inadvertently targeted by the same siRNA designed for this particular target sequence. Another criteria to be considered is whether or not the target sequence includes a known polymorphic site. If so, siRNAs designed to target one particular allele may not effectively target another allele, since single base mismatches between the target sequence and its complementary strand in a given siRNA can greatly reduce the effectiveness of RNAi induced by that siRNA. Given that target sequence and such design tools and design criteria, an ordinarily skilled artisan apprised of the present disclosure should be able to design and synthesized additional siRNA compounds useful in reducing the mRNA level and therefore protein level of one or more interacting protein members of a protein complex identified in the present invention.

### 6.2.3. Antisense Therapy

In another embodiment, antisense compounds specific to nucleic acids encoding one or more interacting protein members of a protein complex identified in the present invention are administered to cells or tissue in vitro or in a patient to be therapeutically or prophylactically treated. The antisense compounds should specifically inhibit the expression of the one or more interacting protein members. Examples of antisense compounds specific to nucleic acids encoding proteins in the tables are found in SEQ ID NO:'s 1-74.

As is known in the art, antisense drugs generally act by hybridizing to a particular target nucleic acid thus blocking gene expression. Methods for designing antisense compounds and using such compounds in treating diseases are well known and well developed in the art. For example, the antisense drug Vitravene® (fomivirsen), a 21-base long oligonucleotide, has been successfully developed and marketed by Isis Pharmaceuticals, Inc. for treating cytomegalovirus (CMV)-induced retinitis.

Any methods for designing and making antisense compounds may be used for the purpose of the present invention. See generally, Sanghvi et al., eds., Antisense Research and Applications, CRC Press, Boca Raton, 1993. Typically, antisense compounds are oligonucleotides designed based on the nucleotide sequence of the mRNA or gene of one or more target proteins, e.g., the interacting protein members of a particular protein complex of the present invention. In particular, antisense compounds can be designed to specifically hybridize to a particular region of the gene sequence or mRNA of one or more of the interacting protein members to modulate (increase or decrease) replication, transcription, or translation. As used herein, the term “specifically hybridize” or paraphrases thereof means a sufficient degree of complementarity or pairing between an antisense oligo and a target DNA or mRNA such that stable and specific binding occurs therebetween. In particular, 100% complementary or pairing is not required. Specific hybridization takes place when sufficient hybridization occurs between the antisense compound and its intended target nucleic acids in the substantial absence of non-specific binding of the antisense compound to non-target sequences under predetermined conditions, e.g., for purposes of in vivo treatment, preferably under physiological conditions. Preferably, specific hybridization results in the interference with normal expression of the target DNA or mRNA.

For example, antisense oligonucleotides can be designed to specifically hybridize to target genes, in regions critical for regulation of transcription; to pre-mRNAs, in regions critical for correct splicing of nascent transcripts; and to mature mRNAs, in regions critical for translation initiation or mRNA stability and localization.

As is generally known in the art, commonly used oligonucleotides are oligomers or polymers of ribonucleotides or deoxyribonucleotides, that are composed of a naturally-occurring nitrogenous base, a sugar (ribose or deoxyribose) and a phosphate group. In nature, the nucleotides are linked together by phosphodiester bonds between the 3′ and 5′ positions of neighboring sugar moieties. However, it is noted that the term “oligonucleotides” also encompasses various non-naturally occurring mimetics and derivatives, i.e., modified forms, of naturally occurring oligonucleotides as described below. Typically an antisense compound of the present invention is an oligonucleotide having from about 6 to about 200, and preferably from about 8 to about 30 nucleoside bases.

The antisense compounds preferably contain modified backbones or non-natural internucleoside linkages, including but not limited to, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Another useful modified oligonucleotide is peptide nucleic acid (PNA), in which the sugar-backbone of an oligonucleotide is replaced with an amide containing backbone, e.g., an aminoethylglycine backbone. See U.S. Pat. Nos. 5,539,082 and 5,714,331; and Nielsen et al., Science, 254, 1497-1500 (1991), all of which are incorporated herein by reference. PNA antisense compounds are resistant to RNase H digestion and thus exhibit longer half-life. In addition, various modifications may be made in PNA backbones to impart desirable drug profiles such as better stability, increased drug uptake, higher affinity to target nucleic acid, etc.

Alternatively, the antisense compounds are oligonucleotides containing modified nucleosides, i.e., modified purine or pyrimidine bases, e.g., 5-substituted pyrimidines, 6-azapyrimidines, and N-2, N-6 and O-substituted purines, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, each of which is incorporated herein by reference in its entirety.

In addition, oligonucleotides with substituted or modified sugar moieties may also be used. For example, an antisense compound may have one or more 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Other types of oligonucleotide modifications are also useful including linking an oligonucleotide to a lipid, phospholipid or cholesterol moiety, cholic acid, thioether, aliphatic chain, polyamine, polyethylene glycol (PEG), or a protein or peptide. The modified oligonucleotides may exhibit increased uptake into cells, and improved stability, i.e., resistance to nuclease digestion and other biodegradations. See e.g., U.S. Pat. No. 4,522,811; Burnham, Am. J. Hosp. Pharm., 15:210-218 (1994).

Antisense compounds can be synthesized using any suitable methods known in the art. In fact, antisense compounds may be custom made by commercial suppliers. Alternatively, antisense compounds may be prepared using DNA synthesizers available commercially from various vendors, e.g., Applied Biosystems Group of Norwalk, Conn.

The antisense compounds can be formulated into a pharmaceutical composition with suitable carriers and administered into cells or tissue in vitro or in a patient using any suitable route of administration. Alternatively, the antisense compounds may also be used in a “gene-therapy” approach. That is, the oligonucleotide is subcloned into a suitable vector and transformed into human cells. The antisense oligonucleotide is then produced in vivo through transcription. Methods for gene therapy are disclosed in Section 6.3.2 below.

### 6.2.4. Ribozyme Therapy

In another embodiment, an enzymatic RNA or ribozyme is designed to target the nucleic acids encoding one or more of the interacting protein members of the protein complexes of the present invention. Ribozymes are RNA molecules possessing enzymatic activity. One class of ribozymes is capable of repeatedly cleaving other separate RNA molecules into two or more pieces in a nucleotide base sequence specific manner. See Kim et al., Proc. Natl. Acad. of Sci. USA, 84:8788 (1987); Haseloff and Gerlach, Nature, 334:585 (1988); and Jefferies et al., Nucleic Acid Res., 17:1371 (1989). Such ribozymes typically have two functional domains: a catalytic domain and a binding sequence that guides the binding of ribozymes to a target RNA through complementary base-pairing. Once a specifically-designed ribozyme is bound to a target mRNA, it enzymatically cleaves the target mRNA, typically reducing its stability and destroying its ability to direct translation of an encoded protein. After a ribozyme has cleaved its RNA target, it is released from that target RNA and thereafter can bind and cleave another target. That is, a single ribozyme molecule can repeatedly bind and cleave new targets. Therefore, one advantage of ribozyme treatment is that a lower amount of exogenous RNA is required as compared to conventional antisense therapies. In addition, ribozymes exhibit less affinity to mRNA targets than DNA-based antisense oligonucleotides, and therefore are less prone to bind to unintended targets.

In accordance with the present invention, a ribozyme may target any portion of the mRNA encoding one or more interacting protein members of the protein complexes formed by the interactions described in the tables. Methods for selecting a ribozyme target sequence and designing and making ribozymes are generally known in the art. See e.g., U.S. Pat. Nos. 4,987,071; 5,496,698; 5,525,468; 5,631,359; 5,646,020; 5,672,511; and 6,140,491, each of which is incorporated herein by reference in its entirety. For example, suitable ribozymes may be designed in various configurations such as hammerhead motifs, hairpin motifs, hepatitis delta virus motifs, group I intron motifs, or RNase P RNA motifs. See e.g., U.S. Pat. Nos. 4,987,071; 5,496,698; 5,525,468; 5,631,359; 5,646,020; 5,672,511; and 6,140,491; Rossi et al., AIDS Res. Human Retroviruses 8:183 (1992); Hampel and Tritz, Biochemistry 28:4929 (1989); Hampel et al., Nucleic Acids Res., 18:299 (1990); Perrotta and Been, Biochemistry 31:16 (1992); and Guerrier-Takada et al., Cell, 35:849 (1983).

Ribozymes can be synthesized by the same methods used for normal RNA synthesis. For example, such methods are disclosed in Usman et al., J. Am. Chem. Soc., 109:7845-7854 (1987) and Scaringe et al., Nucleic Acids Res., 18:5433-5441 (1990). Modified ribozymes may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

Ribozymes of the present invention may be administered to cells by any known methods, e.g., disclosed in International Publication No. WO 94/02595. For example, they can be administered directly to cells or tissue in vitro or in a patient through any suitable route, e.g., intravenous injection. Alternatively, they may be delivered encapsulated in liposomes, by iontophoresis, or by incorporation into other vehicles such as hydrogels, cyclodextrins, biodegradable nanocapsules, and bioadhesive microspheres. In addition, they may also be delivered by a gene therapy approach, using a DNA vector from which the ribozyme RNA can be transcribed directly. Gene therapy methods are disclosed in detail below in Section 6.3.2.

### 6.2.5. Other Methods

The in-patient concentrations and activities of the protein complexes and interacting proteins of the present invention may also be altered by other methods. For example, compounds identified in accordance with the methods described in Section 5 that are capable of interfering with or dissociating protein-protein interactions between the interacting protein members of a protein complex may be administered to cells or tissue in vitro or in a patient. Compounds identified in in vitro binding assays described in Section 5.2 that bind to the protein complexes of the present invention, or the interacting members thereof, may also be used in the treatment. Compounds identified in in vitro binding assays described in Section 5.2 that bind to the protein complexes of the present invention, or the interacting members thereof, may also be used in the treatment.

In addition, potentially useful agents also include incomplete proteins, i.e., fragments of the interacting protein members that are capable of binding to their respective binding partners in a protein complex but are defective with respect to their normal cellular functions. For example, binding domains of the interacting member proteins of a protein complex may be used as competitive inhibitors of the activities of the protein complex. As will be apparent to skilled artisans, derivatives or homologues of the binding domains may also be used. Binding domains can be easily identified using molecular biology techniques, e.g., mutagenesis in combination with yeast two-hybrid assays. Preferably, the protein fragment used is a fragment of an interacting protein member having a length of less than 90%, 80%, more preferably less than 75%, 65%, 50%, or less than 40% of the full length of the protein member. SEQ ID NO:s 75-254 are examples of protein fragments of proteins in the tables that are potentially useful agents.

In one embodiment, a fragment of a protein identified in the tables above is administered. In a specific embodiment, one or more of the interaction domains of a protein identified in the tables, within the regions listed in the tables, is administered to cells or tissue in vitro, or are administered to a patient in need of such treatment. For example, suitable protein fragments can include polypeptides having a contiguous span of 4, 5, 6, 7, 8, 9, 10, 11, 12, 13, 14, 15, 18, 20 or 25, preferably from 4 to 30, 40 or 50 amino acids or more of the sequence of a first protein identified in the tables, that are capable of interacting with a second protein described in the tables. Also, suitable protein fragments can include peptides capable of binding one or more of the proteins described in the tables, and having an amino acid sequence of from 4 to 30 amino acids that is at least 75%, 80%, 82%, 85%, 87%, 90%, 95% or more identical to a contiguous span of amino acids of a protein described in the tables. Alternatively, a polypeptide capable of interacting with a first protein of an interacting pair of proteins of the present invention, and having a contiguous span of 4, 5, 6, 7, 8, 9, 10, 11, 12, 13, 14, 15, 18, 20 or 25, preferably from 4 to 30, 40 or 50 or more amino acids of the amino acid sequence of a second protein of the same interacting pair of proteins, may be administered. Also, other examples of suitable compounds include a peptide capable of binding a first interacting partner of a pair of interacting proteins of the present invention and having an amino acid sequence of from 4 to 30, 40, 50 or more amino acids that is at least 75%, 80%, 82%, 85%, 87%, 90%, 92%, 95% or more identical to a contiguous span of amino acids from a second interacting partner of a pair of interacting proteins of the present invention. In addition, the administered compounds can be an antibody or antibody fragment, preferably a single-chain antibody immunoreactive with any of the proteins listed in the tables, or a protein complex of the present invention.

The protein fragments suitable as competitive inhibitors can be delivered into cells by direct cell internalization, receptor mediated endocytosis, or via a “transporter.” It is noted that when the target proteins or protein complexes to be modulated reside inside cells, the compound administered to cells in vitro or in vivo in the method of the present invention preferably is delivered into the cells in order to achieve optimal results. Thus, preferably, the compound to be delivered is associated with a transporter capable of increasing the uptake of the compound by cells harboring the target protein or protein complex. As used herein, the term “transporter” refers to an entity (e.g., a compound or a composition or a physical structure formed from multiple copies of a compound or multiple different compounds) that is capable of facilitating the uptake of a compound of the present invention by animal cells, particularly human cells. That is, the cell uptake of a compound of the present invention in the presence of a “transporter” is at least 50% higher than the cell uptake of the compound in the absence of the “transporter.” Preferably, a “transporter” is selected such that the cell uptake of a compound of the present invention in the presence of a “transporter” is at least 75% higher, preferably at least 100% or 200% higher, and more preferably at least 300%, 400% or 500% higher than the cell uptake of the compound in the absence of the “transporter.” Methods of assaying cell uptake of a compound should be apparent to skilled artisans. For example, the compound to be delivered can be labeled with a radioactive isotope or another detectable marker (e.g., a fluorescence marker), and added to cultured cells in the presence or absence of a transporter, and incubated for a time period sufficient to allow maximal uptake. Cells can then be separated from the culture medium and the detectable signal (e.g., radioactivity) caused by the compound inside the cells can be measured. The result obtained in the presence of a transporter can be compared to that obtained in the absence of a transporter.

Many molecules and structures known in the art can be used as “transporters.” In one embodiment, a penetratin is used as a transporter. For example, the homeodomain of Antennapedia, a Drosophila transcription factor, can be used as a transporter to deliver a compound of the present invention. Indeed, any suitable member of the penetratin class of peptides can be used to carry a compound of the present invention into cells. Penetratins are disclosed in, e.g., Derossi et al., Trends Cell Biol., 8:84-87 (1998), which is incorporated herein by reference. Penetratins transport molecules attached thereto across cytoplasmic membranes or nuclear membranes efficiently, in a receptor-independent, energy-independent, and cell type-independent manner. Methods for using a penetratin as a carrier to deliver oligonucleotides and polypeptides are also disclosed in U.S. Pat. No. 6,080,724; Pooga et al., Nat. Biotech., 16:857 (1998); and Schutze et al., J. Immunol., 157:650 (1996), all of which are incorporated herein by reference. U.S. Pat. No. 6,080,724 defines the minimal requirements for a penetratin peptide as a peptide of 16 amino acids with 6 to 10 of which being hydrophobic. The amino acid at position 6 counting from either the N- or C-terminus is tryptophan, while the amino acids at positions 3 and 5 counting from either the N- or C-terminus are not both valine. Preferably, the helix 3 of the homeodomain of Drosophila Antennapedia is used as a transporter. More preferably, a peptide having a sequence of amino acid residues 43-58 of the homeodomain Antp is employed as a transporter. In addition, other naturally occurring homologs of the helix 3 of the homeodomain of Drosophila Antennapedia can be used. For example, homeodomains of Fushi-tarazu and Engrailed have been shown to be capable of transporting peptides into cells. See Han et al., Mol. Cells, 10:728-32 (2000). As used herein, the term “penetratin” also encompasses peptoid analogs of the penetratin peptides. Typically, the penetratin peptides and peptoid analogs thereof are covalently linked to a compound to be delivered into cells thus increasing the cellular uptake of the compound.

In another embodiment, the HIV-1 tat protein or a derivative thereof is used as a “transporter” covalently linked to a compound according to the present invention. The use of HIV-1 tat protein and derivatives thereof to deliver macromolecules into cells has been known in the art. See Green and Loewenstein, Cell, 55:1179 (1988); Frankel and Pabo, Cell, 55:1189 (1988); Vives et al., J. Biol. Chem., 272:16010-16017 (1997); Schwarze et al., Science, 285:1569-1572 (1999). It is known that the sequence responsible for cellular uptake consists of the highly basic region, amino acid residues 49-57. See e.g., Vives et al., J. Biol. Chem., 272:16010-16017 (1997); Wender et al., Proc. Nat'l Acad. Sci. USA, 97:13003-13008 (2000). The basic domain is believed to target the lipid bilayer component of cell membranes. It causes a covalently linked protein or nucleic acid to cross cell membrane rapidly in a cell type-independent manner. Proteins ranging in size from 15 to 120 kD have been delivered with this technology into a variety of cell types both in vitro and in vivo. See Schwarze et al., Science, 285:1569-1572 (1999). Any HIV tat-derived peptides or peptoid analogs thereof capable of transporting macromolecules such as peptides can be used for purposes of the present invention. For example, any native tat peptides having the highly basic region, amino acid residues 49-57 can be used as a transporter by covalently linking it to the compound to be delivered. In addition, various analogs of the tat peptide of amino acid residues 49-57 can also be useful transporters for purposes of this invention. Examples of various such analogs are disclosed in Wender et al., Proc. Nat'l Acad. Sci. USA, 97:13003-13008 (2000) (which is incorporated herein by reference) including, e.g., d-Tat₄₉₋₅₇, retro-inverso isomers of 1- or d-Tat₄₉₋₅₇ (i.e., 1-Tat₅₇₋₄₉ and d-Tat₅₇₋₄₉), L-arginine oligomers, D-arginine oligomers, L-lysine oligomers, D-lysine oligomers, L-histine oligomers, D-histine oligomers, L-omithine oligomers, D-ornithine oligomers, and various homologues, derivatives (e.g., modified forms with conjugates linked to the small peptides) and peptoid analogs thereof. Preferably, arginine oligomers are preferred to the other oligomers, since arginine oligomers are much more efficient in promoting cellular uptake. As used herein, the term “oligomer” means a molecule that includes a covalently linked chain of amino acid residues of the same amino acids having a large enough number of such amino acid residues to confer transporter activities on the molecule. Typically, an oligomer contains at least 6, preferably at least 7, 8, or 9 such amino acid residues. In one embodiment, the transporter is a peptide that includes at least six contiguous amino acid residues that are a combination of two or more of L-arginine, D-arginine, L-lysine, D-lysine, L-histidine, D-histine, L-omithine, and D-omithine.

Other useful transporters known in the art include, but are not limited to, short peptide sequences derived from fibroblast growth factor (See Lin et al., J. Biol. Chem., 270:14255-14258 (1998)), Galparan (See Pooga et al., FASEB J 12:67-77 (1998)), and HSV-1 structural protein VP22 (See Elliott and O'Hare, Cell, 88:223-233 (1997)).

As the above-described various transporters are generally peptides, fusion proteins can be conveniently made by recombinant expression to contain a transporter peptide covalently linked by a peptide bond to a competitive protein fragment. Alternatively, conventional methods can be used to chemically synthesize a transporter peptide or a peptide of the present invention or both.

The hybrid peptide can be administered to cells or tissue in vitro or to a patient in a suitable pharmaceutical composition as provided in Section 8.

In addition to peptide-based transporters, various other types of transporters can also be used, including but not limited to cationic liposomes (see Rui et al., J. Am. Chem. Soc., 120:11213-11218 (1998)), dendrimers (Kono et al., Bioconjugate Chem., 10:1115-1121 (1999)), siderophores (Ghosh et al., Chem. Biol., 3:1011-1019 (1996)), etc. In a specific embodiment, the compound according to the present invention is encapsulated into liposomes for delivery into cells.

Additionally, when a compound according to the present invention is a peptide, it can be administered to cells by a gene therapy method. That is, a nucleic acid encoding the peptide can be administered to in vitro cells or to cells in vivo in a human or animal body. Any suitable gene therapy methods may be used for purposes of the present invention. Various gene therapy methods are well known in the art and are described in Section 6.3.2. below. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med., 166:219 (1987).

In yet another embodiment, the gene therapy methods discussed in Section 6.3.2 below are used to “knock out” the gene encoding an interacting protein member of a protein complex, or to reduce the gene expression level. For example, the gene may be replaced with a different gene sequence or a non-functional sequence or simply deleted by homologous recombination. In another gene therapy embodiment, the method disclosed in U.S. Pat. No. 5,641,670, which is incorporated herein by reference, may be used to reduce the expression of the genes for the interacting protein members. Essentially, an exogenous DNA having at least a regulatory sequence, an exon and a splice donor site can be introduced into an endogenous gene encoding an interacting protein member by homologous recombination such that the regulatory sequence, the exon and the splice donor site present in the DNA construct become operatively linked to the endogenous gene. As a result, the expression of the endogenous gene is controlled by the newly introduced exogenous regulatory sequence. Therefore, when the exogenous regulatory sequence is a strong gene expression repressor, the expression of the endogenous gene encoding the interacting protein member is reduced or blocked. See U.S. Pat. No. 5,641,670.

### 6.3. Activation of Protein Complex or Interacting Protein Members Thereof

The present invention also provides methods for increasing in cells or tissue in vitro or in a patient the concentration and/or activity of a protein complex, or of an individual protein member thereof, identified in accordance with the present invention. Such methods can be particularly useful in instances where a reduced concentration and/or activity of a protein complex, or a protein member thereof, is associated with a particular disease or disorder to be treated, or where an increased concentration and/or activity of a protein complex, or a protein member thereof, would be beneficial to the improvement of a cellular function or disease state. By increasing the concentration of the protein complex, or a protein member thereof, and/or stimulating the functional activities of the protein complex or a protein member thereof, the disease or disorder may be treated or prevented.

### 6.3.1. Administration of Protein Complex or Protein Members Thereof

Where the concentration or activity of a particular protein complex of the present invention, or any individual protein constituent of a protein complex in cells or tissue in vitro or in a patient is determined to be low or is desired to be increased, the protein complex, or an individual constituent protein of the protein complex may be administered directly to the patient to increase the concentration and/or activity of the protein complex, or the individual constituent protein. For this purpose, protein complexes prepared by any one of the methods described in Section 2.2 may be administered to the patient, preferably in a pharmaceutical composition as described below. Alternatively, one or more individual interacting protein members of the protein complex may also be administered to the patient in need of treatment. For example, one or more of the individual proteins or the interacting pairs of proteins described in the tables may be given to cells or tissue in vitro or to a patient. Proteins isolated or purified from normal individuals or recombinantly produced can all be used in this respect. Preferably, two or more interacting protein members of a protein complex are administered. The proteins or protein complexes may be administered to a patient needing treatment using any of the methods described in Section 8.

### 6.3.2. Gene Therapy

In another embodiment, the concentration and/or activity of a particular protein complex comprising one or more of the interacting pairs of proteins described in the tables or an individual constituent protein of a protein complex of the present invention is increased or restored in patients, tissue or cells by a gene therapy approach. For example, nucleic acids encoding one or more protein members of a protein complex of the present invention, or portions or fragments thereof are introduced into patients, tissue, or cells such that the protein(s) are expressed from the introduced nucleic acids. For these purposes, nucleic acids encoding one or more of the proteins described in the tables, or fragments, homologues or derivatives thereof can be used in the gene therapy in accordance with the present invention. For example, if a disease-causing mutation exists in one of the protein members in cells or tissue in vitro or in a patient, then a nucleic acid encoding a wild-type protein can be introduced into tissue cells of the patient. The exogenous nucleic acid can be used to replace the corresponding endogenous defective gene by, e.g., homologous recombination. See U.S. Pat. No. 6,010,908, which is incorporated herein by reference. Alternatively, if the disease-causing mutation is a recessive mutation, the exogenous nucleic acid is simply used to express a wild-type protein in addition to the endogenous mutant protein. In another approach, the method disclosed in U.S. Pat. No. 6,077,705 may be employed in gene therapy. That is, the patient is administered both a nucleic acid construct encoding a ribozyme and a nucleic acid construct comprising a ribozyme resistant gene encoding a wild type form of the gene product. As a result, undesirable expression of the endogenous gene is inhibited and a desirable wild-type exogenous gene is introduced. In yet another embodiment, if the endogenous gene is of wild-type and the level of expression of the protein encoded thereby is desired to be increased, additional copies of wild-type exogenous genes may be introduced into the patient by gene therapy, or alternatively, a gene activation method such as that disclosed in U.S. Pat. No. 5,641,670 may be used.

Various gene therapy methods are well known in the art. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med. 166:219 (1987).

Any suitable gene therapy methods may be used for the purposes of the present invention. Generally, a nucleic acid encoding a desirable protein (e.g., one selected from any of the tables) is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In addition, selection markers may also be included in the vector for purposes of selecting, in vitro, those cells that contain the exogenous gene. Various selection markers known in the art may be used including, but not limited to, e.g., genes conferring resistance to neomycin, hygromycin, zeocin, and the like.

In one embodiment, the exogenous nucleic acid (gene) is incorporated into a plasmid DNA vector. Many commercially available expression vectors may be useful for the present invention, including, e.g., pCEP4, pcDNAI, pIND, pSecTag2, pVAX1, pcDNA3.1, and pBI-EGFP, and pDisplay.

Various viral vectors may also be used. Typically, in a viral vector, the viral genome is engineered to eliminate the disease-causing capability of the virus, e.g., the ability to replicate in the host cells. The exogenous nucleic acid to be introduced into cells or tissue in vitro or in a patient may be incorporated into the engineered viral genome, e.g., by inserting it into a viral gene that is non-essential to the viral infectivity. Viral vectors are convenient to use as they can be easily introduced into cells, tissues and patients by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), lentiviruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun., 159:1191-1198 (1984)), gibbon ape leukemia virus (See, Miller et al., J Virology, 65:2220-2224 (1991)), HIV, (See Shimada et al., J. Clin. Invest., 88:1043-1047 (1991)), and avian retroviruses (See Cosset et al., J Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431-434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or taken up by cells.

The exogenous gene fragment or plasmid DNA vector containing the exogenous gene may also be introduced into cells by way of receptor-mediated endocytosis. See e.g., U.S. Pat. No. 6,090,619; Wu and Wu, J. Biol. Chem., 263:14621 (1988); Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor binding moiety (e.g., a cyclic peptide having the sequence Arg-Gly-Asp).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily take it up.

The exogenous gene can be introduced into cells or tissue in vitro or in a patient for purposes of gene therapy by various methods known in the art. For example, the exogenous gene sequences alone or in a conjugated or complex form described above, or incorporated into viral or DNA vectors, may be administered directly by injection into an appropriate tissue or organ of a patient. Alternatively, catheters or like devices may be used to deliver exogenous gene sequences, complexes, or vectors into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

In addition, the exogenous gene or vectors containing the gene can be introduced into isolated cells using any known techniques such as calcium phosphate precipitation, microinjection, lipofection, electroporation, biolystics, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

### 6.4 Small Organic Compounds

Diseases or disorders in cells or tissue in vitro, or in a patient, associated with the decreased concentration or activity of a protein complex of the present invention, or an individual protein constituent of a protein complex identified in accordance with the present invention, can also be ameliorated by administering to the patient a compound identified by the methods described in Sections 5.3.1.4, 5.2, and Section 5.4, which is capable of modulating the functions or intracellular levels of the protein complex or a constituent protein, e.g., by triggering or initiating, enhancing or stabilizing protein-protein interaction between the interacting protein members of the protein complex, or the mutant forms of such interacting protein members found in the patient.

For example, tankyrase's role in diabetes and Alzheimer's disease may be modulated by compounds of the formula:

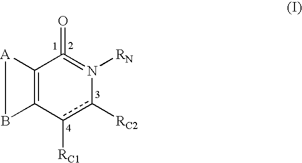

wherein A and B together represent an optionally substituted, fused aromatic ring, the dotted line between the 3 and 4 positions indicates the optional presence of a double bond, at least one of R_{C1} and R_{C2} is independently represented by -L-R_{L}, and if one of R_{C1} and R_{C2} is not represented by -L-R_{L}, then that group is H, where L is of formula: —(CH₂)_{n1-Qn2}-(CH₂)ₙ₃— wherein n1, n2 and n3 are each selected from 0, 1, 2 and 3, the sum of n₁, n₂ and n₃ is 1, 2 or 3 and each Q (if n₂ is greater than 1) is selected from O, S, NR₃, C(═O), or —CR₁R₂—, where R₁ and R₂ are independently selected from hydrogen, halogen or optionally substituted C₁₋₇ alkyl, or may together with the carbon atom to which they are attached form a C₃₋₇ cyclic alkyl group, which may be saturated (a C₃₋₇ cycloalkyl group) or unsaturated (a C₃₋₇ cycloalkenyl group), or one of R₁ and R₂ may be attached to an atom in R_{L} to form an unsaturated C₃₋₇ cycloalkenyl group which comprises the carbon atoms to which R₁ and R₂ are attached in Q, —(CH₂),₃— (if present) and part of R₁, and where R₃ is selected from H or C₁₋₇ alkyl, and R_{L} is selected from optionally substituted C₃₋₂₀ heterocyclyl, C₅₋₂₀ aryl and carbonyl, and RN is selected from hydrogen, optionally substituted C₁₋₇ alkyl, C₃₋₂₀ heterocyclyl, C₅₋₂₀ aryl, hydroxyl, ether, nitro, amino, thioether, sulfoxide and sulfone.

Tankyrase's role in diabetes and Alzheimer's disease may also be modulated by compounds of the formula:

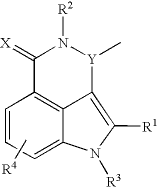

wherein:

R¹ is H, halogen, cyano, an optionally substituted alkyl, alkenyl, alkynyl, cycloalkyl, heterocycloalkyl, aryl, or heteroaryl group, or —C(O)—R₁₀, where R₁₀ is: H, an optionally substituted alkyl, alkenyl, alkynyl, cycloalkyl, heterocycloalkyl, aryl, or heteroaryl group; or OR₁₀₀ or NR₁₀₀R₁₁₀ where R₁₀₀and R₁₁₀ are each independentlyH or an optionally substituted alkyl, alkenyl, alkynyl, cycloalkyl, heterocycloalkyl, aryl, or heteroaryl group;

R² is H or alkyl;

R³ is H or alkyl;

R⁴ is H, halogen or alkyl;

X is O or S;

Y is (CR₅R₆)(CR₇R₈)ₙ or N═C(R₅), where: n is 0 or 1; R₅ and R₆ are each independently H or an optionally substituted alkyl, alkenyl, alkynyl, cycloalkyl, heterocycloalkyl, aryl, or heteroaryl group; and R₇ and R₈ are each independently H or an optionally substituted alkyl, alkenyl, aklynyl, cycloalkyl, heterocycloalkyl, aryl, or heteroaryl group; where when R¹, R⁴, R₅, R₆, and R₇ are each H, and R₈ is not unsubstituted phenyl.

IRAP's role in diabetes and Alzheimer's disease may be modulated by LVV-hemorphin-7 and angiotensin IV. Beta-Spectrins role in diabetes and Alzheimer's disease may be modulated by dibucaine and glutaminyl-tRNA synthetase's role in diabetes and Alzheimer's disease may be modulated by glutaminol adenylate 5. EGR1's role in diabetes and Alzheimer's disease may be modulated by echinomycin, nogalamycin, hedamycin, and chromomycin A3. The function of alpha-glucosidase (GAA) in diabetes and Alzheimer's disease may be modulated by 1-deoxynojirimycin (DNJ), alpha-homonojirimycin, and/or castanospermine. The function of myosin in diabetes and Alzheimer's disease may be modulated by mesitylcarboxylic acid or its derivatives.

The role of AKT1 and AKT2 in diabetes and Alzheimer's disease may be modulated by compounds of the formula:

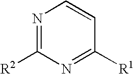

wherein,

each R¹ and R² is independently R₃; R₈, NHR₃, NHR₅, NHR₅, NHR₆, NR₅R₅, NR₅R₆, SR₅, SR₆, OR₅, OR₆, C(O)R₃, heterocyclyl optionally substituted with 1-4 independent P4 on each ring; or C₁₋₁₀ alkyl substituted with 1-4 independent R₄;

each R₃ is independently aryl, phenyl optionally substituted with 1-4 independent R₄, or heteroaryl optionally substituted with 1-4 independent R₄ on each ring;

each m is independently 0, 1, 2, or 3;

each n is independently 1 or 2;

each X is O or S;

each R₄ is independently selected form H, C₁₋₁₀ alkyl, C₂-C₁₀ alkenyl, C2-C₁₀ alkynyl, C_{3-C10} cycloalkyl, C₄₋₁₀ cycloalkenyl, aryl, halo, haloalkyl, CF₃, SR₅, OR₅, OC(O)R₅, NR₅R₅, NR₅R₅, NR₅R₆, COOR₅, NO₂, CN, C(O)R₅, C(O)C(O)R₅, C(O)NR₅R₅, S(O)ₙR₅, S(O)ₙNR₅R₅, C_{1-C10} alkyl substituted with 1-3 independent aryl, C₂₋₁₀ alkenyl substituted with 1-3 independent aryl;

each R₅ is independently H, C₁₋₁₀ alkyl, C₂₋₁₀ alkenyl, C₂₋₁₀ alkynyl, C₃₋₁₀ cycloalkyl, C₄₋₁₀ cycloalkenyl, aryl, haloalkyl, C₁₋₁₀ alkyl substituted with 1-3 independent aryl, C3-10 cycloalkyl substituted with 1-3 indepenet aryl or C₂₋₁ alkenyl substituted with 1-3 independent aryl;

each R₆ is independently C(O)R₅, COOR₅, C(O)NR₅R₅, or S(O)ₙR₅.

PTP1B's role in diabetes and Alzheimer's disease may be modulated by compounds of the formula:

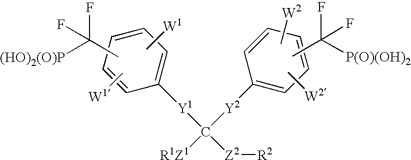

wherein:

R¹ and R² are selected from the group consisting of: C₁₋₁₀alkyl(R⁸)₀₋₇, C₂₋₁₀alkenyl(R¹)₀₋₇, aryl(R¹)₀₋₃ and het(R^{a})_{0-3;}

Each R¹ independently represents a member selected from the group consisting of: aryl, OH, halogen, CN, CO₂H, CO₂C₁₋₆alkyl, CO₂C₂₋₆alkenyl, OC₁₋₁₀alkyl, C(O)C₁₋₆alkyl, C₁₋₆alkyl, C₁₋₆haloalkyl, S(O)_{y}NR³′R⁴′, wherein y is 0, 1, or 2, C(O)NR³′R⁴′, NR3′R4′, and het, wherein each alkyl group in R¹ is optionally substituted with 1-7 groups independently selected from halogen, OC₁₋₃ alkyl, CO₂H, and CO₂C₁₋₃alkyl, and each het and aryl in R¹ is optionally substituted with 1-3 groups independently selected from C₁₋₃alkyl, halogen, OC₁₋₃ alkyl, CO₂H, and CO₂C₁₋₃alkyl;

Aryl is a 6-14 membered carbocyclic aromatic ring system comprising 1-3 phenyl rings, wherein the phenyl rings are fused together when there is more than one aromatic ring;

Het represents a 5-10 membered aromatic ring system comprising one ring or two fused rings, 1-4 heteratoms, 0-4 of which are N atoms and 0-2 of which are O or S(O)_{y} wherein y is 0-2, and 0-2 carboynyl groups;

Y¹, Y², Z¹, and Z² each independently represents —(CR³R⁴)ₐ-X—(CR³R⁴)_{b}— wherein a and b are integers 0-2 such that the sum of a and b equals 0, 1, 2, or 3;

X represents a bond, O, S(O)_{y}, NR³′, C(O), OC(O), C(O)O, C(O)NR³′, NR³C(O) or —CH═CH—, where y is as previously defined;

R³ and R⁴ are independently H, halogen, C₁₋₁₀alkyl or C₁₋₁₀haloalkyl;

R³′ and R⁴′ are each independently selected form the group consisting of: H, C₁₋₆alkyl, C₁₋₆haloalky, OH, C(O)C₁₋₆alkyl, C(O)aryl, C(O)het, C(O)C₁₋₆haloalkyl, aryl and het;

W1 and W2 are each in a position on the aromatic ring adjacent to the —CF2P(O)(OH)₂ substituent and are each independently selected from the group consisting of: OH, CN, halo, OC6alkyl(R^{a})₀₋₇, S(O)_{y}C₁₋₆alkyl(R¹)₀₋₇, with y equal to 0-2, S(O)CH, C₁₋₆alkyl(R¹)₀₋₇, CO₂H, CO₂C₁₋₆alkyl(R^{a})₀₋₇, CO₂C₂₋₆alkenyl(R¹)₀₋₇, C(O)C₁₋₆alkyl(R¹)₀₋₇, C(O)NR³′R⁴′, S(O)_{y}NR³′R⁴′, NR³′R⁴′, aryl, and het, wherein R³′ and R⁴′ are as defined above; and

W¹′ and W²′ are optionally present on any remaining position on the aromatic ring and are each independently selected form H and from the same groups as W¹ and W².

The role of HSP90 and HSP89 in diabetes and Alzheimer's disease may be modulated by geldanamycin, benzoquinon ansamycin, radicicol, novobiocin, 17-allylamino-17-demethoxygeldanamycin, herbimycin A and compounds of the formula:

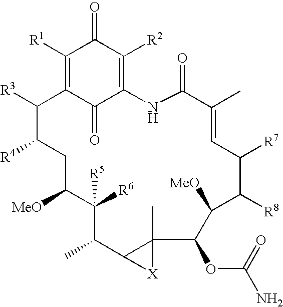

wherein R¹ is MeO, (CH₂)₃N or R⁹—NH, wherein R⁹ is selected from the group consisting of H, substituted or unsubstituted C₁₋₆alkyl, substituted or unsubstituted C1-6alkenyl, substituted or unsubstituted C₁₋₆alkynyl, substituted or unsubstituted C₃₋₆cycloalkyl, piperidinyl, N-alkylpiperidinyl, hexahydropyranyl, furfuryl, tetrahydrofurfuryl, pyrrolidinyl, N-alkylpyrrolidinyl, piperazinylamino, N-alkylpiperazinyl, morpholinyl, N-alkylaziridinylmethyl, (1-azabicyclo[1.3.0]hex-1-yl)ethyl, 2-(N-methyl-pyrrolidin-2-yl)ethyl, 2-(4-imidazolyl)ethyl, 2-(1-methyl-4-imidazolyl-)ethyl, 2-(1-methyl-5-imidazolyl)ethyl, 2-(4-pyridyl)ethyl, and 3-(4-morpholino)-1-propyl, or R⁶ is H and R′ and R⁵ taken together form a group of the formula NH-Z-O, wherein Z is a linker comprised of from 1-6 carbon atoms and 0-2 nitrogen atoms and wherein the 0 is attached at the position of R⁵;

R² is selected form the group consisting of H, halogen, OR¹⁰, NHR¹⁰, SR¹⁰, aryl, and heteroaryl, wherein R¹⁰ is selected from the group consisting of substituted or unsubstituted C₁₋₆alkyl, substituted or unsubstituted C₁₋₆alkenyl, substituted or unsubstituted C₁₋₆alkynyl, and substituted or unsubstituted C₃₋₆cycloalkyl;

R³ is H, OH or OMe;

R⁴ is H or Me;

R⁵ is OH or O—C(═O)—CH₂NH₂ and R⁶ is H, or R⁵ and R⁶ taken together form ═O or ═N—OR¹¹, wherein R¹¹ is selected from the group consisting of H, substituted or unsubstituted C₁₋₆alkyl, substituted or unsubstituted C₁₋₆ alkenyl, substituted or unsubstituted C₁₋₆alkynyl, substituted or unsubstituted C₃₋₆ cycloalkyl, aryl, or heteroaryl;

R⁷ is H and R8 is H or OH, or R⁷ and R⁸ taken together form a bond, and X is 0 or a bond, with the provisos that when R³ is H, R⁴ is Me, and R⁷ is H and R⁸ is H or R⁷ and R⁸ taken together form a bond that either R⁶ is H and R′ and R⁵ taken together form a group of the formula NH-Z-O, wherein Z is a linker comprised of from 1-6 carbon atoms and 0-2 nitrogen atoms and wherein the 0 is attached at the position of R⁵, or that R¹ is (CH₂)₃N or R⁹—NH, wherein R⁹ is selected from the group consisting of piperidinyl, N-alkylpiperidinyl, hexahydropyranyl, furfuryl, tetrahydrofurfuryl, pyrrolidinyl, N-alkylpyrrolidinyl, piperazinylamino, N-alkylpiperazinyl, morpholinyl, N-alkylaziridinylmethyl, (1-azabicyclo[1.3.0]hex-1-yl)ethyl, 2-(N-methyl-pyrrolidin-2-yl)ethyl, 2-(4-imidazolyl)ethyl, 2-(1-methyl-4-imidazolyl-)ethyl, 2-(1-methyl-5-imidazolyl)ethyl, 2-(4-pyridyl)ethyl, and 3-(4-morpholino)-1-propyl, and that when R³ is H and R⁴ is Me that R⁷ is H and R⁸ is OH.

Beta-catenin's role in diabetes and Alzheimer's disease may be modulated by compounds of the formula:

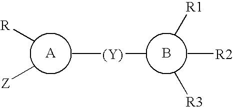

wherein:

(A) is a saturated, partially saturated, carbocyclic or heteroaromatic pentatomic ring;

(B) is a saturated, partially saturated, carbocyclic, aromatic or internally condensed ring;

(Y) is a spacer consisting of about 4-9 chain atoms chosen independently from C, O, N, and S, wherein 2-5 adjacent atoms of the chain may be part of an optionally substituted aryl, heteroaryl, or partially saturated aryl or heteroaryl ring system, which may be either isolated or may include ring (B);

Z is a substituent selected independently from hydrogen, halogen, hydroxyl, cyano, a branched or unbranched C₁₋₄ alkyl group optionally substituted by 1-3 halogen atoms, a branched or unbranched C₁₋₄ alkoxy group, a N(RₐR_{b}) group wherein each of Rₐ and R_{b} independently is selected from hydrogen and C₁₋₄ alkyl, and a NHCOR_{c} or NHSO₂R_{c} group, wherein R_{c} is C₁₋₄ alkyl;

R is independently selected from hydrogen, halogen, cyano, a branched or unbranched C₁₋₄ alkyl group optionally substituted by 1-3 halogen atoms, a branched or unbranched C₁₋₄ alkoxy group, a N(RₐR_{b}) group wherein each of Rₐ and R_{b} independently is selected from hydrogen and C₁₋₄ alkyl, and a NHCOR_{c} or NHSO₂R_{c} group, wherein R_{c} is C₁₋₄ alkyl, or Z and R taken together form an optionally substituted, partially saturated monocyclic or bicyclic ring system;

each of R1, R2, and R3 is independently hydrogen, halogen, cyano, a branched or unbranched C₁₋₄ alkyl group optionally substituted by 1-3 halogen atoms, a branched or unbranched C₁₋₄ alkoxy group, a N(RₐR_{b}) group wherein each of Rₐ and R_{b} independently is selected form hydrogen and C₁₋₄ alkyl, a NHCOR_{c} or NHSO₂R_{c} group wherein R_{c} is C₁₋₄ alkyl, and a C₅₋₆ cycloalkyl-oxy or aryloxy group.

For example, Beta-Catenin's role in diabetes and Alzheimer's disease may be modulated by a compound of the formula:

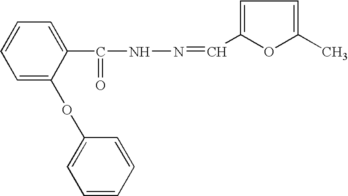

APP's role in diabetes and Alzheimer's disease may be modulated by several compounds. For example, anticholinesterases such as tacrine and phenserine are able to modulate APP's function by reducing the levels of APP. Levels of APP may also be reduced by compounds of formulas I or II:

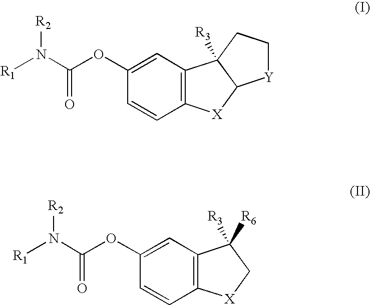

wherein:

R₁ and R₂ are independently hydrogen, branched or unbranched C₁₋₈ alkyl, substituted or unsubstituted aryl, or aralkyl;

R₃ is branched or unbranched C₁₋₄ alkyl or heteroalkyl or C₄₋₈ alkyl or heteroakyl, or substituted or unsubstituted aryl;

X and Y are independently O, S, alkyl, hydrocarbon moiety, C(H)R₄, or NR₅, wherein Rv and Rv are independently hydrogen, oxygen, branched or unbranched C₁₋₈ alkyl, C₂₋₈ alkenyl, or C₂₋₈ alkynyl, aralkyl, or substituted or unsubstituted aryl; and

R₆ is hydrogen, C₁₋₈ alkyl, C₂₋₈ alkenyl, C₂₋₈ alkynyl, aralkyl, or substituted or unsubstituted aryl, or (CH₂)ₙR₇, wherein R₇ is hydroxyl, alkoxy, cyano, ester, carboxylic acid, substituted or unsubstituted amino, and n is from 1-4.

The role of complement proteins (including C1 and C4) in diabetes and Alzheimer's disease may be modulated by compounds of the formula:

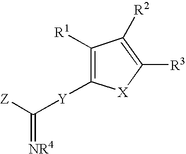

wherein:

X is O, S or NR, where R⁷ is hydrogen, alkyl, aralkyl, hydroxyl(C₂₋₄)alkyl, or alkoxy(C₂₋₄)alkyl;

Y is a direct covalent bond, CH₂ or NH;

Z is NR⁵R⁶, hydrogen or alkyl, provided that Y is NH whenever Z is hydrogen or alkyl;

R¹ is hydrogen, amino, hydroxyl, halogen, cyano, C₁₋₄ alkyl or —CH₂R, where R is hydroxyamino or C₁₋₃ alkoxy;

R² and R³ are independently hydrogen, halogen, hydroxyl, nitro, cyano, amino, monoalkylamino, dialkylamino, monoarylamino, diarylamino, monoalkylmonoarylamino, alkoxycarbonylamino, aralkoxycarbonylamino, aryloxycarbonylamino, alkylsulfonylamino, aralkylsulfonylamino, arylsulfonylamino, formylamino, acylamino, H(S)CNH—, thioacylamino, aminocarbonyl, monoalkylaminocarbonyl, dialkylaminocarbonyl, acyl, aminoacyl, arylaminocarbonyl, aminothiocarbonyl, monoalkylaminothiocarbonyl, diakylaminothiocarbonyl, thioacyl, aminothioacyl, aminocarboynylamino, monoalkylaminothiocarbonyl, dialkylaminothiocarbonyl, thioacyl, aminothioacyl, aminocarbonylamino, mono- and dialkylaminocarbonylamino, mono- and diarylaminocarbonylamino, or mono- and diaralkylaminocarbonylamino, aminocarbonyloxy, mono- and dialkylaminocarbonyloxy, mono- and diarylaminocarbonyloxy, mono and diaralkylaminocarbonyloxy, aminosulfonyl, mon- and dialkylaminosulfonyl, mono- and diarylaminosulfonyl, mono- and diaralkylaminosulfonyl, alkoxy, or alkylthio, wherein the alkyl portion of each group may be optionally substituted, aralkoxy, aryloxy, aralkylthio, arylthio, wherein the aryl portion of each group can be optionally substituted, alkylsulfonyl, wherein the alkyl portion can be optionally substituted, aralkylsulfonyl, or arylsulfonyl, wherein the aryl portion of each group can be optionally substituted, alkenyl, alkynyl, or optionally substituted aryl, alkyl, aralkyl, heterocycle, and cycloalkyl;

R⁴, R⁵, and R⁶ are independently hydrogen, C₁₋₄ alkyl, aryl, hydroxyalkyl, aminoalkyl, monoalkylamino(C₂₋₁₀)alkyl, dialkylamino(C₂₋₁₀)alkyl, carboxyalkyl, cyano, amino, alkoxy, or hydroxyl, or —CO₂R^{w}, where R^{W} is alkyl, cycloalkyl, phenyl, benzyl,

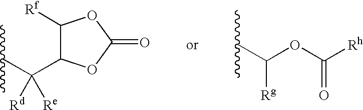

where R^{d} and R^{e} are independently hydrogen, C₁₋₆ alkyl, C₂₋₆ alkenyl or phenyl, R^{f} is hydrogen, C₁₋₆ alkyl, C₂₋₆ alkenyl or phenyl, R^{g} is hydrogen, C₁₋₆ alkyl, C₂₋₆ alkenyl or phenyl, and R^{h} is aralkyl or C₁₋₆ alkyl.

The role of alpha-karyopherin and/or the HIV preintegration complex in diabetes and Alzheimer's disease may be modulated by compounds of the formula:

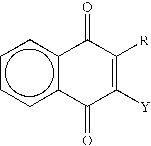

wherein R is —X—CO-Z, wherein Z is branched or unbranched C₁₋₆ alkoxy, wherein X is (CH₂), or —S—(CH₂)ₙ, wherein n is an integer form 0-6, and wherein Y is H 10 or branched or unbranched C₁₆.

### 7. Cell and Animal Models

In another aspect of the present invention, cell and animal models are provided in which one or more of the interacting pairs of proteins described in the tables, or the constituent proteins of the interacting pairs, exhibit aberrant function, activity, or concentration when compared with wild type cells and animals (e.g., increased or decreased concentration, altered interactions between protein complex constituents due to mutations in interaction domains, and/or altered distribution or localization of the protein complexes or constituents thereof in organs, tissues, cells, or cellular compartments). Such cell and animal models are useful tools for studying cellular functions and biological processes associated with the protein complexes of the present invention, or with the constituent proteins identified in the tables. Such cell and animal models are also useful tools for studying disorders and diseases associated with the protein complexes of the present invention, or the constituent proteins of the protein complexes themselves, and for testing various methods for modulating the cellular functions, and for treating the diseases and disorders, associated with aberrations in these protein complexes or the protein constituents thereof.

### 7.1. Cell Models

Cell models having an aberrant form of one or more of the protein complexes of the present invention are provided in accordance with the present invention.

The cell models may be established by isolating, from a patient, cells having an aberrant form of one or more of the protein complexes of the present invention. The isolated cells may be cultured in vitro as a primary cell culture. Alternatively, the cells obtained from the primary cell culture or directly from the patient may be immortalized to establish a human cell line. Any methods for constructing immortalized human cell lines may be used in this respect. See generally Yeager and Reddel, Curr. Opini. Biotech., 10:465-469 (1999). For example, the human cells may be immortalized by transfection of plasmids expressing the SV40 early region genes (See e.g., Jha et al., Exp. Cell Res., 245:1-7 (1998)), introduction of the HPV E6 and E7 oncogenes (See e.g., Reznikoff et al., Genes Dev., 8:2227-2240 (1994)), and infection with Epstein-Barr virus (See e.g., Tahara et al., Oncogene, 15:1911-1920 (1997)). Alternatively, the human cells may be immortalized by recombinantly expressing the gene for the human telomerase catalytic subunit hTERT in the human cells. See Bodnar et al., Science, 279:349-352 (1998).

In alternative embodiments, cell models are provided by recombinantly manipulating appropriate host cells. The host cells may be bacteria cells, yeast cells, insect cells, plant cells, animal cells, and the like. Preferably, the cells are derived from mammals, most preferably humans. The host cells may be obtained directly from an individual, or a primary cell culture, or preferably an immortal stable human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998) and Thomson et al., Science, 282:1145-1147 (1998).

In one embodiment, a cell model is provided by recombinantly expressing one or more of the protein complexes of the present invention in cells that do not normally express such protein complexes. For example, cells that do not contain a particular protein complex may be engineered to express the protein complex. In a specific embodiment, a particular human protein complex is expressed in non-human cells. The cell model may be prepared by introducing into host cells nucleic acids encoding all interacting protein members required for the formation of a particular protein complex, and expressing the protein members in the host cells. For this purpose, the recombinant expression methods described in Section 2.2 may be used. In addition, the methods for introducing nucleic acids into host cells disclosed in the context of gene therapy in Section 6.3.2 may also be used.

In another embodiment, a cell model over-expressing one or more of the protein complexes of the present invention is provided. The cell model may be established by increasing the expression level of one or more of the interacting protein members of the protein complexes. In a specific embodiment, all interacting protein members of a particular protein complex are over-expressed. The over-expression may be achieved by introducing into host cells exogenous nucleic acids encoding the proteins to be over-expressed, and selecting those cells that over-express the proteins. The expression of the exogenous nucleic acids may be transient or, preferably stable. The recombinant expression methods described in Section 2.2, and the methods for introducing nucleic acids into host cells disclosed in the context of gene therapy in Section 6.3.2 may be used. Alternatively, the gene activation method disclosed in U.S. Pat. No. 5,641,670 can be used. Any host cells may be employed for establishing the cell model. Preferably, human cells lacking a protein complex to be over-expressed, or having a normal concentration of the protein complex, are used as host cells. The host cells may be obtained directly from an individual, or a primary cell culture, or preferably a stable immortal human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998), and Thomson et al., Science, 282:1145-1147 (1998).

In yet another embodiment, a cell model expressing an abnormally low level of one or more of the protein complexes of the present invention is provided. Typically, the cell model is established by genetically manipulating cells that express a normal and detectable level of a protein complex identified in accordance with the present invention. Generally the expression level of one or more of the interacting protein members of the protein complex is reduced by recombinant methods. In a specific embodiment, the expression of all interacting protein members of a particular protein complex is reduced. The reduced expression may be achieved by “knocking out” the genes encoding one or more interacting protein members. Alternatively, mutations that can cause reduced expression level (e.g., reduced transcription and/or translation efficiency, and decreased mRNA stability) may also be introduced into the gene by homologous recombination. A gene encoding a ribozyme or antisense compound specific to the mRNA encoding an interacting protein member may also be introduced into the host cells, preferably stably integrated into the genome of the host cells. In addition, a gene encoding an antibody or fragment thereof specific to an interacting protein member may also be introduced into the host cells. The recombinant expression methods described in Sections 2.2, 6.1 and 6.2 can all be used for purposes of manipulating the host cells.

The present invention also contemplates a cell model provided by recombinant DNA techniques that exhibits aberrant interactions between the interacting protein members of a protein complex identified in the present invention. For example, variants of the interacting protein members of a particular protein complex exhibiting altered protein-protein interaction properties and the nucleic acid variants encoding such variant proteins may be obtained by random or site-directed mutagenesis in combination with a protein-protein interaction assay system, particularly the yeast two-hybrid system described in Section 5.3.1. Essentially, the genes encoding one or more interacting protein members of a particular protein complex may be subject to random or site-specific mutagenesis and the mutated gene sequences are used in yeast two-hybrid system to test the protein-protein interaction characteristics of the protein variants encoded by the gene variants. In this manner, variants of the interacting protein members of the protein complex may be identified that exhibit altered protein-protein interaction properties in forming the protein complex, e.g., increased or decreased binding affinity, and the like. The nucleic acid variants encoding such protein variants may be introduced into host cells by the methods described above, preferably into host cells that normally do not express the interacting proteins.

### 7.2. Cell-Based Assays

The cell models of the present invention containing an aberrant form of a protein complex of the present invention are useful in screening assays for identifying compounds useful in treating diseases and disorders involving cellular glucose transport such as diabestes, obesity, Alzheimer's disease and neurodegenerative disorders. In addition, they may also be used in in vitro pre-clinical assays for testing compounds, such as those identified in the screening assays of the present invention.

For example, cells may be treated with compounds to be tested and assayed for the compound's activity. A variety of parameters relevant to particularly physiological disorders or diseases may be analyzed.

### 7.3. Transgenic Animals

In another aspect of the present invention, transgenic non-human animals are created expressing an aberrant form of one or more of the protein complexes of the present invention. Animals of any species may be used to generate the transgenic animal models, including but not limited to, mice, rats, hamsters, sheep, pigs, rabbits, guinea pigs, preferably non-human primates such as monkeys, chimpanzees, baboons, and the like.

In one embodiment, transgenic animals are made to over-express one or more protein complexes formed from a first protein, which is any one of the proteins described in the tables, or a derivative, fragment or homologue thereof (including the animal counterpart of the first protein, i.e., an orthologue) and a second protein, which is any of the proteins described in the tables that interacts with the first protein, or derivatives, fragments or homologues thereof (including orthologues). Over-expression may be directed in a tissue or cell type that normally expresses animal counterparts of such protein complexes. Consequently, the concentration of the protein complex(es) will be elevated to higher levels than normal. Alternatively, the one or more protein complexes are expressed in tissues or cells that do not normally express such proteins and hence do not normally contain the protein complexes of the present invention. In a specific embodiment, a first protein, which is any one of the proteins described in the tables which is a human protein and a second protein, which is any of the proteins described in the tables that interacts with the first protein and is a human protein, are expressed in the transgenic animals.

To achieve over-expression in transgenic animals, the transgenic animals are made such that they contain and express exogenous, orthologous genes encoding a first protein, which is any of the proteins identified in the tables or a homologue, derivative or mutant form thereof, and one or more second proteins, which are any of the proteins described in the tables that interact with the first protein, or homologues, derivatives or mutant forms thereof. Preferably, the exogenous genes are human genes. Such exogenous genes may be operably linked to a native or non-native promoter, preferably a non-native promoter. For example, an exogenous gene encoding one of the proteins described in the tables may be operably linked to a promoter that is not the native promoter of that protein. If the expression of the exogenous gene is desired to be limited to a particular tissue, an appropriate tissue-specific promoter may be used.

Over-expression may also be achieved by manipulating the native promoter to create mutations that lead to gene over-expression, or by a gene activation method such as that disclosed in U.S. Pat. No. 5,641,670 as described above.

In another embodiment, the transgenic animal expresses an abnormally low concentration of the complex comprising at least one of the interacting pairs of proteins described in the tables. In a specific embodiment, the transgenic animal is a “knockout” animal wherein the endogenous gene encoding the animal orthologue of a first protein, which is any of the proteins described in the tables, and/or an endogenous gene encoding an animal orthologue of a second protein, which is any of the proteins identified in the tables that interacts with the first protein, are knocked out. In a specific embodiment, the expression of the animal orthologues of both the first and second proteins are reduced or knocked out. The reduced expression may be achieved by knocking out the genes encoding one or both interacting protein members, typically by homologous recombination. Alternatively, mutations that can cause reduced expression (e.g., reduced transcription and/or translation efficiency, or decreased mRNA stability) may also be introduced into the endogenous genes by homologous recombination. Genes encoding ribozymes or antisense compounds specific to the mRNAs encoding the interacting protein members may also be introduced into the transgenic animal. In addition, genes encoding antibodies or fragments thereof specific to the interacting protein members may also be introduced into the transgenic animal.

In an alternate embodiment, transgenic animals are made in which the endogenous genes encoding the animal orthologues of any of the proteins described in the tables are replaced with orthologous human genes.

In yet another embodiment, the transgenic animal of this invention expresses specific mutant forms of any of the proteins described in the tables that exhibit aberrant interactions. For this purpose, variants of any of the proteins described in the tables exhibiting altered protein-protein interaction properties, and the nucleic acid variants encoding such variant proteins, may be obtained by random or site-specific mutagenesis in combination with a protein-protein interaction assay system, particularly the yeast two-hybrid system described in Section 5.3.1. For example, variants of GLUT4 and PN7065 exhibiting increased, decreased or abolished binding affinity to each other may be identified and isolated. The transgenic animal of the present invention may be made to express such protein variants by modifying the endogenous genes. Alternatively, the nucleic acid variants may be introduced exogenously into the transgenic animal genome to express the protein variants therein. In a specific embodiment, the exogenous nucleic acid variants are derived from orthologous human genes and the corresponding endogenous genes are knocked out.

Any techniques known in the art for making transgenic animals may be used for purposes of the present invention. For example, the transgenic animals of the present invention may be provided by methods described in, e.g., Jaenisch, Science, 240:1468-1474 (1988); Capecchi, et al., Science, 244:1288-1291 (1989); Hasty et al., Nature, 350:243 (1991); Shinkai et al., Cell, 68:855 (1992); Mombaerts et al., Cell, 68:869 (1992); Philpott et al., Science, 256:1448 (1992); Snouwaert et al., Science, 257:1083 (1992); Donehower et al., Nature, 356:215 (1992); Hogan et al., Manipulating the Mouse Embryo; A Laboratory Manual, 2 edition, Cold Spring Harbor Laboratory Press, 1994; and U.S. Pat. Nos. 4,873,191; 5,800,998; 5,891,628, all of which are incorporated herein by reference.

Generally, the founder lines may be established by introducing appropriate exogenous nucleic acids into, or modifying an endogenous gene in, germ lines, embryonic stem cells, embryos, or sperm which are then used in producing a transgenic animal. The gene introduction may be conducted by various methods including those described in Sections 2.2, 6.1 and 6.2. See also, Van der Putten et al., Proc. Natl. Acad. Sci. USA, 82:6148-6152 (1985); Thompson et al., Cell, 56:313-321 (1989); Lo, Mol. Cell. Biol., 3:1803-1814 (1983); Gordon, Trangenic Animals, Intl. Rev. Cytol. 115:171-229 (1989); and Lavitrano et al., Cell, 57:717-723 (1989). In a specific embodiment, the exogenous gene is incorporated into an appropriate vector, such as those described in Sections 2.2 and 6.2, and is transformed into embryonic stem (ES) cells. The transformed ES cells are then injected into a blastocyst. The blastocyst with the transformed ES cells is then implanted into a surrogate mother animal. In this manner, a chimeric founder line animal containing the exogenous nucleic acid (transgene) may be produced.

Preferably, site-specific recombination is employed to integrate the exogenous gene into a specific predetermined site in the animal genome, or to replace an endogenous gene or a portion thereof with the exogenous sequence. Various site-specific recombination systems may be used including those disclosed in Sauer, Curr. Opin. Biotechnol., 5:521-527 (1994); Capecchi, et al., Science, 244:1288-1291 (1989); and Gu et al., Science, 265:103-106 (1994). Specifically, the Cre/lox site-specific recombination system known in the art may be conveniently used which employs the bacteriophage PI protein Cre recombinase and its recognition sequence loxP. See Rajewsky et al., J. Clin. Invest., 98:600-603 (1996); Sauer, Methods, 14:381-392 (1998); Gu et al., Cell, 73:1155-1164 (1993); Araki et al., Proc. Natl. Acad. Sci. USA, 92:160-164 (1995); Lakso et al., Proc. Natl. Acad. Sci. USA, 89:6232-6236 (1992); and Orban et al., Proc. Natl. Acad. Sci. USA, 89:6861-6865 (1992).

The transgenic animals of the present invention may be transgenic animals that carry a transgene in all cells or mosaic transgenic animals carrying a transgene only in certain cells, e.g., somatic cells. The transgenic animals may have a single copy or multiple copies of a particular transgene.

The founder transgenic animals thus produced may be bred to produce various offsprings. For example, they can be inbred, outbred, and crossbred to establish homozygous lines, heterozygous lines, and compound homozygous or heterozygous lines.

### 8. Pharmaceutical Compositions and Formulations

In another aspect of the present invention, pharmaceutical compositions are also provided containing one or more of the therapeutic agents provided in the present invention as described in Section 6. The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

For example, such therapeutic agents include, but are not limited to, (1) small organic compounds selected based on the screening methods of the present invention capable of interfering with the interaction between a first protein which is any of the interacting proteins described in the tables and a second protein which is any of the proteins identified in the tables that interacts with the first protein, (2) antisense compounds specifically hybridizable to nucleic acids (gene or mRNA) encoding the first protein (3) antisense compounds specific to the gene or mRNA encoding the second protein, (4) ribozyme compounds specific to nucleic acids (gene or mRNA) encoding the first protein, (5) ribozyme compounds specific to the gene or mRNA encoding the second protein, (6) antibodies immunoreactive with the first protein or the second protein, (7) antibodies selectively immunoreactive with a protein complex of the present invention, (8) small organic compounds capable of binding a protein complex of the present invention, (9) small peptide compounds as described above (optionally linked to a transporter) capable of interacting with the first protein or the second protein, (10) nucleic acids encoding the antibodies or peptides, (11) siRNA compounds specific to the gene or mRNA encoding the first protein, (12) siRNA compounds specific to the gene or mRNA encoding the second protein, etc.

The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

In the pharmaceutical composition, an active compound identified in accordance with the present invention can be in any pharmaceutically acceptable salt form. As used herein, the term “pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naththylate salts, mesylate salts, tosylate salts, citrate salts, lactate salts, maleate salts, succinate salts, tartrate salts, fumarate salts, and the like. See, e.g., Berge, et al., J. Pharm. Sci., 66:1-19 (1977).

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels is biodegradable or biosorbable. For purposes of this invention, hydrogels made of polyethylene glycols, collagen, or poly(glycolic-co-L-lactic acid) may be useful. See, e.g., Phillips et al., J Pharmaceut. Sci. 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Generally, the toxicity profile and therapeutic efficacy of the therapeutic agents can be determined by standard pharmaceutical procedures in cell models or animal models, e.g., those provided in Section 7. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

It will be apparent to skilled artisans that therapeutically effective amount for each active compound to be included in a pharmaceutical composition of the present invention can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can also be adjusted as the various factors change over time.

### EXAMPLES

1. Yeast Two-Hybrid System

The principles and methods of the yeast two-hybrid system have been described in detail in The Yeast Two-Hybrid System, Bartel and Fields, eds., pages 183-196, Oxford University Press, New York, N.Y., 1997. The following is thus a description of the particular procedure that we used to identify the interactions of the present invention.

The cDNA encoding the bait protein was generated by PCR from cDNA prepared from a desired tissue. The cDNA product was then introduced by recombination into the yeast expression vector pGBT.Q, which is a close derivative of pGBT.C (See Bartel et al., Nat Genet., 12:72-77 (1996)) in which the polylinker site has been modified to include Ml 3 sequencing sites. The new construct was selected directly in the yeast strain PNY200 for its ability to drive tryptophane synthesis (genotype of this strain: MATα trp1-901 leu2-3,112 ura3-52 his3-200 ade2 gal4Δ gal80). In these yeast cells, the bait was produced as a C-terminal fusion protein with the DNA binding domain of the transcription factor Ga14 (amino acids 1 to 147). Prey libraries were transformed into the yeast strain BK100 (genotype of this strain: MATα trp1-901 leu2-3,112 ura3-52 his3-200 gal4Δ gal80 LYS2::GAL-HIS3 GAL2-ADE2 met2::GAL7-lacZ), and selected for the ability to drive leucine synthesis. In these yeast cells, each cDNA was expressed as a fusion protein with the transcription activation domain of the transcription factor Ga14 (amino acids 768 to 881) and a 9 amino acid hemagglutinin epitope tag. PNY200 cells (MATα mating type), expressing the bait, were then mated with BK100 cells (MATa mating type), expressing prey proteins from a prey library. The resulting diploid yeast cells expressing proteins interacting with the bait protein were selected for the ability to synthesize tryptophan, leucine, histidine, and adenine. DNA was prepared from each clone, transformed by electroporation into E. coli strain KC8 (Clontech KC8 electrocompetent cells, Catalog No. C2023-1), and the cells were selected on ampicillin-containing plates in the absence of either tryptophane (selection for the bait plasmid) or leucine (selection for the library plasmid). DNA for both plasmids was prepared and sequenced by the dideoxynucleotide chain termination method. The identity of the bait cDNA insert was confirmed and the cDNA insert from the prey library plasmid was identified using the BLAST program to search against public nucleotide and protein databases. Plasmids from the prey library were then individually transformed into yeast cells together with a plasmid driving the synthesis of lamin and 5 other test proteins, respectively, fused to the Ga14 DNA binding domain. Clones that gave a positive signal in the β-galactosidase assay were considered false-positives and discarded. Plasmids for the remaining clones were transformed into yeast cells together with the original bait plasmid. Clones that gave a positive signal in the β-galactosidase assay were considered true positives.

Bait sequences indicated in the tables were used in the yeast two-hybrid system described above. The isolated prey sequences are summarized in the tables. The GenBank Accession Nos. for the bait and prey proteins are also provided in the tables, upon which the bait and prey sequences are aligned.

2. Production of Antibodies Selectively Immunoreactive with Protein Complex

The GLUT4-interacting region of PN7065 and the PN7065-interacting region of GLUT4 are indicated in Table 1. Both regions, or fragments thereof, are recombinantly-expressed in E. coli. and isolated and purified. Mixing the two purified interacting regions forms a protein complex. A protein complex is also formed by mixing recombinantly expressed intact complete GLUT4 and PN7065. The two protein complexes are used as antigens in immunizing a mouse. mRNA is isolated from the immunized mouse spleen cells, and first-strand cDNA is synthesized using the mRNA as a template. The V_{H} and V_{K} genes are amplified from the thus synthesized cDNAs by PCR using appropriate primers.

The amplified V_{H} and V_{K} genes are ligated together and subcloned into a phagemid vector for the construction of a phage display library. E. coli. cells are transformed with the ligation mixtures, and thus a phage display library is established. Alternatively, the ligated V_{H} and Vₖ genes are subcloned into a vector suitable for ribosome display in which the V_{H}-Vₖ sequence is under the control of a T7 promoter. See Schaffitzel et al., J Immun. Meth., 231:119-135 (1999).

The libraries are screened for their ability to bind GLUT4-PN7065 complex and GLUT4 or PN7065, alone. Several rounds of screening are generally performed. Clones corresponding to scFv fragments that bind the GLUT4-PN7065 complex, but not isolated GLUT4 or PN7065 are selected and purified. A single purified clone is used to prepare an antibody selectively immunoreactive with the complex comprising GLUT4 and PN7065. The antibody is then verified by an immunochemistry method such as RIA and ELISA.

In addition, the clones corresponding to scFv fragments that bind the complex comprising GLUT4 and PN7065, and also bind isolated GLUT4 and/or PN7065 may be selected. The scFv genes in the clones are diversified by mutagenesis methods such as oligonucleotide-directed mutagenesis, error-prone PCR (See Lin-Goerke et al., Biotechniques, 23:409 (1997)), dNTP analogues (See Zaccolo et al., J. Mol. Biol., 255:589 (1996)), and other methods. The diversified clones are further screened in phage display or ribosome display libraries. In this manner, scFv fragments selectively immunoreactive with the complex comprising GLUT4 and PN7065 may be obtained.

3. Yeast Screen To Identify Small Molecule Inhibitors Of The Interaction Between GLUT4 and PN7065

Beta-galactosidase is used as a reporter enzyme to signal the interaction between yeast two-hybrid protein pairs expressed from plasmids in Saccharomyces cerevisiae. Yeast strain MY209 (ade2 his3 leu2 trp1 cyh2 ura3::GAL1p-lacZ gal4 gal80 lys2::GAL1p-HIS3) bearing one plasmid with the genotype of LEU2 CEN4 ARS1 ADH1p-SV40NLS-GAL4 (768-881)-PN7065-PGK1t AmpR ColE1_ori, and another plasmid having a genotype of TRP1 CEN4 ARS ADH1p-GAL4(1-147)-GLUT4-ADH1t AmpR ColE1_ori is cultured in synthetic complete media lacking leucine and tryptophan (SC-Leu-Trp) overnight at 30° C. The GLUT4 and PN7065 nucleic acids in the plasmids can code for the full-length GLUT4 and PN7065 proteins, respectively, or fragments thereof. This culture is diluted to 0.01 OD₆₃₀ units/ml using SC-Leu-Trp media. The diluted MY209 culture is dispensed into 96-well microplates. Compounds from a library of small molecules are added to the microplates; the final concentration of test compounds is approximately 60 μM. The assay plates are incubated at 30° C. overnight.

The following day an aliquot of concentrated substrate/lysis buffer is added to each well and the plates incubated at 37° C. for 1-2 hours. At an appropriate time an aliquot of stop solution is added to each well to halt the beta-galactosidase reaction. For all microplates an absorbance reading is obtained to assay the generation of product from the enzyme substrate. The presence of putative inhibitors of the interaction between GLUT4 and PN7065 results in inhibition of the beta-galactosidase signal generated by MY209. Additional testing eliminates compounds that decreased expression of beta-galactosidase by affecting yeast cell growth and non-specific inhibitors that affected the beta-galactosidase signal generated by the interaction of an unrelated protein pair.

Once a hit, i.e., a compound which inhibits the interaction between the interacting proteins, is obtained, the compound is identified and subjected to further testing wherein the compounds are assayed at several concentrations to determine an IC₅₀ value, this being the concentration of the compound at which the signal seen in the two-hybrid assay described in this Example is 50% of the signal seen in the absence of the inhibitor.

4. Enzyme-Linked Immunosorbent Assay (ELISA)

pGEX5X-2 (Amersham Biosciences; Uppsala, Sweden) is used for the expression of a GST-PN7065 fusion protein. The pGEX5X-2-PN7065 construct is transfected into Escherichia coli strain DH5α (Invitrogen; Carlsbad, Calif.) and fusion protein is prepared by inducing log phase cells (O.D. 595=0.4) with 0.2 mM isopropyl-β-D-thiogalactopyranoside (IPTG). Cultures are harvested after approximately 4 hours of induction, and cells pelleted by centrifugation. Cell pellets are resuspended in lysis buffer (1% nonidet P-40 [NP-40], 150 mM NaCl, 10 mM Tris pH 7.4, 1 mM ABESF [4-(2-aminoethyl) benzenesulfonyl fluoride]), lysed by sonication and the lysate cleared of insoluble materials by centrifugation. Cleared lysate is incubated with Glutathione Sepharose beads (Amersham Biosciences; Uppsala, Sweden) followed by thorough washing with lysis buffer. The GST-PN7065 fusion protein is then eluted from the beads with 5 mM reduced glutathione. Eluted protein is dialyzed against phosphate buffer saline (PBS) to remove the reduced glutathione.

A stable Drosophila Schneider 2 (S2) myc-GLUT4 expression cell line is generated by transfecting S2 cells with pCoHygro (Invitrogen; Carlsbad, Calif.) and an expression vector that directs the expression of the myc-GLUT4 fusion protein. Briefly, S2 cells are washed and re-suspended in serum free Express Five media (Invitrogen; Carlsbad, Calif.). Plasmid/liposome complexes are then added (NovaFECTOR, Venn Nova; Pompano Beach, Fla.) and allowed to incubate with cells for 12 hours under standard growth conditions (room temperature, no CO₂ buffering). Following this incubation period fetal bovine serum is added to a final concentration of 20% and cells are allowed to recover for 24 hours. The media is replaced and cells are grown for an additional 24 hours. Transfected cells are then selected in 350 μg/ml hygromycin for three weeks. Expression of myc-GLUT4 is confirmed by Western blotting. This cell line is referred to as S2-myc-GLUT4.

GST-PN7065 fusion protein is immobilized to wells of an ELISA plate as follows: Nunc Maxisorb 96 well ELISA plates (Nalge Nunc International; Rochester, N.Y.) are incubated with 100 μl of 10 μg/ml of GST-PN7065 in 50 mM carbonate buffer (pH 9.6) and stored overnight at 4° Celsius. This plate is referred to as the ELISA plate.

A compound dilution plate is generated in the following manner. In a 96 well polypropylene plate (Greiner, Germany) 50 μl of DMSO is pipetted into columns 2-12. In the same polypropylene plate pipette, 10 μl of each compound being tested for its ability to modulate protein-protein interactions is plated in the wells of column 1 followed by 90 μl of DMSO (final volume of 100 μl). Compounds selected from primary screens or from virtual screening, or designed based on the primary screen hits are then serially diluted by removing 50 μl from column 1 and transferring it to column 2 (50:50 dilution). Serial dilutions are continued until column 10. This plate is termed the compound dilution plate.

Next, 12 μl from each well of the compound dilution plate is transferred into its corresponding well in a new polypropylene plate. 108 μl of S2-myc-GLUT4-containing lysate (1×10⁶ cell equivalents/ml) in phosphate buffered saline is added to all wells of columns 1-11. 108 μl of phosphate buffered saline without lysate is added into all wells of column 12. The plate is then mixed on a shaker for 15 minutes. This plate is referred to as the compound preincubation plate.

The ELISA plate is emptied of its contents and 400 μl of Superblock (Pierce Endogen; Rockford, Ill.) is added to all the wells and allowed to sit for 1 hour at room temperature. 100 μl from all columns of the compound preincubation plate are transferred into the corresponding wells of the ELISA binding plate. The plate is then covered and allowed to incubate for 1.5 hours room temperature.

The interaction of the myc-tagged GLUT4 with the immobilized GST-PN7065 is detected by washing the ELISA plate followed by an incubation with 100 μl/well of 1 μg/ml of mouse anti-myc IgG (clone 9E10; Roche Applied Science; Indianapolis, Ind.) in phosphate buffered saline. After 1 hour at room temperature, the plates are washed with phosphate buffered saline and incubated with 100 μl/well of 250 ng/ml of goat anti-mouse IgG conjugated to horseradish peroxidase in phosphate buffer saline. Plates are then washed again with phosphate buffered saline and incubated with the fluorescent substrate solution Quantiblu (Pierce Endogen; Rockford, Ill.). Horseradish peroxidase activity is then measured by reading the plates in a fluorescent plate reader (325 nm excitation, 420 nm emission).

5. Effects of Test Compounds on Blood Glucose Levels

Mice with a mutation in the leptin receptor on a C57BLKS background produce db/db mice, which are hyperglycemic, hyperlipidemic, obese, and insulin resistant. Db/db mice are used as a model of Type 2 diabetes and can be used to investigated glucose metabolism. Nine week old male db/db mice and heterozygous db/wt littermates are divided into matched groups with the same average blood glucose levels. Once a week the test groups are treated with intraperional injections of test compounds and the control groups are injected with a saline solution. Db/db mice are treated with doses of 10, 25, or 50 mg/kg while heterozygous db/wt mice are dosed with 50 or 100 mg/kg doses. Dosage occurs for 4 weeks and blood glucose levels being measured at day 0, 7, 14,21, and 28.

6. Effects of Test Compounds on Plasma Insulin Levels

Mice with a mutation in the leptin receptor on a C57BLKS background produce db/db mice, which are hyperglycemic, hyperlipidemic, obese, and insulin resistant. Db/db mice are used as a model of Type 2 diabetes and can be used to investigate glucose metabolism. Nine week old male db/db mice and heterozygous db/wt littermates are divided into matched groups with the same average blood glucose levels. Once a week the test groups are treated with intraperional injections of test compounds and the control groups are injected with a saline solution. Db/db mice are treated with doses of 10, 25, or 50 mg/kg while heterozygous db/wt mice were dosed with 50 or 100 mg/kg doses. Dosage occurs for 4 weeks and plasma insulin levels are measured on day 0, 7, 14, 21, and 28.

7. GLUT4 Translocation Assay

The GLUT4 glucose transporter is expressed predominantly in adipose and muscle tissues, where it accounts for the bulk of insulin-stimulated glucose uptake. In the presence of insulin, GLUT4 is redistributed from an intracellular compartment to the plasma membrane, where it facilitates the diffusion of glucose into the cell. Insulin increases the rate of GLUT4 exocytosis, with little or no decrease in its rate of endocytosis, so that in adipocytes the proportion of GLUT4 at the cell surface increases from <10% in the absence of insulin to 35 to 50% in its presence (see Bogen, et al., Mol. Cell. Biol. 21:4785-4806 (2001), and references therein).

A relevant assay for certain forms of Type II diabetes is to measure changes in the proportion of GLUT4 on the cell surface upon insulin signaling. A recently developed assay that employs undifferentiated 3T3-L1 and CHO cells can be used for this purpose, avoiding the use of difficult-to-manipulate 3T3-L1 adipocytes (Bogen, et al., Mol. Cell. Biol. 21:4785-4806 (2001)). The assay entails stably transfecting either 3T3-L1 or CHO cells with an expression construct that directs the expression of a GLUT4 fusion protein containing both c-myc and GFP tags. The presence of the tags on the recombinant GLUT4 protein allows for the quantitation of both total and cell surface GLUT4 concentrations by flow cytometry. This experimental system, which was developed by Bogen and colleagues, enables the analysis of effects of modulation of protein activity on GLUT4 transporter trafficking. This system, which is significantly more sensitive than previous methods, enables GLUT4 to be tracked from various intracellular compartments to a specialized insulin-responsive compartment, and finally to the plasma membrane. Details of the assay system are presented in Bogen, et al., Mol. Cell. Biol. 21:4785-4806 (2001), which is incorporated herein by reference.

Evaluation of proteins in the insulin resistance network using this and other relevant assays will allow the selection of the most promising targets for drug discovery.

8. Effects of Antisense Inhibitors on Protein Expression

The effects of antisense inhibitors on protein expression can be measured by a variety of methods known in the art. A preferred method is to measure mRNA levels using real-time quantitative polymerase chain reaction (PCR) methods. Real-time PCR can be performed using the ABI PRISM™ 7700 Sequence Detection System according to the manufacturer's instructions. The ABI PRISM™ 7700 Sequence Detection System is available from PE-APPLIED Biosystems, Foster City, Calif.

Other methods of measuring mRNA levels may also be used to determine the effects of anitisense inhibitors on proteins. For example competitive PCR and Northern blot analysis are well known in the art and may be performed to determine mRNA levels. Specifically, methods of RNA isolation and Northern blot analysis may be performed according to Ausubel, F. M. et al., Current Protocols in Molecular Biology, Volume 1, pp. 4.1.1-4.2.9 and 4.5.1-4.5.3, John Wiley & Sons, Inc., 1993.

The effects of antisense inhibitors on protein expression may also be determined by measuring protein levels of the proteins of interest. Various methods known in the art may be used, such as immunoprecipitation, Western blot analysis, ELISA, or fluorescence-activated cell sorting (FACS). Antibodies to the proteins of interest are often commercially available, and may be found by such sources as the MSRS catalogue of antibodies (Aerie Corporation, Birmingham, Mich.). Antibodies can also be prepared through conventional antibody generation methods, such as found in Ausubel, F. M. et al., Current Protocols in Molecular Biology, Volume 2, pp. 11.12.1-11.12.9 and 11.4.1-11.11.5 John Wiley & Sons, Inc., 1997. Furthermore, immunoprecipitation analysis can be performed according to Ausubel, F. M. et al., Current Protocols in Molecular Biology, Volume 2, pp. 10.16.1-10.16.11, John Wiley & Sons, Inc., 1997, and ELISA can be performed according to Ausubel, F. M. et al., Current Protocols in Molecular Biology, Volume 2, pp. 11.1.1-11.2.22, John Wiley & Sons, Inc., 1997 or as described in Example 4, above.

Although the foregoing invention has been described in some detail by way of illustration and example for purposes of clarity of understanding, it will be obvious that certain changes and modifications may be practiced within the scope of the appended claims.

In various parts of this disclosure, certain publications or patents are discussed or cited. The mere discussion of, or reference to, such publications or patents is not intended as admission that they are prior art to the present invention.

What is claimed is:

1. A method for selecting a modulator of PN7065 comprising:
contacting a test compound with PN7065 and measuring the activity of PN7065 in the presence of said test compound.

2. The method of claim 1, wherein the measuring step comprises measuring the kinase activity of PN7065.

3. The method of claim 1, wherein said measuring step comprises detecting the binding of PN7065 to a ligand of PN7065.

4. The method of claim 3, wherein said ligand of PN7065 is GLUT4.

5. The method of claim 1, wherein the selected test compound is further tested in an assay for its ability to modulate glucose uptake by cells.

6. The method of claim 5, wherein said assay for measuring glucose uptake comprises measuring the blood glucose levels of an animal.

7. The method of claim 5, wherein said assay for measuring glucose uptake is performed in vitro.

8. The method of claim 1, wherein the selected test compound is further tested in an assay for its ability to modulate plasma insulin levels.


SEQUENCE LISTING (267 sequences)

SEQ ID NO: 1 - DNA, 16 bp, Homo sapiens
tcgccatgccgcaaag

SEQ ID NO: 2 - DNA, 16 bp, Homo sapiens
gctccattttccccct

SEQ ID NO: 3 - DNA, 16 bp, Homo sapiens
gcagcataggctcgct

SEQ ID NO: 4 - DNA, 16 bp, Homo sapiens
cttccatgagggtagc

SEQ ID NO: 5 - DNA, 16 bp, Homo sapiens
cgctcatggtgcccga

SEQ ID NO: 6 - DNA, 16 bp, Homo sapiens
tctccatgacgggcca

SEQ ID NO: 7 - DNA, 16 bp, Homo sapiens
cattcatggtggcagc

SEQ ID NO: 8 - DNA, 17 bp, Homo sapiens
ttatccatgactggatg

SEQ ID NO: 9 - DNA, 16 bp, Homo sapiens
ggccgggaccggggcc

SEQ ID NO: 10 - DNA, 16 bp, Homo sapiens
gcagcatcgcgaccct

SEQ ID NO: 11 - DNA, 17 bp, Homo sapiens
caggcatcttggctaag

SEQ ID NO: 12 - DNA, 16 bp, Homo sapiens
gacgccgccatcttcg

SEQ ID NO: 13 - DNA, 18 bp, Homo sapiens
gattcgcatttccagacg

SEQ ID NO: 14 - DNA, 16 bp, Homo sapiens
tcgtcatcttgaactg

SEQ ID NO: 15 - DNA, 17 bp, Homo sapiens
tcagccatgtttgaaag

SEQ ID NO: 16 - DNA, 16 bp, Homo sapiens
gtgccatggctgctct

SEQ ID NO: 17 - DNA, 16 bp, Homo sapiens
cccccatgtcgctcgc

SEQ ID NO: 18 - DNA, 16 bp, Homo sapiens
ctcccatcctcgctgt

SEQ ID NO: 19 - DNA, 15 bp, Homo sapiens
cagtcattttgactc

SEQ ID NO: 20 - DNA, 16 bp, Homo sapiens
cggtcatcttggtgct

SEQ ID NO: 21 - DNA, 16 bp, Homo sapiens
tagccattgtccacgc

SEQ ID NO: 22 - DNA, 16 bp, Homo sapiens
tgtccatggcggccac

SEQ ID NO: 23 - DNA, 16 bp, Homo sapiens
agttcatgatgatgtc

SEQ ID NO: 24 - DNA, 16 bp, Homo sapiens
ccatcatgagcagtgg

SEQ ID NO: 25 - DNA, 16 bp, Homo sapiens
cgtccattgtgtgtgg

SEQ ID NO: 26 - DNA, 16 bp, Homo sapiens
cttccatttcatctat

SEQ ID NO: 27 - DNA, 16 bp, Homo sapiens
catccatagcggcagc

SEQ ID NO: 28 - DNA, 16 bp, Homo sapiens
caaccatggtggcagc

SEQ ID NO: 29 - DNA, 16 bp, Homo sapiens
ctgtcattgtcgctgt

SEQ ID NO: 30 - DNA, 16 bp, Homo sapiens
ccgacattacatgctc

SEQ ID NO: 31 - DNA, 16 bp, Homo sapiens
cagtcatggtacaagt

SEQ ID NO: 32 - DNA, 16 bp, Homo sapiens
cggccatcctggacga

SEQ ID NO: 33 - DNA, 16 bp, Homo sapiens
tgcccatggcgccggc

SEQ ID NO: 34 - DNA, 17 bp, Homo sapiens
ccgccatcttgccaaag

SEQ ID NO: 35 - DNA, 19 bp, Homo sapiens
cttattcataaaatttctg

SEQ ID NO: 36 - DNA, 16 bp, Homo sapiens
gagtcatcctgttgct

SEQ ID NO: 37 - DNA, 16 bp, Homo sapiens
cctccatcaagttcca

SEQ ID NO: 38 - DNA, 16 bp, Homo sapiens
taaccatggctccgcg

SEQ ID NO: 39 - DNA, 16 bp, Homo sapiens
ggttcatgctgaacgc

SEQ ID NO: 40 - DNA, 16 bp, Homo sapiens
gcagcccccttgaaat

SEQ ID NO: 41 - DNA, 16 bp, Homo sapiens
tggccatggctgtggc

SEQ ID NO: 42 - DNA, 16 bp, Homo sapiens
gtgtcatcttggtgat

SEQ ID NO: 43 - DNA, 16 bp, Homo sapiens
tcgccatggcccctga

SEQ ID NO: 44 - DNA, 16 bp, Homo sapiens
ccatcatgttggctga

SEQ ID NO: 45 - DNA, 16 bp, Homo sapiens
cggacatctctgcagc

SEQ ID NO: 46 - DNA, 16 bp, Homo sapiens
aggccatggccgggcc

SEQ ID NO: 47 - DNA, 16 bp, Homo sapiens
aggccatggccgggcc

SEQ ID NO: 48 - DNA, 16 bp, Homo sapiens
gcatcattcaggaggc

SEQ ID NO: 49 - DNA, 16 bp, Homo sapiens
ctcccatggttggaga

SEQ ID NO: 50 - DNA, 16 bp, Homo sapiens
cggccatggccctcgg

SEQ ID NO: 51 - DNA, 16 bp, Homo sapiens
acgccatggccgcggc

SEQ ID NO: 52 - DNA, 16 bp, Homo sapiens
tatccattcctcttcg

SEQ ID NO: 53 - DNA, 16 bp, Homo sapiens
ttgtcattccctgact

SEQ ID NO: 54 - DNA, 16 bp, Homo sapiens
gctccatggtggcaag

SEQ ID NO: 55 - DNA, 16 bp, Homo sapiens
tctccatgagggcacc

SEQ ID NO: 56 - DNA, 16 bp, Homo sapiens
ccttcatcttccctcc

SEQ ID NO: 57 - DNA, 16 bp, Homo sapiens
ggctcatggtgacggc

SEQ ID NO: 58 - DNA, 16 bp, Homo sapiens
tggtcatatttcaggc

SEQ ID NO: 59 - DNA, 16 bp, Homo sapiens
agagccgccattgcag

SEQ ID NO: 60 - DNA, 16 bp, Homo sapiens
gactcatcctgttgct

SEQ ID NO: 61 - DNA, 17 bp, Homo sapiens
cagtcatgactttggat

SEQ ID NO: 62 - DNA, 17 bp, Homo sapiens
gtcatcatttacagtat

SEQ ID NO: 63 - DNA, 16 bp, Homo sapiens
tccccatggcgactca

SEQ ID NO: 64 - DNA, 16 bp, Homo sapiens
cagacatcttggctcg

SEQ ID NO: 65 - DNA, 16 bp, Homo sapiens
cttccatgagggtagc

SEQ ID NO: 66 - DNA, 17 bp, Homo sapiens
gaagtcatgctccctct

SEQ ID NO: 67 - DNA, 16 bp, Homo sapiens
gacggcatctcgtctt

SEQ ID NO: 68 - DNA, 16 bp, Homo sapiens
ctgaagcacatcgtga

SEQ ID NO: 69 - DNA, 16 bp, Homo sapiens
gcactcatgtttgcaa

SEQ ID NO: 70 - DNA, 16 bp, Homo sapiens
gggctccatggcagcg

SEQ ID NO: 71 - DNA, 16 bp, Homo sapiens
ttgcccatggctgcgg

SEQ ID NO: 72 - DNA, 16 bp, Homo sapiens
tttcggacattggccg

SEQ ID NO: 73 - DNA, 16 bp, Homo sapiens
cccccataacctgggg

SEQ ID NO: 74 - DNA, 16 bp, Homo sapiens
gtcagaactcatcact

SEQ ID NO: 75 - protein, 34 aa, Homo sapiens
AEELKTQANDYFKAKDYENAIKFYSQAIELNPSN

SEQ ID NO: 76 - protein, 34 aa, Homo sapiens
AIYYGNRSLAYLRTECYGYALGDATRAIELDKKY

SEQ ID NO: 77 - protein, 34 aa, Homo sapiens
IKGYYRRAASNMALGKFRAALRDYETVVKVKPHD

SEQ ID NO: 78 - protein, 60 aa, Homo sapiens
MEPFTNDRLQLPRNMIENSMFEEEPDVVDLAKEPCLHPLEPDEVEYEPRGSRLLVRGLGE

SEQ ID NO: 79 - protein, 60 aa, Homo sapiens
AVSVIMVIYLLPRCTFTKEGCHKKNQSIGLIQPFATNGKLFPWAQIRLPTAVVPLRYELS

SEQ ID NO: 80 - protein, 60 aa, Homo sapiens
KLGYMDLASRLVTRVFKLLQNQIQQQTWTDEGTPSMRELRSALLEFACTHNLGNCSTTAM

SEQ ID NO: 81 - protein, 65 aa, Homo sapiens
YTIQNIVAGSTYLFSTKTHLSEVQAFFENQSEATFRLRCVQEALEVIQLNIQWMEKNLKSLTWWL

SEQ ID NO: 82 - protein, 34 aa, Homo sapiens
ADAYSNMGNTLKEMQDVQGALQCYTRAIQINPAF

SEQ ID NO: 83 - protein, 34 aa, Homo sapiens
ADAHSNLASIHKDSGNIPEAIASYRTALKLKPDF

SEQ ID NO: 84 - protein, 106 aa, Homo sapiens
AIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGL

SEQ ID NO: 85 - protein, 51 aa, Homo sapiens
AIVKEGWLHKRGEYIKTWRPRYFLLKNDGTFIGYKERPQDVDQREAPLNNF

SEQ ID NO: 86 - protein, 51 aa, Homo sapiens
DGTFIGYKERPQDVDQREAPLNNFSVAQCQLMKTERPRPNTFIIRCLQWTT

SEQ ID NO: 87 - protein, 51 aa, Homo sapiens
CQLMKTERPRPNTFIIRCLQWTTVIERTFHVETPEEREEWTTAIQTVADGL

SEQ ID NO: 88 - protein, 60 aa, Homo sapiens
PPPEHIPPPPRPPKRILEPHNGKCREFFPNHQWVKEETQEDKDCPIKEEKGSPLNAAPYG

SEQ ID NO: 89 - protein, 75 aa, Homo sapiens
IESMSQDTEVRSRVVGGSLRGAQAASPAKGEPSLPEKDEDHALSYWKPFLVNMCVATVLTAGAYLCYRFLFNSNT

SEQ ID NO: 90 - protein, 106 aa, Homo sapiens
SVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSL

SEQ ID NO: 91 - protein, 51 aa, Homo sapiens
SVIKEGWLHKRGEYIKTWRPRYFLLKSDGSFIGYKERPEAPDQTLPPLNNF

SEQ ID NO: 92 - protein, 51 aa, Homo sapiens
DGSFIGYKERPEAPDQTLPPLNNFSVAECQLMKTERPRPNTFVIRCLQWTT

SEQ ID NO: 93 - protein, 51 aa, Homo sapiens
CQLMKTERPRPNTFVIRCLQWTTVIERTFHVDSPDEREEWMRAIQMVANSL

SEQ ID NO: 94 - protein, 29 aa, Homo sapiens
IEQKTEGAEKKQQMAREYREKIETELRDI

SEQ ID NO: 95 - protein, 25 aa, Homo sapiens
YREKIETELRDICNDVLSLLEKFLI

SEQ ID NO: 96 - protein, 23 aa, Homo sapiens
ESKVFYLKMKGDYYRYLAEVAAG

SEQ ID NO: 97 - protein, 27 aa, Homo sapiens
AYQEAFEISKKEMQPTHPIRLGLALNF

SEQ ID NO: 98 - protein, 27 aa, Homo sapiens
SVFYYEILNSPEKACSLAKTAFDEAIA

SEQ ID NO: 99 - protein, 60 aa, Homo sapiens
HCTVKLRQTTGLMEYCLEVIKENDPSGFLQISDALIRRVHLTEDQWGKGTLTPRMTTDFD

SEQ ID NO: 100 - protein, 30 aa, Homo sapiens
PPLSTVPADGYILELDDGNGGQFREVYVGK

SEQ ID NO: 101 - protein, 60 aa, Homo sapiens
KYVRAEQKDRQHTLKHFEHVRMVDPKKAAQIRSQVMTHLRVIYERMNQSLSLLYNVPAVA

SEQ ID NO: 102 - protein, 26 aa, Homo sapiens
EEIQDEVDELLQKEQNYSDDVLANMI

SEQ ID NO: 103 - protein, 52 aa, Homo sapiens
DDLQPWHSFGADSVPANTENEVEPVDARPAADRGLTTRPGSGLTNIKTEEIS

SEQ ID NO: 104 - protein, 22 aa, Homo sapiens
LPEDEEEKKKQEEKKTKFENLC

SEQ ID NO: 105 - protein, 123 aa, Homo sapiens
HLQTCRLLLSYGSDPSIISLQGFTAAQMGNEAVQQILSESTPIRTSDVDYRLLEASKAGDLETVKQLCSSQNVNCRDLEGRHSTPLHFAAGYNRVSVVEYLLHHGADVHAKDKGGLVPLHNAC

SEQ ID NO: 106 - protein, 60 aa, Homo sapiens
CMSCSSYRESQEKVMNDSDTHENSLMDQNNPISYSLSENSEEDNRVTSVSSDSQTGMDRS

SEQ ID NO: 107 - protein, 36 aa, Homo sapiens
IQTGSALLPLSPESKAWAVLTSDEESGSGQGTSDSL

SEQ ID NO: 108 - protein, 47 aa, Homo sapiens
AEGLESEKKAVIPLVIVSALTFICLVVLVGILIYWRKCFQTAHFYLE

SEQ ID NO: 109 - protein, 29 aa, Homo sapiens
GPLKSTAEDFWRMIWEHNVEVIVMITNLV

SEQ ID NO: 110 - protein, 29 aa, Homo sapiens
IQALGTQVRQLQEDAARLQAAYAGDKADD

SEQ ID NO: 111 - protein, 44 aa, Homo sapiens
MAEAKTHWLGAALSLIPLIFLISGAEAASFQRNQLLQKEPDLRL

SEQ ID NO: 112 - protein, 120 aa, Homo sapiens
YIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFK

SEQ ID NO: 113 - protein, 34 aa, Homo sapiens
GIQEEDLRKESKDQLSDDVSKVIAYLKRLVNAAG

SEQ ID NO: 114 - protein, 67 aa, Homo sapiens
EEWYVSYITRPEAEAALRKINQDGTFLVRDSSKKTTTNPYVLMVLYKDKVYNIQIRYQKESQVYLLG

SEQ ID NO: 115 - protein, 23 aa, Homo sapiens
ESKVFYLKMKGDYYRYLAEVASG

SEQ ID NO: 116 - protein, 27 aa, Homo sapiens
AYKEAFEISKEQMQPTHPIRLGLALNF

SEQ ID NO: 117 - protein, 27 aa, Homo sapiens
SVFYYEIQNAPEQACLLAKQAFDDAIA

SEQ ID NO: 118 - protein, 30 aa, Homo sapiens
ELDTLNEDSYKDSTLIMQLLRDNLTLWTSD

SEQ ID NO: 119 - protein, 50 aa, Homo sapiens
LQIARAEIITKEREVSEWKDKYEESRREVMEMRKIVAEYEKTIAQMIEDE

SEQ ID NO: 120 - protein, 43 aa, Homo sapiens
VQQLVLEKEQALADLNSVEKSLADLFRRYEKMKEVLEGFRKNE

SEQ ID NO: 121 - protein, 36 aa, Homo sapiens
AQEYLSRVKKEEQRYQALKVHAEEKLDRANAEIAQV

SEQ ID NO: 122 - protein, 62 aa, Homo sapiens
NTCEECGKPIGCDCKDLSYKDRHWHEACFHCSQCRNSLVDKPFAAKEDQLLCTDCYSNEYSS

SEQ ID NO: 123 - protein, 62 aa, Homo sapiens
SKCQECKKTIMPGTRKMEYKGSSWHETCFICHRCQQPIGTKSFIPKDNQNFCVPCYEKQHAM

SEQ ID NO: 124 - protein, 60 aa, Homo sapiens
MQCVQCKKPITTGGVTYREQPWHKECFVCTACRKQLSGQRFTARDDFAYCLNCFCDLYAK

SEQ ID NO: 125 - protein, 61 aa, Homo sapiens
KKCAGCTNPISGLGGTKYISFEERQWHNDCFNCKKCSLSLVGRGFLTERDDILCPDCGKDI

SEQ ID NO: 126 - protein, 106 aa, Homo sapiens
RNEVLRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERV

SEQ ID NO: 127 - protein, 85 aa, Homo sapiens
NTSLINQKKKMESDLTQLQSEVEEAVQECRNAEEKAKKAITDAAMMAEELKKEQDTSAHLERMKKNMEQTIKDLQHRLDEAEQIA

SEQ ID NO: 128 - protein, 29 aa, Homo sapiens
LKGGKKQLQKLEARVRELEGELEAEQKRN

SEQ ID NO: 129 - protein, 21 aa, Homo sapiens
MRKSERRIKELTYQTEEDKKN

SEQ ID NO: 130 - protein, 47 aa, Homo sapiens
LLRLQDLVDKLQLKVKAYKRQAEEAEEQANTNLSKFRKVQHELDEAE

SEQ ID NO: 131 - protein, 41 aa, Homo sapiens
DVHNRIVIRGLNTIPLFVQLLYSPIENIQRVAAGVLCELAQ

SEQ ID NO: 132 - protein, 41 aa, Homo sapiens
DKEAAEAIEAEGATAPLTELLHSRNEGVATYAAAVLFRMSE

SEQ ID NO: 133 - protein, 22 aa, Homo sapiens
AEIYEIELVDIEKAIAHYEQSA

SEQ ID NO: 134 - protein, 22 aa, Homo sapiens
LSLLRAETDRLREILAGKEREY

SEQ ID NO: 135 - protein, 105 aa, Homo sapiens
VIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEEL

SEQ ID NO: 136 - protein, 29 aa, Homo sapiens
EIQLKEAKHIAEEADRKYEEVARKLVIIE

SEQ ID NO: 137 - protein, 24 aa, Homo sapiens
DLERTEERAELAESKCSELEEELK

SEQ ID NO: 138 - protein, 26 aa, Homo sapiens
KLKEAETRAEFAERSVAKLEKTIDDL

SEQ ID NO: 139 - protein, 44 aa, Homo sapiens
SKTCVQEITAHRKKLDESIYDVAFHSSKAYIASAGADALAKVFV

SEQ ID NO: 140 - protein, 152 aa, Homo sapiens
EDQQREEATRLQGELEKLRKEWNALETECHSLKRENVLLSSELQRQEKELHNSQKQSLELTSDLSILQMSRKELENQVGSLKEQHLRDSADLKTLLSKAENQAKDVQKEYEKTQTVLSELKLKFEMTEQEKQSITDELKQCKNNLKLLREKG

SEQ ID NO: 141 - protein, 24 aa, Homo sapiens
ELQRQEKELHNSQKQSLELTSDLS

SEQ ID NO: 142 - protein, 26 aa, Homo sapiens
LLSKAENQAKDVQKEYEKTQTVLSEL

SEQ ID NO: 143 - protein, 25 aa, Homo sapiens
YRQMVETELKLICCDILDVLDKHLI

SEQ ID NO: 144 - protein, 23 aa, Homo sapiens
ESKVFYYKMKGDYHRYLAEFATG

SEQ ID NO: 145 - protein, 27 aa, Homo sapiens
AYKAASDIAMTELPPTHPIRLGLALNF

SEQ ID NO: 146 - protein, 27 aa, Homo sapiens
SVFYYEILNSPDRACRLAKAAFDDAIA

SEQ ID NO: 147 - protein, 28 aa, Homo sapiens
ELDTLSEESYKDSTLIMQLLRDNLTLWT

SEQ ID NO: 148 - protein, 22 aa, Homo sapiens
AKNLGTALAELRTAAQKAQEAC

SEQ ID NO: 149 - protein, 29 aa, Homo sapiens
MDSALSVVQNLEKDLQEVKAAARDGKLKP

SEQ ID NO: 150 - protein, 81 aa, Homo sapiens
EELAQLAPTPGDAIISLDFGNQNFEESSAYGKAILPPSQPWATELRSHSTQSEAGSLPAFTVPQAAAPGSTTPSATSSSSS

SEQ ID NO: 151 - protein, 64 aa, Homo sapiens
CSTPNSPEDYYTSLDNDLKIEVIEKLFAMDTEAKDQCSTQTDFNELDLETLAPYIPMDGEDFQL

SEQ ID NO: 152 - protein, 108 aa, Homo sapiens
QHLITEVTTDTFWEVVLQKQDVLLLYYAPWCGFCPSLNHIFIQLARNLPMDTFTVARIDVSQNDLPWEFMVDRLPTVLFFPCNRKDLSVKYPEDVPITLPNLLRFILH

SEQ ID NO: 153 - protein, 50 aa, Homo sapiens
ISHLEREIQKLRAEISSLQRAQVQVESQLSSARRDEHRLRQQQRALEEQH

SEQ ID NO: 154 - protein, 36 aa, Homo sapiens
SEQLQALYEQKTRELQELARKLQELADASENLLTEN

SEQ ID NO: 155 - protein, 44 aa, Homo sapiens
EELLLLMEKIRDPFIHEISKIAMGMRSASQFTRDFIRDSGVVSL

SEQ ID NO: 156 - protein, 60 aa, Homo sapiens
IETLLNYPSSRVRTSFLENMIRMAPPYPNLNIIQTYICKVCEETLAYSVDSPEQLSGIRM

SEQ ID NO: 157 - protein, 54 aa, Homo sapiens
ESSLQVEDESIIGSWFWTEEEASMGTGASSKSRPRTDGERIGDSLFGAREKTSM

SEQ ID NO: 158 - protein, 109 aa, Homo sapiens
QVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYP

SEQ ID NO: 159 - protein, 90 aa, Homo sapiens
LERQVETIRNLVDSYVAIINKSIRDLMPKTIMHLMINNTKAFIHHELAYLYSSADQSSLMEESADQAQRRDDMLRMYHALKEALNIIGDI

SEQ ID NO: 160 - protein, 22 aa, Homo sapiens
NKDVRKRRQVLKDLVKVIQQES

SEQ ID NO: 161 - protein, 61 aa, Homo sapiens
GDKTKMTNHCVFSANEDHETIRNYAQVFNKLIRRYKYLEKAFEDEMKKLLLFLKAFSETEQ

SEQ ID NO: 162 - protein, 29 aa, Homo sapiens
NEAFKKQAESASEAAKKYMEENDQLKKGA

SEQ ID NO: 163 - protein, 42 aa, Homo sapiens
EVKLEEENRSLKADLQKLKDELASTKQKLEKAENEVLAMRKQ

SEQ ID NO: 164 - protein, 22 aa, Homo sapiens
LKHLKGQNEAALECLRKAEELI

SEQ ID NO: 165 - protein, 34 aa, Homo sapiens
ATMCNLLAYLKHLKGQNEAALECLRKAEELIQQE

SEQ ID NO: 166 - protein, 41 aa, Homo sapiens
DYDEQALGIMQTLGVDRQRTVESLQNSSYNHFAAIYYLLLE

SEQ ID NO: 167 - protein, 94 aa, Homo sapiens
RVRLYKYGTEKPLGFYIRDGSSVRVTPHGLEKVPGIFISRLVPGGLAQSTGLLAVNDEVLEVNGIEVSGKSLDQVTDMMIANSRNLIITVRPAN

SEQ ID NO: 168 - protein, 53 aa, Homo sapiens
ISRGLLYPQACVCISHRKKESKDIASKYLTSHQPILCLLTTPNCKGCWEKKSI

SEQ ID NO: 169 - protein, 57 aa, Homo sapiens
LGAVESEKQKLRAQVRRLVQENQWLREELAGTQQKLQRSEQAVAQLEEEKQHLLFMS

SEQ ID NO: 170 - protein, 22 aa, Homo sapiens
RYEVAVPLCKQALEDLEKTSGH

SEQ ID NO: 171 - protein, 168 aa, Homo sapiens
AQVRRLVQENQWLREELAGTQQKLQRSEQAVAQLEEEKQHLLFMSQIRKLDEDASPNEEKGDVPKDTLDDLFPNEDEQSPAPSPGGGDVSGQHGGYEIPARLRTLHNLVIQYASQGRYEVAVPLCKQALEDLEKTSGHDHPDVATMLNILALVYRDQNKYKEAAHLLN

SEQ ID NO: 172 - protein, 36 aa, Homo sapiens
MRRLKKQLADERSNRDELELELAENRKLLTEKDAQI

SEQ ID NO: 173 - protein, 43 aa, Homo sapiens
LEELRDKNESLTMRLHETLKQCQDLKTEKSQMDRKINQLSEEN

SEQ ID NO: 174 - protein, 35 aa, Homo sapiens
LREFASHLQQLQDALNELTEEHSKATQEWLEKQAQ

SEQ ID NO: 175 - protein, 29 aa, Homo sapiens
TRKLMEECKRLQGEMMKLSEENRHLRDEG

SEQ ID NO: 176 - protein, 33 aa, Homo sapiens
LESTAIHWASRGGNLDVLKLLLNKGAKISARDK

SEQ ID NO: 177 - protein, 33 aa, Homo sapiens
LLSTALHVAVRTGHYECAEHLIACEADLNAKDR

SEQ ID NO: 178 - protein, 33 aa, Homo sapiens
EGDTPLHDAVRLNRYKMIRLLIMYGADLNIKNC

SEQ ID NO: 179 - protein, 49 aa, Homo sapiens
TLSNAVSSLASTGLSLTKVDEREKQAALEEEQARLKALKEQRLKELAKK

SEQ ID NO: 180 - protein, 75 aa, Homo sapiens
IKDSTAASRATVSTSAGGIMTAPAIDIFSTPSSSNSTSKLPNDLLDLQQPTFHPSVHPMSTASQVASTWGDPFSA

SEQ ID NO: 181 - protein, 49 aa, Homo sapiens
LTNSHDGLDGPTYKKRRLDECEEAFQGTKVFVMPNGMLKSNQQLVDIIE

SEQ ID NO: 182 - protein, 68 aa, Homo sapiens
SYLDQISRYYITRAKLVSKIAKYPHVEDYRRTVTEIDEKEYISLRLIISELRNQYVTLHDMILKNIEK

SEQ ID NO: 183 - protein, 84 aa, Homo sapiens
PEEPRLGVLTVTDTTPDSMRLSWSVAQGPFDSFVVQYEDTNGQPQALLVDGDQSKILISGLEPSTPYRFLLYGLHEGKRLGPLS

SEQ ID NO: 184 - protein, 89 aa, Homo sapiens
SRPRLSQLSVTDVTTSSLRLNWEAPPGAFDSFLLRFGVPSPSTLEPHPRPLLQRELMVPGTRHSAVLRDLRSGTLYSLTLYGLRGPHKA

SEQ ID NO: 185 - protein, 58 aa, Homo sapiens
PPYVPGVVGGTLQAATICASSHQFLSTHYNLHNLYGLTEAIASHRALVKARGTRPFVI

SEQ ID NO: 186 - protein, 118 aa, Homo sapiens
ETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQ

SEQ ID NO: 187 - protein, 44 aa, Homo sapiens
VFEGRRGEKYGYLSLPKPMKNNANLKSLKPDKPDSEEQAKIAKL

SEQ ID NO: 188 - protein, 48 aa, Homo sapiens
SSMAYSLYLFTRGEGPLKTSQDLIHQLEVFAAEGLKLTSSVQAFSKQL

SEQ ID NO: 189 - protein, 17 aa, Homo sapiens
RFQPVHDLTIGVEFGAR

SEQ ID NO: 190 - protein, 23 aa, Homo sapiens
ITIDGKQIKLQIWDTAGQESFRS

SEQ ID NO: 191 - protein, 14 aa, Homo sapiens
SNMVIMLIGNKSDL

SEQ ID NO: 192 - protein, 23 aa, Homo sapiens
FMETSAKTASNVEEAFINTAKEI

SEQ ID NO: 193 - protein, 36 aa, Homo sapiens
VKELQQKTENYEQRIREQQEQLSLQQTIIDKLKSQL

SEQ ID NO: 194 - protein, 23 aa, Homo sapiens
ESKVFYLKMKGDYFRYLSEVASG

SEQ ID NO: 195 - protein, 27 aa, Homo sapiens
AYQEAFEISKKEMQPTHPIRLGLALNF

SEQ ID NO: 196 - protein, 27 aa, Homo sapiens
SVFYYEILNSPEKACSLAKTAFDEAIA

SEQ ID NO: 197 - protein, 30 aa, Homo sapiens
ELDTLNEESYKDSTLIMQLLRDNLTLWTSE

SEQ ID NO: 198 - protein, 64 aa, Homo sapiens
KNDLIAKVDELTCEKDVLQGELEAVKQAKLKLEEKNRELEEELRKARAEAEDARQKAKDDDDSD

SEQ ID NO: 199 - protein, 22 aa, Homo sapiens
MARVLMERNQYKERLMELQEAV

SEQ ID NO: 200 - protein, 50 aa, Homo sapiens
KAELQDRVTELSALLTQSQKQNEDYEKMIKALRETVEILETNHTELMEHE

SEQ ID NO: 201 - protein, 25 aa, Homo sapiens
VQDLRQQLAGCQEAVNLLQQQHDQW

SEQ ID NO: 202 - protein, 47 aa, Homo sapiens
LEKLRQRIHDKAVALERAIDEKFSALEEKEKELRQLRLAVRERDHDL

SEQ ID NO: 203 - protein, 22 aa, Homo sapiens
VEQLSTTCQNLQWLKEEMETKF

SEQ ID NO: 204 - protein, 22 aa, Homo sapiens
IQQLQTSLHDRNKEVEDLSATL

SEQ ID NO: 205 - protein, 22 aa, Homo sapiens
AEELCQRLQRKERMLQDLLSDR

SEQ ID NO: 206 - protein, 22 aa, Homo sapiens
QAAAEKLVQALMERNSELQALR

SEQ ID NO: 207 - protein, 49 aa, Homo sapiens
LARIDLERRIESLNEEIAFLKKVHEEEIRELQAQLQEQQVQVEMDMSKP

SEQ ID NO: 208 - protein, 96 aa, Homo sapiens
VSDLTQAANKNNDALRQAKQEMMEYRHQIQSYTCEIDALKGTNDSLMRQMRELEDRFASEASGYQDNIARLEEEIRHLKDEMARHLREYQDLLNVK

SEQ ID NO: 209 - protein, 82 aa, Homo sapiens
NSLQTRIVIQARAVPIFIELLSSEFEDVQEQAVWALGNIAGDSTMCRDYVLDCNILPPLLQLFSKQNRLTMTRNAVWALSNL

SEQ ID NO: 210 - protein, 41 aa, Homo sapiens
KSPPPEFAKVSPCLNVLSWLLFVSDTDVLADACWALSYLSD

SEQ ID NO: 211 - protein, 41 aa, Homo sapiens
PNDKIQAVIDAGVCRRLVELLMHNDYKVVSPALRAVGNIVT

SEQ ID NO: 212 - protein, 41 aa, Homo sapiens
DDIQTQVILNCSALQSLLHLLSSPKESIKKEACWTISNITA

SEQ ID NO: 213 - protein, 41 aa, Homo sapiens
NRAQIQTVIDANIFPALISILQTAEFRTRKEAAWAITNATS

SEQ ID NO: 214 - protein, 33 aa, Homo sapiens
SAEQIKYLVELGCIKPLCDLLTVMDSKIVQVAL

SEQ ID NO: 215 - protein, 29 aa, Homo sapiens
NEAFKKQAESASEAAKKYMEENDQLKKGA

SEQ ID NO: 216 - protein, 64 aa, Homo sapiens
EVKLEEENRSLKADLQKLKDELASTKQKLEKAENQVLAMRKQSEGLTKEYDRLLEEHAKLQAAV

SEQ ID NO: 217 - protein, 39 aa, Homo sapiens
DSDYYNMLLKKLAPPLVTLLSGEPEVQYVALRNINLIVQ

SEQ ID NO: 218 - protein, 54 aa, Homo sapiens
ANLHSSSSTFSTTSSTVSAPPPVVSVSSSLNSGSSLGLSLGSNSTVTASTRSSV

SEQ ID NO: 219 - protein, 68 aa, Homo sapiens
LNPALPPGYSYTSLPYYTGVPGLPSTFQYGPAVFPVAPTSSKQHGVNVSVNASATPFQQPSGYGSHGY

SEQ ID NO: 220 - protein, 56 aa, Homo sapiens
CGFPPRPAHGDVSVTDLHPGGTATFHCDSGYQLQGEETLICLNGTRPSWNGETPSC

SEQ ID NO: 221 - protein, 124 aa, Homo sapiens
CGGTIHNATLGRIVSPEPGGAVGPNLTCRWVIEAAEGRRLHLHFERVSLDEDNDRLMVRSGGSPLSPVIYDSDMDDVPERGLISDAQSLYVELLSETPANPLLLSLRFEAFEEDRCFAPFLAHG

SEQ ID NO: 222 - protein, 45 aa, Homo sapiens
LLQEFYETTLEALKDAKNDRLWFKTNTKLGKLYLEREEYGKLQKI

SEQ ID NO: 223 - protein, 52 aa, Homo sapiens
DDGEDDLKKGTQLLEIYALEIQMYTAQKNNKKLKALYEQSLHIKSAIPHPLI

SEQ ID NO: 224 - protein, 29 aa, Homo sapiens
IKMLGELLEESQMEATRLRQKAEELVKDN

SEQ ID NO: 225 - protein, 50 aa, Homo sapiens
TSILQTLCEQLRKENEALKAKLDKGLEQRDQAAERLREENLELKKLLMSN

SEQ ID NO: 226 - protein, 22 aa, Homo sapiens
LGAAEKKVKMLEQQRSELLEVN

SEQ ID NO: 227 - protein, 36 aa, Homo sapiens
KQQYEQKITELRQKLADLQKQVTDLEAEREQKQRDF

SEQ ID NO: 228 - protein, 36 aa, Homo sapiens
LLLAKSKIEMEETDKEQLTAEAKELRQKVKYLQDQL

SEQ ID NO: 229 - protein, 22 aa, Homo sapiens
REYQEKEIQRLNKALEEALSIQ

SEQ ID NO: 230 - protein, 35 aa, Homo sapiens
KQELVTQNELLKQQVKIFEEDFQRERSDRERMNEE

SEQ ID NO: 231 - protein, 21 aa, Homo sapiens
KKTRDLRRQLRKAVMDHISDS

SEQ ID NO: 232 - protein, 25 aa, Homo sapiens
QVRVLTEAVDDITSVDDFLSVSENH

SEQ ID NO: 233 - protein, 25 aa, Homo sapiens
NEFIDASRLVYDGVRDIRKAVLMIR

SEQ ID NO: 234 - protein, 48 aa, Homo sapiens
IALHHGLAVFQDEGAEPLKQRVEASISKANSFLGEKASAGLLGAHAAA

SEQ ID NO: 235 - protein, 47 aa, Homo sapiens
LGVAHNNLMAMAQETGDNLYWGSVTGSQSNAVSPTPAPRNPSDPMPQ

SEQ ID NO: 236 - protein, 51 aa, Homo sapiens
LQAEIEELRATLEQTERSRKIAEQELLDASERVQLLHTQNTSLINTKKKLE

SEQ ID NO: 237 - protein, 48 aa, Homo sapiens
IIQEARNAEEKAKKAITDAAMMAEELKKEQDTSAHLERMKKNLEQTVK

SEQ ID NO: 238 - protein, 53 aa, Homo sapiens
EFEVVTPEEQNSPESSSHANAMALGPLPREDGNLMLHLQRLETTLSVCAEEPD

SEQ ID NO: 239 - protein, 54 aa, Homo sapiens
FDRKLLLAKSKIEMEETDKEQLTAEAKELRQKVKYLQDQLSPLTRQREYQEKEI

SEQ ID NO: 240 - protein, 28 aa, Homo sapiens
FAVLLLGFFIFTFLRVPETRGRTFDQIS

SEQ ID NO: 241 - protein, 29 aa, Homo sapiens
AAFHRTPSLLEQEVKPSTELEYLGPDEND

SEQ ID NO: 242 - protein, 120 aa, Homo sapiens
MCFSFIMPPAMADILDIWAVDSQIASDGSIPVDFLLPTGIYIQLEVPREATISYIKQMLWKQVHNYPMFNLLMDIDSYMFACVNQTAVYEELEDETRRLCDVRPFLPVLKLVTRSCDPGE

SEQ ID NO: 243 - protein, 47 aa, Homo sapiens
EDKLYGGKLIVAVHFENCQDVFSFQVSPNMNPIKVNELAIQKRLTIH

SEQ ID NO: 244 - protein, 35 aa, Homo sapiens
RALYDYKKEREEDIDLHLGDILTVNKGSLVALGFS

SEQ ID NO: 245 - protein, 54 aa, Homo sapiens
DYTLTLRKGGNNKLIKIFHRDGKYGFSDPLTFSSVVELINHYRNESLAQYNPKL

SEQ ID NO: 246 - protein, 48 aa, Homo sapiens
NENTEDQYSLVEDDEDLPHHDEKTWNVGSSNRNKAENLLRGKRDGTFL

SEQ ID NO: 247 - protein, 27 aa, Homo sapiens
GFRQGGASQSDKTPEELFHPLGADSQV

SEQ ID NO: 248 - protein, 37 aa, Homo sapiens
DLQEMKEESRQMMREKKVTILELFRSPAYRQPILIAV

SEQ ID NO: 249 - protein, 120 aa, Homo sapiens
QSQLLSIEKEVEEYKNFRPDDPARKTKALLQMVQQFAVDFEKRIEGSGDQIDTYELSGGAQSQLLSIEKEVEEYKNFRPDDPARKTKALLQMVQQFAVDFEKRIEGSGDQIDTYELSGGA

SEQ ID NO: 250 - protein, 55 aa, Homo sapiens
ELACETQEEVDSWKASFLRAGVYPERVGDKEKASETEENGSDSFMHSMDPQLERQ

SEQ ID NO: 251 - protein, 60 aa, Homo sapiens
INVGGKYVKLQIWDTAGQERFRSVTRSYYRGAAGALLVYDITSRETYNALTNWLTDARML

SEQ ID NO: 252 - protein, 33 aa, Homo sapiens
ERMGSGIQYGDAALRQLRSPRRTQAPNAQECGC

SEQ ID NO: 253 - protein, 30 aa, Homo sapiens
DVEHVLIDVGTGYYVEKTAEDAKDFFKRKI

SEQ ID NO: 254 - DNA, 2352 bp, Homo sapiens
atggttatcatgtcggagttcagcgcggaccccgcgggccagagtcagggccagcagaagtccctccgggtgggtttttacgacatcgagcggaccctgggcaaaggcaacttcgcggtggtgaagctggcgcggcatcgagtcaccaaaacgcaggttgcaataaaaataattgataaaacacgattagattcaagcaatttggagaaaatctatcgtgaggttcagctgatgaagcttctgaaccatccacacatcataaagctttaccaggttatggaaacaaaggacatgctttacatcgtcactgaatttgctaaaaatggagaaatgtttgattatttgacttccaacgggcacctgagtgagaacgaggcgcggaagaagttctggcaaatcctgtcggccgtggagtactgtcacgaccatcacatcgtccaccgggacctcaagaccgagaacctcctgctggatggcaacatggacatcaagctggcagattttggatttgggaatttctacaagtcaggagagcctctgtccacgtggtgtgggagccccccgtatgccgccccggaagtctttgaggggaaggagtatgaaggcccccagctggacatctggagcctgggcgtggtgctgtacgtcctggtctgcggttctctccccttcgatgggcctaacctgccgacgctgagacagcgggtgctggagggccgcttccgcatccccttcttcatgtctcaagactgtgagagcctgatccgccgcatgctggtggtggaccccgccaggcgcatcaccatcgcccagatccggcagcaccggtggatgcgggctgagccctgcttgccgggacccgcctgccccgccttctccgcacacagctacacctccaacctgggcgactacgatgagcaggcgctgggtatcatgcagaccctgggcgtggaccggcagaggacggtggagtcactgcaaaacagcagctataaccactttgctgccatttattacctcctccttgagcggctcaaggagtatcggaatgcccagtgcgcccgccccgggcctgccaggcagccgcggcctcggagctcggacctcagtggtttggaggtgcctcaggaaggtctttccaccgaccctttccgacctgccttgctgtgcccgcagccgcagaccttggtgcagtccgtcctccaggccgagatggactgtgagctccagagctcgctgcagtggcccttgttcttcccggtggatgccagctgcagcggagtgttccggccccggcccgtgtccccaagcagcctgctggacacagccatcagtgaggaggccaggcaggggccgggcctagaggaggagcaggacacgcaggagtccctgcccagcagcacgggccggaggcacaccctggccgaggtctccacccgcctctccccactcaccgcgccatgtatagtcgtctccccctccaccacggcaagtcctgcagagggaaccagctctgacagttgtctgaccttctctgcgagcaaaagccccgcggggctcagtggcaccccggccactcaggggctgctgggcgcctgctccccggtcaggctggcctcgcccttcctggggtcgcagtccgccaccccagtgctgcaggctcaggggggcttgggaggagctgttctgctccctgtcagcttccaggagggacggcgggcgtcggacacctcactgactcaagggctgaaggcctttcggcagcagctgaggaagaccacgcggaccaaagggtttctgggactgaacaaaatcaaggggctggctcgccaggtgtgccaggtccctgccagccgggccagcaggggcggcctgagccccttccacgcccctgcacagagcccaggcctgcacggcggcgcagccggcagccgggagggctggagcctgctggaggaggtgctagagcagcagaggctgctccagttacagcaccacccggccgctgcacccggctgctcccaggccccccagccggcccctgccccgtttgtgatcgccccctgtgatggccctggggctgccccgctccccagcaccctcctcacgtcggggctcccgctgctgccgcccccactcctgcagaccggcgcgtccccggtggcctcagcggcgcagctcctggacacacacctgcacattggcaccggccccaccgccctccccgctgtgcccccaccacgcctggccaggctggccccaggttgtgagcccctggggctgctgcagggggactgtgagatggaggacctgatgccctgctccctaggcacgtttgtcctggtgcagtga

SEQ ID NO: 255 - protein, 783 aa, Homo sapiens
MVIMSEFSADPAGQSQGQQKSLRVGFYDIERTLGKGNFAVVKLARHRVTKTQVAIKIIDKTRLDSSNLEKIYREVQLMKLLNHPHIIKLYQVMETKDMLYIVTEFAKNGEMFDYLTSNGHLSENEARKKFWQILSAVEYCHDHHIVHRDLKTENLLLDGNMDIKLADFGFGNFYKSGEPLSTWCGSPPYAAPEVFEGKEYEGPQLDIWSLGVVLYVLVCGSLPFDGPNLPTLRQRVLEGRFRIPFFMSQDCESLIRRMLVVDPARRITIAQIRQHRWMRAEPCLPGPACPAFSAHSYTSNLGDYDEQALGIMQTLGVDRQRTVESLQNSSYNHFAAIYYLLLERLKEYRNAQCARPGPARQPRPRSSDLSGLEVPQEGLSTDPFRPALLCPQPQTLVQSVLQAEMDCELQSSLQWPLFFPVDASCSGVFRPRPVSPSSLLDTAISEEARQGPGLEEEQDTQESLPSSTGRRHTLAEVSTRLSPLTAPCIVVSPSTTASPAEGTSSDSCLTFSASKSPAGLSGTPATQGLLGACSPVRLASPFLGSQSATPVLQAQGGLGGAVLLPVSFQEGRRASDTSLTQGLKAFRQQLRKTTRTKGFLGLNKIKGLARQVCQVPASRASRGGLSPFHAPAQSPGLHGGAAGSREGWSLLEEVLEQQRLLQLQHHPAAAPGCSQAPQPAPAPFVIAPCDGPGAAPLPSTLLTSGLPLLPPPLLQTGASPVASAAQLLDTHLHIGTGPTALPAVPPPRLARLAPGCEPLGLLQGDCEMEDLMPCSLGTFVLVQ

SEQ ID NO: 256 - DNA, 640 bp, Homo sapiens
cgccggtggatgcgggctgagccctgcttgccgggacccgcctgccccgccttctccgcacacagctacacctccaacctgggcgactacgatgagcaggcgctgggtatcatgcagaccctgggcgtggaccggcagaggacggtggagtcactgcaaaacagcagctataaccactttgctgccatttattacctcctccttgagcggctcaaggagtatcggaatgcccagtgcgcccgccccgggcctgccaggcagccgcggcctcggagctcggacctcagtggtttggaggtgcctcaggaaggtctttccaccgaccctttccgacctgccttgctgtgcccgcagccgcagaccttggtgcagtccgtcctccaggccgagatggactgtgggctccagagctcgctgcagtggcccttgttcttcccggtggatgccagctgcagcggagtgttccggccccggcccgtgtccccaagcagcctgctggacacagccatcagtgaggaggccaggcaggggccgggcctagaggaggagcaggacacgcaggagtccctgcccagcagcacgggccgggggcacaccctggccgaggtctccacccgcctctccccactcaccgcgccag

SEQ ID NO: 257 - protein, 213 aa, Homo sapiens
RRWMRAEPCLPGPACPAFSAHSYTSNLGDYDEQALGIMQTLGVDRQRTVESLQNSSYNHFAAIYYLLLERLKEYRNAQCARPGPARQPRPRSSDLSGLEVPQEGLSTDPFRPALLCPQPQTLVQSVLQAEMDCGLQSSLQWPLFFPVDASCSGVFRPRPVSPSSLLDTAISEEARQGPGLEEEQDTQESLPSSTGRGHTLAEVSTRLSPLTAP

SEQ ID NO: 258 - DNA, 1119 bp, Homo sapiens
atgaaccgcagccaccggcacggggcgggcagcggctgcctgggcactatggaggtgaagagcaagtttggagctgaatttcgtcggttttcgctggaaagatcaaaacctggaaaatttgaggagttttatggattactacaacatgttcataagatccccaatgttgacgttttggtaggctatgcagacatccatggagacttactacctataaataatgatgataattatcacaaagctgtttcaacggccaatccactgcttaggatatttatacaaaagaaggaagaagcagactacagtgcctttggtacagacacgctaataaagaagaagaatgttttaaccaacgtattgcgtcctgacaaccatagaaaaaagccacatatagtcattagtatgccccaagactttagacctgtgtcttctattatagacgtggatattctcccagaaacgcatcgtagggtacgtctttacaaatacggcacggagaaacccctaggattctacatccgggatggctccagtgtcagggtaacaccacatggcttagaaaaggttccagggatctttatatccaggcttgtcccaggaggtctggctcaaagtacaggactattagctgttaatgatgaagttttagaagttaatggcatagaagtttcagggaagagccttgatcaagtaacagacatgatgattgcaaatagccgtaacctcatcataacagtgagaccggcaaaccagaggaataatgttgtgaggaacagtcggacttctggcagttccggtcagtctactgataacagccttcttggctacccacagcagattgaaccaagctttgagccagaggatgaagacagcgaagaagatgacattatcattgaagacaatggagtgccacagcagattccaaaagctgttcctaatactgagagcctggagtcattaacacagatagagctaagctttgagtctggacagaatggctttattccctctaatgaagtgagcttagcagccatagcaagcagctcaaacacggaatttgaaacacatgctccagatcaaaaactcttagaagaagatggaacaatcataacattatga

SEQ ID NO: 259 - protein, 372 aa, Homo sapiens
MNRSHRHGAGSGCLGTMEVKSKFGAEFRRFSLERSKPGKFEEFYGLLQHVHKIPNVDVLVGYADIHGDLLPINNDDNYHKAVSTANPLLRIFIQKKEEADYSAFGTDTLIKKKNVLTNVLRPDNHRKKPHIVISMPQDFRPVSSIIDVDILPETHRRVRLYKYGTEKPLGFYIRDGSSVRVTPHGLEKVPGIFISRLVPGGLAQSTGLLAVNDEVLEVNGIEVSGKSLDQVTDMMIANSRNLIITVRPANQRNNVVRNSRTSGSSGQSTDNSLLGYPQQIEPSFEPEDEDSEEDDIIIEDNGVPQQIPKAVPNTESLESLTQIELSFESGQNGFIPSNEVSLAAIASSSNTEFETHAPDQKLLEEDGTIITL

SEQ ID NO: 260 - DNA, 239 bp, Homo sapiens
tctggacagaatggctttattccctctaatgaagtgagcttagcagccatagcaagcagctcaaacacggaatttgaaacacatgctccagatcaaaaactcttagaagaagatggaacaatcataacattatgaaaccgtggtttgaatgttttcagagtgaggatgccatgaggacttgtacatttggctagtttaggccaatgttgacgttttggtaggctatgcagacatccatg

SEQ ID NO: 261 - protein, 319 aa, Homo sapiens
ANVDVLVGYADIHGDLLPINNDDNYHKAVSTANPLLRIFIQKKEEADYSAFGTDTLIKKKNVLTNVLRPDNHRKKPHIVISMPQDFRPVSSIIDVDILPETHRRVRLYKYGTEKPLGFYIRDGSSVRVTPHGLEKVPGIFISRLVPGGLAQSTGLLAVNDEVLEVNGIEVSGKSLDQVTDMMIANSRNLIITVRPANQRNNVVRNSRTSGSSGQSTDNSLLGYPQQIEPSFEPEDEDSEEDDIIIEDNGVPQQIPKAVPNTESLESLTQIELSFESGQNGFIPSNEVSLAAIASSSNTEFETHAPDQKLLEEDGTIITL

SEQ ID NO: 262 - DNA, 1869 bp, Homo sapiens
atggccatgatggtgtttccgcgggaggagaagctgagccaggatgagatcgtgctgggcaccaaggctgtcatccagggactggagactctgcgtggggagcatcgtgccctgctggctcctctggttgcacctgaggccggcgaagccgagcctggctcgcaggagcgctgcatcctcctgcgtcgctccctggaagccattgagcttgggctgggggaggcccaggtgatcttggcattgtcgagccacctgggggctgtagaatcagagaagcagaagctgcgggcgcaggtgcggcgtctggtgcaggagaaccagtggctgcgtgaggagctggcggggacacagcagaagctgcagcgcagtgagcaggccgtggcccagctcgaggaggagaagcagcacttgctgttcatgagccagatccgcaagttggatgaagacgcctcccctaacgaggagaagggggacgtccccaaagacacactggatgacctgttccccaatgaggatgagcagagcccagcccctagcccaggaggaggggatgtgtctggtcagcatgggggctacgagatcccggcccggctccgcaccctgcacaacctggtgatccaatacgcctcacagggccgctacgaggtagctgtgccactctgcaagcaggcactcgaagacctggagaagacgtcaggccacgaccaccctgacgttgccaccatgctgaacatcctggcactggtctatcgggatcagaacaagtacaaggaggctgcccacctgctcaatgatgctctggccatccgggagaaaacactgggcaaggaccacccagccgtggctgcgacactaaacaacctggcagtcctgtatggcaagaggggcaagtacaaggaggctgagccattgtgcaagcgggcactggagatccgggagaaggtcctgggcaagtttcacccagatgtggccaagcagctcagcaacctggccctgctgtgccagaaccagggcaaagctgaggaggtggaatattactatcggcgggcactggagatctatgctacacgcctcgggcccgatgaccccaatgtggccaagaccaagaacaacctggcttcctgctacctgaagcagggcaagtaccaggatgcggagaccttgtacaaggagatcctcacccgcgctcatgagaaagagtttggctctgtcaatggggacaacaagcccatctggatgcacgcagaggagcgggaggaaagcaaggataagcgccgggacagcgccccctatggggaatacggcagctggtacaaggcctgtaaagtagacagccccacagtcaacaccaccctgcgcagcttgggggccctataccggcgccagggcaagctggaagccgcgcacacactagaggactgtgccagccgtaaccgcaagcagggtttggaccccgcaagccagaccaaggtggtagaactgctgaaagatggcagtggcaggcggggagaccgccgcagcagccgagacatggctgggggtgccgggcctcggtctgagtctgacctcgaggacgtgggacctacagctgagtggaatggggatggcagtggctccttgaggcgcagcggttcctttgggaaactccgggatgccctgaggcgcagcagtgagatgctggtaaagaagctgcaggggggcaccccccaggagccccctaaccccaggatgaagcgggccagttccctcaacttcctcaacaagagcgtggaagagccgacccagcctggaggcacaggtctctctgacagccgcactctcagctccagctccatggacctctcccgacgaagctccctggtgggctaa

SEQ ID NO: 263 - protein, 622 aa, Homo sapiens
MAMMVFPREEKLSQDEIVLGTKAVIQGLETLRGEHRALLAPLVAPEAGEAEPGSQERCILLRRSLEAIELGLGEAQVILALSSHLGAVESEKQKLRAQVRRLVQENQWLREELAGTQQKLQRSEQAVAQLEEEKQHLLFMSQIRKLDEDASPNEEKGDVPKDTLDDLFPNEDEQSPAPSPGGGDVSGQHGGYEIPARLRTLHNLVIQYASQGRYEVAVPLCKQALEDLEKTSGHDHPDVATMLNILALVYRDQNKYKEAAHLLNDALAIREKTLGKDHPAVAATLNNLAVLYGKRGKYKEAEPLCKRALEIREKVLGKFHPDVAKQLSNLALLCQNQGKAEEVEYYYRRALEIYATRLGPDDPNVAKTKNNLASCYLKQGKYQDAETLYKEILTRAHEKEFGSVNGDNKPIWMHAEEREESKDKRRDSAPYGEYGSWYKACKVDSPTVNTTLRSLGALYRRQGKLEAAHTLEDCASRNRKQGLDPASQTKVVELLKDGSGRRGDRRSSRDMAGGAGPRSESDLEDVGPTAEWNGDGSGSLRRSGSFGKLRDALRRSSEMLVKKLQGGTPQEPPNPRMKRASSLNFLNKSVEEPTQPGGTGLSDSRTLSSSSMDLSRRSSLVG

SEQ ID NO: 264 - DNA, 861 bp, Homo sapiens
agggaggagaagctgagccaggatgagatcgtgctgggcaccaaggctgtcatccagggactggagactctgcgtggggagcatcgtgccctgctggctcctctggttgcacctgaggccggcgaagccgagcctggctcgcaggagcgctgcatcctcctgcgtcgctccctggaagccattgagcttgggctgggggaggcccaggtgatcttggcattgtcgagccacctgggggctgtagaatcagagaagcagaagctgcgggcgcaggtgcggcgtctggtgcaggagaaccagtggctgcgtgaggagctgccggggacacagcakaagctgcagcgcagtgagcaggccgtggcccagctcgaggaggagaagcagcacttgctgttcatgarccagatccgcagttggatgaagacgcctyccctaacgaggagaagggggacgtccccaaagacacactggatgacctgttccccaatgaggatgagcagagcccagcccctagcccaggaggaggggatgtgtctggtcagcatgggggatacgagatcccggcccggctccgcaccctgcacaactggtgatccaatacgcctcacagggccgctacgaggtagctgtgccactctgcaagcaggcactcgaagactggagaagacgtcaggccacgaccaccctgacgttgccaccatgctgaacatcctggcactggtctatcgggatcagaacaagtacaaggaggctgcccacctgctcaatgatgctctggccatccgggagaaaacactgggcaaggaccacccagccgtggctgcgacactaaacaacctggcagtcctgtatagcgcagag

SEQ ID NO: 265 - protein, 287 aa, Homo sapiens
  misc_feature (111)..(111): Xaa can be any naturally occurring amino acid
REEKLSQDEIVLGTKAVIQGLETLRGEHRALLAPLVAPEAGEAEPGSQERCILLRRSLEAIELGLGEAQVILALSSHLGAVESEKQKLRAQVRRLVQENQWLREELPGTQXKLQRSEQAVAQLEEEKQHLLFMXQIRSWMKTPXLTRRRGTSPKTHWMTCSPMRMSRAQPLAQEEGMCLVSMGDTRSRPGSAPCTTGDPIRLTGPLRGSCATLQAGTRRLEKTSGHDHPDVATMLNILALVYRDQNKYKEAAHLLNDALAIREKTLGKDHPAVAATLNNLAVLYSAE

SEQ ID NO: 266 - DNA, 601 bp, Homo sapiens
atttcaagggggctgctgtacccccaggcatgtgtctgtatatcgcacaggaagaaggaaagtaaggacattgccagcaaatatcttacatctcatcagcctatactgtgtctcctgaccactcctaactgcaaaggatgctgggaaaaaaagagcattgtagcttttccagcctctgtggtaggcgcagataagggattagagttgggtgttactgaatcaatgtatcagacacttctcagtcaggctagagccagatttaactagatttagcaggaaaagtatgtttctttcacctgcatgtaatgaaggaaatctatgtccttcatacacttaataaacctgtaagtctctactatgggcaggtactgtgctagctagacattacaatgtgtgggggcagacacaaagatgggaacagtagacactggggaaccctagaggggggaggttgggagtaggggaagggttgaaaaatgattgggtactatgctcactacctgggtgatgggatcatttgtacaccaaatgccagcaacacacaatttacccgtgtaacacaccggcacgtgtaccccctgaacctaagatgaaagccgaa

SEQ ID NO: 267 - protein, 88 aa, Homo sapiens [sequence omitted]